SEQUENCE LISTING (6 sequences)

SEQ ID NO: 1 - protein, 26 aa, Artificial Sequence
  C-terminal sortase A recognition sequence
GGGGSGGGGSLPETGGHHHHHHHHHH

SEQ ID NO: 2 - protein, 192 aa, Artificial Sequence
  sortase A
TGSHHHHHHGSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 3 - protein, 233 aa, Artificial Sequence
  His6-TEVsite-GGG-IL15Ra-IL15
MGSSHHHHHHSSGENLYFQGGGITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - protein, 268 aa, Artificial Sequence
  anti-4-1BB PF31
DIVMTQSPPTLSLSPGERVTLSCRASQSISDYLHWYQQKPGQSPRLLIKYASQSISGIPARFSGSGSGTDFTLTISSLEPEDFAVYYCQDGHSFPPTFGGGTKVEIKGGGGSGGGGSGGGGSGGGGSQVQLVQSGAEVKKPGASVKVSCKASGYTFSSYWMHWVRQAPGQRLEWMGEINPGNGHTNYSQKFQGRVTITVDKSASTAYMELSSLRSEDTAVYYCARSFTTARAFAYWGQGTLVTVSSGGGGSGGGGSLPETGGHHHHHH

SEQ ID NO: 5 - protein, 132 aa, Artificial Sequence
  SYR-(G4S)3-IL15 (PF18)
SYRGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 229 aa, Artificial Sequence
  SYR-(G4S)3-IL15Ra-linker-IL15 (PF26)
SYRGGGGSGGGGSGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

(19) United States
(12) Patent Application Publication
(10) Pub. No.: US 2025/0262314 A1
(43) Pub. Date: Aug. 21, 2025

(54) MULTIFUNCTIONAL ANTIBODIES

(71) Applicant: Synaffix B.V., Oss (NL)

(72) Inventors: Remon VAN GEEL, Lithoijen (NL); Maria Antonia WIJDEVEN, Oss (NL); Willem Johannes Petrus VUGS, Boxtel (NL); Laureen DE BEVER, Eindhoven (NL); Sorraya POPAL, Waalwijk (NL); Jorin HOOGENBOM, Wageningen (NL); Sander Sebastiaan VAN BERKEL, Oss (NL); Floris Louis VAN DELFT, Nijmegen (NL)

(73) Assignee: Synaffix B.V., Oss (NL)

(21) Appl. No.: 18/264,570

(22) PCT Filed: Feb. 8, 2022
(86) PCT No.: PCT/EP2022/053024

§ 371 Date: Aug. 7, 2023

(30) Foreign Application Priority Data
Feb. 8, 2021 (EP) 21155884.6

(51) Int. Cl. A61K 47/68; C07K 16/28

(52) U.S. Cl. CPC A61K 47/68031; A61K 47/6813; A61K 47/6849; A61K 47/6889; C07K 16/2809; C07K 16/2878; C07K 2317/24; C07K 2317/41; C07K 2317/622

(57) ABSTRACT

The present invention concerns multifunctional antibody construct containing at least one antibody Ab and two distinct payloads D¹ and D² of structure (1) or (2). Wherein L¹, L², L³, L⁴ and L⁵ are linkers; x1 and x2 are each individually an integer in the range of 1-8, wherein x1+x2=2-10; BM is a branching moiety; m and n are each independently 0 or 1; x3 is an integer in the range of 1-4; and D¹ and D² are two distinct payloads selected from the group consisting of polypeptides, small molecules, cytotoxins and oligonucleotides, wherein at least one of D¹ and D² is a polypeptide. The multifunctional antibody construct according to invention are suitable for use in medicine, such as for use in the treatment of cancer, a viral infection, a bacterial infection, a neurological disease, an autoimmune disease, an eye disease, hypercholesterolemia and amyloidosis.

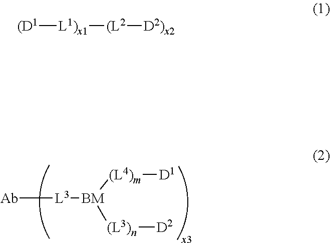

### FIELD OF THE INVENTION

The present invention relates to antibodies with multiple functionalities. More specifically the invention relates to constructs and compositions wherein two (or more) functional moieties are attached to an antibody without requiring genetic engineering of the antibody before such attachment, wherein the functional moiety may comprise a cytotoxin, a polypeptide or other payloads. The resulting multifunctional antibody constructs can be useful, for example, in therapy.

### BACKGROUND OF THE INVENTION

Monoclonal antibodies as protein ligands for a carefully selected biological receptor provide an ideal delivery platform for selective in vivo targeting to an area of disease or a specific pathogen. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to an antibody by recombinant DNA technology, for example the N-terminus or the C-terminus on the light chain or the heavy chain (or both), or in between two antibody domains. In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

A monoclonal antibody may also be genetically modified in the protein sequence itself to modify its structure and thereby introduce (or remove) specific properties. For example, mutations can be made in the antibody Fc-fragment in order to nihilate binding to Fc-gamma receptors, binding to the FcRn receptor or binding to a specific cancer target may be modulated, or antibodies can be engineered to lower the pl and control the clearance rate from circulation.

An emerging strategy in therapeutic treatment involves the use of an antibody that is able to bind simultaneously to multiple antigens or epitopes, a so-called bispecific antibody (simultaneously addressing two different antigens or epitopes), or a trispecific antibody (addressing three different antigens of epitopes), and so forth, as summarized in Kontermann and Brinkmann, Drug Discov. Today 2015, 20, 838-847, incorporated by reference. A bispecific antibody with ‘two-target’ functionality can interfere with multiple surface receptors or ligands associated, for example with cancer, proliferation or inflammatory processes. Bispecific antibodies can also place targets into close proximity, either to support protein complex formation on one cell, or to trigger contacts between cells. Examples of ‘forced-connection’ functionalities are bispecific antibodies that support protein complexation in the clotting cascade, or tumour-targeted immune cell recruiters and/or activators. Depending on the production method and structure, bispecific antibodies vary in the number of antigen-binding sites, geometry, half-life in the blood serum, and effector function. In this regard, bispecific is not to be confused with bivalent, which refers to the ability of a symmetrical IgG to bind to two identical targets at the same time via each of the two identical CDRs.

A bispecific or trispecific antibody may also contain an additional functionality, for example a cytotoxic agent, a polypeptide cytokine, an oligonucleotide, an antibiotic or an antiviral agent. Thereby effectively converting a bispecific antibody into a trifunctional molecule, a trispecific antibody into a tetrafunctional molecule, and so forth. The different functionalities in a multifunctional antibody each have a specific biological function, which includes but is not limited to binding, signalling, immune cell engagement, induction of effector function, checkpoint inhibition, cellular activation, cellular down-regulation, cell-killing, gene silencing, gene activation. The different functionalities can act independently to induce a specific biological response (additive effect) or can mutually enhance their activity (synergistic effect).

A wide range of different formats for multifunctional antibodies has been developed over the years, which can be roughly divided into IgG-like (bearing a Fc-fragment) and non-IgG-like (lacking a Fc-fragment) formats, as summarized by Kontermann and Brinkmann, Drug Discov. Today 2015, 20, 838-847 and Yu and Wang, J. Cancer Res. Clin. Oncol. 2019, 145, 941-956, incorporated by reference. Most bispecific antibodies are generated by one of three methods by somatic fusion of two hybridoma lines (quadroma), by genetic (protein/cell) engineering, or by chemical conjugation with cross-linkers, totalling more than 60 different technological platforms today.

IgG-like formats based on full IgG molecular architectures include but are not limited to IgG with dual-variable domain (DVD-Ig), Duobody technology, knob-in-hole (KIH) technology, common light chain technology and cross-mAb technology, while truncated IgG versions include ADAPTIR, XmAb and BEAT technologies. Non-IgG-like approaches include but are not limited BITE, DART, TandAb and ImmTAC technologies. Bispecific or trispecific antibodies can also be generated by fusing different antigen-binding moieties (e.g., scFv or Fab) to other protein domains, which enables further functionalities to be included. For example, two scFv fragments have been fused to albumin, which endows the antibody fragments with the long circulation time of serum albumin, as demonstrated by Müller et al., J. Biol. Chem. 2007, 282, 12650-12660, incorporated by reference. Another example is the ‘dock-and-lock’ approach based on heterodimerization of cAMP-dependent protein kinase A and protein A kinase-anchoring protein, as reported by Rossi et al., Proc. Nat. Acad. Sci. 2006, 103, 6841-6846, incorporated by reference. These domains can be linked to Fab fragments and entire antibodies to form multivalent bispecific antibodies, as shown by Rossi et al., Bioconj. Chem. 2012, 23, 309-323. The dock-and-lock strategy requires the generation of a fusion protein between the targeting antibody and a peptide fragment for docking onto the protein A kinase-anchoring protein. Therapeutic Ab fragments (scFv, diabody) may also be fused with albumin or proteins that bind albumin, which increases the half-life of the drug in the blood up to five to six times. The construction of such molecules gives unpredictable results, thereby bispecific antibodies generated as the result of different Ab-fragment fusion or binding of Abs to other proteins have limited application in research and development of new therapeutic molecules.

Although not commonly employed in this meaning, any symmetrical, Y-shaped IgG antibody could be considered as a bispecific antibody, in case it harbours in its Fc-domain a complex N-glycan. Such an antibody is able to bind simultaneously to (a) a specific antigen via its polypeptide complement-dependent region (CDR) and (b) to various Fc-gamma receptors I, II and III, also known as CD64, CD32 and CD16, through its N-glycan. For example, trastuzumab is an antibody that binds to the HER2-antigen on cancer cells and (at least partially) exerts its biological effect by effector function, i.e. antibody-dependent cellular cytotoxicity (ADCC), antibody-dependent cellular phagocytosis (ADCP) and complement-dependent cytotoxicity (CDC), all of which is primed by binding of its complex N-glycan to specific proteins or receptors. By the same token, any bispecific antibody can be considered as a trispecific if it is able to bind to (a) two different specific antigens (or epitopes) and (b) an immune cell receptor (its glycan). For example, catumaxomab is a known antibody that is able to bind simultaneously to the cell surface receptors EpCam and CD3 and for that reason is a bispecific antibody. Nevertheless, it is often referred to as a trispecific antibody due to its ability to bind at the same time to CD16 on NK cells by virtue of its complex N-glycan attached to the antibody Fc-part. For the avoidance of doubt, a bispecific antibody would never be considered as trispecific if the antibody has been mutated in its Fc-region to nihilate binding of the N-glycan to Fc-gamma receptors or rendered Fc-silent by another means, such as complete removal of the N-glycan.

Multifunctional antibodies in the true meaning, i.e. not referring to binding of the Fc-glycan but having CDRs to binding to multiple targets simultaneously are known in the art, for example Wu et al. Nature Cancer 2020, 1, 86-98, incorporated by reference, have described a trispecific, trifunctional fusion IgG antibody binding to CD38, CD3 and CD28. Similarly, CDR-Life (https://www.cdr-life.com/science/#pipeline) has in development a trispecific, trifunctional antibody targeting BCMA, PD-L1 and CD3. Numab Therapeutics (www.numab.com) has described a tetravalent, tetrafunctional antibody, binding to PD-L1, HER-2, CD-3 and HSA.

Bispecific, trifunctional antibodies are also known in the art, for example Affimed (www.affimed.com) have developed aTriFlex technology based on antibodies able to bind to two different antigens (e.g. CD200 and BCMA) as well as CD16. Similarly, GT Biopharma is developing a bispecific, trifunctional protein based on fusion of two antibody fragments (anti-CD33 and anti-CD16) fused to a cytokine (IL-15), as described by Vallera et al. Clin. Cancer Res. 2016, 22, 3440-3451, incorporated by reference. A similar trifunctional construct binding to B7-H3 and CD16 fused to IL-15 has been described by Vallera et al. Cancers 2020, 12, 2659-2677, incorporated by reference. Bispecific, trifunctional antibody-drug conjugates are also known in the art, as for example reported by Li et al., Cancer Cell 2016, 29, 117-129, incorporated by reference, for MEDI4276. Another example of a bispecific, trifunctional antibody-drug conjugate is ZW49, as developed by Zymeworks (www.zymeworks.com), based on a biparatopic antibody targeting two different epitopes on HER-2 and conjugated to auristatin payload. A third example is M1231, a bispecific antibody targeting MUC-1 and EGFR and conjugated to hemiasterlin cytotoxic payload, developed by Merck-Serono.

Herein, any monospecific IgG antibody that is also able to bind to Fc-gamma receptors will be referred to as monospecific, bifunctional, and a bispecific antibody as bispecific, trifunctional. By the same line of reasoning, a monospecific or bispecific IgG antibody that is Fc-silent will be referred to as monospecific, monofunctional antibody or bispecific, bifunctional antibody, respectively. A monospecific antibody (of any type) conjugated to a cytokine, an oligonucleotide or a cytotoxic payload will be referred to as bifunctional, and a bispecific IgG antibody conjugated to a cytokine, an oligonucleotide or a cytotoxic payload as trifunctional. A monospecific antibody whereto is covalently attached two different small molecules, two different oligonucleotides or two different peptide fragments, or a combination of these, will be referred to as monospecific, trifuctional.

Chemical conjugation to generate a non-IgG-type bispecific antibody was used for the first time by Brennan et al., Science 1985, 229, 81-83, incorporated by reference: two Fab₂ fragments obtained by pepsinolysis of rabbit IgG were reduced and then oxidized, resulting in bispecific Fab₂. Similarly, homo- and heterobifunctional reagents interacting with cysteine residues was reported by Glennie et al. 1987, 139, 2367-2375, incorporated by reference. Chemical conjugation of Abs against CD3 and CD20 (rituximab) was used to obtain T cells with bispecific antibody-coated surfaces, as shown by Gall et al., Exp. Hematol. 2005, 33, 452-459, incorporated by reference. Generation of the bispecific CD20×CD3 was ensured by treatment of OKT3 (anti-CD3) with Traut's reagent, followed by mixing with maleimide-functionalized rituximab (obtained by pretreament of rituximab with sulfosuccinimidyl 4-(N-maleimidomethyl)cyclohexane-1-carboxylate (sulfo-SMCC). By virtue of the random chemical conjugation of both antibodies, followed by random heterodimerization, the bispecific antibody is inevitable obtained as a highly heterogeneous mixture (also containing multimers). The only chemical method reported to date that is also site-specific is the CovX-Body technology, as reported by Doppalapudi et al., Bioorg. Med. Chem. Lett. 2007, 17, 501-506, incorporated by reference, based on the instalment of an aldolase catalytic antibody site into the targeting antibody, followed by treatment with peptide fragment chemically modified with an azetidinone-motif, leading to spontaneous ligation. Bispecific antibodies were produced by the addition of two short peptides that inhibited VEGF or angiopoietin 2 with a branched linker and then with the Abs, as reported by Doppalapudi et al., Proc. Nat. Acad. Sci. 2010, 107, 22611-22616, incorporated by reference.

Formats of bispecific antibody generation based on chemical Ab or Ab-fragment conjugation today are not in use, in particular due to the low yield of product (of low purity) and high cost-of-goods. Besides, the advance in recombinant DNA technologies enabled the efficient generation of fusion proteins and positive clinical results were obtained therewith. Regardless, a non-genetic chemical modification approach could significantly accelerate time-to-clinic, in case proper control of site-specificity of stoichiometry can be ensured.

Examples of bispecific antibodies that have been or are currently under clinic development, all of which are Fc-silent and therefore also bifunctional, are blinatumomab (CD19×CD3), GBR1302 (Her2×CD3), MEDI-565 (CEA×CD3), BAY2010112 (PSMA×CD3), RG7221 (angiopoietin×VEGF), RG6013 (FIX×FX), RG7597 (Her×Her3), MCLA128 (Her2×Her3), MM111 (Her2×Her3), MM141 (IGF1R×Her3), ABT122 (TNFalpha×IL17), ABT981 (IL1a×Il1b), ALX0761 (IL17A×IL17F), SAR156597 (IL4×IL13), AFM13 (CD30×CD16) and LY3164530 (Her×cMET).

A popular strategy in the field of cancer therapy employs the use of a bispecific antibody comprising of one CDR binding to an upregulated tumour-associated antigen (TAA or simply target) and one CDR to a receptor present on a cancer-destroying immune cell. e.g. a T cell or an NK cell. Such bispecific antibodies are also known as T cell or NK cell-redirecting antibodies, respectively. Although the approach of immune cell redirecting is already more than 30 years old, new technologies are overcoming the limitations of the 1^{st} generation immune cell-redirecting antibodies, especially extending half-life to allow intermittent dosing, reducing immunogenicity and improving the safety profile. Currently, there is one approved drug (blinatumomab or Blincyto®) and more than 30 other bispecific formats in various stages of clinical development.

Like other methods of therapy for severe diseases, therapeutic bispecific antibodies cause different side effects, the most common of which are nausea, vomiting, abdominal pain, fatigue, leukopenia, neutropenia, and thrombopenia. In many patients, Abs against therapeutic bispecific antibodies appear in the blood during treatment. Most adverse events occur during the beginning of therapy, and in most cases side effects normalize under continued treatment. The majority of data on therapeutic BsAb adverse effects are available on blinatumomab and catumaxomab, since these drugs have undergone numerous clinical trials. A common side effect of blinatumomab and catumaxomab therapy is “cytokine storm”, elevation of cytokine levels and some neurological events. Cytokine release-related symptoms are general side effects of many therapeutic mAbs and occur due to specific mechanisms of action: use of cytotoxic T cells as effectors. Minimizing cytokine-release syndrome is possible with a low initial dose of the drug in combination with subsequent high doses, as well as corticosteroid (dexamethasone) and antihistamine premedication.

One way to mitigate the adverse events associated with immune cell engagement therapy, in particular cytokine release syndrome, and to avoid the use of step-up-dosing regimens, was reported by Bacac et al., Clin. Cancer Res. 2018, 24, 4785-4797, incorporated by reference. It was shown that with significantly higher potency and safer administration could be achieved by generating a CD20×CD3 T cell engager with a 2:1 molecular format, i.e. bivalent binding to CD20 and monovalent binding to CD3, which is achieved by insertion of the anti-CD3 fragment in one of the Fab arms of the full-IgG anti-CD20 antibody. The resulting bispecific antibody is associated with a long half-life and high potency enabled by high-avidity bivalent binding to CD20 and head-to-tail orientation of B- and T cell-binding domains in a 2:1 molecular format. A heterodimeric human IgG1 Fc region carrying the “PG LALA” mutations was incorporated to abolish binding to Fcg receptors and to complement component C1q while maintaining neonatal Fc receptor (FcRn) binding, enabling a long circulatory half-life. The bispecific CD20-T cell engagers displays considerably higher potency than other CD20-TCB antibodies in clinical development and is efficacious on tumour cells expressing low levels of CD20. CD20-TCB also displays potent activity in primary tumour samples with low effector:target ratios.

By far the most investigated receptor for the purpose of T cell-engagement involves the CD3 receptor on activated T cells. T cell-redirecting bispecific antibodies are amongst the most used approaches in cancer treatment and the first report in which bispecific antibodies specifically engaged CD3 on T cells on one side and the antigens of cancer cells independent of their T cell receptor (TCR) on the other side, was published 30 years ago. T cell-redirecting antibodies have made considerable progress in hematological malignancies and solid tumour treatments in the past 10 years. Catumaxomab is the first bispecific antibody of its kind targeting epithelial cell adhesion molecule (EpCAM) and CD3, which is actually trispecific as it also binds to CD16 through its glycan. Catumaxomab was approved in Europe (2009) for the treatment of malignant ascites (but withdrawn in 2017 for commercial reasons). This discovery was followed by another successful bispecific targeting CD19 and CD3 (blinatumomab), which was given marketing permission by the FDA for relapsed or refractory precursor B-cell acute lymphoblastic leukaemia (ALL) treatment in 2014. At present, although many patients benefit from blinatumomab, there are a number of T cell-redirecting antibodies with different formats and characteristics showing potential anti-tumour efficacy in clinical studies.

Antibodies known to bind T cells are known in the art, highlighted by Martin et al., Clin. Immunol. 2013, 148, 136-147 and Rossi et al., Int. Immunol. 2008, 20, 1247-1258, both incorporated by reference, for example OKT3, UCHT3, BMA031 and humanized versions thereof. Antibodies known to bind to Vγ9Vδ2 T cells are also known, see for example de Bruin et al., J. Immunol. 2017, 198, 308-317, incorporated by reference.

The concept of redirecting T cells to the tumour is currently expanded to other receptors, which are at the same time costimulatory, such as CD28, CD137 (4-1BB), CD134 (OX40), CD27 or ICOS.

Similar to T cell engagement, NK cell recruitment to the tumour microenvironment is under broad investigation. NK cell engagement is typically based on binding CD16, CD56, NKp46, or other NK cell-specific receptors, as summarized in Konjevic et al., 2017, http://dx.doi.org/10.5772/intechopen.69729, incorporated by reference. NK cell engagers can be generated by fusion or insertion of an NK-binding antibody (fragment) to a full IgG binding to a tumour-associated antigen. Alternatively, specific cytokines can also be employed, given that NK cell antitumor activity is regulated by numerous activating and inhibitory NK cell receptors, alterations in NK cell receptor expression and signalling underlie diminished cytotoxic NK cell function. Based on this and on predictive in vitro findings, cytokines including IFNα, IL-2, IL-12, IL-15, and IL-18 have been used systemically or for ex vivo activation and expansion of NK cells and have led to improved NK cells antitumor activity by increasing the expression of NK cell activating receptors and by inducing cytotoxic effector molecules. Moreover, this cytokine-based therapy enhances NK cell proliferation and regulatory function, and it has been shown that it induces NK cells exhibiting cytokine induced memory-like properties that represent a newly defined NK cell subset with improved NK cell activity and longevity. Both for cancer therapy as well as for the treatment of chronic inflammation, several cytokine payloads have been developed and tested in preclinical trials. Proinflammatory cytokines such as IL-2, TNF and IL-12 have been investigated for tumour therapy, as they have been found to increase and activate the local infiltrate of leukocytes at the tumour site. For example, IL-2 monotherapy has been approved as aldesleukin (Proleukin®) and is in phase III clinical trials in combination with nivolumab (NKTR-214). Similarly, various recombinant versions of IL-15 are under clinical evaluation (rhlL-15 or ALT-803). Specific mutants of IL-15 have been reported, for example by Beher et al., Prot. Engin. Des. Sel. 2011, 24, 283-290 and Silva et al., Nature 2019, 565, 186-191, both incorporated by reference, and the complex of IL-15 with IL-15 receptor (IL-15R), as reported by Rubinstein et al., Proc. Nat. Acad. Sci. 2006, 103, 9166-9171, incorporated by reference and fusion constructs of IL-15 and IL-15R (Sushi domain) have also been evaluated for antitumor activity, see for example Bessard et al., Mol. Canc. Ther. 2009, 8, 2736-2745, incorporated by reference. In addition, antibodies have been developed, as for example reported by Boyman et al., Science 2006, 311, 1924-1927, Arenas-Ramirez et al., Sci. Transl. Med. 2016, 8, DOI: 10.1126/scitranslmed.aag3187, Lee et al, Oncoimmunology 2020, 9, e1681869, DOI: 10.1080/2162402X.2019.1681869, WO 2017070561, WO2018217058, WO2016005950, all incorporated by reference, for recruitment of endogenous IL-2, most favorably by binding to a IL-2 domain that normally binds to IL-2Rα, thereby leading to selective activation of CD8+ T cells without activation of Treg. By contrast immunosuppressive cytokines such as IL-10 may be considered as payloads for the treatment of chronic inflammatory conditions or of other diseases (e.g. endometriosis).

Systemic administration of pro-inflammatory cytokines can lead to severe off-target-related adverse effects, which may limit the dose and prevent escalation to therapeutically active regimens. Certain cytokine products (e.g., IL-2, TNF, IL-12) have exhibited recommended doses in the single-digit milligram range (per person) or even below. Adverse effects associated with the intravenous administration of pro-inflammatory cytokines may include hypotension, fever, nausea or flu-like symptoms, and may occasionally also cause serious haematologic, endocrine, autoimmune or neurologic events. In view of these considerations, there is a clear biomedical need for the development of ‘next-generation’ cytokine products, which are better tolerated and which display a preferential action at the site of disease, helping to spare normal tissues, as summarized in Murer and Neri, New Biotechnol. 2019, 52, 42-53, incorporated by reference. Thus, the targeted delivery of cytokines to the tumour aims at inducing a local pro-inflammatory environment, which may activate and recruit immune cells. A list of antibody-cytokine fusions described in the literature has been reported by Hutmacher and Neri, Adv. Drug Deliv. Rev. 2018, 141, 67-91, incorporated by reference. A list of clinical cytokine fusions is provided in Murer and Neri, New Biotechnol. 2019, 52, 42-53, incorporated by reference. Various IL-15 fusions proteins are under preclinical evaluation, as summarized in “T-cell & NK-Cell Engaging Bispecific Antibodies 2019: A Business, Stakeholder, Technology and Pipeline Analysis”, 2019, released by La Merie publishing, incorporated by reference, for example OXS-3550 (CD33-IL-15-CD16 fusion) prepared by Trike technology is currently in phase I.

A common strategy in the field of immune cell engagement employs nihilation or removal of binding capacity of the antibody to Fc-gamma receptors, which has multiple pharmaceutical implications. The first consequence of removal of binding to Fc-gamma receptors is the reduction of Fc-gamma receptor-mediated uptake of antibodies by e.g. macrophages or megakaryocytes, which may lead to dose-limiting toxicity as for example reported for Kadcyla® (trastuzumab-DM1) and LOP628. Selective deglycosylation of antibodies in vivo affords opportunities to treat patients with antibody-mediated autoimmunity. Removal of high-mannose glycoform in a recombinant therapeutic glycoprotein may be beneficial, since high-mannose glycoforms are known to compromise therapeutic efficacy by a specific uptake by endogenous mannose receptors and leading to rapid clearance, as for example described by Gorovits and Krinos-Fiorotti, Cancer Immunol. Immunother. 2013, 62, 217-223 and Goetze et al, Glycobiology 2011, 21, 949-959 (both incorporated by reference). In addition, Van de Bovenkamp et al, J. Immunol. 2016, 196, 1435-1441 (incorporated by reference) describe how high mannose glycans can influence immunity. It was described by Reusch and Tejada, Glycobiology 2015, 25, 1325-1334 (incorporated by reference), that inappropriate glycosylation in monoclonal antibodies could contribute to ineffective production from expressed Ig genes.

In the field of immune therapy, binding of glycosylated antibodies to Fc-gamma receptors on immune cells may induce systemic activation of the immune system, prior to binding of the antibody to the tumour-associated antigen, leading to cytokine storm (cytokine release syndrome, CRS). Therefore, in order to reduce the risk of CRS, the vast majority of immune cell engagers in the clinic are based on Fc-silenced antibodies, lacking the capacity to bind to Fc-gamma receptors. In addition, various companies in the field of bispecific antibodies are tailoring molecular architectures with defined ratios with regard to target-binding versus immune cell-engaging antibody domains. For example, Roche is developing T cell-engagers based on asymmetric monoclonal antibodies that retain bivalent binding capacity to the TAA (for example CD20 or CEA) by both CDRs, but with an additional anti-CD3 fragment engineered into one of the two heavy chains only (2:1 ratio of target-binding:CD3-binding). Similar strategies can be employed for engagement/activation of T cells with anti-CD137 (4-1BB), anti-OX40, anti-CD27 or NK cell-engagement/activation with anti-CD16, CD56, NKp46, or other NK cell specific receptors.

Abrogation of binding to Fc-gamma receptor can be achieved in various ways, for example by specific mutations in the antibody (specifically the Fc-fragment) or by removal of the glycan that is naturally present in the Fc-fragment (C_{H}2 domain, around N297). Glycan removal can be achieved by genetic modification in the Fc-domain, e.g. a N297Q mutation or T299A mutation, or by enzymatic removal of the glycan after recombinant expression of the antibody, using for example PNGase F or an endoglycosidase. For example, endoglycosidase H is known to trim high-mannose and hybrid glycoforms, while endoglycosidase S is able to trim complex type glycans and to some extent hybrid glycan. Endoglycosidase S2 is able to trim both complex, hybrid and high-mannose glycoforms. Endoglycosidase F2 is able to trim complex glycans (but not hybrid), while endoglycosidase F3 can only trim complex glycans that are also 1,6-fucosylated. Another endoglycosidase, endoglycosidase D is able to hydrolyse Man5 (M5) glycan only. An overview of specific activities of different endoglycosidases is disclosed in Freeze et al. in Curr. Prot. Mol. Biol., 2010, 89:17.13A.1-17, incorporated by reference herein. An additional advantage of deglycosylation of proteins for therapeutic use is the facilitated batch-to-batch consistency and significantly improved homogeneity.

Inspiration may be taken from the field of ADC technologies to prepare antibody-protein conjugates for the generation of bispecific antibodies or antibody-cytokine fusions.

Many technologies are known for bioconjugation, as summarized in G. T. Hermanson, “Bioconjugate Techniques”, Elsevier, 3^{rd} Ed. 2013, incorporated by reference. Two main technologies can be recognized for the preparation of ADCs by random conjugation, either based on acylation of lysine side-chain or based on alkylation of cysteine side-chain. Acylation of the ε-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacturing of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Another important technology for conjugation to cysteine side-chain is by means of disulphide bond, a bioactivatable connection that has been utilized for reversibly connecting protein toxins, chemotherapeutic drugs, and probes to carrier molecules (see for example Pillow et al., Chem. Sci. 2017, 8, 366-370. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on Michael addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. nt. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/ (accessed Jan. 7, 2020). Reaction with methylsulfonylphenyloxadiazole has also been reported for cysteine conjugation by Toda et al., Angew. Chem. nt. Ed. 2013, 52, 12592-12596, incorporated by reference.

A number of processes have been developed that enable the generation of an antibody-drug conjugate with defined drug-to-antibody ratio (DAR), by site-specific conjugation to a (or more) predetermined site(s) in the antibody. Site-specific conjugation is typically achieved by engineering of a specific amino acid (or sequence) into an antibody, serving as the anchor point for payload attachment, see for example Aggerwal and Bertozzi, Bioconj. Chem. 2014, 53, 176-192, incorporated by reference, most typically engineering of cysteine. Besides, a range of other site-specific conjugation technologies has been explored in the past decade, most prominently genetic encoding of a non-natural amino acid, e.g. p-acetophenylalanine suitable for oxime ligation, or p-azidomethylphenylalanine suitable for click chemistry conjugation. The majority of approaches based on genetic reengineering of an antibody lead to ADCs with a DAR of ˜2. An alternative approach to antibody conjugation without reengineering of antibody involves the reduction of interchain disulphide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ˜4 (DAR4).

Ruddle et al., ChemMedChem 2019, 14, 1185-1195 have recently shown that DAR1 conjugates can be prepared from antibody Fab fragments (prepared by papain digestion of full antibody or recombinant expression) by selective reduction of the C_{H}1 and C_{L} interchain disulphide chain, followed by rebridging the fragment by treatment with a symmetrical PDB dimer containing two maleimide units. The resulting DAR1-type Fab fragments were shown to be highly homogeneous, stable in serum and show excellent cytotoxicity. In a follow-up publication, White et al., MAbs 2019, 11, 500-515, and also in WO2019034764, incorporated by reference, it was shown that DAR1 conjugates can also be prepared from full IgG antibodies, after prior engineering of the antibody: either an antibody is used which has only one intrachain disulphide bridge in the hinge region (Flexmab technology, reported in Dimasi et al., J. Mol. Biol. 2009, 393, 672-692, incorporated by reference) or an antibody is used which has an additional free cysteine, which may be obtained by mutation of a natural amino acid (e.g. HC-S239C) or by insertion into the sequence (e.g. HC-i239C, reported by Dimasi et al., Mol. Pharmaceut. 2017, 14, 1501-1516). Either engineered antibody was shown to enable the generation of DAR1 ADCs by reaction of the resulting cysteine-engineered ADC with a bis-maleimide derived PBD dimer. It was shown that the Flexmab-derived DAR1 ADCs was highly resistant to payload loss in serum and exhibited potent antitumor activity in a HER2-positive gastric carcinoma xenograft model. Moreover, this ADC was tolerated in rats at twice the dose compared to a site-specific DAR2 ADC prepared using a single maleimide-containing PBD dimer. However, no improvement in therapeutic window was noted, since the minimal effective dose (MED) of the DAR1 ADC versus the DAR2 ADC increased with the same factor 2.

It has been shown in WO2014065661, and by van Geel et al., Bioconj. Chem. 2015, 26, 2233-2242, both incorporated by reference, that antibodies can be site-specifically conjugated based on enzymatic remodeling of the native antibody glycan at N297 (trimming by endoglycosidase and introduction of azido-modified GalNAc derivative under the action of a glycosyltransferase) followed by attachment of a cytotoxic payload using click chemistry. It was demonstrated by and Verkade et al., Antibodies 2018, 7, 12, that the introduction of an acylated sulfamide further improves the glycan remodelling technology in terms of therapeutic index and the DAR of the resulting antibody-drug conjugates could be tailored towards DAR2 or DAR4 by choice of specific linker. It was also demonstrated that glycan trimming before conjugation leads to nihilation of binding of the resulting antibody-drug conjugates (ADCs) to Fc-gamma receptors (Fc-silencing). ADCs prepared by this technology were found to display a significantly expanded therapeutic index versus a range of other conjugation technologies and the technology of glycan-remodelling conjugation currently clinically applied in for example ADCT-601 (ADC Therapeutics).

A similar enzymatic approach to convert an antibody into an azido-modified antibody with concomitant Fc-silencing, reported by Lhospice et al., Mol. Pharmaceut. 2015, 12, 1863-1871, incorporated by reference, employs the bacterial enzyme transglutaminase (BTG or TGase). It was shown that deglycosylation of the native glycosylation site N297 with PNGase F liberates the neighbouring N295 to become a substrate for TGase-mediated introduction, which converts the deglycosylated antibody into a bis-azido antibody upon subjection to an azide-bearing molecule in the presence of TGase. Subsequently, the bis-azido antibody was reacted with DBCO-modified cytotoxins to produce ADCs with DAR2. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

Besides the attachment of small molecules, it has also been amply demonstrated that various click chemistries are suitable for the generation of protein-protein conjugates. For example, Witte et al., Proc. Nat. Acad. Sci. 2012, 109, 11993-11998, incorporated by reference, have shown the unnatural N-to-N and C-to-C protein dimers can be obtained by a combination of sortase-mediated introduction of two complementary click probes (azide and DBCO) into two different proteins, followed by seamless ligation based on metal-free click chemistry (strain-promoted azide-alkyne cycloaddition or SPAAC). Wagner et al., Proc. Nat. Acad. Sci. 2014, 111, 16820-16825, incorporated by reference, have applied this approach to prepare a bispecific antibody based on C-terminal sortagging with an anti-influenza scFv, which was further extended to metal-free click chemistry based on inverse electron-demand Diels-Alder cycloaddition with tetrazines by Bartels et al., Methods 2019, 154, 93-101, incorporated by reference. Tetrazine ligation had been applied earlier also by for example Devaraj et al., Angew. Chem. Int. Ed. 2009, 48, 7013-7016 and Robillard et al., Angew. Chem. Ed. Engl. 2010, 49, 3375-3378, both incorporated by reference, for antibody modification by first (random) chemical installment of a trans-cyclooctene (TCO) onto an antibody. In contrast, site-specific introduction of TCO (or tetrazine or cyclopropene other click moieties for tetrazine ligation) onto antibodies can be achieved by a multitude of methods based on prior genetic modification of the antibody as described above and for example reported by Lang et al., J. Am. Chem. Soc. 2012, 134, 10317-10320, Seitchik et al., J. Am. Chem. Soc. 2012, 134, 2898-2901 and Oller-Salvia, Angew. Chem. Int. Ed. 2018, 57, 2831-2834, all incorporated by reference.

Sortase is a suitable enzyme for site-specific modification of proteins after prior introduction of a sortase recognition sequence, as first reported by Popp et al., Nat. Chem. Biol. 2007, 3, 707-708). Many other enzyme-enzyme recognition sequence combinations are also known for site-specific protein modification, as for example summarized by Milczek, Chem. Rev. 2018, 118, 119-141, incorporated by reference, and specifically applied to antibodies as summarized by Falck and Mûller, Antibodies 2018, 7, 4 (doi:10.3390/antib7010004) and van Berkel and van Delft, Drug Discov. Today: Technol. 2018, 30, 3-10, both incorporated by reference. Besides, a wide array of methods is available for non-genetic modification of native proteins, as summarized by Koniev and Wagner, Chem. Soc. Rev. 2015, 44, 5495-5551, incorporated by reference and for N-terminal modification by Rosen and Francis, Nat. Chem. Biol. 2017, 13, 697-705 and Chen et al., Chem. Sci. 2017, 8, 27172722, both incorporated by reference. Any of the above approaches could be employed to install a proper click probe into a polypeptide/protein, as for example summarized by Chen et al., Acc. Chem. Res. 2011, 44, 762-773 and Jung and Kwon, Polymer Chem. 2016, 7, 4585-4598, both incorporated by reference, and applied to an immune cell engager or a cytokine. Upon installation of the complementary click probe into the antibody targeting the tumour-associated antigen, an immune cell engager can be readily generated while the stoichiometry of tumour-binding antibody to immune cell binder can be tailored by proper choice of technology.

It has also been shown by Bruins et al., Bioconjugate Chem. 2017, 28, 1189-1193, incorporated by references, that antibodies can be site-specifically conjugated to cytotoxic payload by tyrosinase-mediated oxidation of a suitably positioned tyrosine through an intermediate 1,2-quinone that subsequently can undergo cycloaddition with a strained alkyne or alkene. The technology is referred to as strain-promoted oxidation-controlled quinone-alkyne cycloaddition (SPOCQ).

Chemical approaches have also been developed for site-specific modification of antibodies without prior genetic modification, as for example highlighted by Yamada and Ito, ChemBioChem. 2019, 20, 2729-2737.

Chemical conjugation by affinity peptide (CCAP) for site-specific modification has been developed by Kishimoto et al., Bioconj. Chem. 2019, by using a peptide that binds with high affinity to human IgG-Fc, thereby enabling selective modification of a single lysine in the Fc-fragment with a biotin moiety or a cytotoxic payload. Similarly, Yamada et al., Angew. Chem. nt. Ed. 2019, 58, 5592-5597 and Matsuda et al., ACS Omega 2019, 4, 20564-20570, both incorporated by reference, have demonstrated that a similar approach (AJICAP™ technology) can be applied for the site-specific introduction of thiol groups on a single lysine in the antibody heavy chain. CCAP or AJICAP™ technology may also be employed for the site-specific introduction of azide groups or other functionalities.

As is clear, genetic fusion of an immune cell engager or cytokine to an IgG leads to homogenous products. Chemical conjugation of immune cell engagers to antibodies has been applied but leads to heterogeneous mixtures. To date, no methods have been reported for the preparation of homogenous bispecific antibodies or antibody-cytokine fusions that do not require prior reengineering of the full-length IgG and/or enables tailoring of the number of immune cell-engaging polypeptides as well as the spacer length and structure between IgG and polypeptide. In addition, no non-genetic methods have been reported to convert an IgG into a bispecific antibody that is Fc-silent.

Besides, no methods have been reported to chemically conjugate to an IgG in controlled fashion two different functionalities, thereby converting any monofunctional antibody into a trifunctional antibody, or a bispecific, bifunctional antibody into a tetrafunctional antibody.

### SUMMARY OF THE INVENTION

The inventors have developed multifunctional antibody construct by attachment of at least two different functionalities (small molecule, polypeptide, oligonucleotide, fluorophore, radiolabel, etc.) without requiring genetic modification of the IgG. Typically, the antibody is specific for a tumour cell and the polypeptide payload is specific for an immune cell. The invention enables tailoring of the molecular format of the resulting multifunctional antibody to a defined ratio, i.e. the ratio of complement-dependent regions in the full IgG CDR versus newly installed functional label. For example, a monospecific, monofunctional full-length IgG antibody can be converted into a trifunctional construct of 2:1:1 ratio by installation of precisely two (one each) of two different functional molecules or can be converted into a trifunctional construct of 2:4:1 ratio by installation of four (4) molecules of one functionality and one of another functionality. Further, the invention is also suitable for application to an IgG that is already bispecific (i.e. with two different CDRs, for example a Duobody or a bispecific IgG obtained by knob-in-hole technology or controlled Fab-exchange technology), thereby generating a tetrafunctional antibody of a given format, for example 1:1:1:1 which refer to the two complement-dependent regions in full IgG CDR (1:1) plus two newly installed, different functional molecules. Finally, in case the functionalization of the IgG antibody involves an endoglycosidase-mediated complete deglycosylation step or an endoglycosidase-mediated trimming step, the resulting multifunctional antibody construct will no longer be able to bind to Fc-gamma receptors (Fc-silent), without reengineering of the antibody. The invention further concerns the medical use of the multifunctional antibody constructs according to the invention.

### DESCRIPTION OF THE FIGURES

FIG. 1 shows a representative (but not comprehensive) set of functional groups (F) in a biomolecule, either naturally present or introduced by engineering, which upon reaction with a reactive group lead to connecting group Z. Functional group F may be naturally present or artificially introduced (engineered) into a biomolecule at any position of choice. The pyridazine connecting group is the product of the rearrangement of the tetrazabicyclo[2.2.2]octane connecting group, formed upon reaction of tetrazine with alkyne, with loss of N₂. Connecting groups Z are preferred connecting groups to be used in the present invention.

FIG. 2 shows the general process for non-genetic conversion of a monoclonal antibody into an antibody containing reactive sites for conjugation (F), where the reactive site may be a click probe or a thiol group. The reactive site may be on various positions in the antibody and with a given ratio with regard to antibody, depending on the technology employed. The antibody may also be converted into an antibody containing two different reactive sites probes (structure below) or three different reactive sites (bottom on the right), each with a given ratio with regard to antibody. For amino groups as reactive site, no antibody modification is necessary, as this is naturally present as a side-chain in lysine.

FIG. 3 (top) depicts how an IgG antibody modified with a given number (x) of probes (F), which may be a natural functionality or a click probe installed onto the antibody, can react with the complementary probe (Q) containing two different functional molecules A and B via a branched structure, to form a stable bond (Q) upon reaction, thereby forming a trifunctional antibody. Probes for conjugation may be elected from any suitable combination depicted in FIG. 1. Stoichiometry of the resulting bispecific antibody depends on the number of probes F that were naturally present or installed prior to conjugation, whereby not necessarily all occurrences of F will react. A symmetrical, bivalent IgG may be employed (CDR1=CDR2), thus leading to a trifunctional antibody with a 2:x:x molecular format. A non-symmetrical antibody may also be employed (CDR1≠CDR2), thus leading to a tetrafunctional antibody with a 1:1:x:x molecular format. If two different probes F₁ and F₂ are present or installed onto the antibody (bottom), these can react with two different complementary probes (Q₁ and Q₂) each containing a functional moiety (A or B, respectively) via a linear structure, the molecular format may be further varied with regard to stoichiometry of A versus B, leading to for example a 2:x:y molecular format. Combinations of these two strategies is also possible (not depicted).

FIG. 4 shows three alternative methods to install precisely one occurrence of A and one occurrence of B onto a full-length antibody (defined 2:1:1 molecular format). The full-length antibody therefore has first been modified with two click probes F₁. In one approach (arrow down), the IgG(F₁)₂ is subjected to a construct consisting of two different functional molecules A and B, connected via a branched linker to two complementary click probes Q₁, both of which will react with one occurrence of F₁ on the antibody. In the second approach (arrow right), the IgG(F₁)₂ is subjected to a trivalent construct containing three complementary probes Q₁ of which two will react with IgG(F₁)₂, leaving one unit of Q₁ free for subsequent reaction with F₁-modified branched construct containing one occurrence of A and B each. In the third approach (diagonal arrow), the IgG(F₁)₂ is subjected to a trivalent construct containing two complementary probes Q₁ and one non-reactive click probe F₂ (which is also different from F₁). The two click probes Q will react with IgG(F1)₂, leaving F₂ for subsequent reaction with Q₂-modified construct containing A and B.

FIG. 5 shows cyclooctynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 6 shows a range of antibody variants as starting materials for subsequent conversion to antibody conjugates.

FIG. 7 shows several structures of derivatives of UDP sugars of galactosamine, which may be modified with e.g. a 3-mercaptoalkanoyl group (11a), an azidoacetyl group (11b), an azidodifluoroacetyl group (11c), an alkyne group (11f) or an oxo-alkyl group (11g) at the 2-position, or with an azido(alkyl) group (11d), a mercapto(alkyl) group (11e) or an alkyne group (11h) at the 6-position of N-acetyl galactosamine. The monosaccharide (i.e. with UDP removed) are preferred moieties Su to be used in the present invention.

FIG. 8 depicts a specific example of forming a bispecific antibody of 2:2 molecular format based on glycan remodeling of a full-length IgG and azide-cyclooctyne click chemistry. The IgG is first enzymatically remodeled by endoglycosidase-mediated trimming of all different glycoforms, followed by glycosyltransferase-mediated transfer of azido-sugar onto the core GlcNAc liberated by endoglycosidase. In the next step, the azido-remodeled IgG is subjected to a polypeptide, e.g. an immune cell-engaging polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 9 is an illustration of how an azido-sugar remodeled antibody can be converted into a bispecific with a 2:1 molecular format by subjecting first to trivalent cyclooctyne construct suitable for clipping onto bis-azido antibody, leaving one cyclooctyne free for subsequent SPAAC with azido-modified polypeptide, effectively installing only one polypeptide onto the IgG. The latter polypeptide may also be modified with other complement click probes for reaction with cyclooctyne, e.g. a tetrazine moiety for inverse electron-demand Diels-Alder cycloaddition. Any combinations of F and Q (FIG. 1) can be envisaged here.

FIG. 10 shows various options for trivalent constructs for reaction with a bis-azidosugar modified mAb. The trivalent construct may be homotrivalent or heterotrivalent (2+1 format). A homotrivalent contract (X=Y) may consist of 3× cyclooctyne or 3× acetylene or 3× maleimide or 3× other thiol-reactive group. A heterotrivalent construct (X≠Y) may for example consist of two cyclooctyne groups and one maleimide group or two maleimides groups and one trans-cyclooctene group. The heterotrivalent construct may exist of any combination of X and Y unless X and Y and reactive with each other (e.g. maleimide+thiol).

FIG. 11 shows the general concept of sortase-mediated ligation of proteins (capital letters for common amino acid abbreviations) for C-terminal (top) or N-terminal (bottom) ligation to a protein of interest. For C-terminal ligation, a LPXTGG sequence recombinantly fused to the C-terminus of a protein of interest, where X can be any amino acid except proline and GG may be further fused to other amino acids (sequences), and sortase-mediated ligation is achieved by treatment with substrate GGG-R (with R is functionality of interest) to form a new peptide bond. Similarly, for N-terminal ligation, a GGG sequence is fused to the N-terminus of a protein of interest, for ligation with an LPXTGG sequence, where the leucine is modified with functionality of interest R, X can be any amino acid except proline and GG may be further fused to other amino acids (sequences).

FIG. 12 shows a range of bivalent BCN reagents (105, 107, 118, 125, 129, 134), trivalent BCN reagents (143, 145, 150), and monovalent BCN reagents for sortagging (154, 157, 161, 163, 168).

FIG. 13 shows a range of metal-free click reagents equipped with N-terminal GGG (169-171 and 176) or C-terminal LPETGG (172-175), suitable for sortagging of proteins.

FIG. 14 shows a range of bis-BCN-modified cytotoxic drugs based on MMAE or MMAF.

FIG. 15 shows a range of additional bis-BCN-modified cytotoxic drugs based on MMAE (303), PBD dimer (304), calicheamicin (305) or PNU159,682 (306).

FIG. 16 shows a range of bivalent cytotoxic drugs with various cyclooctynes (BCN, DIBO, DBCO, with various inter-cyclooctyne linker variations) or azide or maleimide, based on MMAE or MMAF.

FIG. 17 shows the structure of three monovalent, linear linker-drugs based on MMAE (312 and LD14) or MMAF (313).

FIG. 18 shows a range of bivalent or trivalent cross-linkers (XL01-XL09, XL11, XL12, XL14).

FIG. 19 shows structures of scFv's hOKT3 (200), mOKT3 (PF04) and α-4-1BB (PF31) equipped with C-terminal LPETGG, C-terminal G₄SY, N-terminal SLR (or both), possibly also G₄S spacer. Structures 201-204 and PF01, PF02, PF04-PF09 are derivatives of 200, PF04 or PF31, equipped with a suitable click probe (BCN, tetrazine or azide) obtained by enzymatic or chemical derivatization.

FIG. 20 shows bivalent, bis-BCN-modified derivatives of 200.

FIG. 21 shows structures of various mutants of IL-15 (PF18) or IL-15R-IL-15 fusion protein (207, 208 and PF26, IL-15R=Sushi domain of IL-15 receptor) and derivatives thereof equipped with a suitable click probe (BCN, tetrazine or azide) or maleimide, in each case modified at its N-terminus to enable site-specific modification.

FIG. 22 shows bivalent derivatives of PF26, equipped with bis-BCN (PF27 and PF29) or bis-maleimide (PF28), as well as bis-BCN-modified IL-15 (PF30), derived from PF18.

FIG. 23 shows a range of functionalized protein fragments: PF32 is obtained by reaction of XL11 with PF03, which upon further reaction with PF19 provides PF34. Reaction of trivalent BCN-reagent 105 with PF18 provides PF33, while PF35 is obtained by reaction of PF09 with PF27.

FIG. 24 shows SDS-PAGE analysis: Lane 1—rituximab; Lane 2—rit-via; Lane 3—rit-v1a-145; Lane 4—rit-v1a-(201)₂; Lane 5—rit-v1a-145-204; Lane 6—rit-v1a-145-PF01; Lane 7-rit-v1a-145-PF02. Gels were stained with coomassie to visualize total protein. Samples were analyzed on a 6% SDS-PAGE under non-reducing conditions (left) and 12% SDS-PAGE under reducing conditions (right).

FIG. 25 shows SDS-PAGE analysis on a 6% gel under non-reducing conditions: Lane 1—trast-via; Lane 2—trast-v1a-(PF07)₁; Lane 3—trast-v1a-(PF07)₁—(PF33)₁; Lane 4—trast-v1a-(PF07)₁-(LD11)₁. Gels were stained with coomassie to visualize total protein.

FIG. 26 shows SDS-PAGE analysis on a 6% gel under non-reducing conditions: Lane 1—rit-v1a-145-(PF02)₁; Lane 2—rit-v10-[145-PF02]-[LD09]; Lane 3—rit-v10-[145-PF02]—[XL01]; Lane 4—rit-v10-[145-PF02]—[XL01-PF19]; Lane 5—rit-v10-[145-PF02]—[XL01-PF13]. Gels were stained with coomassie to visualize total protein.

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application. When the structure of a compound is depicted as a specific cis or trans isomer, it is to be understood that the invention of the present application is not limited to that specific cis or trans isomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “monosaccharide” is herein used in its normal scientific meaning and refers to an oxygen-containing heterocycle resulting from intramolecular hemiacetal formation upon cyclisation of a chain of 5-9 (hydroxylated) carbon atoms, most commonly containing five carbon atoms (pentoses), six carbon atoms (hexose) or nine carbon atoms (sialic acid). Typical monosaccharides are ribose (Rib), xylose (Xyl), arabinose (Ara), glucose (Glu), galactose (Gal), mannose (Man), glucuronic acid (GlcA), N-acetylglucosamine (GlcNAc), N-acetylgalactosamine (GalNAc) and N-acetylneuraminic acid (NeuAc).

The term “cytokine” is herein used in its normal scientific meaning and are small molecule proteins (5-20 kDa) that modulate the activity of immune cells by binding to their cognate receptors and by triggering subsequent cell signalling. Cytokines include chemokines, interferons (IFN), interleukins, monokines, lymphokines, colony-stimulating factors (CSF) and tumour necrosis factors (TNF). Examples of cytokines are IL-1 alpha (IL1a), IL-1 beta (IL1 b), IL-2 (IL2), IL-4 (IL4), IL-5 (IL5), IL-6 (IL6), IL8 (IL-8), IL-10 (IL10), IL-12 (IL12), IL-15 (IL15), IFN-alpha (IFNA), IFN-gamma (IFN-G), and TNF-alpha (TNFA).

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multi-specific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, cetuximab, omalizumab, bevacizumab, natalizumab, ranibizumab, panitumumab, eculizumab, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multifunctional antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

The term “antibody construct” is herein defined as the covalently linked combination of two or more different proteins, wherein one protein is an antibody or an antibody fragment and the other protein (or proteins) is a polypeptide, such as an antibody, an antibody fragment or a cytokine.

Typically, one of the proteins is an antibody or antibody fragments with high affinity for a tumour-associated receptor or antigen, while one (or more) of the other proteins is an antibody, antibody fragment or polypeptide with high affinity for a receptor or antigen on an immune cell.

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “bispecific” is herein defined as an antibody construct with affinity for two different receptors or antigens (or different epitopes on a single antigen), which may be present on a tumour cell or an immune cell, wherein the bispecific may be of various molecular formats and may have different valencies.

The term “trispecific” is herein defined as an antibody construct with affinity for three different receptors or tumour-associated antigens (or different epitopes on a single antigen), which may be present on a tumour cell or an immune cell, wherein the trispecific may be of various molecular formats and may have different valencies.

The term “multispecific” is herein defined as an antibody construct with affinity for at least two different receptors or antigens (or different epitopes on one or more single antigen), which may be present on a tumour cell or an immune cell, wherein the multispecific may be of various molecular formats and may have different valencies.

The term “biparatopic” is herein defined as an antibody with affinity for two distinctly different epitopes, however both epitopes are present on the same receptor or tumour-associated antigen.

The term “bifunctional” is herein defined as an antibody with two distinctly different properties for example an antibody able to bind to two distinctly different epitopes or tumour-associated antigens or an antibody that is able to bind to a specific epitope or tumour-associated antigens and also carries a small-molecule payload

The term “trifunctional” is herein defined as an antibody with three distinctly different properties for example an antibody able to bind to three distinctly different epitopes or tumour-associated antigens or an antibody that is able to bind to two distinctly different epitopes or tumour-associated antigens and also carries a small-molecule payload or an antibody that is able to bind to a specific epitope or tumour-associated antigen and also carries two different small-molecule payloads.

The term “multifunctional” is herein defined as an antibody with a multitude of distinctly different properties for example an able to bind to multiple distinctly different epitopes or tumour-associated antigen or an antibody that is able to bind to a specific epitope or tumour-associated antigen and also carries multiple small-molecule payloads or a variation thereof.

The term “Fc-silent” is herein defined as an antibody with significantly decreased or nihilated binding to Fc-gamma receptors III (CD16).

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “polar linker” is herein defined as a linker that contains structural elements with the specific aim to increase polarity of the linker, thereby improving aqueous solubility. A polar linker may for example comprise one or more units, or combinations thereof, selected from ethylene glycol, a carboxylic acid moiety, a sulfonate moiety, a sulfone moiety, an acylated sulfamide moiety, a phosphate moiety, a phosphinate moiety, an amino group or an ammonium group.

A “spacer” or spacer-moiety (Sp) is herein defined as a moiety that spaces (i.e. provides distance between) and covalently links together two (or more) parts of a linker. The linker may be part of e.g. a linker-construct, the linker-conjugate or a bioconjugate, as defined below. A spacer may be a covalent bond or a chain of at least 1, preferably 2 to 50, atoms selected from C, N, O, S and P. Herein, the chain of atoms refers to the shortest chain of atoms going from the extremities of the spacer. The atoms within the chain may also be referred to as backbone atoms. As the skilled person will appreciate, atoms having more than two valencies, such as C, N and P, may be appropriately functionalized in order to complete the valency of these atoms.

A “bioconjugate” is herein defined as a compound wherein a biomolecule is covalently connected to a payload via a linker. A bioconjugate comprises one or more biomolecules and/or one or more target molecules.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody. Payload thus refers to the monovalent moiety having one open end which is covalently attached to the targeting moiety via a linker. A payload may be small molecule or a biomolecule.

The term “molecular format” refers to the number and relative stoichiometry of different functionalities on an antibody, with 2:1:1 molecular format denoting a trifunctional antibody with two CDR domain binding to the same target and with one occurrence of another functional molecule attached, and with 1:1:2:2 molecular format denoting a tetrafunctional antibody based on a bispecific antibody with two different CDR domains binding to two different targets (or two targets on the same epitope) and with one occurrence of another functional molecule attached. One (or both) of the functional moieties themselves can be a polypeptide fragment able to bind a specific target as well, which would be different from the target of the antibody scaffold. The term “2:1 molecular format” refer to a protein conjugate consisting of a bivalent monoclonal antibody conjugated to a single functional payload.

The term “complement-dependent region” or “CDR” refers to the variable fragment of an antibody that is able to bind a specific receptor or antigen.

### The Invention

The present inventors have developed multifunctional antibody constructs, which are on one hand specific for a tumour cell and on the other hand specific for an immune cell, such as a T cell, an NK cell, a monocyte, a macrophage, a granulocyte. The multifunctional antibody construct of the invention may furthermore contain one or more different small molecule payloads for specific release in the tumour microenvironment or the tumour lysosome. For the first time, it has become possible to prepare such bifunctional or multifunctional antibody constructs with full control of molecular format and without the need of genetic engineering. The present invention concerns the multifunctional antibody constructs as well as the (medical) use of the multifunctional antibody constructs according to the invention.

Here below, molecular moieties are defined in starting materials, intermediates and final products. The skilled person understands that any definition of preferred embodiment of either one of those equally applies to the other compounds, as long as that part of the molecule is not affected during the conversion. Likewise, anything structurally defined for the process according to the invention equally applies to the compounds according to the invention.

### The Multifunctional Antibody Construct

In a first aspect, the invention concerns a multifunctional antibody construct containing at least one antibody (Ab) and two distinct payloads (D¹) and (D²). Herein, at least one of the payloads is a polypeptide, and the other payload may be another polypeptide, a cytotoxin, another small molecule or an oligonucleotide. In one embodiment, the multifunctional antibody construct may contain a third distinct payload (D³).

The multifunctional antibody constructs according to the invention may have structure (1) or (2).

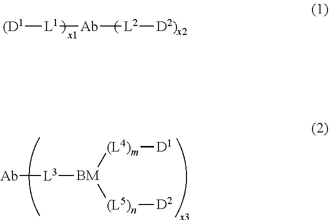

### Herein:

Ab is an antibody;
L¹, L², L³, L⁴ and L⁵ are linkers;
x1 and x2 are each individually an integer in the range of 1-8, wherein x1+x2=2-10;
BM is a branching moiety;
m and n are each independently 0 or 1;
x3 is an integer in the range of 1-4;
D¹ and D² are two distinct payloads selected from the group consisting of polypeptides, small molecules, cytotoxins and oligonucleotides, wherein at least one of D¹ and D² is a polypeptide.

The multifunctional antibody construct according to structure (1) has both distinct payloads connected to separate attachment points on the antibody. The multifunctional antibody construct according to structure (2) has both distinct payloads connected to the same branched linker, which linker may have a single or multiple attachment points on the antibody. As described in more detail below, the method for attaching the payloads to the antibody can be any conjugation technology known in the art. Preferably, for the multifunctional antibody construct according to structure (1), two different conjugation techniques are employed for the two distinct payloads, whereas the multifunctional antibody construct according to structure (2) can readily be prepared by a single conjugation technique which connects a linker-payload construct bearing two distinct payloads.

### The Antibody

Ab is an antibody. Antibodies are known in the art and include IgA, IgD, IgE, IgG, IgM, Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides. Preferably, the antibody is a monoclonal antibody, more preferably selected from the group consisting of IgA, IgD, IgE, IgG and IgM antibodies. Even more preferably Ab is an IgG antibody. The IgG antibody may be of any IgG isotype. The antibody may be any IgG isotype, e.g. IgG1, IgG2, IgI3 or IgG4. Preferably Ab is a full-length antibody, but Ab may also be a Fc fragment.

The antibody Ab is typically specific for an extracellular receptor on a tumour cell, preferably wherein the extracellular receptor on the tumour cell is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Ra, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, β-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin.

In the multifunctional antibody construct according to structure (1), the antibody is functionalized with x1 occurrences of payload D¹ and x2 occurrences of payload D². Herein, x1 and x2 are each individually an integer in the range of 1-8, wherein x1+x2=2-10. The exact number of x1 and x2 determines the molecular format ratio and is governed by the conjugation technique used. In one preferred embodiment, x1 and x2 are the same and are both an integer in the range of 1-4, preferably both or 1 or 2, most preferably both are 1. In an alternative preferred embodiment, x1 is an integer in the range of 1-8, and x2 is an integer in the range of 1-4, preferably x2 is 1 or 2, most preferably x2 is 2. Alternatively, x1 is an integer in the range of 1-4, and x2 is an integer in the range of 1-8, preferably x1 is 1 or 2, most preferably x1 is 2.

In the multifunctional antibody construct according to structure (2), the antibody is functionalized with x3 occurrences of a linker-construct bearing both payload D¹ and payload D². Herein, x3 is an integer in the range of 1-4, preferably x3 is 1, 2 or 4, more preferably x3 is 1 or 2.

### The Payloads

At least one of the payloads, typically D¹, is a polypeptide. The polypeptide payload has affinity for a different target than the antibody Ab. As such, the antibody construct according to the invention is multifunctional and targets at least two distinct targets. Preferably, the polypeptide is selected from an immune cell engager, a checkpoint inhibitor and a binder of a cell surface receptor, preferably wherein the polypeptide is an immune cell-engaging polypeptide or a checkpoint inhibiting polypeptide.

Immune cell-engaging polypeptides are known in the art and any known such polypeptide may be used in the context of the present invention. The immune cell-engaging polypeptide is preferably specific for a cellular receptor on a T cell, an NK cell, a monocyte, a macrophage or a granulocyte, or specific for IL2 or IL15. Herein, it is preferred that the cellular receptor on a T cell is selected from the group consisting of CD3, CD28, CD137 (4-1BB), CD134, CD27, Vγ9Vδ2 and ICOS; that the cellular receptor on a NK cell is selected from the group consisting of CD16, CD56, CD335, CD336, CD337, CD28, NKG2A, NKG2D, NKp46, KIR, DNAM-1 and CD161; that the cellular receptor on the monocyte or macrophage is CD64; and that the cellular receptor on the granulocyte is CD89.

Checkpoint inhibiting polypeptides are known in the art and include and any known such polypeptide may be used in the context of the present invention. Preferably, the checkpoint inhibiting polypeptide is specific for CTLA-4, PD-1, PD-L1, TIGIT, TIM-3, LAG-3 or VISTA

Polypeptides that bind to cell surface receptors are known in the art and any known such polypeptide may be used in the context of the present invention.

In an especially preferred embodiment, the polypeptide is selected from the group consisting of OKT3, UCHT1, BMA031, VHH 6H4, IL2, IL15, IL15/IL15R complex, IL15/IL15R fusion, an antibody specific for IL2 and an antibody specific for IL15. Most preferably, the polypeptide is OKT3, IL15/IL15R fusion, IL15, mAb602, Nara1 or TCB2.

The second payload, typically D², may also be a polypeptide, or may be a cytotoxin, another small molecule or an oligonucleotide. If the second payload is also a polypeptide, it is a distinct polypeptide from the first payload. The second payload may be a small molecule, such as a cytotoxin or other small molecule. Small molecules typically have a low to medium molecular weight compounds, such as about 100 to about 2500 Da, preferably about 300 to about 1750 Da. These may include active substances (e.g. cytotoxin) and reporter molecules (e.g. fluorophore, radiolabel) as defined below. In a preferred embodiment, the second payload is a polypeptide, a cytotoxin, or an oligonucleotide, more preferably a polypeptide or a cytotoxin. In one embodiment, the second payload is a polypeptide. In another embodiment, the second payload is a cytotoxin.

Cytotoxins are well-known in the field of antibody-conjugates, especially for the treatment of cancer. Antibody-drug conjugates typically have a cytotoxic payload, and any such cytotoxic payload can be used in the context of the present invention. Herein, “cytotoxin” may also be referred to as “anti-cancer agent”. The cytotoxin may be a drug or a prodrug, and is selected from the group consisting of pharmaceutically active compounds, in particular low to medium molecular weight compounds (e.g. about 200 to about 2500 Da, preferably about 300 to about 1750 Da).

Examples of cytotoxins include colchicine, vinca alkaloids, anthracyclines, camptothecins, doxorubicin, daunorubicin, taxanes, calicheamicins, tubulysins, irinotecans, an inhibitory peptide, amanitin, eribulin, deBouganin, duocarmycins, maytansines, auristatins, enediynes, pyrrolobenzodiazepines (PBDs) or indolinobenzodiazepine dimers (IGN) or PNU159,682. Especially preferred cytotoxins include auristatin, such as MMAE, maytansinoids, calicheamicins and camptothecins.

Preferred combinations of payloads D¹ and D² are as follows:
(i) D¹ is a CD3-targeting polypeptide and D² is a CD28-targeting polypeptide;
(ii) D is IL15 or an IL15-targeting polypeptide and D² is a CD16-targeting polypeptide;
(iii) D is IL2 or an IL2-targeting polypeptide and D² is a CD16-targeting polypeptide;
(iv) D is an NKp46-targeting polypeptide and D² is a CD16-targeting polypeptide;
(v) D is a cytotoxin and D² is a checkpoint inhibitor, preferably selected from polypeptides targeting CTLA-4, TIGIT, LAG-3, TIM-3, VISTA, PD-1 or PD-L1;
(vi) D is an OX40-targeting polypeptide and D² is a CD137-targeting polypeptide;
(vii) D is a PD-L1-targeting polypeptide and D² is a CD137-targeting polypeptide;
(viii) D is a cytotoxin and D² is IL15 or an IL15-targeting polypeptide;
(ix) D is a cytotoxin and D² is IL2 or an IL2-targeting polypeptide;
(x) D is a cytotoxin and D² is a CD16-targeting polypeptide; or
(xi) D is a TLR7-agonist or a TRL8-agonist and D² is a CD16-targeting polypeptide.

Any checkpoint inhibitor can be used in the embodiment (v). Preferred checkpoint inhibitors include PD-1 targeting polypeptides and PD-L1-targeting polypeptide. In current cancer treatment, antibody-drug conjugates are often co-administered with checkpoint inhibitors. The present invention provides a versatile method to combine antibody-drug conjugates with checkpoint inhibitors in a single multifunctional antibody construct, which greatly facilitates the combination treatment currently employed. Thus, the combination of a cytotoxin and a checkpoint inhibitor is especially preferred in the context of the present invention.

The multifunctional antibody construct according to the invention may contain a third distinct payload, for example when one of the linker contains an (additional) branching moiety, which is connected, typically via a linker, to a third distinct payload D³. Payload molecules are well-known in the art, especially in the field of antibody conjugates, as the moiety that is covalently attached to the antibody and that is released therefrom upon uptake of the conjugate and/or cleavage of the linker. In a preferred embodiment, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a cytotoxin, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine. When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterial agents, peptides and oligonucleotides. Examples of cytotoxins include colchicine, vinca alkaloids, anthracyclines, camptothecins, doxorubicin, daunorubicin, taxanes, calicheamicins, tubulysins, irinotecans, an inhibitory peptide, amanitin, eribulin, deBouganin, duocarmycins, maytansines, auristatins, enediynes, pyrrolobenzodiazepines (PBDs) or indolinobenzodiazepine dimers (IGN) or PNU159,682 and derivatives thereof. Preferred payloads are selected from MMAE, MMAF, exatecan, SN-38, DXd, maytansinoids, calicheamicin, PNU159,685 and PBD dimers. Especially preferred payloads are PBD, MMAE, exatecan or DXd. In one embodiment, the payload is a maytansinoid. In one embodiment, the payload is exatecan or DXd. In one embodiment, the payload is MMAE. In one embodiment, the payload is a PDB dimer.

The term “reporter molecule” herein refers to a molecule whose presence is readily detected, for example a diagnostic agent, a dye, a fluorophore, a radioactive isotope label, a contrast agent, a magnetic resonance imaging agent or a mass label. A wide variety of fluorophores, also referred to as fluorescent probes, is known to a person skilled in the art. Several fluorophores are described in more detail in e.g. G. T. Hermanson, “Bioconjugate Techniques”, Elsevier, 3^{rd} Ed. 2013, Chapter 10: “Fluorescent probes”, p. 395-463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, ¹¹¹In, ^{114m}In, ¹¹⁵In, ¹⁸F, ¹⁴C, ⁶⁴Cu, ¹³¹I, ¹²⁵I, ¹²³I, ²¹²Bi, ⁸⁸Y ⁹⁰Y, ⁶⁷Cu, ¹⁸⁶Rh, ¹⁸⁸Rh, ⁶⁶Ga, ⁶⁷Ga and ¹⁰B, which is optionally connected via a chelating moiety such as e.g. DTPA (diethylenetriaminepentaacetic anhydride), DOTA (1,4,7,10-tetraazacyclododecane-N,N′,N″,N′″tetraacetic acid), NOTA (1,4,7-triazacyclononane N,N′,N″-triacetic acid), TETA (1,4,8,11-tetraazacyclotetradecane-N,N′,N″,N′″tetraacetic acid), DTTA (N′-(p-isothiocyanatobenzyl)-diethylenetriamine-N¹,N²,N³,N³-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide). Isotopic labelling techniques are known to a person skilled in the art, and are described in more detail in e.g. G. T. Hermanson, “Bioconjugate Techniques”, Elsevier, 3^{rd} Ed. 2013, Chapter 12: “Isotopic labelling techniques”, p. 507-534, incorporated by reference.

Polymers suitable for use as a payload D³ in the compound according to the invention are known to a person skilled in the art, and several examples are described in more detail in e.g. G. T. Hermanson, “Bioconjugate Techniques”, Elsevier, 3^{rd} Ed. 2013, Chapter 18: “PEGylation and synthetic polymer modification”, p. 787-838, incorporated by reference. When payload D³ is a polymer, payload D³ is preferably independently selected from the group consisting of a poly(ethyleneglycol) (PEG), a polyethylene oxide (PEO), a polypropylene glycol (PPG), a polypropylene oxide (PPO), a 1,q-diaminoalkane polymer (wherein q is the number of carbon atoms in the alkane, and preferably q is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D³ are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium or zinc surface), a metal alloy surface (wherein the alloy is from e.g. aluminum, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D³ is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D³ are known to a person skilled in the art. A variety of suitable micro- and nanoparticles is described in e.g. G. T. Hermanson, “Bioconjugate Techniques”, Elsevier, 3^{rd} Ed. 2013, Chapter 14: “Microparticles and nanoparticles”, p. 549-587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D³ is a micro- or a nanoparticle, the micro- or nanoparticle is for example a polymeric micro- or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmetacrylae) [poly(EDGMA/HEMA)]. Optionally, the surface of the micro- or nanoparticles is modified, e.g. with detergents, by graft polymerization of secondary polymers or by covalent attachment of another polymer or of spacer moieties, etc.

Payload D³ may also be a biomolecule. Biomolecules, and preferred embodiments thereof, are described in more detail below. When payload D³ is a biomolecule, it is preferred that the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides. In a preferred embodiment, payload D³ is an oligonucleotide.

In the context of the present invention, cytotoxic payloads are especially preferred. Thus, D² and/or D³ is preferably a cytotoxin, more preferably selected from the group consisting of colchicine, vinca alkaloids, anthracyclines, camptothecins, doxorubicin, daunorubicin, taxanes, calicheamycins, tubulysins, irinotecans, an inhibitory peptide, amanitins, amatoxins, eribulin, deBouganin, duocarmycins, epothilones, mytomycins, combretastatins, maytansines, auristatins, enediynes, pyrrolobenzodiazepines (PBDs) or indolinobenzodiazepine dimers (IGN) or PNU159,682. In an especially preferred embodiment, the payload is MMAE, calicheamicin or exatecan.

### The Conjugation Technique

Any conjugation technique known in the art can be employed to prepare the multifunctional antibody constructs according to the invention. Suitable conjugation techniques include thiol ligation, lysine ligation, cycloadditions (e.g. copper-catalysed click reaction, strain-promoted azide-alkyne cycloaddition, strain-promoted quinone-alkyne cycloaddition). Preferred conjugation techniques used in the context of the present invention include nucleophilic reactions and cycloadditions, preferably wherein the cycloaddition is a [4+2]cycloaddition or a [3+2]cycloaddition and the nucleophilic reaction is a Michael addition or a nucleophilic substitution. Suitable conjugation techniques are for example disclosed in G. T. Hermanson, “Bioconjugate Techniques”, Elsevier, 3rd Ed. 2013 (ISBN:978-0-12-382239-0), WO 2014/065661, van Geel et al., Bioconj. Chem. 2015, 26, 2233-2242, PCT/EP2021/050594, PCT/EP2021/050598 and NL 2026947.

Thus, in a preferred embodiment of the conjugation process according to the invention, conjugation is accomplished via a nucleophilic reaction, such as a nucleophilic substitution or a Michael reaction. A preferred nucleophilic reaction is the acylation of a primary amino group with an activated ester. A preferred Michael reaction is the maleimide-thiol reaction, which is widely employed in bioconjugation.

Thus, in a preferred embodiment of the conjugation process according to the invention, conjugation is accomplished via a cycloaddition. Preferred cycloadditions are a (4+2)-cycloaddition (e.g. a Diels-Alder reaction) or a (3+2)-cycloaddition (e.g. a 1,3-dipolar cycloaddition). Preferably, the conjugation reaction is the Diels-Alder reaction or the 1,3-dipolar cycloaddition. The preferred Diels-Alder reaction is the inverse-electron demand Diels-Alder cycloaddition. In another preferred embodiment, the 1,3-dipolar cycloaddition is used, more preferably the alkyne-azide cycloaddition, and most preferably wherein Q is or comprises an alkyne group and F is an azido group. Cycloadditions, such as Diels-Alder reactions and 1,3-dipolar cycloadditions are known in the art, and the skilled person knowns how to perform them.

### Connection Groups Z

The exact nature of the conjugation technique used determines the exact structure of the linkers that are present in the multifunctional antibody constructs according to the invention. Typically, the linkers that are connected to the antibody Ab contain a connecting group Z that is formed during the conjugation reaction. The term “connecting group” refers to a structural element connecting one part of a compound and another part of the same compound. For example, Z may connect the antibody Ab, optionally via a linker such as L⁸, to payload or a branching moiety BM, optionally via a linker such as L⁷. As will be understood by the person skilled in the art, the nature of a connecting group depends on the type of reaction with which the connection between the parts of said compound was obtained. As an example, when the carboxyl group of R—C(O)—OH (or an activated ester derivative thereof) is reacted with the amino group of H₂N—R′ to form R—C(O)—N(H)—R′, R is connected to R′ via connecting group Z, and Z is represented by the group —C(O)—N(H)—. Since connecting group Z originates from the reaction between Q and F, it can take any form. Moreover, for the working of the present invention, the nature of connecting group Z is not crucial.

Linkers L¹, L² and L³ typically contain a connecting group Z that is obtainable by the conjugation reaction. In case the multifunctional antibody construct has structure (1), it is preferred that the structure of connecting group Z in linker L¹ differs from the one in linker L², which simplifies the synthesis of the multifunctional antibody constructs. Two distinct conjugation techniques can be used to connect the two distinct payloads. However, synthetic approach can also be developed wherein the same conjugation technique is used for both payloads, and the structure of Z will be the same in both linkers L¹ and L².

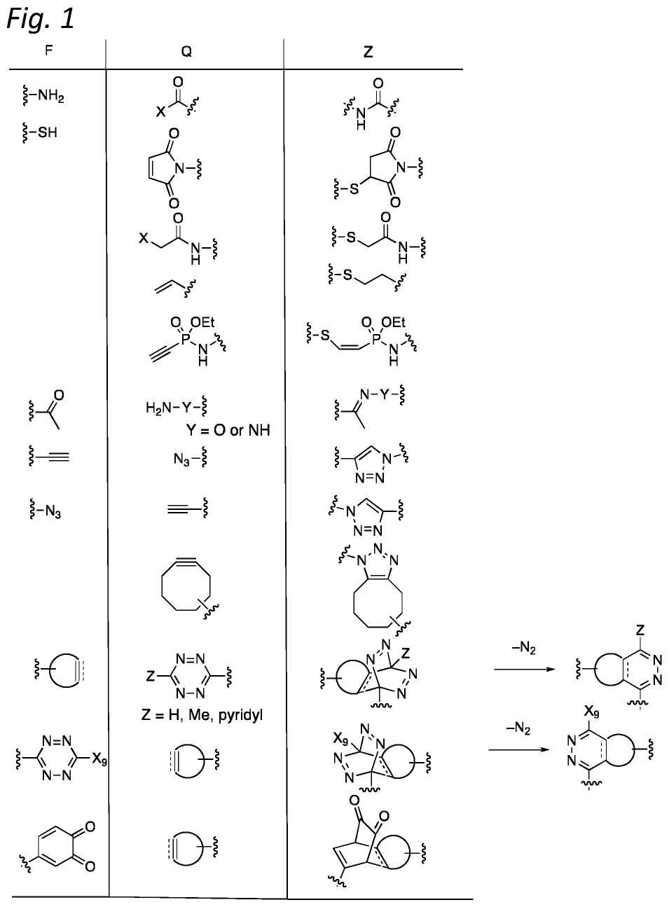

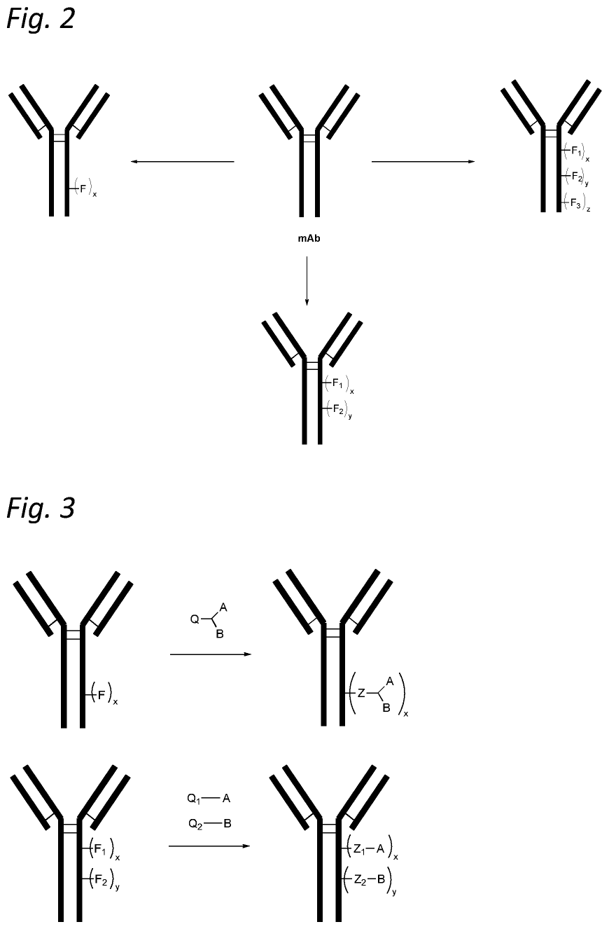

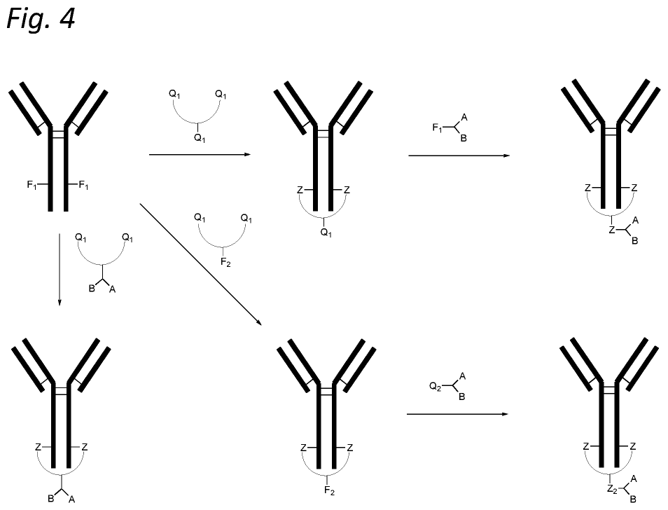

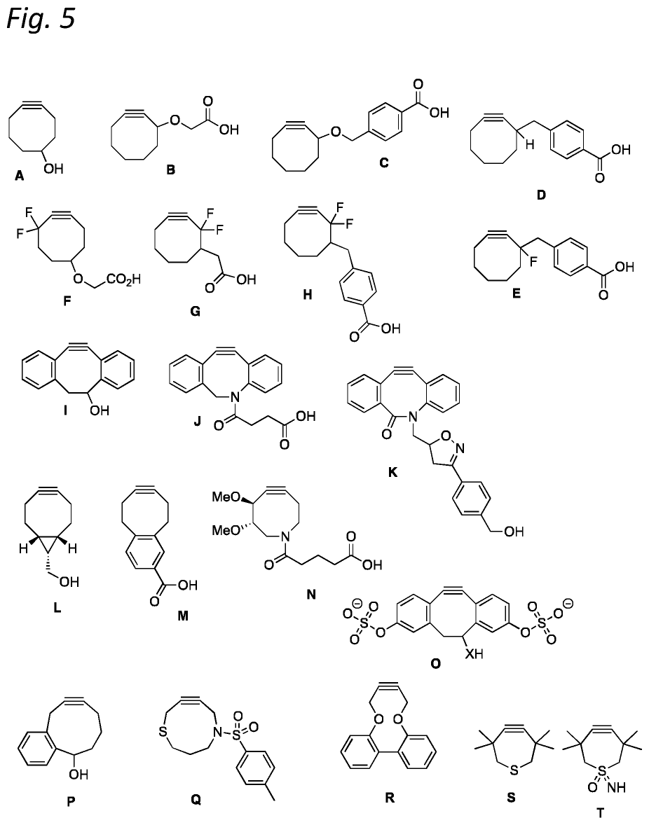

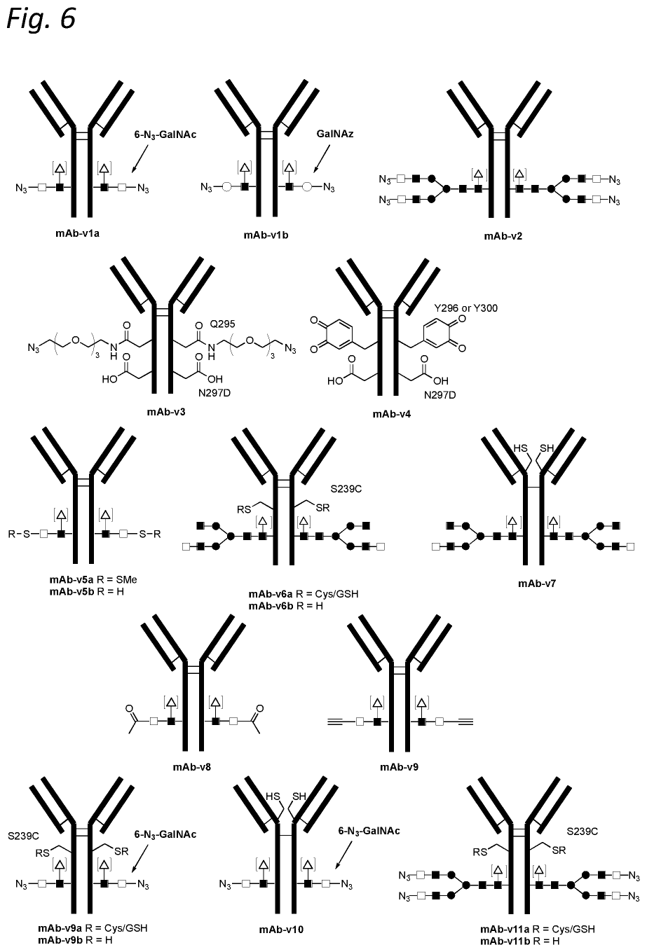

Connecting groups are typically obtained by reacting an antibody containing a reactive moiety F with a payload-linker-construct containing a reactive moiety Q. Herein, reactive moiety F and reactive moiety Q are complementary, meaning that Q reacts F to form a covalently bonded construct in the form of connecting group Z. A large number of organic reactions are available for connecting a reactive group Q to a reactive moiety F. Consequently, there is a large variety of connecting groups Z available. For example, Z may be obtainable by a cycloaddition or a nucleophilic reaction, preferably wherein the cycloaddition is a [4+2]cycloaddition or a 1,3-dipolar cycloaddition and the nucleophilic reaction is a Michael addition or a nucleophilic substitution. Conjugation reactions are known to the skilled person, and the skilled person is capable of selecting appropriate reaction partners F and Q, and will understand the nature of the resulting connecting group Z. Some exemplary options for reactive group Q are provided in FIG. 5, and some exemplary combinations of Q and F, and the corresponding connecting group Z, are provided in FIG. 1. Further guidance is provided in G. T. Hermanson, “Bioconjugate Techniques”, Elsevier, 3rd Ed. 2013 (ISBN:978-0-12-382239-0), in particular in Chapter 3, pages 229-258, incorporated by reference.

For example, when F comprises or is a thiol group, complementary groups Q include N-maleimidyl groups and alkenyl groups, and the corresponding connecting groups Z are as shown in FIG. 1. When F comprises or is a thiol group, complementary groups Q also include allenamide groups and phosphonamidite groups.

For example, when F comprises or is a ketone group, complementary groups Q include (O-alkyl)hydroxylamino groups and hydrazine groups, and the corresponding connecting groups Z are as shown in FIG. 1.

For example, when F comprises or is an alkynyl group, complementary groups Q include azido groups, and the corresponding connecting group Z is as shown in FIG. 1.

For example, when F comprises or is an azido group, complementary groups Q include alkynyl groups, and the corresponding connecting group Z is as shown in FIG. 1.

For example, when F comprises or is a cyclopropenyl group, a trans-cyclooctene group or a cycloalkyne group, complementary groups Q include tetrazinyl groups, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

For example, when F comprises or is a tetrazinyl group, complementary groups Q include a cyclopropenyl group, a trans-cyclooctene group or a cycloalkyne group, and the corresponding connecting group Z is as shown in FIG. 1. In particular cases, Z is only an intermediate structure and will expel N₂, thereby generating a dihydropyridazine (from the reaction with alkene) or pyridazine (from the reaction with alkyne).

Additional suitable combinations of F and Q, and the nature of resulting connecting group Z are known to a person skilled in the art, and are e.g. described in G. T. Hermanson, “Bioconjugate Techniques”, Elsevier, 3rd Ed. 2013 (ISBN:978-0-12-382239-0), Chapter 3, pages 229-258, incorporated by reference. A list of complementary reactive groups suitable for bioconjugation processes is disclosed in Table 3.1, pages 230-232 of Chapter 3 of G. T. Hermanson, “Bioconjugate Techniques”, Elsevier, 3rd Ed. 2013 (ISBN:978-0-12-382239-0), and the content of this Table is expressly incorporated by reference herein.

In a preferred embodiment, each Z is independently selected from the group consisting of —O—, —S—, —S—S—, —NR²—, —N═N—, —C(O)—, —C(O)—NR²—, —O—C(O)—, —O—C(O)—O—, —O—C(O)—NR², —NR₂—C(O)—, —NR²—C(O)—O—, —NR²—C(O)—NR²—, —S—C(O)—, —S—C(O)—O—, —S—C(O)—NR²—, —S(O)—, —S(O)₂—, —O—S(O)₂—, —O—S(O)₂—O—, —O—S(O)₂—NR²—, —O—S(O)—, —O—S(O)—O—, —O—S(O)—NR²—, —O—NR²—C(O)—, —O—NR²—C(O)—O—, —O—NR²—C(O)—NR²—, —NR²—O—C(O)—, —NR²—O—C(O)—O—, —NR²—O—C(O)—NR₂—, —O—NR²—C(S)—, —O—NR²—C(S)—O—, —O—NR²—C(S)—NR²—, —NR²—O—C(S)—, —NR²—O—C(S)—O—, —NR²—O—C(S)—NR²—, —O—C(S)—, —O—C(S)—O—, —O—C(S)—NR²—, —NR²—C(S)—, —NR²—C(S)—O—, —NR²—C(S)—NR²—, —S—S(O)₂—, —S—S(O)₂—O—, —S—S(O)₂—NR²—, —NR²—O—S(O)—, —NR₂—O—S(O)—O—O, —NR²—O—S(O)—NR²—, —NR²—O—S(O)₂—, —NR²—O—S(O)₂—O—, —NR²—O—S(O)₂—NR²—, —O—NR₂—S(O)—, —O—NR²—S(O)—O—, —O—NR²—S(O)—NR²—, —O—NR²—S(O)₂—O—, —O—NR²—S(O)₂—NR²—, —O—NR²—S(O)₂—, —O—P(O)(R²)₂—, —S—P(O)(R²)₂—, —NR²—P(O)(R²)₂— and the moieties represented by any one of (Z1)-(Z71). Herein, R² is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

More preferably, each Z contains a moiety selected from the group consisting of a succinimide, a triazole, a cyclohexene, a cyclohexadiene, an isoxazoline, an isoxazolidine, a pyrazoline, a piperazine, a thioether, an amide or an imide group. Preferably, Z comprises a moiety selected from selected from the group consisting of a triazole, a cyclohexene, a cyclohexadiene, an isoxazoline, an isoxazolidine, a pyrazoline, a piperazine, a thioether, an amide or an imide group. In an especially preferred embodiment, Z comprises a triazole moiety or a succinimide moiety. Triazole moieties are especially preferred to be present in Z.

In a first preferred embodiment, Z is formed by a cycloaddition. Preferred cycloadditions are a (4+2)-cycloaddition (e.g. a Diels-Alder reaction) or a (3+2)-cycloaddition (e.g. a 1,3-dipolar cycloaddition). Preferably, the conjugation reaction is the Diels-Alder reaction or the 1,3-dipolar cycloaddition. The preferred Diels-Alder reaction is the inverse-electron demand Diels-Alder cycloaddition. In another preferred embodiment, the 1,3-dipolar cycloaddition is used, more preferably the alkyne-azide cycloaddition, and most preferably wherein Q is or comprises an alkyne group and F is an azido group. Cycloadditions, such as Diels-Alder reactions and 1,3-dipolar cycloadditions are known in the art, and the skilled person knowns how to perform them.

Preferably, Z contains a moiety selected from the group consisting of a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, an amide or an imide group. Triazole moieties are especially preferred to be present in Z. In one embodiment, Z comprises a (hetero)cycloalkene moiety, i.e. formed from Q comprising a (hetero)cycloalkyne moiety. In an alternative embodiment, Z comprises a (hetero)cycloalkane moiety, i.e. formed from Q comprising a (hetero)cycloalkene moiety.

In a preferred embodiment, Z has the structure (Z1):

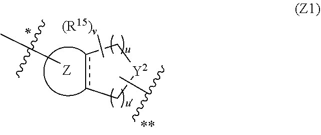

Herein, the bond depicted as [symbol] is a single bond or a double bond. Furthermore:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)═NR³¹ or NR³¹, wherein S⁽⁻⁾ is a cationic sulphur atom counterbalanced by B⁽⁻⁾, wherein B⁽⁻⁾ is an anion, and wherein each R³¹ individually is R¹⁵ or a connection with Q² or D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16;
Ring Z is formed by the cycloaddition and is preferably selected from (Za)-(Zn) defined below.

In case the bond depicted as [symbol] is a double bond, it is preferred that u+u′=4, 5, 6, 7 or 8. Preferably, the wavy bond labelled with * is connected to the antibody Ab, optionally via a linker, and the wavy bond labelled with ** is connected to the payload, optionally via a linker.

Ring Z is formed by the cycloaddition reaction, and preferably is a triazole, a cyclohexene, a cyclohexadiene, a [2.2.2]-bicyclooctadiene, a [2.2.2]-bicyclooctene, an isoxazoline, an isoxazolidine, a pyrazoline or a piperazine. Most preferably, ring Z is a triazole ring. Ring Z preferably has the structure selected from (Za)-(Zn) depicted below, wherein the carbon atoms labelled with ** correspond to the two carbon atoms of the (hetero)cycloalkane or (hetero)cycloalkene ring to which ring Z is fused. In case ring Z is fused to a (hetero)cycloalkene ring, the bond depicted above as [symbol] in (Z1) is a double bond, and ring Z is preferably selected from (Za)-(Zj) depicted below. Preferably, ring Z is according to (Za), (Zi) or (Zj), most preferably according to (Za).

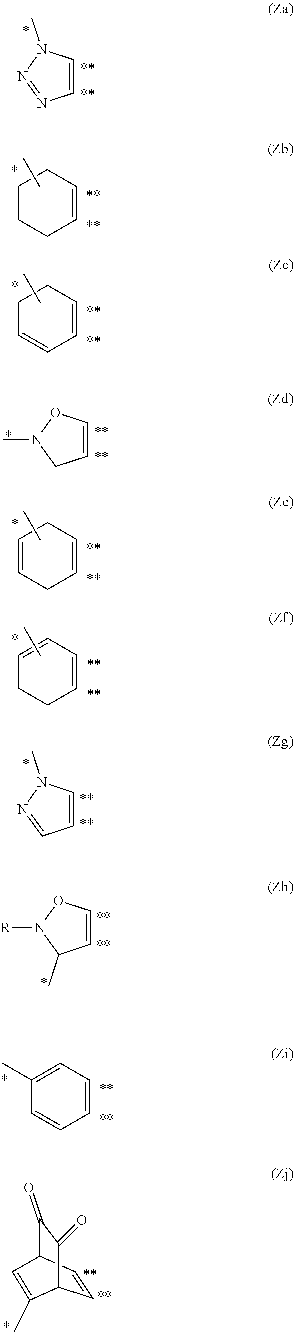

In case ring Z is fused to a (hetero)cycloalkane ring, the bond depicted above as [symbol] in (Z1) is a single bond, and ring Z is preferably selected from (Zk)-(Zn) depicted below. Preferably, ring Z is according to (Zn).

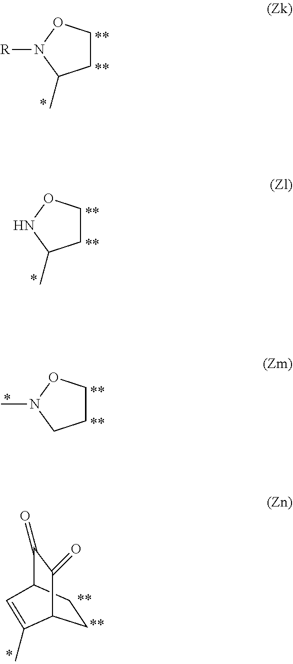

It is especially preferred that Z comprises a (hetero)cycloalkene moiety, i.e. the bond depicted as [symbol] is a double bond. In a preferred embodiment, Z is selected from the structures (Z2)-(Z20), depicted here below:

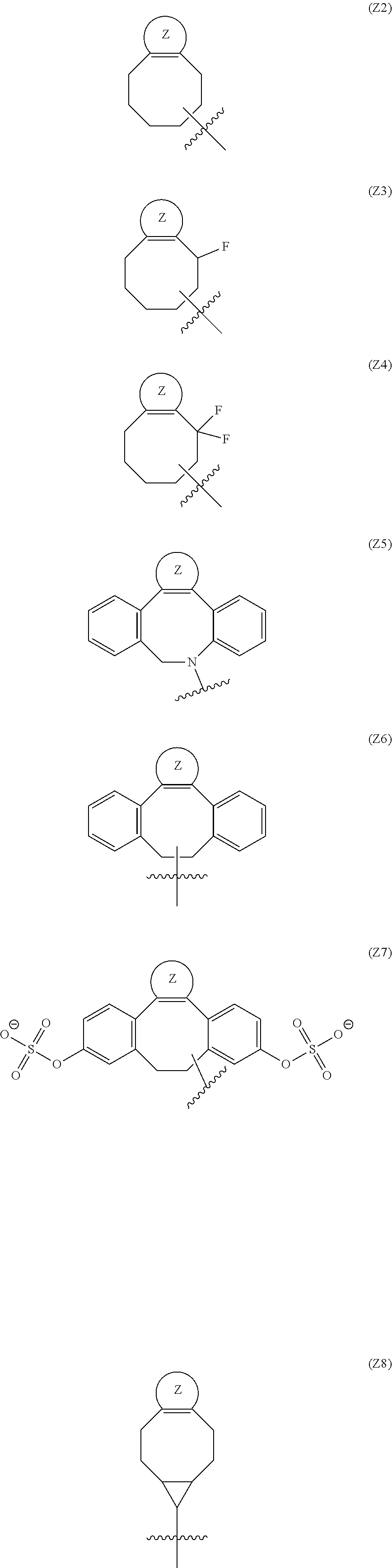

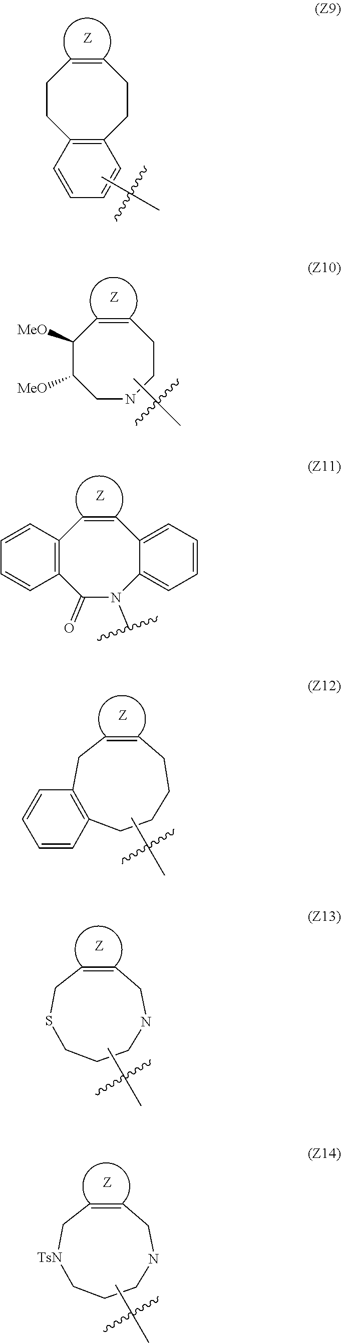

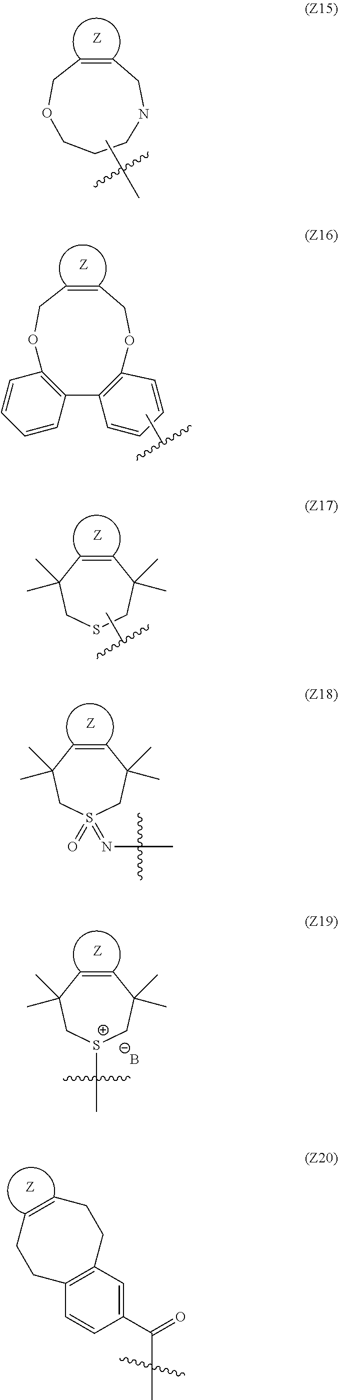

Herein, the connection to the payload, typically via a linker, is depicted with the wavy bond. B⁽⁻⁾ is an anion, preferably a pharmaceutically acceptable anion. Ring Z is formed by the cycloaddition reaction, and preferably has the structure selected from (Za)-(Zj) depicted above, wherein the carbon atoms labelled with ** correspond to the two carbon atoms of the (hetero)cycloalkene ring of (Z2)-(Z20) to which ring Z is fused.

In a further preferred embodiment, Z is selected from the structures (Z21)-(Z38), depicted here below:

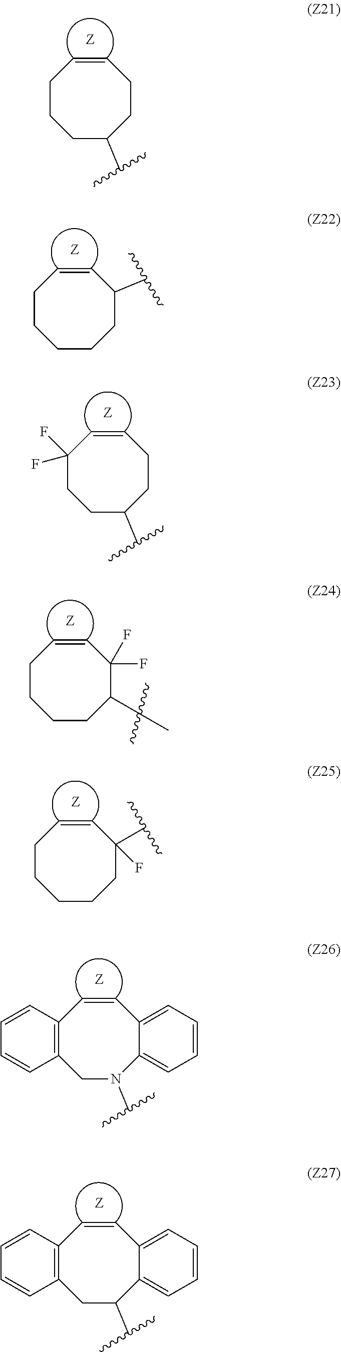

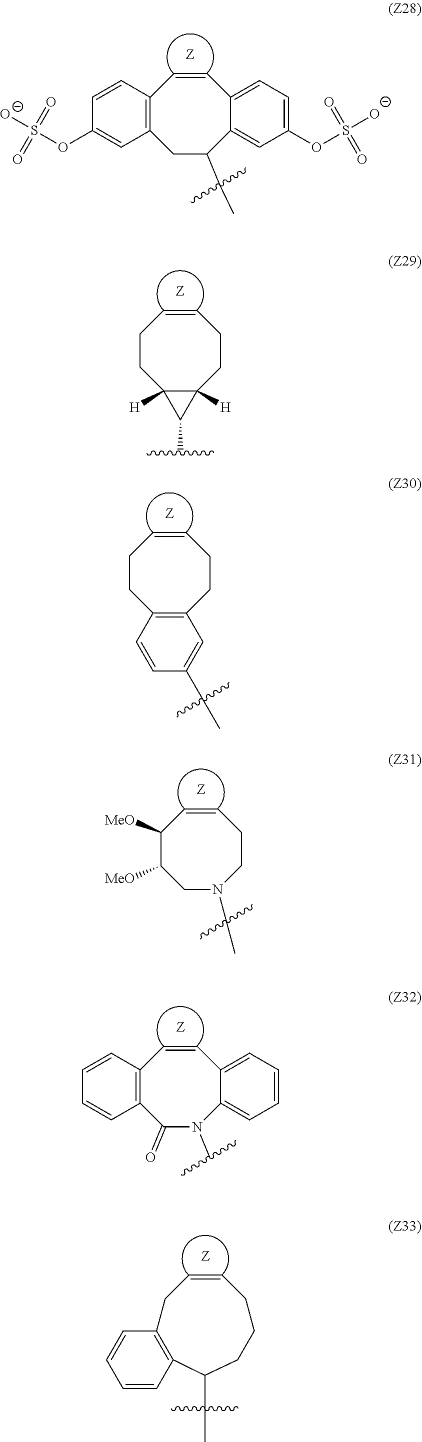 [SMILES: *C1Cc2cc(OS(=O)(=O)[O-])ccc2/C=C\c2ccc(OS(=O)(=O)[O-])cc21 ; *[C@H]1[C@H]2CC/C=C\CC[C@@H]12 ; *c1ccc2c(c1)CC/C=C\CC2 ; *N1CC/C=C\[C@H](OC)[C@@H](OC)C1 ; *N1C(=O)c2ccccc2/C=C\c2ccccc21 ; *C1CCC/C=C\Cc2ccccc21]

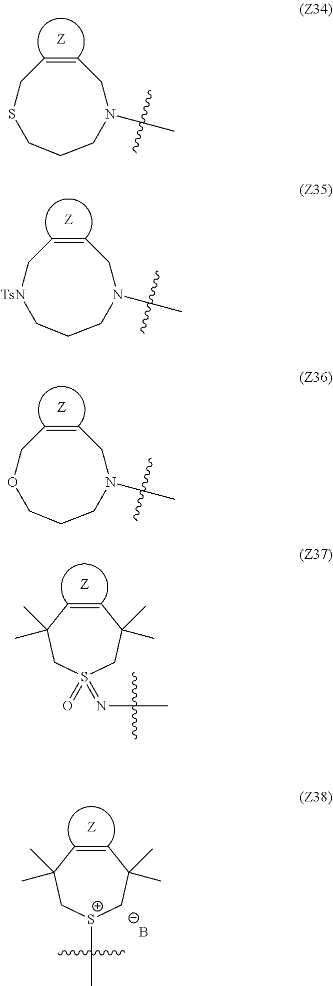

Herein, the connection to the payload, optionally via a linker, is depicted with the wavy bond. In structure (Z38), B⁽⁻⁾ is an anion, preferably a pharmaceutically acceptable anion. Ring Z is selected from structures (Za)-(Zj), as defined above. Ring Z is formed by the cycloaddition reaction, and preferably has the structure selected from (Za)-(Zj) depicted above, wherein the carbon atoms labelled with ** correspond to the two carbon atoms of the (hetero)cycloalkene ring of (Z21)-(Z38) to which ring Z is fused.

In a preferred embodiment, Z comprises a (hetero)cyclooctene moiety according to structure (Z8), more preferably according to (Z29), which is optionally substituted. In the context of the present embodiment, Z preferably comprises a (hetero)cyclooctene moiety according to structure (Z39) as shown below, wherein V is (CH₂)ₗ and l is an integer in the range of 0 to 10, preferably in the range of 0 to 6. More preferably, l is 0, 1, 2, 3 or 4, more preferably l is 0, 1 or 2 and most preferably l is 0 or 1. In the context of group (Z39), l is most preferably 1. Most preferably, Z is according to structure (Z42), defined further below.

In an alternative preferred embodiment, Z comprises a (hetero)cyclooctene moiety according to structure (Z26), (Z27) or (Z28), which are optionally substituted. In the context of the present embodiment, Z preferably comprises a (hetero)cyclooctene moiety according to structure (Z40) or (Z41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Z40) are optionally O-sulfonylated at one or more positions, whereas the rings of (Z41) may be halogenated at one or more positions. Most preferably, Z is according to structure (Z43), defined further below.

In an alternative preferred embodiment, Z comprises a heterocycloheptenyl group and is according to structure (Z37).

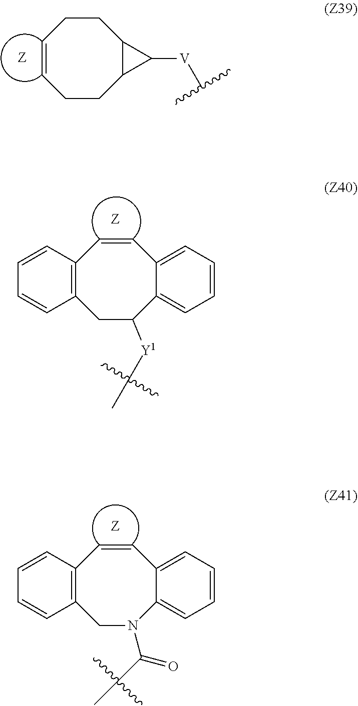

In an especially preferred embodiment, Z comprises a cyclooctenyl group and is according to structure (Z42):

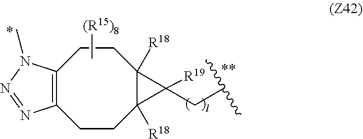

### Herein:

the bond labelled with * is connected to the antibody, optionally via a linker, and the wavy bond labelled with ** is connected to the payload, optionally via a linker;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₆-C₂₄ alkyl(hetero)aryl groups and C₆-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a further occurrence of a payload, such as D³, connected via a spacer moiety; and
l is an integer in the range 0 to 10.

In a preferred embodiment of the group according to structure (Z42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the group according to structure (Z42), R¹⁸ is independently selected from the group consisting of hydrogen, C₁-C₆ alkyl groups, most preferably both R¹⁸ are H. In a preferred embodiment of the group according to structure (Z42), R¹⁹ is H. In a preferred embodiment of the group according to structure (Z42), l is 0 or 1, more preferably l is 1.

In an especially preferred embodiment, Z comprises a (hetero)cyclooctenyl group and is according to structure (Z43):

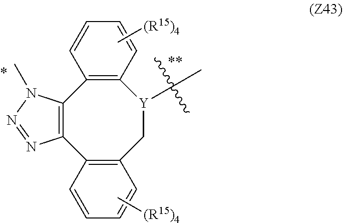

### Herein:

the bond labelled with * is connected to antibody, optionally via a linker, and the wavy bond labelled with ** is connected to the payload, optionally via a linker;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the group according to structure (Z43), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the group according to structure (Z43), Y is N or CH, more preferably Y═N.

In an alternative preferred embodiment, Z comprises a (hetero)cycloalkane moiety, i.e. the bond depicted as [symbol] is a single bond. The (hetero)cycloalkane group may also be referred to as a heterocycloalkanyl group or a cycloalkanyl group, preferably a cycloalkanyl group, wherein the (hetero)cycloalkanyl group is optionally substituted. Preferably, the (hetero)cycloalkanyl group is a (hetero)cyclopropanyl group, a (hetero)cyclobutanyl group, a norbornane group, a norbornene group, a (hetero)cycloheptanyl group, a (hetero)cyclooctanyl group, a (hetero)cyclononnyl group or a (hetero)cyclodecanyl group, which may all optionally be substituted. Especially preferred are (hetero)cyclopropanyl groups, (hetero)cycloheptanyl group or (hetero)cyclooctanyl groups, wherein the (hetero)cyclopropanyl group, the trans-(hetero)cycloheptanyl group or the (hetero)cyclooctanyl group is optionally substituted.

Preferably, Z comprises a cyclopropanyl moiety according to structure (Z44), a hetereocyclobutane moiety according to structure (Z45), a norbornane or norbornene group according to structure (Z46), a (hetero)cycloheptanyl moiety according to structure (Z47) or a (hetero)cyclooctanyl moiety according to structure (Z48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropanyl group is according to structure (Z49). In another preferred embodiment, the (hetero)cycloheptane group is according to structure (Z50) or (Z51). In another preferred embodiment, the (hetero)cyclooctane group is according to structure (Z52), (Z53), (Z54), (Z55) or (Z56).

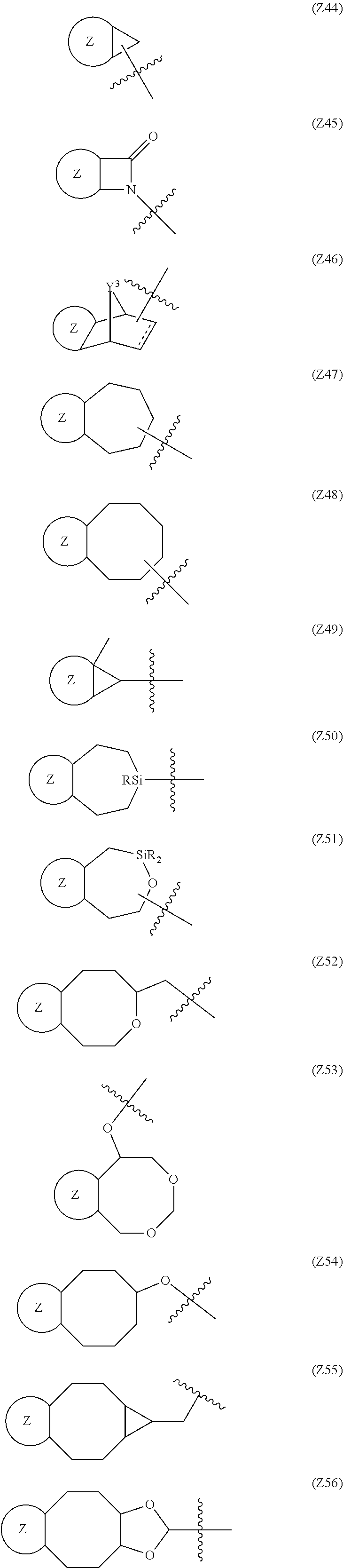

Herein, the R group(s) on Si in (Z50) and (Z51) are typically alkyl or aryl, preferably C₁-C₆ alkyl. Ring Z is formed by the cycloaddition reaction, and preferably has the structure selected from (Zk)-(Zn) depicted above, wherein the carbon atoms labelled with ** correspond to the two carbon atoms of the (hetero)cycloalkane ring of (Z44)-(Z56) to which ring Z is fused.

In a second preferred embodiment, Z is formed by a nucleophilic reaction, preferably by a nucleophilic acylation or a Michael addition, preferably by a Michael addition. A preferred Michael reaction is the thiol-maleimide ligation, most preferably wherein Q is maleimide and F is a thiol group. In a preferred embodiment, connection group Z comprises a succinimidyl ring or its ring-opened succinic acid amide derivative. Preferred options for connection group Z comprise a moiety selected from (Z57)-(Z71) depicted here below.

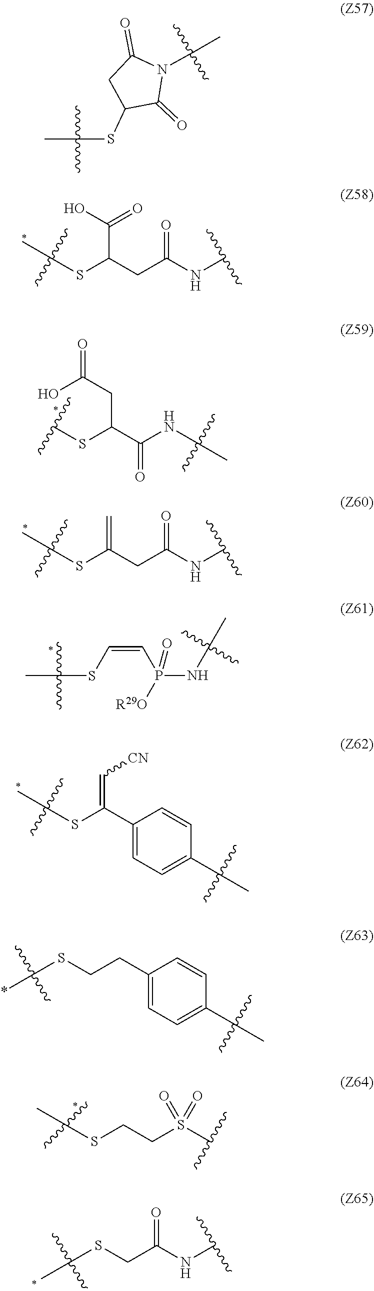

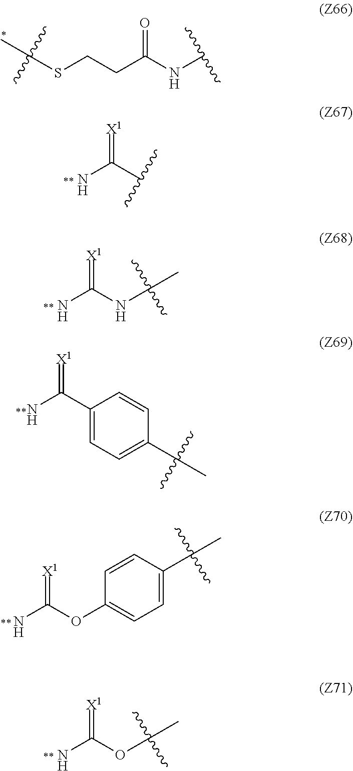

Herein, the wavy bond(s) labelled with an * is connected to the antibody Ab, optionally via a linker, and the wavy bond without label to the payload, optionally via a linker. In addition, R²⁹ is C₁₋₁₂ alkyl, preferably C₁₋₄ alkyl, most preferably ethyl, and X¹ is O or S, preferably X¹═O. The nitrogen atom labelled with ** in (Z67)-(Z71) corresponds to the nitrogen atom of the side chain of a lysine residue of the antibody. The carbon atoms of the phenyl group of (Z69) and (Z70) are optionally substituted, preferably optionally fluorinated.

In a preferred embodiment, connection group Z comprise a moiety selected from (Z1)-(Z71).

### Linkers

The multispecific antibody construct according to the invention contains several linkers. Linkers, also referred to as linking units, are well known in the art and any suitable linker may be used. In the multispecific antibody construct of structure (1), linker L¹ connects the antibody Ab with payload D¹ and linker L² connects the antibody Ab with payload D². In the multispecific antibody construct of structure (2), linker L³ connects the antibody Ab with branching moiety BM, linker L⁴ connects branching moiety BM with payload D¹ and linker L⁵ connects branching moiety BM with payload D². The linkers may be cleavable or non-cleavable. The linker may contain one or more branch-points (in addition to BM) for attachment of multiple payloads D to antibody Ab.

Each linker defined for the multifunctional antibody construct according to the invention independently is a chain of at least 1, preferably 5 to 100, atoms selected from C, N, O, S and P. Herein, the chain of atoms refers to the shortest chain of atoms going from the extremities of the linking unit. The atoms within the chain may also be referred to as backbone atoms. As the skilled person will appreciate, atoms having more than two valencies, such as C, N and P, may be appropriately functionalized in order to complete the valency of these atoms. In other words, the backbone atoms are optionally functionalized. In a preferred embodiment, each of L¹, L² and L³, as well as L⁴ and L⁵ if present, is independently a chain of at least 5 to 50, preferably 6 to 25 atoms selected from C, N, O, S and P. The backbone atoms are preferably selected from C, N and O.

Linkers may for example be selected from the group consisting of linear or branched C₁-C₂₀₀alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups and combinations thereof. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

Each linker may be cleavable or non-cleavable. Especially the linkers directly attached to the payloads, i.e. L¹ and/or L² or L⁴ and/or L⁵, may be cleavable. Cleavable linkers are especially preferred in case the payload they are attached to is a cytotoxin or any small molecule payload able to enter the tumour cell by passive diffusion. Herein, cleavable linkers are preferably cleavable in the tumour lysosome or the tumour microenvironment. Thus, in one embodiment, the cleavable linker is cleavable in the tumour microenvironment. Linkers that are cleavable in the tumour microenvironment are especially advantageous when a cytotoxin payload is combined with a polypeptide targeting a receptor on the outside surface of the tumour cell. As such, the multifunctional antibody construct binds to the tumour cell in the tumour microenvironment, where the linker to the cytotoxin will be cleaved and the cytotoxin is released in close proximity to the tumour cell. Internalisation of the cytotoxin may then lead to cell death of the tumour cell. Linkers that are cleavable in the tumour microenvironment are known to the skilled person and are typically susceptible to hydrolysis by a proteolytic enzyme, such as FAP (fibroblast activation protein), proprotein convertase subtilisins, furins, elastases, legumains, fibroblast activation proteins, tissue-type plasminogen activators, urokinases, matrix metallo proteases (MMPs) and matriptases. Linkers that are cleavable in the tumour lysosome are known to the skilled person and are typically susceptible to hydrolysis by a proteolytic enzyme, such as granzymes, cathepsins, proprotein convertase subtilisins, furins, legumains, caspases and kallikereins.

Such cleavable linkers are known in the art, and typically contain a peptide spacer or derivative (e.g. cyclobutane-1,1-dicarboxamide-Cit). It is preferred that the cleavable linker contains a peptide spacer of structure (26):

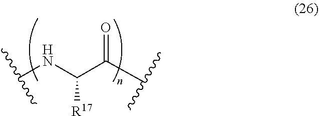

Herein, R¹⁷ represents an amino acid side chain and n is an integer in the range of 1-10, preferably n=1-8, more preferably n=1-5, most preferably n=1-4, preferably n=2.

Preferably, the peptide spacer is a dipeptide or tripeptide spacer as known in the art, preferably a dipeptide spacer. Suitable peptide spacers are selected from the group consisting of Val-Cit, Val-Ala, Val-Lys, Val-Arg, Val-Gln, AcLys-Val-Cit, AcLys-Val-Ala, Phe-Cit, Phe-Ala, Phe-Lys, Phe-Arg, Phe-Gln, Ala-Lys, Leu-Cit, Leu-Gln, Ile-Cit, Trp-Cit, Ala-Ala-Asn, Ala-Asn, Pro-Leu-Gly, Asn-Pro-Val, Lys-Ser-Gly-Arg-Ser-Asp-Asn-His, Pro-Val-Gly-Leu-Ile-Gly, Val-Lys-Gly, Gly-Gly-Gly, Gly-Gly-Phe-Gly and Lys, preferably from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, Ala-Asn, Pro-Leu-Gly, Asn-Pro-Val, Lys-Ser-Gly-Arg-Ser-Asp-Asn-His, Gly-Gly-Phe-Gly and Lys, preferably the peptide spacer is Val-Cit, Val-Ala or Ala-Ala-Asn. In one embodiment, the peptide spacer is Val-Cit. In one embodiment, the peptide spacer is Val-Ala.

In a preferred embodiment, the peptide spacer is represented by general structure (27):

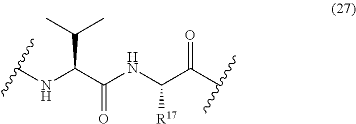

Herein, R¹⁷ represents an amino acid side chain, preferably R¹⁷=CH₃ (Val) or CH₂CH₂CH₂NHC(O)NH₂ (Cit).

The wavy lines in structure (26) and (27) indicate the connection to the remainder of the molecule, preferably the peptide spacer is connected via NH the antibody, typically via a linker, and via C(O) to the payload, typically via a linker.

Linkers directly attached to the antibody Ab may have a single attachment point to the antibody, or may also have more than one, typically two, attachment points. This applies to linkers L¹, L² and L³. Although it is possible that both linker L¹ and linker L² have more than one attachment point, it is preferred that one of linker L¹ and linker L² has only one attachment point, and the other has one or two attachment points.

Linkers with two attachment points to the antibody Ab are preferably represented by structure (L-A):

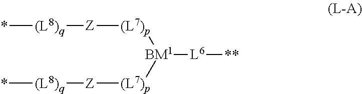

### Herein:

L⁶, L⁷ and L⁸ are linkers;
p and q are each individually 1 or 0;
BM¹ is a branching moiety;
Z are connecting groups.

In a preferred embodiment, linker L¹ or linker L³ has structure (L-A). In case linker L¹ has structure (L-A), it is preferred that x1 is 1 or 2, preferably x1 is 1. In case linker L³ has structure (L-A), it is preferred that x3 is 1 or 2, preferably x3 is 1.

Branching moiety BM¹ is defined in the same way as branching moiety BM, including preferred embodiments thereof. In case the multifunctional antibody construct according to the invention contains both branching moiety BM and BM¹, both branching moieties may be the same or different.

Connecting group Z is further defined above. Preferably, both occurrences of Z are the same. In the context of the present embodiment, connecting group Z is preferably obtained by a cycloaddition as defined above. In case Z is obtained by a cycloaddition, both linkers L⁸ are preferably present and q=1. Both linkers L⁷ are preferably present, and p=1. Most preferably, q=p=1.

L⁶ preferably has structure (L-D):

**-(L²¹)ₙ-(L²²)ₒ-(L²³)ₚ-(L²⁴)_{q}-*** (L-D)

### Herein

the bond labelled ** is connected to BM¹, and the bond labelled *** is connected to BM;
n, o, p and q are each independently 0 or 1;
L²¹, L²², L²³ and L²⁴ are linkers.

Linker L⁶ may contain a connecting group Z that is formed during the synthesis of the multifunctional antibody construct. The payload-construct containing BM, D¹ and D² may be connected to the linker construct comprising BM¹, which may either be before or after reaction of the linker construct (in particular reactive moieties Q) with a functionalized antibody (in particular reactive moieties F). Two representative synthetic approaches are depicted in schemes 1 and 2 below. The connecting group within linker L³ may be formed at the junction any of the linking units L²¹, L²², L²³ and L²⁴, or may separately be present within linker L³. As such, linker L³ may be represented by -Sp-Z-Sp-, wherein Sp are individually spacers. These spacers typically contain part of the linker defined by structure (L-D). For example, L³ may be represented by —Z-(L²¹)ₙ-(L²²)ₒ-(L²³)ₚ-(L²⁴)_{q}- or -(L²¹)ₙ-Z-(L²²)ₒ-(L²³)ₚ-(L²⁴)_{q}-. Herein, Z may take any form, and is preferably as defined above.

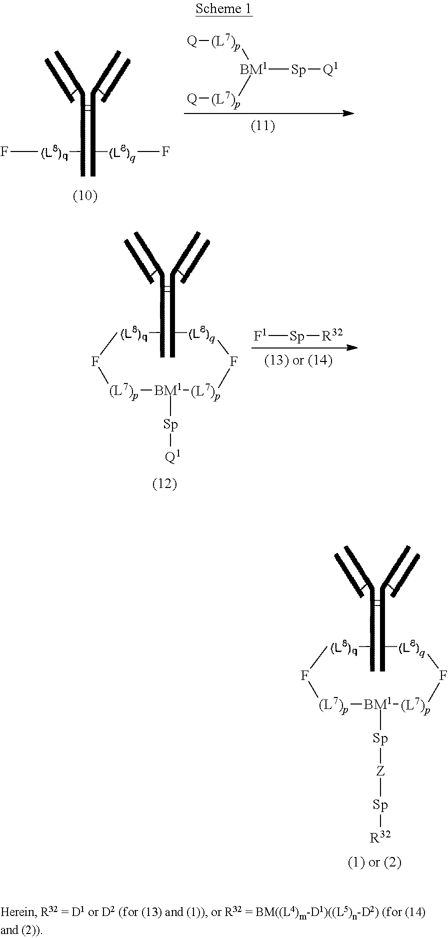

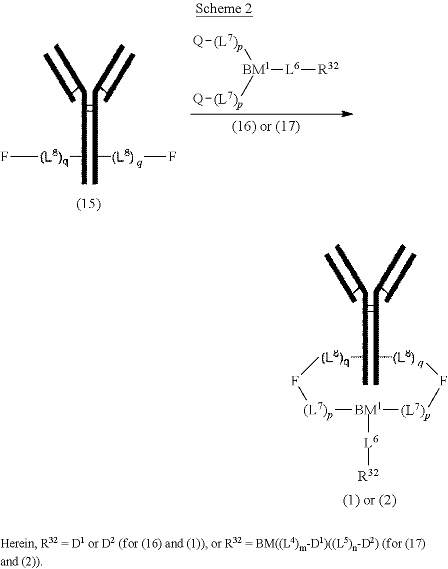

The linker construct may in turn be prepared by a conjugation reaction as follows:

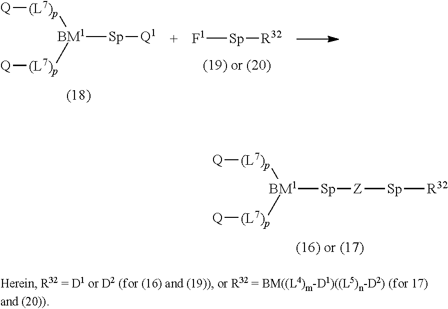

Linkers L⁷ connect BM with connecting groups Z. It is preferred that L⁷ are both present, i.e. both occurrences of p are 1, more preferably they are the same. Each L⁷ may be independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, each L⁷ is independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, each L⁷ is independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, each L⁷ is independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR³, wherein R³ is independently selected from the group consisting of hydrogen, C₁-C₂₄ alkyl groups, C₂-C₂₄ alkenyl groups, C₂-C₂₄ alkynyl groups and C₃-C₂₄ CyCloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR³, preferably O, wherein R³ is independently selected from the group consisting of hydrogen and C₁-C₄ alkyl groups, preferably hydrogen or methyl.

Most preferably, each L⁷ is independently selected from the group consisting of linear or branched C₁-C₂₀ alkylene groups, the alkylene groups being optionally substituted and optionally interrupted by one or more heteroatoms selected from the group of O, S and NR³, wherein R³ is independently selected from the group consisting of hydrogen, C₁-C₂₄ alkyl groups, C₂-C₂₄ alkenyl groups, C₂-C₂₄ alkynyl groups and C₃-C₂₄ CyCloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR³, preferably O and/or or S—S, wherein R³ is independently selected from the group consisting of hydrogen and C₁-C₄ alkyl groups, preferably hydrogen or methyl.

Preferred linkers L⁷ include —(CH₂)ₙ₁—, —(CH₂CH₂)ₙ₁—, —(CH₂CH₂O)ₙ₁—, —(OCH₂CH₂)ₙ₁—, —(CH₂CH₂O)ₙ₁CH₂CH₂—, —CH₂CH₂(OCH₂CH₂)ₙ₁—, —(CH₂CH₂CH₂O)ₙ₁—, —(OCH₂CH₂CH₂)ₙ₁—, —(CH₂CH₂CH2O)ₙ₁CH₂CH₂CH₂— and —CH₂CH₂CH₂(OCH₂CH₂CH₂)ₙ₁—, wherein n1 is an integer in the range of 1 to 50, preferably in the range of 1 to 40, more preferably in the range of 1 to 30, even more preferably in the range of 1 to 20 and yet even more preferably in the range of 1 to 15. More preferably n1 is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

Preferably, the linker with structure (L-A) is connected to two glycosylation sites of the antibody Ab, such that linkers L⁸ in part form the (modified) glycan of the antibody. Linkers L⁸ preferably have the structure (L-E):

*-GlcNAc(Fuc)_{d}-(G)ₑ-Su-** (L-E)

### Herein:

the bond labelled * is connected to an amino acid of the antibody Ab, and the bond labelled ** is connected to Z;
d is 0 or 1;
e is an integer in the range of 0-10;
Su is a monosaccharide;
G is a monosaccharide moiety;
GlcNAc is an N-acetylglucosamine moiety;
Fuc is a fucose moiety.

Linkers with one attachment point to the antibody Ab are preferably represented by structure (L-B) or (L-C):

*-GlcNAc(Fuc)_{d}-(G)ₑ-Su-Z-(L²¹)-(L²²)-(L²³)-(L²⁴)-** (L-B)

*—Z-(L²¹)ₙ-(L²²)ₒ-(L²³)ₚ-(L²⁴)_{q}-** (L-C)

### Herein:

the bonds labelled * are connected to two distinct amino acids of the antibody Ab, and the bond labelled ** is connected to the payload, optionally via a linker;
d, n, o, p and q are each independently 0 or 1;
e is an integer in the range of 0-10;
L²¹, L²², L²³ and L²⁴ are linkers;
Su is a monosaccharide;
G is a monosaccharide moiety;
GlcNAc is an N-acetylglucosamine moiety;
Fuc is a fucose moiety;
Z is a connecting group.

In a preferred embodiment, linker L¹ and/or L², preferably both, has structure (L-B) or (L-C). Linkers with structure (L-B) are preferred linkers with one attachment point to the antibody Ab. Linker (L-B) is preferably attached to a glycosylation site of the antibody Ab, mostly an asparagine amino acid, and in part forms the (modified) glycan of the antibody. Linker (L-C) is preferably attached to a tyrosine, cysteine or lysine amino acid of the antibody, most preferably a cysteine amino acid.

Connecting groups Z are further defined above. In case the linker has structure (L-B), it is preferred that connecting group Z is obtained by a cycloaddition as defined above. In case the linker has structure (L-C) and is attached to a cysteine or lysine amino acid, it is preferred that connecting group Z is obtained by a nucleophilic reaction as defined above. In case the linker has structure (L-C) and is attached to a tyrosine amino acid, it is preferred that connecting group Z is obtained by a cycloaddition as defined above.

Linkers with one attachment point to the antibody and linkers with two attachment points can be attached to an antibody of structure Ab((L⁸)_{q}-F)ₓ, wherein the value of x depends on the exact structure of the multifunctional antibody construct to be prepared. For example, x may be 2 or 4.

The antibody of structure Ab((L⁸)_{q}-F)ₓ may be prepared by any means known in the art. For example, reduction of interchain disulphide bonds of an antibody already gives thiol groups for F. If such reduction is followed by reaction with a defined number of reactive moiety F containing maleimide constructs (or other thiol-reactive constructs), any reactive moiety F can be connected to the antibody. A more controlled, site-specific process of antibody conjugation can be achieved for example by genetic engineering of the antibody to contain two unpaired cysteines (one per heavy chain or one per light chain), to provide exactly two reactive moieties F onto the antibody upon subjection of the antibody to F containing maleimide constructs. Genetic encoding enables the direct expression of an antibody to contain a predefined number of reactive moieties F at specific sites by applying the AMBER stop codon. A range of enzymatic approaches have been also been reported to install a defined number of reactive moieties F onto an antibody, for example based on transglutaminase (TGase), sortase, formyl-glycine generating enzyme (FGE) and others. An alternative enzymatic approach converts the side chains of available tyrosine residues ortho-quinone moieties F by the oxidative action of tyrosinase. The formed ortho-quinone moieties are reactive towards alkenes and alkynes in a cycloaddition. Thus, in one embodiment, the functionalized antibody is prepared by reduction of interchain disulphide bonds followed by reaction with F-containing thiol-reactive constructs, introduction of unpaired cysteine residues followed by reaction with F-containing thiol-reactive constructs, enzymatic introduction of reactive moieties F, and introduction of reactive moieties by genetic engineering. The use of genetic engineering is least preferred in the context of the present application, while enzymatic introduction of reactive moieties F is most preferred.

In a preferred embodiment, GlycoConnect technology (see e.g. WO 2014/065661 and Van Geel et al., Bioconj. Chem. 2015, 26, 2233-2242, incorporated by reference) utilizes the naturally present glycans at the heavy chain of monoclonal antibodies to introduce a fixed number of click probes, in particular azides. Thus, in a preferred embodiment, the functionalized antibody is prepared by (i) optionally trimming of the native glycan with a suitable endoglycosidase, thereby liberating the core GlcNAc, which is typically present on Asn-297, followed by (ii) transfer of an unnatural, azido-bearing sugar substrate from the corresponding UDP-sugar under the action of a suitable glycosyltransferase, for example transfer of GalNAz with galactosyltransferase mutant Gal-T(Y289L) or 6-azidoGalNAc with GalNAc-transferase (GalNAc-T). Alternatively, GalNAc-T can also be applied to install GalNAc derivatives onto the core-GlcNAc harbouring aromatic moieties or thiol function on the Ac group. The multifunctional antibody constructs according to the invention can be obtained with this technology, wherein trimming step (i) may be employed to obtain functionalized antibodies having e=0, or can be omitted to obtain functionalized antibodies having e=1-10. Preferably, a trimming step is performed and e=0.

In a preferred embodiment, the glycan of the antibody is used to attach one or more payloads. In the context of the present embodiment, the multifunctional antibody construct according to the invention has a glycan of structure -GlcNAc(Fuc)_{b}-(G)ₑ-, to which monosaccharide Su is added. Su is a functionalized monosaccharide, comprising a reactive groups F (prior to conjugation) or a connecting groups Z (after conjugation). Hence, Su can be viewed as a monosaccharide derivative, and is further defined below. In view of the monosaccharide core structure of Su, it could be seen as part of the glycan. However, the glycan of structure -GlcNAc(Fuc)_{b}-(G)ₑ- originates from the original glycan of the antibody, to which Su is attached.

The -GlcNAc(Fuc)_{b}-(G)ₑ- of the glycan thus typically originates from the original antibody, wherein GlcNAc is an N-acetylglucosamine moiety and Fuc is a fucose moiety. Fuc is typically bound to GlcNAc via an α-1,6-glycosidic bond. Normally, antibodies may (b=1) or may not be fucosylated (b=0). The GlcNAc residue may also be referred to as the core-GlcNAc residue and is the monosaccharide that is directly attached to the peptide part of the antibody.

Each of the two GlcNAc moieties are preferably present at a native N-glycosylation site in the Fc-fragment of antibody Ab. Preferably, said GlcNAc moieties are attached to an asparagine amino acid in the region 290-305 of Ab. In a further preferred embodiment, the antibody is an IgG type antibody, and, depending on the particular IgG type antibody, said GlcNAc moieties are present on amino acid asparagine 297 (Asn297 or N297) of the antibody.

G is a monosaccharide moiety and e is an integer in the range of 0-10. G is preferably selected from the group consisting of glucose (Glc), galactose (Gal), mannose (Man), fucose (Fuc), N-acetylglucosamine (GlcNAc), N-acetylgalactosamine (GalNAc), N-acetylneuraminic acid (NeuNAc) and sialic acid and xylose (Xyl). More preferably, G is selected from the group consisting of glucose (Glc), galactose (Gal), mannose (Man), fucose (Fuc), N-acetylglucosamine (GlcNAc) and N-acetylgalactosamine (GalNAc).

In a preferred embodiment, e is 0 and G is absent. G is typically absent when the glycan of the antibody is trimmed. Trimming refers to treatment with endoglycosidase, such that only the optionally fucosylated core GlcNAc moiety of the glycan remains.

In another preferred embodiment, e is an integer in the range of 1-10. In this embodiment it is further preferred that G is selected from the group consisting of glucose (Glc), galactose (Gal), mannose (Man), fucose (Fuc), N-acetylglucosamine (GlcNAc), N-acetylgalactosamine (GalNAc), N-acetylneuraminic acid (NeuNAc) or sialic acid and xylose (Xyl), more preferably from the group consisting of glucose (Glc), galactose (Gal), mannose (Man), fucose (Fuc), N-acetylglucosamine (GlcNAc) and N-acetylgalactosamine (GalNAc).

When e is 3-10, (G)ₑ may be linear or branched. Preferred examples of branched oligosaccharides (G)ₑ are (a), (b), (c), (d), (e), (f), (h) and (h) as shown below.

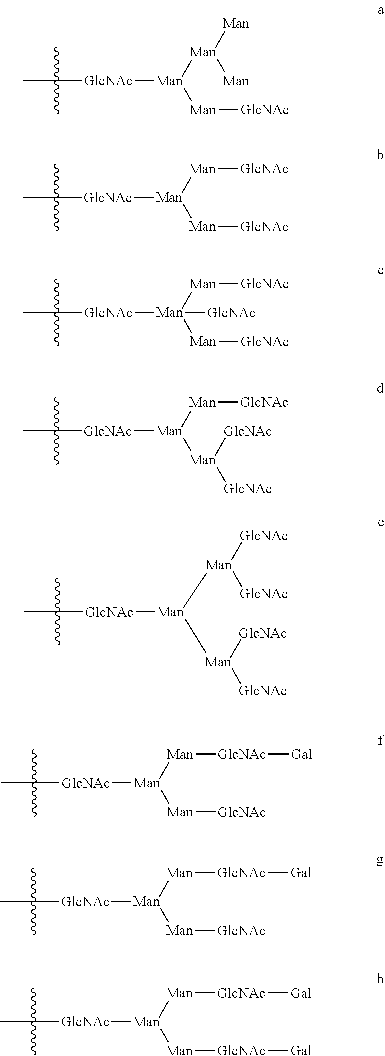

In case G is present, it is preferred that it ends in GlcNAc. In other words, the monosaccharide residue directly connected to Su is GlcNAc. The presence of a GlcNAc moiety facilitates the synthesis of the functionalized antibody, as monosaccharide derivative Su can readily be introduced by glycosyltransfer onto a terminal GlcNAc residue. In the above preferred embodiments for (G)ₑ, having structure (a)-(h), moiety Su may be connected to any of the terminal GlcNAc residues, i.e. not the one with the wavy bond, which is connected to the core GlcNAc residue on the antibody.

It is particularly preferred that G is absent, i.e. that e=0. An advantage of a multifunctional antibody construct wherein e=0 is that when such conjugate is used clinically, binding to Fc gamma receptors CD16, CD32 and CD64 is significantly reduced or fully abrogated.

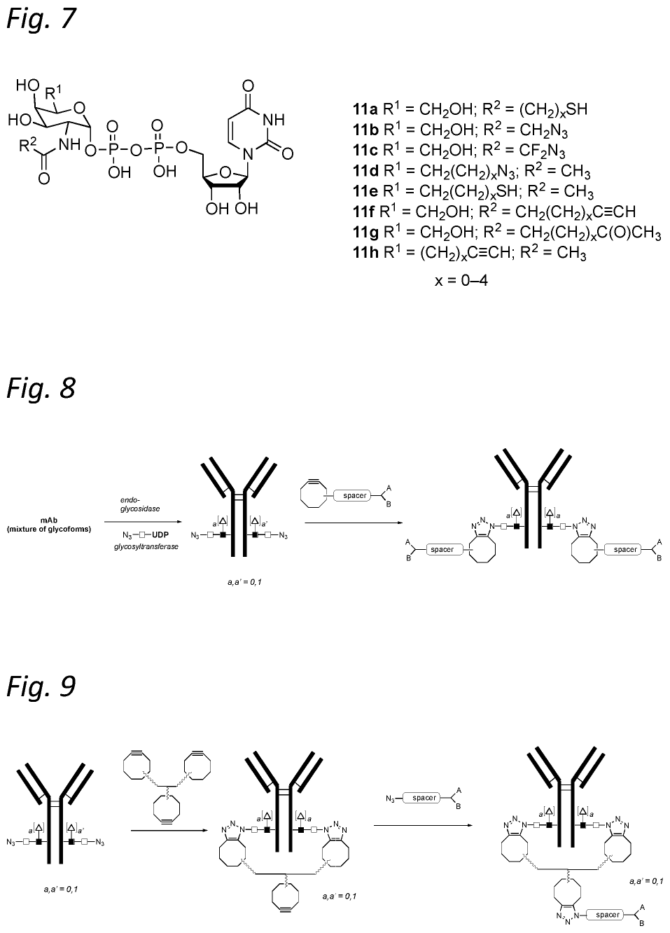

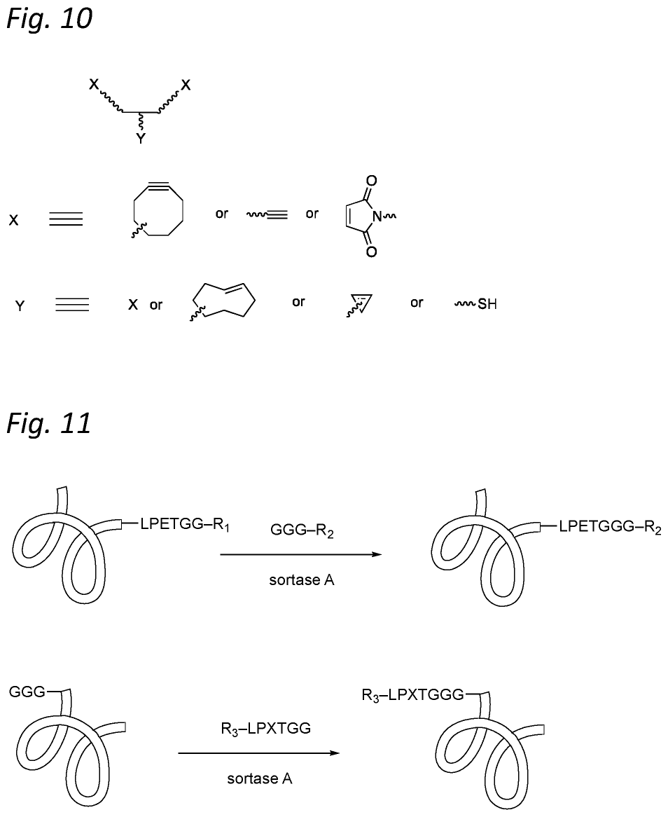

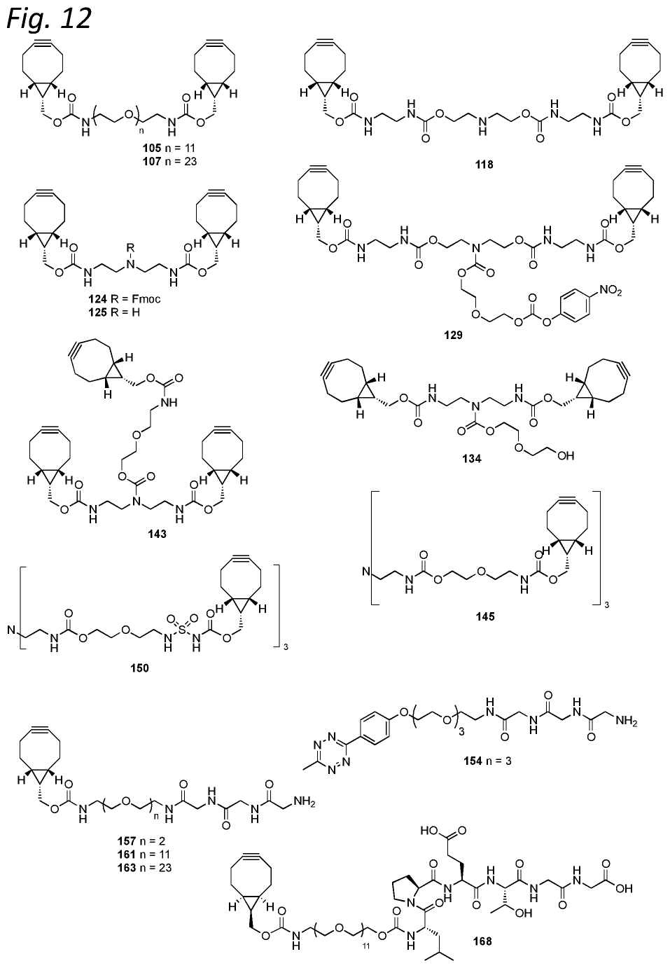

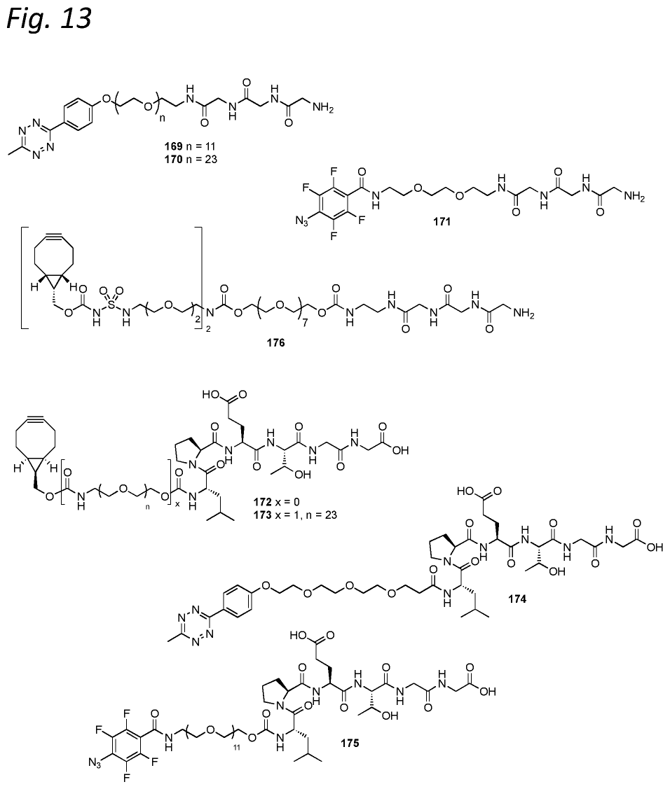

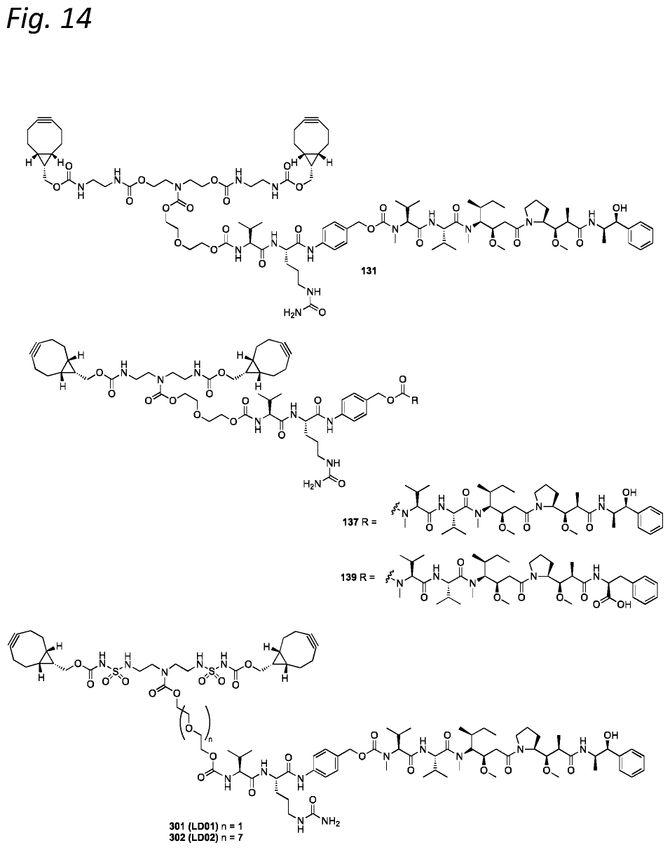

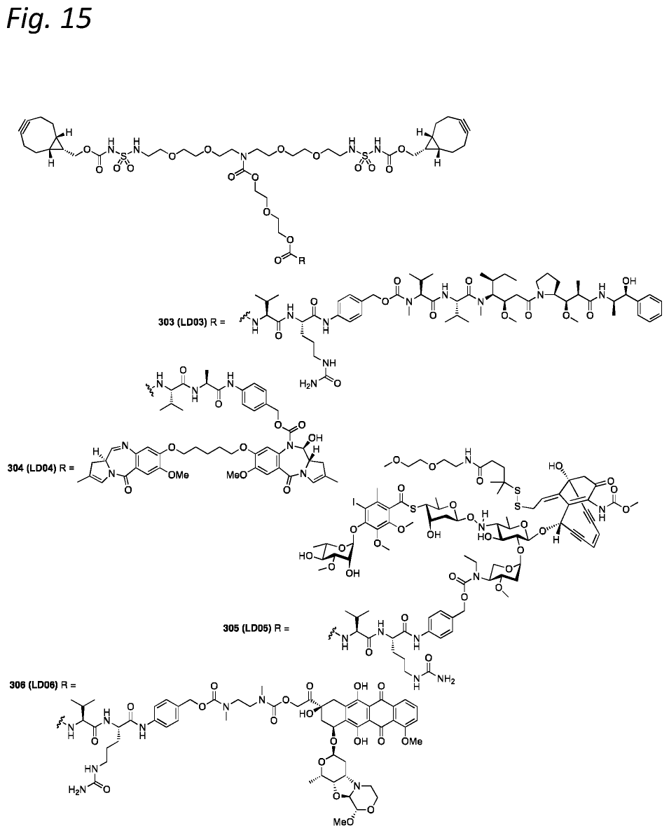

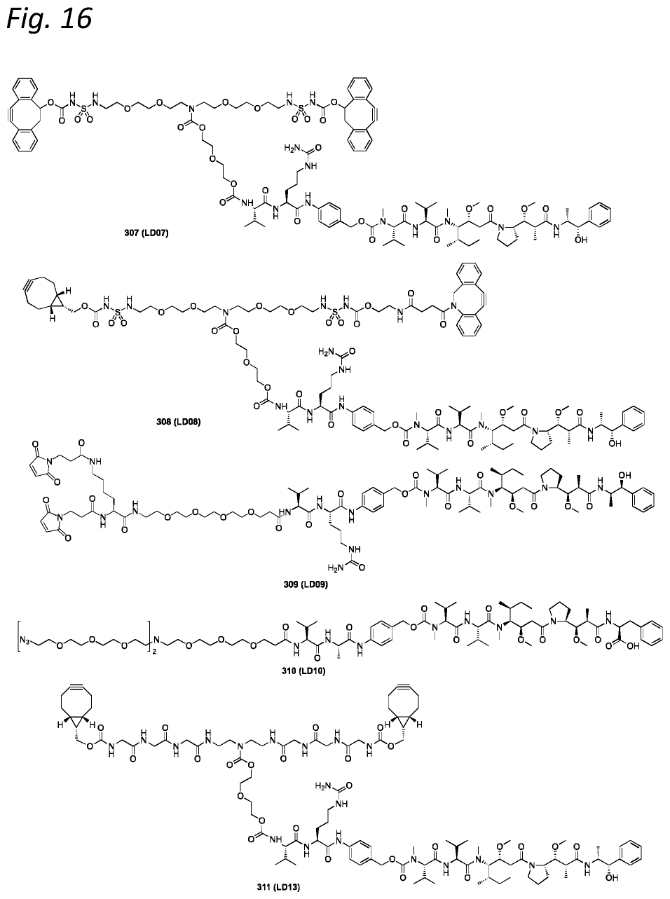

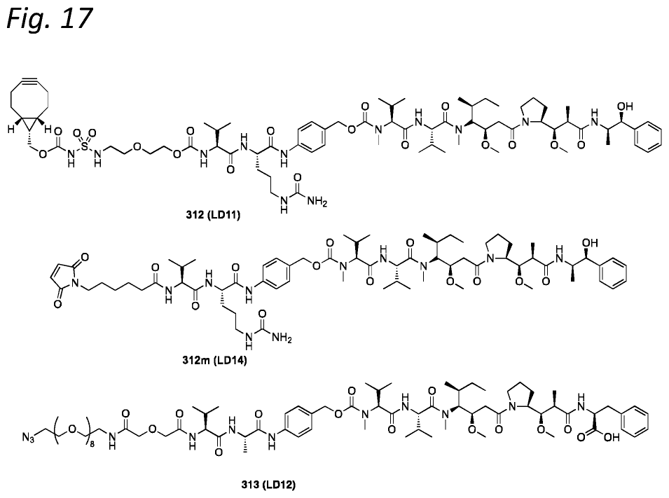

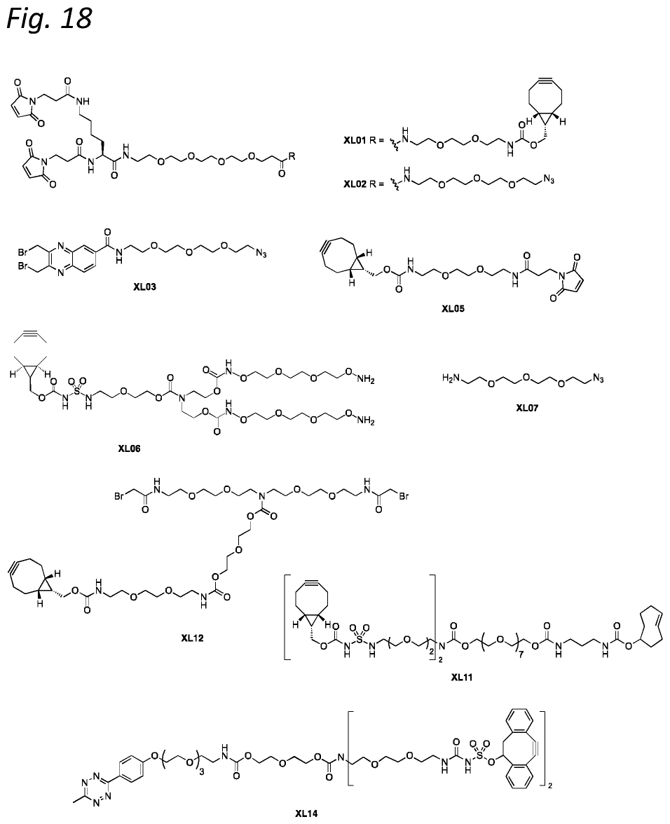

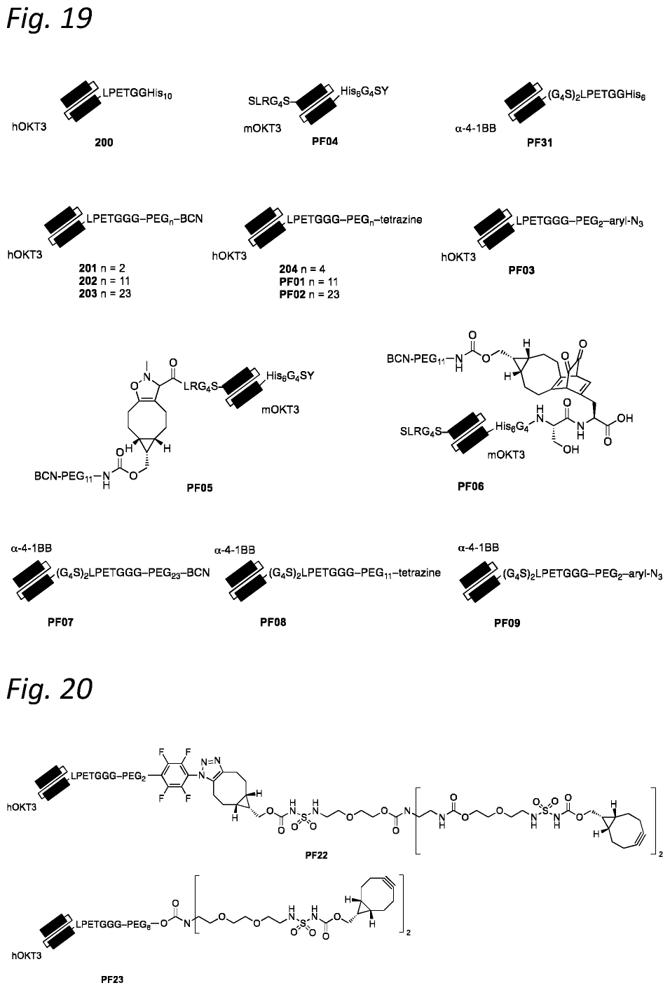

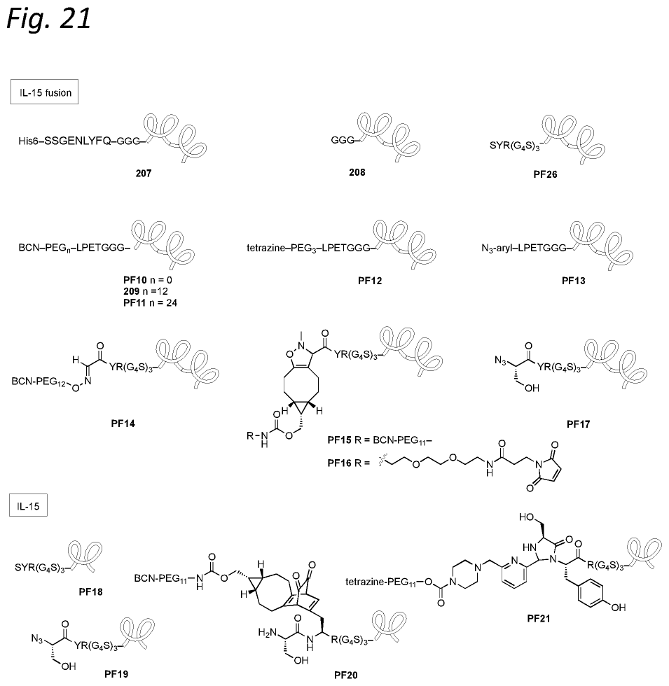

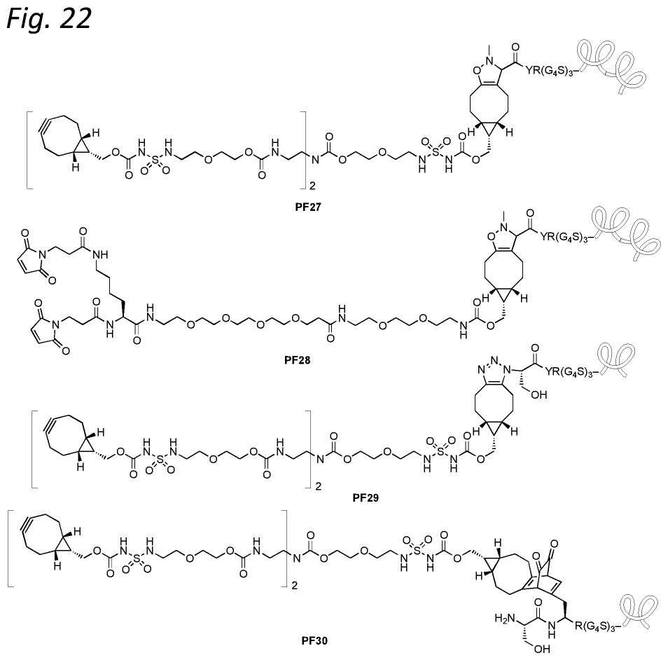

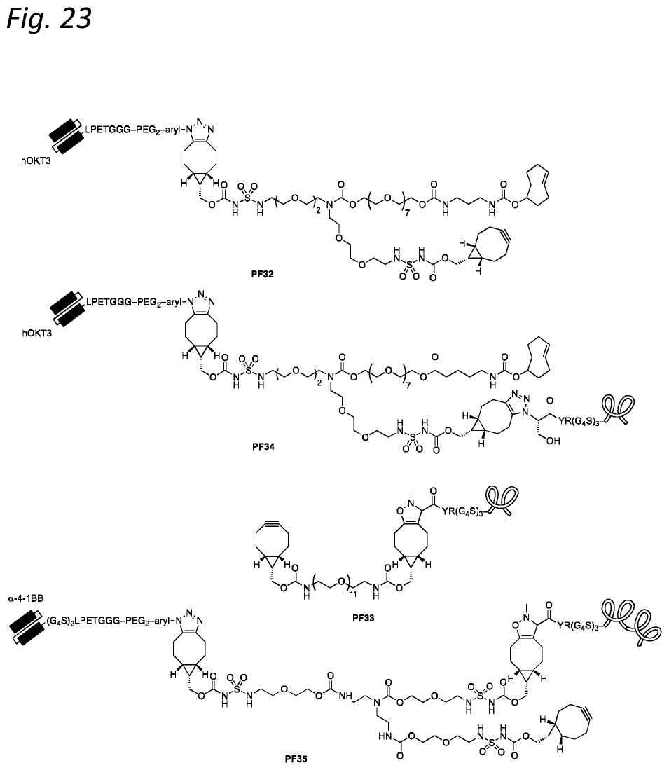

Su is a monosaccharide derivative, also referred to as sugar derivative. Preferably, the sugar derivative is able to be incorporated into the functionalized antibody by means of glycosyltransfer. More preferably, Su is Gal, Glc, GalNAc or GlcNAc, more preferably Gal or GalNAc, most preferably GalNAc. See FIG. 7 for some preferred examples of nucleotide-sugar derivatives that can be introduced. The term derivative refers to the monosaccharide being appropriately functionalized in order to connect to (G)ₑ and F.

Linker L⁴ connects BM with payload D¹. Linker L⁴ may be present (m=1) or absent (m=0), preferably L⁴ is present, i.e. m=1. Linker L⁵ connects BM with payload D². Linker L⁵ may be present (n=1) or absent (n=0), preferably L⁵ is present, i.e. n=1. Although linker L⁴ and L⁵ may be the same, it is preferred they are not, as they are used to connect two distinct payloads to BM.

In a preferred embodiment, L⁴ and L⁵ both individually have the structure (L-D), which is further defined below.

**(L²¹)ₙ-(L²²)ₒ-(L²³)ₚ-(L²⁴)_{q}-*** (L-D)

### Herein

the bond labelled ** is connected to BM, and the bond labelled *** is connected to D¹ or D²;
n, o, p and q are each independently 0 or 1;
L²¹, L²², L²³ and L²⁴ are linkers.

Like linker L⁶, linkers L⁴ and L⁵ may contain a connecting group Z that is formed during the synthesis of the multifunctional antibody construct. The payload may be connected to the linker-construct containing BM, which may either be before or after reaction of the linker-construct containing BM¹ or with a functionalized antibody. The connecting group within linker L⁴ and L⁵ may be formed at the junction any of the linking units L²¹, L²², L²³ and L²⁴, or may separately be present within linker L⁴ and L⁵. As such, linker L⁴ and L⁵ may be represented by -Sp-Z-Sp-, wherein Sp are individually spacers. These spacers typically contain part of the linker defined by structure (L-D). For example, L⁴ and L⁵ may be represented by -(L²¹)ₙ-(L²²)ₒ-(L²³)ₚ-Z-(L²⁴)_{q}- or -(L²¹)ₙ-(L²²)ₒ-(L²³)ₚ-(L²⁴)_{q}-Z—. Herein, Z may take any form, and is preferably as defined above.

Each of L⁴, L⁵ and L⁶ may contain one or more of L²¹, L²², L²³ and L²⁴. L²¹, L²², L²³ and L²⁴ are linkers that together form linker L⁴, L⁵ or L⁶ as further defined here below; n, o, p and q are individually 0 or 1. In a preferred embodiment, at least linkers L²¹ and L²² are present (i.e. n=1; o=1; p=0 or 1; q=0 or 1), more preferably linkers L²¹, L²² and L²³ are present and L²⁴ is either present or not (i.e. n=1; o=1; p=1; q=0 or 1). In one embodiment, linkers L²¹, L²², L²³ and L²⁴ are present (i.e. n=1; o=1; p=1; q=1). In one embodiment, linkers L²¹, L²² and L²³ are present and L²⁴ is not (i.e. n=1; o=1; p=1; q=0). In one embodiment n+o+p+q=1, 2, 3 or 4, preferably 2, 3 or 4, more preferably 3 or 4. In a preferred embodiment, L²² and L²³ are both present, i.e. o+p=2. Most preferably, n+o+p+q=3 or 4.

Linker L²¹ is either absent (n=0) or present (n=1). Preferably, linker L²¹ is present and n=1. L²¹ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups.

Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y} and NR¹⁵, wherein y is 0, 1 or 2, preferably y=2, and R¹⁵ is independently selected from the group consisting of hydrogen, halogen, C1-C₂₄ alkyl groups, C₆-C₂₄ (hetero)aryl groups, C₇-C₂₄ alkyl(hetero)aryl groups and C₇-C₂₄ (hetero)arylalkyl groups.

L²¹ may contain (poly)ethylene glycoldiamines (e.g. 1,8-diamino-3,6-dioxaoctane or equivalents comprising longer ethylene glycol chains), polyethylene glycol or polyethylene oxide chains, polypropylene glycol or polypropylene oxide chains and 1,z-diaminoalkanes wherein z is the number of carbon atoms in the alkane (z may for example be an integer in the range of 1-10).

In a preferred embodiment, Linker L²¹ comprises an ethylene glycol group, a carboxylic acid moiety, a sulfonate moiety, a sulfone moiety, a phosphate moiety, a phosphinate moiety, an amino group, an ammonium group or a sulfamide group.

In a preferred embodiment, Linker L²¹ comprises a sulfamide group, preferably a sulfamide group according to structure (23):

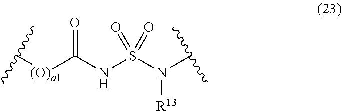

The wavy lines represent the connection to the remainder of the compound, for L⁴ and L⁵ typically to BM and L²², L²³, L²⁴, D¹ or D², preferably to BM and L²². Preferably, the (O)ₐC(O) moiety is connected to BM and the NR¹³ moiety to L²², L²³, L²⁴, D¹ or D², preferably to L²²; and for L⁶ typically to BM¹ and BM. Preferably, the (O)ₐC(O) moiety is connected to BM¹ and the NR¹³ moiety to BM.

In structure (23), a1=0 or 1, preferably a1=1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups.

Alternatively, R¹³ is connected to a payload, possibly via a spacer moiety. In one embodiment, R¹³ is also connected to payload D¹ or D², such that a cyclic structure is formed. For example, N is part of a piperazine moiety, which is connected to D¹ or D² via a carbon atom or nitrogen atom, preferably via the second nitrogen atom of the piperazine ring. Preferably, the cyclic structure, e.g. the piperazine ring, is connected to D¹ or D² via —(B)ₑ₁-(A)_{f1}-(B)_{g1}—C(O)— or via —(B)ₑ₁-(A)_{f1}-(B)_{g1}—C(O)-(L²²)ₒ-(L²³)ₚ-(L²⁴)_{q}-, as further defined below.

In a preferred embodiment, R¹³ is hydrogen or a C₁-C₂₀ alkyl group, more preferably R¹³ is hydrogen or a C₁-C₁₆ alkyl group, even more preferably R¹³ is hydrogen or a C₁-C₁₀ alkyl group, wherein the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or methyl, and most preferably R¹³ is hydrogen.

In a preferred embodiment, L²¹ is according to structure (24):

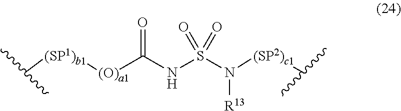

Herein, a and R¹³ are as defined above, Sp¹ and Sp² are independently spacer moieties and b1 and c1 are independently 0 or 1. Preferably, b1=0 or 1 and c1=1, more preferably b1=0 and c1=1. In one embodiment, spacers Sp¹ and Sp² are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR¹⁶, preferably O, wherein R¹⁶ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR¹⁶, preferably O and/or or S—S, wherein R³ is independently selected from the group consisting of hydrogen and C₁-C₄ alkyl groups, preferably hydrogen or methyl.

Preferred spacer moieties Sp¹ and Sp² thus include —(CH₂)ᵣ—, —(CH₂CH₂)ᵣ—, —(CH₂CH₂O)ᵣ, —(OCH₂CH₂)ᵣ, —(CH₂CH₂O)ᵣCH₂CH₂—, —CH₂CH₂(OCH₂CH₂)ᵣ, —(CH₂CH₂CH₂O)ᵣ, —(OCH₂CH₂CH₂)ᵣ, —(CH₂CH₂CH₂O)ᵣCH₂CH₂CH₂— and —CH₂CH₂CH₂(OCH₂CH₂CH₂)ᵣ—, wherein r is an integer in the range of 1 to 50, preferably in the range of 1 to 40, more preferably in the range of 1 to 30, even more preferably in the range of 1 to 20 and yet even more preferably in the range of 1 to 15. More preferably r is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

Alternatively, preferred linkers L²¹ may be represented by —(W)ₖ₁-(A)_{d1}-(B)ₑ₁-(A)_{f1}-(C(O))_{g1}—, wherein:
d1=0 or 1, preferably d1=1;
e1=an integer in the range 0-10, preferably e1=0, 1, 2, 3, 4, 5 or 6, preferably an integer in the range 1-10, most preferably e1=1, 2, 3 or 4;
f1=0 or 1, preferably f1=0;
wherein d1+e1+f1 is at least 1, preferably in the range 1-5; and preferably wherein d1+f1 is at least 1, preferably d1+f1=1.
g1=0 or 1, preferably g1=1;
k1=0 or 1, preferably k1=1;
A is a sulfamide group according to structure (23);
B is a —CH₂—CH₂—O— or a —O—CH₂—CH₂— moiety, or (B)ₑ₁ is a —(CH₂—CH₂—O)ₑ₃—CH₂—CH₂-moiety, wherein e3 is defined the same way as e1;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—, preferably wherein W is —OC(O)NH—, —C(O)(CH₂)ₘC(O)NH— or —C(O)NH—, and wherein m is an integer in the range 0-10, preferably m=0, 1, 2, 3, 4, 5 or 6, most preferably m=2 or 3;
preferably wherein L²¹ is connected to BM or BM¹ via (A)_{d1}-(B)ₑ₁ and to (L²²)ₒ via (C(O))_{g1}, preferably via C(O).

In the context of the present embodiment, the wavy lines in structure (23) represent the connection to the adjacent groups such as (W)ₖ₁, (B)ₑ₁ and (C(O))_{g1}. It is preferred that A is according to structure (23), wherein a1=1 and R¹³=H or a C₁-C₂₀ alkyl group, more preferably R¹³=H or methyl, most preferably R¹³=H.

Preferred linkers L²¹ are as follows:
(a) k1=0; d1=1; g1=1; f1=0; B=—CH₂—CH₂—O—; e1=1, 2, 3 or 4, preferably e1=2;
(b) k1=1; W=—C(O)(CH₂)ₘC(O)NH—; m=2; d1=0; (B)ₑ₁=—(CH₂—CH₂—O)ₑ₃—CH₂—CH₂—; f1=0; g1=1; e3=1, 2, 3 or 4, preferably e1=1.
(c) k1=1; W=—OC(O)NH—; d1=0; B=—CH₂—CH₂—O—; g1=1; f1=0; e1=1, 2, 3 or 4, preferably e1=2.
(d) k1=1; W=—C(O)(CH₂)ₘC(O)NH—; m=2; d1=0; (B)ₑ₁=—(CH₂—CH₂—O)ₑ₃—CH₂—CH₂—; f1=0; g1=1; e3=1, 2, 3 or 4, preferably e3=4.
(e) k1=1; W=—OC(O)NH—; d1=0; (B)ₑ₁=—(CH₂—CH₂—O)ₑ₃—CH₂—CH₂—; g1=1; f1=0; e3=1, 2, 3 or 4, preferably e3=4.
(f) k1=1; W=−(4-Ph)CH₂NHC(O)(CH₂)ₘC(O)NH—, m=3; d1=0; (B)ₑ₁=—(CH₂—CH₂—O)ₑ₃—CH₂—CH₂—; g1=1; f1=0; e3=1, 2, 3 or 4, preferably e3=4.
(g) k1=0; d1=0; g1=1; f1=0; B=—CH₂—CH₂—O—; e1=1, 2, 3 or 4, preferably e1=2.
(h) k1=1; W=—C(O)NH—; d1=0; g1=1; f1=0; B=—CH₂—CH₂—O—; e1=1, 2, 3 or 4, preferably e1=2.

In one embodiment, linker L²¹ comprises a branching nitrogen atom, which is located in the backbone between BM or BM¹ and (L²²)ₒ and which contains a further moiety D as substituent, which is preferably linked to the branching nitrogen atom via a linker. An example of a branching nitrogen atom is the nitrogen atom NR¹³ in structure (23), wherein R¹³ is connected to a second occurrence of D via a spacer moiety. Alternatively, a branching nitrogen atoms may be located within L²¹ according to structure —(W)ₖ₁-(A)_{d1}-(B)ₑ₁-(A)_{f1}-(C(O))_{g1}—. In one embodiment, L²¹ is represented by —(W)ₖ₁-(A)_{d1}-(B)ₑ₁-(A)_{f1}-(C(O))_{g1}—N*[-(A)_{d1}-(B)ₑ₁-(A)_{f1}-(C(O))_{g1}-]2, wherein A, B, W, d1, e1, f1, g1 and k1 are as defined above and individually selected for each occurrence, and N* is the branching nitrogen atoms, to which two instances of -(A)_{d1}-(B)ₑ₁-(A)_{f1}-(C(O))_{g1}— are connected. Herein, both (C(O))_{g1} moieties are connected to -(L²²)ₒ-(L²³)ₚ-(L²⁴)_{q}-D, wherein L²², L²³, L²⁴, o, p, q and D are as defined above and are each selected individually. In a most preferred embodiment, such a branching atom is not present and linker L²¹ does not contain a connection to a further payload.

Linker L²² is either absent (o=0) or present (o=1). Preferably, linker L²² is present and o=1. Linker L²² is a peptide spacer as known in the art, or derivative thereof (e.g. cyclobutane-1,1-dicarboxamide-Cit), preferably comprising 2-5 amino acids. The peptide spacer is preferably of structure (26):

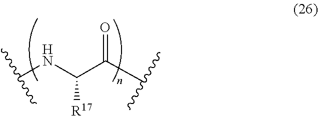

Herein, R¹⁷ represents an amino acid side chain and n is an integer in the range of 1-10, preferably n=1-8, more preferably n=1-5, most preferably n=1-4, preferably n=2.

Preferably, the peptide spacer is a dipeptide or tripeptide spacer as known in the art, preferably a dipeptide spacer. Suitable peptide spacers are selected from the group consisting of Val-Cit, Val-Ala, Val-Lys, Val-Arg, Val-Gln, AcLys-Val-Cit, AcLys-Val-Ala, Phe-Cit, Phe-Ala, Phe-Lys, Phe-Arg, Phe-Gln, Ala-Lys, Leu-Cit, Leu-Gln, Ile-Cit, Trp-Cit, Ala-Ala-Asn, Ala-Asn, Pro-Leu-Gly, Asn-Pro-Val, Lys-Ser-Gly-Arg-Ser-Asp-Asn-His, Pro-Val-Gly-Leu-Ile-Gly, Val-Lys-Gly, Gly-Gly-Gly, Gly-Gly-Phe-Gly and Lys, preferably from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, Ala-Asn, Pro-Leu-Gly, Asn-Pro-Val, Lys-Ser-Gly-Arg-Ser-Asp-Asn-His, Gly-Gly-Phe-Gly and Lys, preferably the peptide spacer is Val-Cit, Val-Ala or Ala-Ala-Asn. In one embodiment, the peptide spacer is Val-Cit. In one embodiment, the peptide spacer is Val-Ala.

These linkers are typically cleavable by a proteolytic enzyme, preferably a proteolytic enzyme selected from the group of cathepsins, granzymes, caspases, kallikereins, proprotein convertase subtilisins, furins, elastases, legumains, fibroblast activation proteins, tissue-type plasminogen activators, urokinases, matrix metallo proteases and matriptases.

In a preferred embodiment, the peptide spacer is represented by general structure (27):

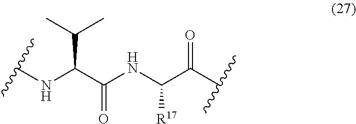

Herein, R¹⁷ represents an amino acid side chain, preferably R¹⁷=CH₃ (Val) or CH₂CH₂CH₂NHC(O)NH₂ (Cit).

The wavy lines in structure (26) and (27) indicate the connection to the remainder of the molecule, preferably the peptide spacer is connected via NH to (L²¹), typically via a linker, and via C(O) to the payload, typically via a linker.

The wavy lines indicate the connection to (L²¹)ₙ and (L²³)ₚ, preferably L²² according to structure (26) or (27) is connected to (L²¹)ₙ via NH and to (L²³)ₚ via C(O).

Linker L²³ is either absent (p=0) or present (p=1). Preferably, linker L²³ is present and p=1. Linker L²³ is a self-cleavable spacer, also referred to as self-immolative spacer. Preferably, L²³ is para-aminobenzyloxy (PAB) derivative, more preferably a PAB derivative according to structure (25).

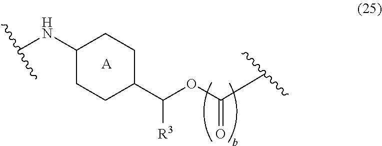

### Herein:

A is an optionally substituted 5- or 6-membered aromatic or heteroaromatic ring;
b is 0 or 1;
R³ is H, R⁴ or C(O)R⁴, wherein R⁴ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR⁵ wherein R⁵ is independently selected from the group consisting of hydrogen and C₁-C₄ alkyl groups.

Herein, the wavy lines indicate the connection to (L²²)ₒ and to (L²⁴)_{q}. Typically, the PAB derivative is connected via NH to (L²²)ₒ, and via O to (L²⁴)_{q}

R³ is H, R⁴ or C(O)R⁴, wherein R⁴ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR⁵ wherein R⁵ is independently selected from the group consisting of hydrogen and C₁-C₄ alkyl groups. Preferably, R⁴ is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R³ is H or C(O)R⁴, wherein R⁴=4-methyl-piperazine or morpholine. Most preferably, R³ is H. Preferably, A is a phenyl ring and b=1.

Linker L²⁴ is either absent (q=0) or present (q=1). Preferably, linker L²⁴ is present and q=1. Linker L²⁴ is an aminoalkanoic acid spacer, i.e. —N—(Cₕ-alkylene)-C(O)—, wherein h is an integer in the range 1 to 20, preferably 1-10, most preferably 1-6. Herein, the aminoalkanoic acid spacer is typically connected to L²³ via the nitrogen atom and to the payload via the carbonyl moiety. Preferred linkers L²⁴ are selected from 6-aminohexanoic acid (Ahx, h=6), β-alanine (h=2) and glycine (Gly, h=1), even more preferably 6-aminohexanoic acid or glycine. In one embodiment, L²⁴=6-aminohexanoic acid. In one embodiment, L²⁴=glycine. Alternatively, linker L²⁴ is a an ethyleneglycol spacer according to the structure —N—(CH₂—CH₂—O)ₑ₆—(CH₂)ₑ₇—(C(O)—, wherein e6 is an integer in the range 1-10 and e7 is an integer in the range 1-3.

### Branching Moiety

A “branching moiety” in the context of the present invention refers to a moiety that is embedded in a linker connecting three moieties. In other words, the branching moiety comprises at least three bonds to other moieties. Branching moiety BM comprises one bond to payload D¹, typically via linker L⁴, one bond to payload D², typically via linker L⁵, and one bond to antibody Ab via linker L³. Branching moiety BM¹ comprises one bond to branching moiety BM via linker L⁶, and two bonds to antibody Ab via (L⁸)_{q}-Z-(L⁷)ₚ. The branching moiety defined here below equally applies to BM and BM¹.

Any moiety that contains at least three bonds to other moieties is suitable as branching moiety in the context of the present invention. Suitable branching moieties include a carbon atom (BM-1), a nitrogen atom (BM-3), a phosphorus atom (phosphine (BM-5) and phosphine oxide (BM-6)), aromatic rings such as a phenyl ring (e.g. BM-7) or a pyridyl ring (e.g. BM-9), a (hetero)cycle (e.g. BM-11 and BM-12) and polycyclic moieties (e.g. BM-13, BM-14 and BM-15). Preferred branching moieties are selected from carbon atoms, nitrogen atoms and phenyl rings, most preferably the branching moiety is a carbon atom or a nitrogen atom. Structures (BM-1) to (BM-15) are depicted here below, wherein the three branches, i.e. bonds to other moieties as defined above, are indicated by * (a bond labelled with *).

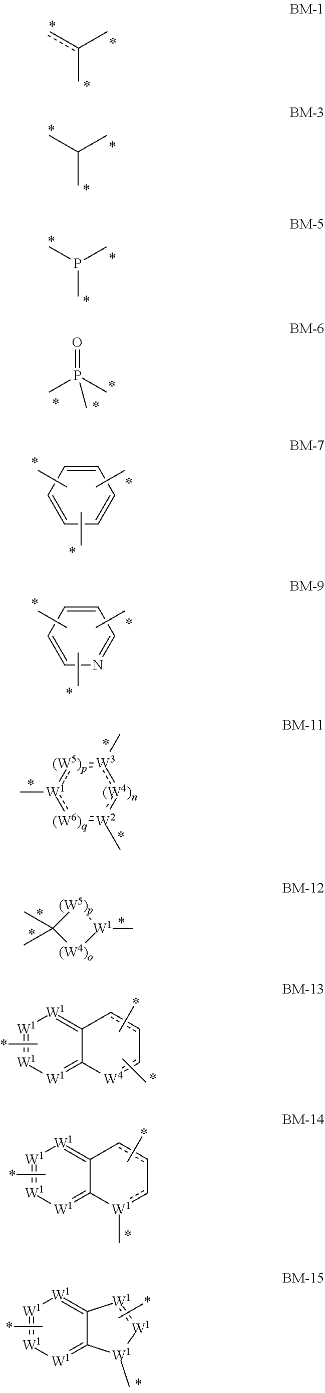

In (BM-1), one of the branches labelled with * may be a single or a double bond, indicated with [symbol] In (BM-11) to (BM-15), the following applies:
each of n, p, q and q is individually an integer in the range of 0-5, preferably 0 or 1, most preferably 1;
each of W¹, W² and W³ is independently selected from C(R²¹)_{w} and N;
each of W⁴, W⁵ and W⁶ is independently selected from C(R²¹)_{w+1}, N(R²²)_{w}, O and S;
each [symbol] represents a single or double bond;
w is 0 or 1 or 2, preferably 0 or 1;
each R²¹ is independently selected from the group consisting of hydrogen, OH, C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR³ wherein R³ is independently selected from the group consisting of hydrogen and C₁-C₄ alkyl groups; and
each R²² is independently selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR³ wherein R³ is independently selected from the group consisting of hydrogen and C₁-C₄ alkyl groups.

The skilled person appreciates that the values of w and the bond order of the bonds represented by [symbol] are interdependent. Thus, whenever an occurrence of W is bonded to an endocyclic double bond, w=1 for that occurrence of W, while whenever an occurrence of W is bonded to two endocyclic single bonds, w=0 for that occurrence of W. For BM-12, at least one of o and p is not 0.

Representative examples of branching moieties according to structure (BM-11) and (BM-12) include cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, cyclooctenyl, aziridine, azetidine, diazetidine, oxetane, thietane, pyrrolidine, dihydropyrrolyl, tetrahydrofuranyl, dihydrofuranyl, thiolanyl, imidazolinyl, pyrazolidinyl, oxazolidinyl, isoxazolidinyl, thiazolidinyl, isothiazolidinyl, dioxolanyl, dithiolanyl, piperidinyl, oxanyl, thianyl, piperazinyl, morpholino, thiomorpholino, dioxanyl, trioxanyl, dithyanyl, trithianyl, azepanyl, oxepanyl and thiepanyl. Preferred cyclic moieties for use as branching moiety include cyclopropenyl, cyclohexyl, oxanyl (tetrahydropyran) and dioxanyl. The substitution pattern of the three branches determines whether the branching moiety is of structure (BM-11) or of structure (BM-12).

Representative examples of branching moieties according to structure (BM-13) to (BM-15) include decalin, tetralin, dialin, naphthalene, indene, indane, isoindene, indole, isoindole, indoline, isoindoline, and the like.

In a preferred embodiment, the branching moiety is a carbon atom. In case the carbon atom is according to structure (BM-1) and has all four bonds to distinct moieties, the carbon atom is chiral. The stereochemistry of the carbon atom is not crucial for the present invention, and may be S or R. The same holds for the phosphine (BM-6). Most preferably, the carbon atom is according to structure (BM-1). One of the branches indicated with * in the carbon atom according to structure (BM-1) may be a double bond, in which case the carbon atom may be part of an alkene or imine.

In case the branching moiety is a carbon atom, the carbon atom may be part of a larger functional group, such as an acetal, a ketal, a hemiketal, an orthoester, an orthocarbonate ester, an amino acid and the like. This also holds in case the branching moiety is a nitrogen or phosphorus atom, in which case it may be part of an amide, an imide, an imine, a phosphine oxide (as in BM-6) or a phosphotriester.

In a preferred embodiment, the branching moiety is a phenyl ring. Most preferably, the phenyl ring is according to structure (BM-7). The substitution pattern of the phenyl ring may be of any regiochemistry, such as 1,2,3-substituted phenyl rings, 1,2,4-substituted phenyl rings, or 1,3,5-substituted phenyl rings. To allow optimal flexibility and conformational freedom, it is preferred that the phenyl ring is according to structure (BM-7), most preferably the phenyl ring is 1,3,5-substituted. The same holds for the pyridine ring of (BM-9).

In a preferred embodiment, the branching moiety is selected from a carbon atom, a nitrogen atom, a phosphorus atom, a (hetero)aromatic ring, a (hetero)cycle or a polycyclic moiety.

### Preferred Multifunctional Antibody Constructs

Especially preferred multifunctional antibody constructs according to the invention have structure (3), (4), (5), (6) or (7). In one embodiment, the multifunctional antibody construct are according to structure (3), (4), (5) or (6). In one embodiment, the multifunctional antibody construct are according to structure (3), (4) or (6). In one embodiment, the multifunctional antibody construct are according to structure (3) or (4).

In one embodiment, the multifunctional antibody construct has structure (3):

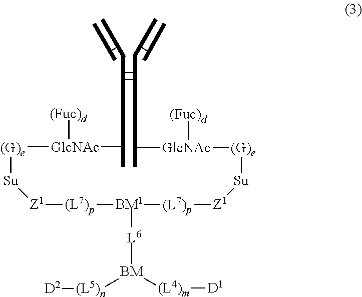

wherein:
BM and BM¹ are branching moieties;
d, m, n and p are each independently 0 or 1;
D¹ and D² are two distinct payloads selected from the group consisting of polypeptides, small molecules, cytotoxins and oligonucleotides, wherein at least one of D¹ and D² is a polypeptide;
e is an integer in the range of 0-10;
L⁴, L⁵, L⁶ and L⁷ are linkers;
Su is a monosaccharide;
G is a monosaccharide moiety;
GlcNAc is an N-acetylglucosamine moiety;
Fuc is a fucose moiety;
Z¹ is a connecting groups.

For the multifunctional antibody construct has structure (3), it is preferred that n=m=1.

In one embodiment, the multifunctional antibody construct has structure (4):

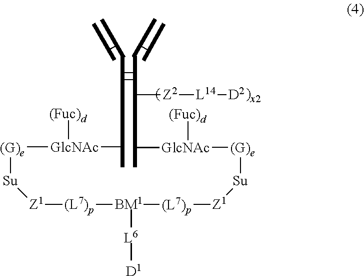

wherein:
D¹ and D² are two distinct payloads selected from the group consisting of polypeptides, small molecules, cytotoxins and oligonucleotides, wherein at least one of D¹ and D² is a polypeptide;
d and p are each independently 0 or 1;
e is an integer in the range of 0-10;
x2 is an integer in the range of 1-8;
L⁶, L⁷ and L¹⁴ are linkers;
Su is a monosaccharide;
G is a monosaccharide moiety;
GlcNAc is an N-acetylglucosamine moiety;
Fuc is a fucose moiety;
BM¹ is a branching moiety;
Z¹ and Z² are connecting groups.

For the multifunctional antibody construct has structure (4), it is preferred that L¹⁴ is according to structure (L-D) as defined above. It is further preferred that connecting group Z¹ is obtained by a cycloaddition and connecting group Z² via a nucleophilic reaction.

In one embodiment, the multifunctional antibody construct has structure (5):

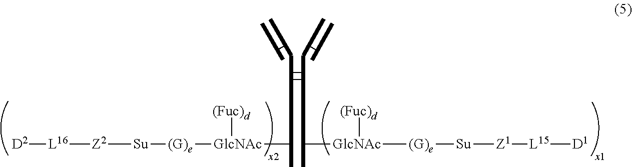

wherein:
D¹ and D² are two distinct payloads selected from the group consisting of polypeptides, small molecules, cytotoxins and oligonucleotides, wherein at least one of D¹ and D² is a polypeptide;
x1 and x2 are each independently 1, 2 or 3.
each d is independently 0 or 1;
e is an integer in the range of 0-10;
L¹⁵ and L¹⁶ are linkers;
Su is a monosaccharide;
G is a monosaccharide moiety;
GlcNAc is an N-acetylglucosamine moiety;
Fuc is a fucose moiety;
Z¹ and Z² are connecting groups.

For the multifunctional antibody construct has structure (5), it is preferred that L¹⁵ and L¹⁶ are both individually according to structure (L-D) as defined above. It is further preferred that connecting group Z¹ and Z² are both individually obtained by a cycloaddition. Typically, the sum of x1 and x2 is at most 4, preferably 2 or 4. More preferably, x1 and x2 are the same and are 1 or 2.

In one embodiment, the multifunctional antibody construct has structure (6):

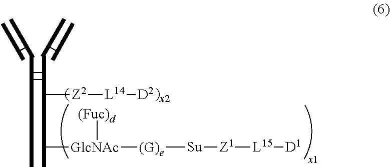

wherein:
D¹ and D² are two distinct payloads selected from the group consisting of polypeptides, small molecules, cytotoxins and oligonucleotides, wherein at least one of D¹ and D² is a polypeptide;
x1 is an integer in the range of 1-8 and x2 is an integer in the range of 1-4, wherein x1+x2=2-10
each d is independently 0 or 1;
e is an integer in the range of 0-10;
L¹⁴ and L¹⁵ are linkers;
Su is a monosaccharide;
G is a monosaccharide moiety;
GlcNAc is an N-acetylglucosamine moiety;
Fuc is a fucose moiety;
Z¹ and Z² are connecting groups.

For the multifunctional antibody construct has structure (6), it is preferred that L¹⁴ and L¹⁵ are both individually according to structure (L-D) as defined above. It is further preferred that connecting group Z¹ is obtained by a cycloaddition and connecting group Z² via a nucleophilic reaction. x2 is an integer in the range of 1-4, preferably x2 is 1 or 2, most preferably x2 is 2.

In one embodiment, the multifunctional antibody construct has structure (7):

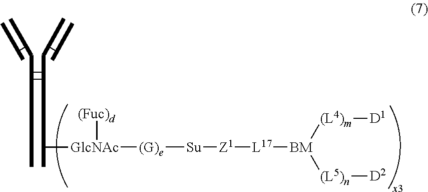

wherein:
D¹ and D² are two distinct payloads selected from the group consisting of polypeptides, small molecules, cytotoxins and oligonucleotides, wherein at least one of D¹ and D² is a polypeptide;
x3 is an integer in the range of 1-4;
each d is independently 0 or 1;
e is an integer in the range of 0-10;
L⁴, L⁵ and L¹⁷ are linkers;
BM is a branching moiety;
Su is a monosaccharide;
G is a monosaccharide moiety;
GlcNAc is an N-acetylglucosamine moiety;
Fuc is a fucose moiety;
Z¹ and Z² are connecting groups.

For the multifunctional antibody construct has structure (7), it is preferred that L¹⁷ is according to structure (L-D) as defined above. It is further preferred that connecting group Z¹ is obtained by a cycloaddition. x2 is an integer in the range of 1-4, preferably x3 is 2 or 4, most preferably x3 is 2. Further, it is preferred that m=n=1.

### Application

The multifunctional antibody constructs according to the invention are especially suitable in the treatment of e.g. cancer, by combining multiple modes-of-action against the cancer in a single molecule. The invention thus further concerns the use of the multifunctional antibody construct according to the invention in medicine. In a further aspect, the invention also concerns a method of treating a subject in need thereof, comprising administering the multifunctional antibody construct according to the invention to the subject. The method according to this aspect can also be worded as the multifunctional antibody construct according to the invention for use in treatment. The method according to this aspect can also be worded as use of the multifunctional antibody construct according to the invention for the manufacture of a medicament. Herein, administration typically occurs with a therapeutically effective amount of the multifunctional antibody construct according to the invention.

Because of the multifunctional nature of the antibody constructs according to the invention, treatment will bring both payloads D¹ and D² towards the tumour, because of the targeting nature of the antibody. As such, co-administration of one drug based on payload D¹, such as an antibody-drug conjugate, and a separately administered additional drug (e.g. a chemotherapy drug or checkpoint inhibitor) is no longer needed. In addition, the multifunctional antibody constructs according to the invention allow a carefully tailored stoichiometry with regards to binding to the tumour-associated antigen, the number of immune cell-engaging polypeptides or checkpoint inhibitors and the number of other payloads such as small molecule payloads. Finally, the multifunctional antibody constructs according to the invention may be particularly suitable for the treatment of patients that have become insensitive to treatment with conventional therapy due to multidrug-resistance of the tumour. Hence, in one especially preferred embodiment, the subject is a cancer patient who has developed a multidrug-resistance towards conventional cancer therapy.

The invention further concerns a method for the treatment of a specific disease in a subject in need thereof, comprising the administration of the multifunctional antibody construct according to the invention as defined above. The specific disease may be selected from cancer, a viral infection, a bacterial infection, a neurological disease, an autoimmune disease, an eye disease, hypercholesterolemia and amyloidosis, more preferable from cancer and a viral infection, most preferably the disease is cancer. The subject in need thereof is typically a cancer patient. The use of antibody-conjugates is well-known in such treatments, especially in the field of cancer treatment, and the multifunctional antibody constructs according to the invention are especially suited in this respect. In the method according to this aspect, the multifunctional antibody construct is typically administered in a therapeutically effective amount. The present aspect of the invention can also be worded as a multifunctional antibody construct according to the invention for use in the treatment of a specific disease in a subject in need thereof, preferably for the treatment of cancer. In other words, this aspect concerns the use of a multifunctional antibody construct according to the invention for the preparation of a medicament or pharmaceutical composition for use in the treatment of a specific disease in a subject in need thereof, preferably for use in the treatment of cancer.

It is preferred that the multifunctional antibody construct according to the invention is Fc-silent, i.e. does not significantly bind to Fc gamma receptors CD16 when used in clinically. This is the case when G is absent, i.e. that e=0. Preferably, also the binding towards CD32 and CD64 is significantly reduced.

The invention further concerns a method for associating an immune cell with a tumour cell. A sample comprising the immune cell and the tumour cell is contacted with the multifunctional antibody construct according to the invention. The immune cell binds to the immune cell-engaging peptide and the tumour cell to the antibody, as such forming a complex association of tumour cell, immune cell and multifunctional antibody construct. In an alternative embodiment, the method according to the invention is for simultaneous binding to a tumour cell and a checkpoint inhibitor. Herein, a sample comprising the checkpoint inhibitor and the tumour cell is contacted with the multifunctional antibody construct according to the invention. The checkpoint inhibitor binds to the checkpoint inhibitor-targeting polypeptide and the tumour cell to the antibody, as such forming a complex association of tumour cell, checkpoint inhibitor and multifunctional antibody construct. This contacting may take place in a sample in vitro, e.g. taking from a subject, or in vivo within a subject, in which case the multifunctional antibody construct according to the invention is administered to the subject.

Administration in the context of the present invention refers to systemic administration. Hence, in one embodiment, the methods defined herein are for systemic administration of the multifunctional antibody construct. In view of the specificity of the multifunctional antibody constructs, they can be systemically administered, and yet exert their activity in or near the tissue of interest (e.g. a tumour). Systemic administration has a great advantage over local administration, as the drug may also reach tumour metastasis not detectable with imaging techniques and it may be applicable to haematological tumours.

The invention further concerns a pharmaceutical composition comprising the antibody-payload conjugate according to the invention and a pharmaceutically acceptable carrier.

### EXAMPLES

The invention is illustrated by the following examples.

### General Procedures

Chemicals were purchased from commonly used suppliers (Sigma-Aldrich, Acros, Alfa Aesar, Fluorochem, Apollo Scientific Ltd and TCI) and were used without further purification. Solvents (including dry solvents) for chemical transformations, work-up and chromatography were purchased from Aldrich (Dorset, UK) at HPLC grade, and used without further distillation. Silica gel 60 F254 analytical thin layer chromatography (TLC) plates were from Merck (Darmstadt, Germany) and visualized under UV light, with potassium permanganate stain or anisaldehyde stain. Chromatographic purifications were performed using Acros silica gel (0.06-0.200, 60A) or prepacked columns (Screening Devices) in combination with a Buchi Sepacore C660 fraction collector (Flawil, Switzerland). Reversed phase HPLC purifications were performed using an Agilent 1200 system equipped with a Waters Xbridge C18 column (5 μm OBD, 30×100 mm, PN186002982). Deuterated solvents used for NMR spectroscopy were obtained from Cambridge Isotope Laboratories. Bis-mal-Lys-PEG₄-TFP ester (177) was obtained from Quanta Biodesign, 0-(2-aminoethyl)-O′-(2-azidoethyl)diethylene glycol (XL07) and compounds 344 and 179 were obtained from Broadpharm, 2,3-bis(bromomethyl)-6-quinoxalinecarboxylic acid (178) was obtained from ChemScene and 32-azido-5-oxo-3,9,12,15,18,21,24,27,30-nonaoxa-6-azadotriacontanoic acid (348) was obtained from Carbosynth. Compound 313m (LD14, maleimidocaproyl-Val-Cit-PABC-MMAE) was obtained from Levena Biopharma. Adcetris and Kadcyla were obtained from the pharmacy.

### General Procedure for Mass Spectral Analysis of Monoclonal Antibodies and ADCs

Prior to mass spectral analysis, IgG was treated with IdeS (Fabricator™) for analysis of the Fc/2 fragment. A solution of 20 μg (modified) IgG was incubated for 1 hour at 37° C. with 0.5 μL IdeS (50 U/μL) in phosphate-buffered saline (PBS) pH 6.6 in a total volume of 10 μL. Samples were diluted to 40 μL followed by electrospray ionization time-of-flight (ESI-TOF) analysis on a JEOL AccuTOF. Deconvoluted spectra were obtained using Magtran software.

### General Procedure for Analytical RP-HPLC

Prior to RP-HPLC analysis, IgG was treated with IdeS, which allows analysis of the Fc/2 fragment. A solution of (modified) IgG (100 μL, 1 mg/mL in PBS pH 7.4) was incubated for 1 hour at 37° C. with 1.5 μL IdeS/Fabricator™ (50 U/μL) in phosphate-buffered saline (PBS) pH 6.6. The reaction was quenched by adding 49% acetonitrile, 49% water, 2% formic acid (100 μL). RP-HPLC analysis was performed on an Agilent 1100 series (Hewlett Packard). The sample (10 μL) was injected with 0.5 mL/min onto a ZORBAX Poroshell 300SB-C8 column (1×75 mm, 5 μm, Agilent) with a column temperature of 70° C. A linear gradient was applied in 25 minutes from 30 to 54% acetonitrile and water in 0.1% TFA.

### General Procedure for Analytical HPLC-SEC

HPLC-SEC analysis was performed on an Agilent 1100 series (Hewlett Packard). The sample (4 μL, 1 mg/mL) was injected with 0.86 mL/min onto a Xbridge BEH200A (3.5 μM, 7.8×300 mm, PN186007640 Waters) column. Isocratic elution using 0.1 M sodium phosphate buffer pH 6.9 (NaH₂PO₄/Na₂HPO₄) was performed for 16 minutes.

### Example 1. Synthesis of Compound 102

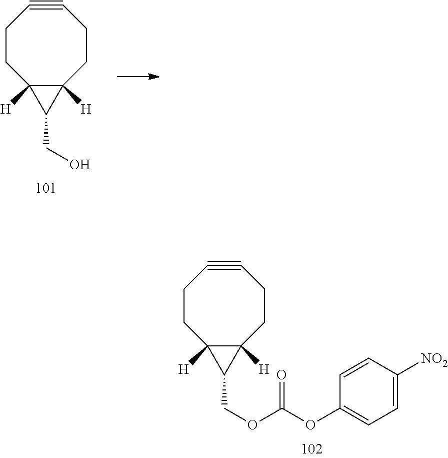 [SMILES: OC[C@H]1[C@H]2CCC#CCC[C@@H]12 ; O=C(OC[C@@H]1[C@@H]2CCC#CCC[C@@H]21)Oc1ccc([N+](=O)[O-])cc1]

To a cooled (0° C.) solution of 4-nitrophenyl chloroformate (30.5 g, 151 mmol) in DCM (500 mL) was added pyridine (24.2 mL, 23.7 g, 299 mmol). A solution of BCN-OH (101, 18.0 g, 120 mmol) in DCM (200 mL) was added dropwise to the reaction mixture. After the addition was completed, a saturated aqueous solution of NH₄Cl (500 mL) and water (200 mL) were added. After separation, the aqueous phase was extracted with DCM (2×500 mL). The combined organic phases were dried (Na₂SO₄) and concentrated. The crude material was purified by silica gel chromatography and the desired product 102 was obtained as an off-white solid (18.7 g, 59 mmol, 39%). ¹H NMR (400 MHz, CDCl₃) δ (ppm) 8.32-8.23 (m, 2H), 7.45-7.34 (m, 2H), 4.40 (d, J=8.3 Hz, 2H), 2.40-2.18 (m, 6H), 1.69-1.54 (m, 2H), 1.51 (quintet, J=9.0 Hz, 1H), 1.12-1.00 (m, 2H)

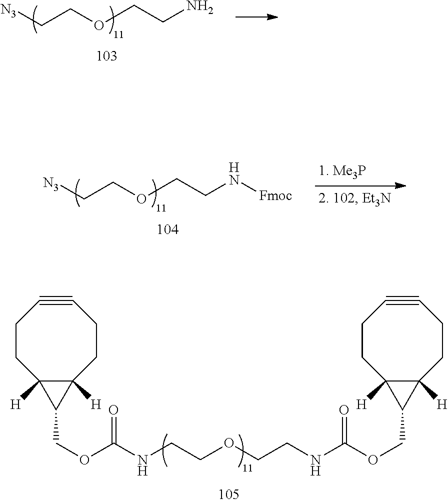

### Example 2. Synthesis of Compound 104

To a cooled solution (−5° C.) of azido-PEG₁₁-amine (103) (182 mg, 0.319 mmol) in THE (3 mL) were added a 10% aqueous NaHCO₃ solution (1.5 mL) and 9-fluorenylmethoxycarbonyl chloride (99 mg, 0.34 mmol) dissolved in THE (2 mL). After 2 h, EtOAc (20 mL) was added and the mixture was washed with brine (2×6 mL), dried over MgSO₄, and concentrated. Purification by silica gel column chromatography (0→11% MeOH in DCM) gave 104 as a clear oil in 98% yield (251 mg, 0.316 mmol). LCMS (ESI+) calculated for C₃₉H₆₀N₄O₁₃⁺ (M+Na⁺) 815.42 found 815.53.

### Example 3. Synthesis of Compound 105

A solution of 104 (48 mg, 0.060 mmol) in THE (3 mL) and water (0.2 mL) was prepared and cooled down to 0° C. Trimethylphosphine (1 M in toluene, 0.24 mL, 0.24 mmol) was added and the mixture was left stirring for 23 h. The water was removed via extraction with DCM (6 mL). To this solution, (1R,8S,9s)-bicyclo[6.1.0]non-4-yn-9-ylmethyl (4-nitrophenyl) carbonate (102) (25 mg, 0.079 mmol) and triethylamine (10 μL, 0.070 mmol) were added. After 27 h, the mixture was concentrated and the residue was dissolved in DMF (3 mL), followed by the addition of piperidine (400 μL). After 1 h, the mixture was concentrated and the residue was purified by silica gel column chromatography (0→21% MeOH in DCM), which gave 105 as a colorless oil (8.3 mg, 0.0092 mmol). LCMS (ESI+) calculated for C₄₆H₇₆N₂O₁₅⁺ (M+NH₄⁺) 914.52 found 914.73.

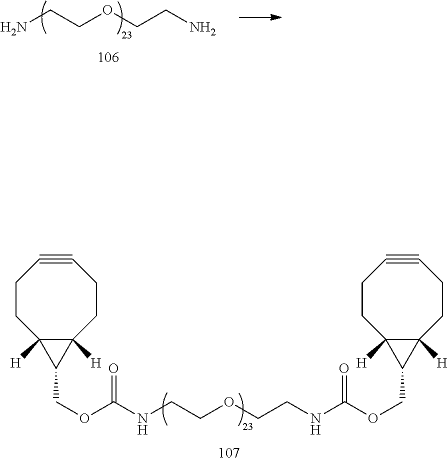

### Example 4. Synthesis of Compound 107

A solution of (1R,8S,9s)-bicyclo[6.1.0]non-4-yn-9-ylmethyl (4-nitrophenyl) carbonate (102) (4.1 mg, 0.013 mmol) in dry DCM (500 μL) was slowly added to a solution of amino-PEG23-amine (106) (12.3 mg, 0.0114 mmol) in dry DCM (500 μL). After 20 h, the mixture was concentrated and the residue was purified by silica gel column chromatography (0→25% MeOH in DCM) which gave the desired compound 107 in 73% yield (12 mg, 0.0080 mmol). LCMS (ESI+) calculated for C₇₀H₁₂₄N₂O₂₇⁺ (M+NH₄⁺) 1443.73 found 1444.08.

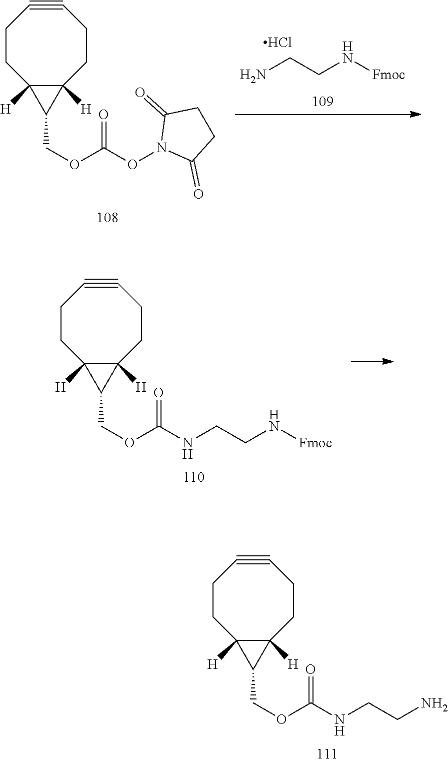

### Example 5. Synthesis of Compound 108

To a solution of BCN-OH (101, 21.0 g, 0.14 mol) in MeCN (450 mL) were added disuccinimidyl carbonate (53.8 g, 0.21 mol) and triethylamine (58.5 mL, 0.42 mol). After the mixture was stirred for 140 minutes, it was concentrated in vacuo and the residue was co-evaporated once with MeCN (400 mL). The residue was dissolved in EtOAc (600 mL) and washed with H₂O (3×200 mL). The organic layer was dried over Na₂SO₄ and concentrated in vacuo. The residue was purified by silica gel column chromatography (0→4% EtOAc in DCM) and gave 108 (11.2 g, 38.4 mmol, 27% yield) as a white solid. ¹H NMR (400 MHz, CDCl₃): δ (ppm) 4.45 (d, 2H, J=8.4 Hz), 2.85 (s, 4H), 2.38-2.18 (m, 6H), 1.65-1.44 (m, 3H), 1.12-1.00 (m, 2H).

### Example 6. Synthesis of Compound 110

To a solution of (1R,8S,9s)-bicyclo[6.1.0]non-4-yn-9-ylmethyl N-succinimidyl carbonate (108) (500 mg, 1.71 mmol) in DCM (15 mL) were added triethylamine (718 uL, 5.14 mmol) and mono-Fmoc ethylenediamine hydrochloride (109) (657 mg, 2.06 mmol). The mixture was stirred for 45 min, diluted with EtOAc (150 mL) and washed with a 50% saturated aqueous NH₄Cl solution (50 mL). The aqueous layer was extracted with EtOAc (50 mL) and the combined organic layers were washed with H₂O (10 mL). The combined organic extracts were concentrated in vacuo and the half of the residue was purified by silica gel column chromatography (0→3% MeOH in DCM) which gave the desired compound 110 in 42% yield (332 mg, 0.72 mmol). ¹H NMR (400 MHz, CDCl₃) δ (ppm) 7.77 (d, J=7.5 Hz, 2H), 7.59 (d, J=7.4 Hz, 2H), 7.44-7.37 (m, 2H), 7.36-7.28 (m, 2H), 5.12 (br s, 1H), 4.97 (br s, 1H), 44.41 (d, J=6.8 Hz, 2H), 4.21 (t, J=6.7 Hz, 1H), 4.13 (d, J=8.0 Hz, 2H), 3.33 (br s, 4H), 2.36-2.09 (m, 6H), 1.67-1.45 (m, 2H), 1.33 (quintet, J=8.6 Hz, 1H), 1.01-0.85 (m, 2H). LCMS (ESI+) calculated for C₂₈H₃₁N₂O₄⁺ (M+H⁺) 459.23 found 459.52.

### Example 7. Synthesis of Compound 111

Compound 110 (327 mg, 0.713 mmol) was dissolved in DMF (6 mL) and piperidine (0.5 mL) was added. After 2 h, the mixture was concentrated and the residue was purified by silica gel column chromatography (0→32% 0.7 N NH₃ MeOH in DCM), which gave the desired compound 111 as a yellow oil (128 mg, 0.542 mmol, 76%). ¹H-NMR (400 MHz, CDCl₃) δ (ppm, rotamers) 5.2 (bs, 1H), 4.15 (d, J=8.0 Hz, 2H), 3.48-3.40 (m, % H), 3.33-3.27 (m, % H), 3.27-3.19 (m, 1% H), 2.85-2.80 (m, 1% H), 2.36-2.17 (m, 6H), 1.67-1.50 (m, 2H), 1.36 (quintet, J=8.5 Hz, 1H), 1.01-0.89 (m, 2H).

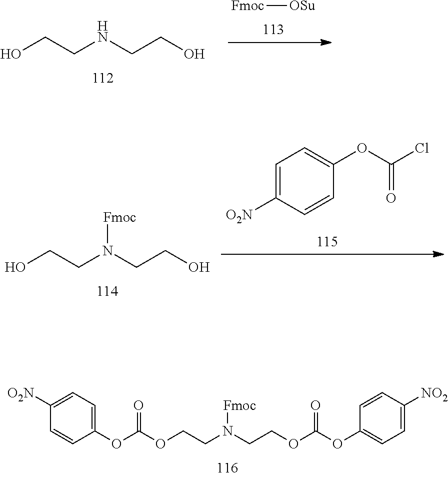

### Example 8. Synthesis of Compound 114

To a solution of diethanolamine (112) (208 mg, 1.98 mmol) in water (20 mL) were added MeCN (20 mL), NaHCO₃ (250 mg, 2.97 mmol) and a solution of Fmoc-OSu (113) (601 mg, 1.78 mmol) in MeCN (20 mL). The mixture was stirred for 2 h and DCM (50 mL) was added. After separation, the organic phase was washed with water (20 mL), dried (Na₂SO₄) and concentrated. The desired product 114 was obtained as a colorless thick oil (573 mg, 1.75 mmol, 98%). ¹H NMR (400 MHz, CDCl₃) δ (ppm) 7.79-7.74 (m, 2H), 7.60-7.54 (m, 2H), 7.44-7.37 (m, 2H), 7.36-7.30 (m, 2H), 4.58 (d, J=5.4 Hz, 2H), 4.23 (t, J=5.3 Hz, 1H), 3.82-3.72 (m, 2H), 3.48-3.33 (m, 4H), 3.25-3.11 (m, 2H).

### Example 9. Synthesis of Compound 116

To a solution of 114 (567 mg, 1.73 mmol) in DCM (50 mL) were added 4-nitrophenyl chloroformate (115) (768 mg, 3.81 mmol) and Et₃N (1.2 mL, 875 mg). The mixture was stirred for 18 h and concentrated. The residue was purified by silica gel chromatography (0%→10% MeOH in DCM, then 20%→70% EtOAc in heptane, which afforded 32 mg (49 μmol, 2.8%) of the desired product 116. ¹H NMR (400 MHz, CDCl₃) δ (ppm) 8.31-8.20 (m, 4H), 7.80-7.74 (m, 2H), 7.59-7.54 (m, 2H), 7.44-7.37 (m, 2H), 7.37-7.29 (m, 6H), 4.61 (d, J=5.4 Hz, 2H), 4.39 (t, J=5.1 Hz, 2H), 4.25 (t, J=5.5 Hz, 1H), 4.02 (t, J=5.0 Hz, 2H), 3.67 (t, J=4.8 Hz, 2H), 3.45 (t, J=5.2 Hz, 2H).

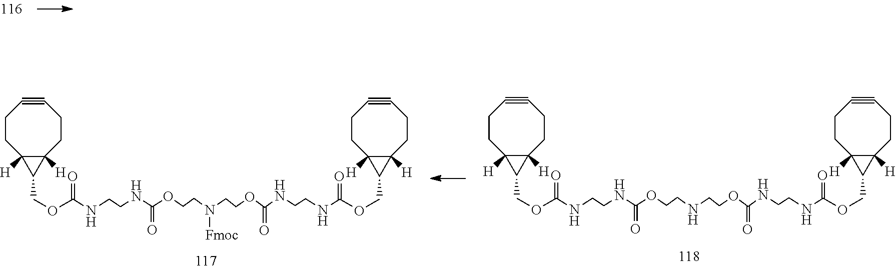

### Example 10. Synthesis of Compound 117

To a solution of 116 (34 mg, 0.050 mmol) in DCM (2 mL) were added 111 (49 mg, 0.21 mmol) and triethylamine (20 μL, 0.14 mmol). The mixture was left stirring overnight at room temperature. After 23 h, the mixture was concentrated. Purification by silica gel column chromatography (0→40% MeOH in DCM) gave 117 as a white solid in 61% yield (27 mg, 0.031 mmol). LCMS (ESI+) calculated for C₄₇H₅₇N₅O₁₀⁺ (M+H⁺) 851.41 found 852.49.

### Example 11. Synthesis of Compound 118

Compound 118 was obtained during the preparation of 117 (3.8 mg, 0.0060 mmol). LCMS (ESI+) calculated for C₃₂H₄₇N₅O₈ (M+H⁺) 629.34 found 630.54.

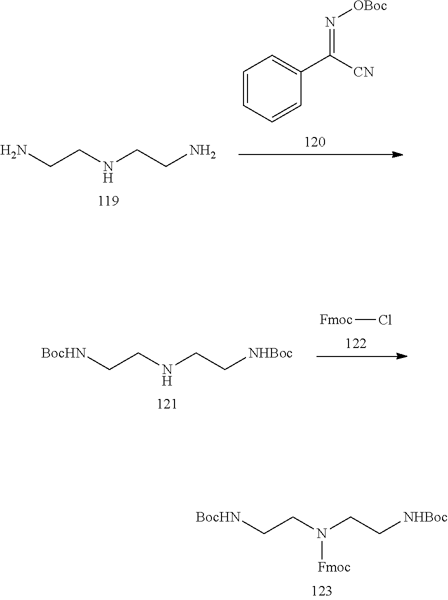

### Example 12. Synthesis of Compound 121

A solution of diethylenetriamine (119) (73 μL, 0.67 mmol) and triethylamine (283 μL, 2.03 mmol) in THE (6 mL) was cooled down to −5° C. and placed under a nitrogen atmosphere. 2-(Boc-oxyimino)-2-phenylacetonitrile (120) (334 mg, 1.35 mmol) was dissolved in THE (4 mL) and slowly added to the cooled solution. After 2.5 h, the ice bath was removed and the mixture was stirred for an additional of 2.5 h at room temperature, and concentrated in vacuo. The residue was redissolved in DCM (15 mL) and washed with a 5% aqueous NaOH solution (2×5 mL), brine (2×5 mL) and dried over MgSO₄. Purification by silica gel column chromatography (0→14% MeOH in DCM) gave 121 as a colorless oil in 91% yield (185 mg, 0.610 mmol). ¹H-NMR (400 MHz, CDCl₃) δ (ppm) 5.08 (s, 2H), 3.30-3.12 (m, 4H), 2.74 (t, J=5.9 Hz, 4H), 1.45 (s, 18H).

### Example 13. Synthesis of Compound 123

To a cooled solution (−10° C.) of 121 (33.5 mg, 0.110 mmol) in THE (2 mL) were added a 10% aqueous NaHCO₃ solution (500 μL) and 9-fluorenylmethoxycarbonyl chloride (122) (34 mg, 0.13 mmol) dissolved in THE (1 mL). After 1 h, the mixture was concentrated and the residue was redissolved in EtOAc (10 mL), washed with brine (2×5 mL), dried over Na₂SO₄, and concentrated. Purification by silica gel column chromatography (0→50% MeOH in DCM) gave 123 in 86% yield (50 mg, 0.090 mmol). ¹H-NMR (400 MHz, CDCl₃) δ (ppm) 7.77 (d, J=7.4 Hz, 2H), 7.57 (d, J=7.4 Hz, 2H), 7.43-7.38 (m, 2H), 7.36-7.31 (m, 2H), 5.57 (d, J=5.2 Hz, 2H), 4.23 (t, J=5.1 Hz, 1H), 3.40-2.83 (m, 8H), 1.41 (s, 18H).

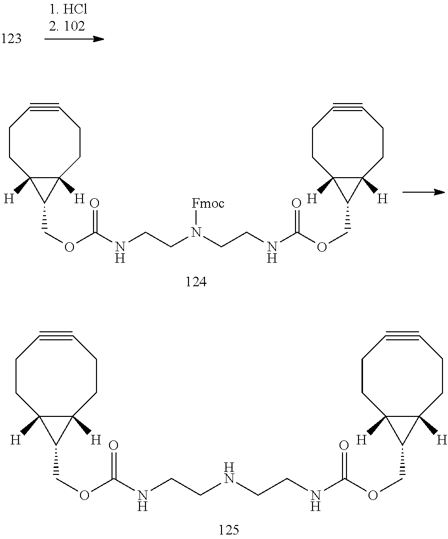

### Example 14. Synthesis of Compound 124

To a solution of 123 (50 mg, 0.095 mmol) in DCM (3 mL) was added 4 M HCl in dioxane (200 μL). The mixture was stirred for 19 h, concentrated and a white solid was obtained (35 mg). without purification, the deprotected intermediate and (1R,8S,9s)-bicyclo[6.1.0]non-4-yn-9-ylmethyl (4-nitrophenyl) carbonate (102) (70 mg, 0.22 mmol) were dissolved in DMF (3 mL) and triethylamine (34 μL, 0.24 mmol) was added. After 2 h, the mixture was concentrated and the residue was purified by silica gel column chromatography (0→25% MeOH in DCM) to yield 124 in 48% (31 mg, 0.045 mmol). LCMS (ESI+) calculated for C₄₁H₄₇N₃O₆⁺ (M+H⁺) 677.35 found 678.57.

### Example 15. Synthesis of Compound 125

To a solution of 124 (10 mg, 0.014 mmol) in DMF (500 μL) was added piperidine (20 μL). After 3.5 h, the mixture was concentrated. Purification by silica gel column chromatography (0→20% MeOH in DCM) gave 125 in 58% yield (3.7 mg, 0.0080 mmol). LCMS (ESI+) calculated for C₂₆H₃₇N₃O₄⁺ (M+H⁺) 455.28 found 456.41.

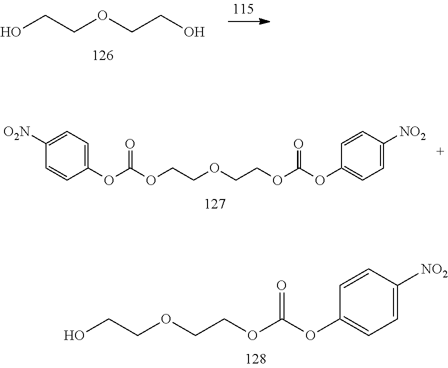 [SMILES: OCCOCCO ; O=C(OCCOCCOC(=O)Oc1ccc([N+](=O)[O-])cc1)Oc1ccc([N+](=O)[O-])cc1 ; O=C(OCCOCCO)Oc1ccc([N+](=O)[O-])cc1]

### Example 16. Synthesis of Compound 127 and 128

To a solution of diethyleneglycol (126) (446 μL, 0.50 g, 4.71 mmol) in DCM (20 mL) were added 4-nitrophenol chloroformate (115) (1.4 g, 7.07 mmol) and Et₃N (3.3. mL, 2.4 g, 23.6 mmol). The mixture was stirred, filtered and concentrated in vacuo (at 55° C.). The residue was purified by silica gel chromatography (15%→75% EtOAc in heptane) and two products were isolated. Product 127 was obtained as a white solid (511 mg, 1.17 mmol, 25%).1H NMR (400 MHz, CDCl₃) δ (ppm) 8.31-8.23 (m, 4H), 7.43-7.34 (m, 4H), 4.54-4.44 (m, 4H), 3.91-3.83 (m, 4H). Product 128 was obtained as a colorless oil (321 mg, 1.18 mmol, 25%).1H NMR (400 MHz, CDCl₃) δ (ppm) 8.32-8.24 (m, 2H), 7.43-7.36 (m, 2H), 4.50-4.44 (m, 2H), 3.86-3.80 (m, 2H), 3.81-3.74 (m, 2H), 3.69-3.64 (m, 2H).

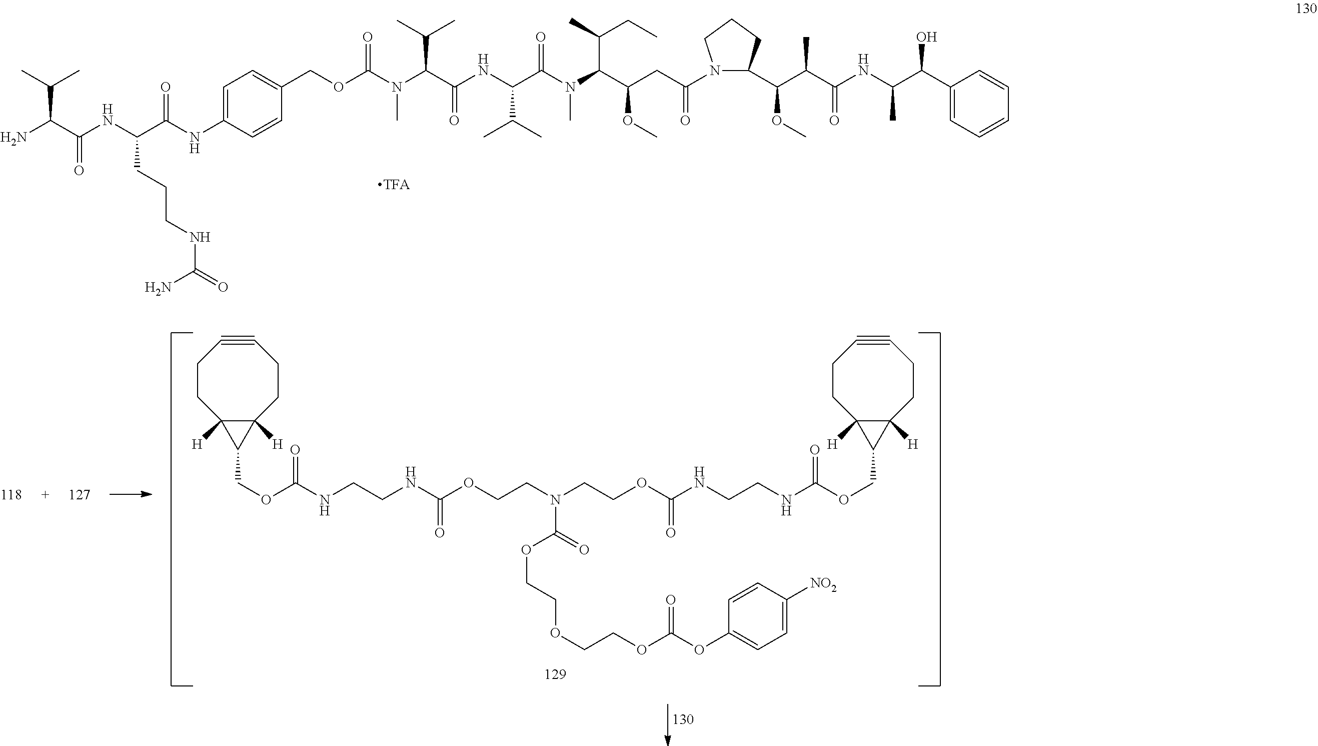

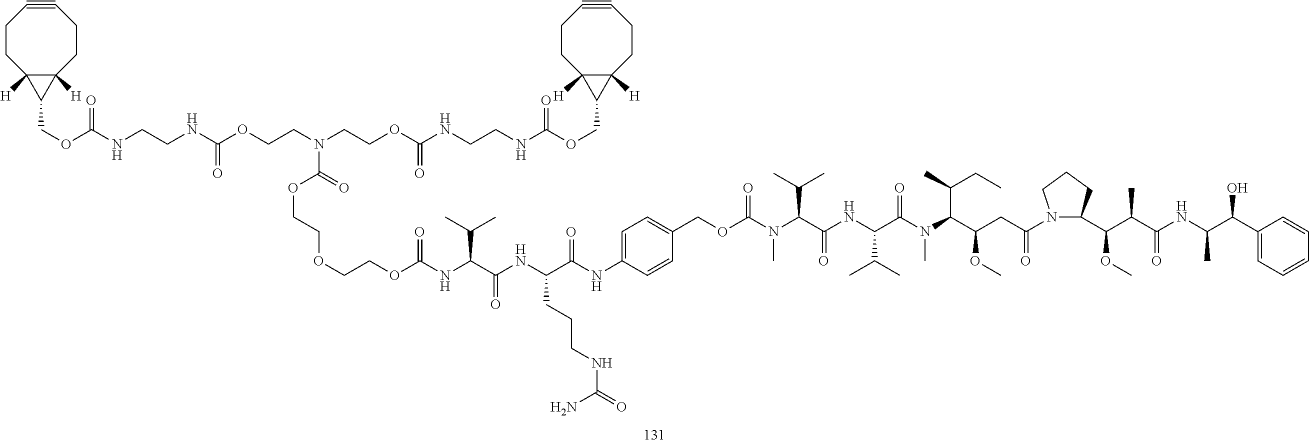 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCCOCCOC(=O)N(CCOC(=O)NCCNC(=O)OC[C@@H]2[C@@H]3CCC#CCC[C@@H]32)CCOC(=O)NCCNC(=O)OC[C@@H]2[C@@H]3CCC#CCC[C@@H]32)C(C)C)cc1)C(C)C]

### Example 17. Synthesis of Compound 131

To a solution of 118 (2.3 mg, 3.7 μmol) in DMF (295 μL) was added a solution of 127 (3.2 mg, 7.4 μmol) in DMF (65 μL) and Et₃N (1.6 μL, 1.1 mg, 11.1 μmol). The mixture was left standing for 17 h and a solution of HOBt (0.5 mg, 3.7 umol) in DMF (14 μL) was added. After 4 h, Et₃N (5.2 μL, 3.8 mg, 37 μmol) and a solution of vc-PABC-MMAE. TFA (130, 13.8 mg, 11 μmol) in DMF (276 μL) were added. After 3 d, the mixture was purified by RP HPLC (C18, 30%→90% MeCN (1% AcOH) in water (1% AcOH). The desired product 131 was obtained as a colorless film (1.5 mg, 0.78 μmol, 21%). LCMS (ESI+) calculated for C₉₆H₁₄₈N₁₅O₂₅⁺ (M+H⁺) 1911.08 found 1912.08.

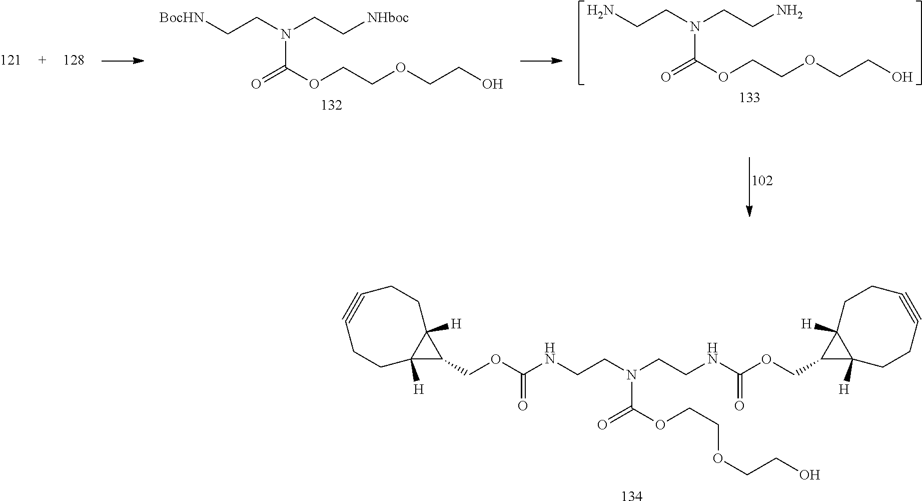

### Example 18. Synthesis of Compound 132

To a solution of 121 (168 mg, 0.554 mmol) in DCM (2 mL), were added a solution of 128 (240 mg, 0.89 mmol) in DCM (1 mL), DCM (1 mL) and Et₃N (169 mg, 233 μL). The mixture was stirred for 17 h, concentrated and purified by silica gel chromatography (gradient of EtOAc in heptane). The desired product 132 was obtained as a slightly yellow oil (85 mg, 0.20 mmol, 35%). ¹H NMR (400 MHz, CDCl₃) δ (ppm) 5.24-5.02 (m, 2H), 4.36-4.20 (m, 3H), 3.84-3.67 (m, 4H), 3.65-3.58 (m, 2H), 3.47-3.34 (m, 4H), 3.34-3.18 (m, 4H), 1.44 (bs, 18H).

### Example 19. Synthesis of Compound 134

To a solution of 132 (81 mg, 0.19 mmol) in DCM (3 mL) was added 4 N HCl in dioxane (700 μL). The mixture was stirred for 19 h, concentrated and the residue was taken up in DMF (0.5 mL). Et₃N (132 μL, 96 mg, 0.95 mmol), DMF (0.5 mL) and (1R,8S,9s)-bicyclo[6.1.0]non-4-yn-9-ylmethyl (4-nitrophenyl) carbonate (102) (132 mg, 0.42 mmol) were added and the resulting mixture was stirred for 2 h. The mixture was concentrated and the residue was purified by silica gel chromatography (0%→3% MeOH in DCM). The desired product 134 was obtained as a colorless film (64 mg, 0.11 mmol, 57%). ¹H NMR (400 MHz, CDCl₃) δ (ppm) 4.31-4.23 (m, 2H), 4.22-4.08 (m, 4H), 3.80-3.68 (m, 4H), 3.66-3.58 (m, 2H), 3.50-3.28 (m, 8H), 2.80-2.65 (m, 1H), 2.40-2.10 (m, 12H), 1.68-1.48 (m, 4H), 1.35 (quintet, J=8.1 Hz, 1H), 1.02-0.87 (m, 2H). LCMS (ESI+) calculated for C₃₁H₄₆N₃O₈⁺ (M+H⁺) 588.33 found 588.43.

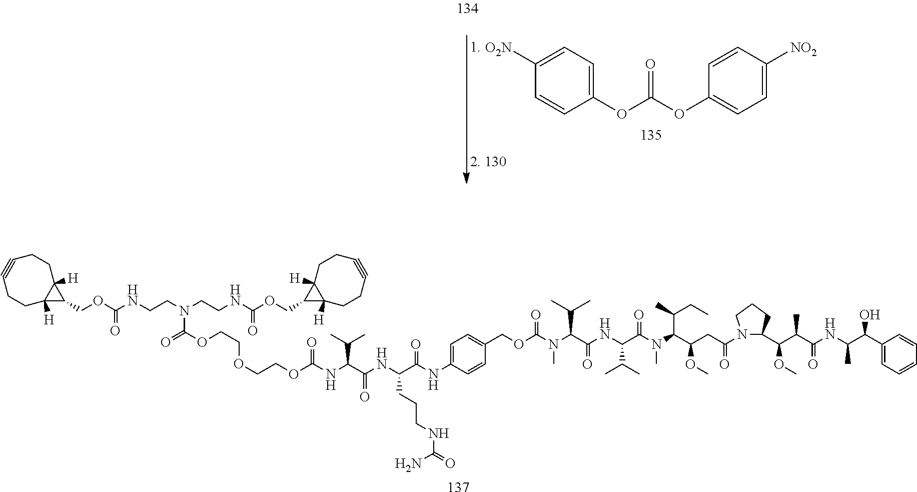 [SMILES: O=C(Oc1ccc([N+](=O)[O-])cc1)Oc1ccc([N+](=O)[O-])cc1 ; "135" ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCCOCCOC(=O)N(CCNC(=O)OC[C@@H]2[C@@H]3CCC#CCC[C@@H]32)CCNC(=O)OC[C@@H]2[C@@H]3CCC#CCC[C@@H]32)C(C)C)cc1)C(C)C ; "137"]

### Example 20. Synthesis of Compound 137

To a solution of 134 (63 mg, 0.11 mmol) in DCM (1 mL) was added bis(4-nitrophenyl) carbonate (35) (32.6 mg, 0.107 mmol) and Et₃N (32.5 mg, 45 μL, 0.32 mmol). After 2 h, 77 μL was removed from the main reaction mixture, a solution of vc-PABC-MMAE. TFA (130, 10 mg, 8.1 μmol) in DMF (200 μL) and Et₃N (3.4 μL, 2.5 mg, 24 μmol) were added. After 18 h, 2,2′-(ethylenedioxy)bis(ethylamine) (4.9 μL, 5.0 mg, 34 μmol) was added and the mixture was left standing for 45 min. The mixture was purified by RP HPLC (C18, 30%→90% MeCN (1% AcOH) in water (1% AcOH). The desired product 137 was obtained as a colorless film (8.7 mg, 5.0 μmol, 61%). LCMS (ESI+) calculated for C₉₀H₁₃₈N₁₃O₂₁⁺ (M+H⁺) 1737.01 found 1738.01.

### Example 21. Synthesis of Compound 139

To a solution of 134 (63 mg, 0.11 mmol) in DCM (1 mL) was added bis(4-nitrophenyl) carbonate (35) (32.6 mg, 0.107 mmol) and Et₃N (32.5 mg, 45 μL, 0.32 mmol). After 20 h, 77 μL was removed from the main reaction mixture, a solution of vc-PABC-MMAF. TFA (138, 9.6 mg, 8.2 μmol) in DMF (240 μL) and Et₃N (3.4 μL, 2.5 mg, 24 μmol) were added. After 3 h, 2,2′-(ethylenedioxy)bis(ethylamine) (20 μL, 20 mg, 0.14 mmol) was added and the mixture was left standing for 20 min. The mixture was purified by RP HPLC (C18, 30%→90% MeCN (1% AcOH) in water (1% AcOH). The desired product 139 was obtained as a colorless film (5.3 mg, 3.2 μmol, 39%). LCMS (ESI+) calculated for C₈₇H₁₃₀N₁₁O₂₁⁺ (M+H⁺) 1664.94 found 1665.99.

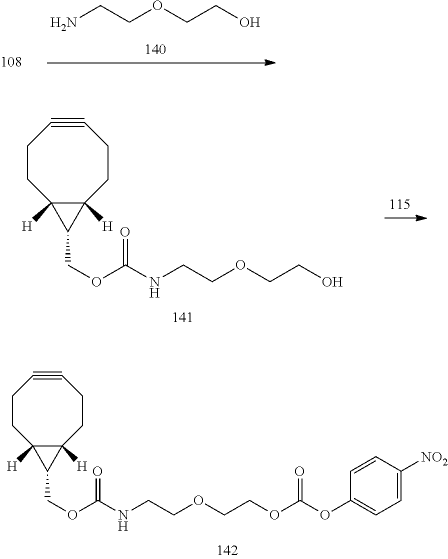 [SMILES: NCCOCCO ; O=C(NCCOCCO)OC[C@@H]1[C@@H]2CCC#CCC[C@@H]21 ; O=C(NCCOCCOC(=O)Oc1ccc([N+](=O)[O-])cc1)OC[C@@H]1[C@@H]2CCC#CCC[C@@H]21]

### Example 22. Synthesis of Compound 141

To a solution of (1R,8S,9S)-bicyclo[6.1.0]non-4-yn-9-ylmethyl N-succinimidyl carbonate (108) (16.35 g, 56.13 mmol) in DCM (400 ml) were added 2-(2-aminoethoxy)ethanol (140) (6.76 ml, 67.35 mmol) and triethylamine (23.47 ml, 168.39 mmol). The resulting pale yellow solution was stirred at rt for 90 min. The mixture was concentrated in vacuo and the residue was co-evaporated once with acetonitrile (400 mL). The resulting oil was dissolved in EtOAc (400 mL) and washed with H₂O (3×200 mL). The organic layer was concentrated in vacuo. The residue was purified by silica gel column chromatography (50%→88% EtOAc in heptane) and gave 141 (11.2 g, 39.81 mmol, 71% yield) as a pale yellow oil. ¹H-NMR (400 MHz, CDCl₃): δ (ppm) 5.01 (br s, 1H), 4.17 (d, 2H, J=12.0 Hz), 3.79-3.68 (m, 2H), 3.64-3.50 (m, 4H), 3.47-3.30 (m, 2H), 2.36-2.14 (m, 6H), 1.93 (br s, 1H), 1.68-1.49 (m, 2H), 1.37 (quintet, 1H, J=8.0 Hz), 1.01-0.89 (m, 2H).

### Example 23. Synthesis of Compound 142

To a solution of 141 (663 mg, 2.36 mmol) in DCM (15 mL) were added triethylamine (986 uL, 7.07 mmol) and 4-nitrophenyl chloroformate (115) (712 mg, 3.53 mmol). The mixture was stirred for 4 h and concentrated in vacuo. Purification by silica gel column chromatography (0→20% EtOAc in heptane) gave 142 (400 mg, 0.9 mmol, yield 38%) as a pale yellow oil. ¹H-NMR (400 MHz, CDCl₃) δ (ppm) 8.29 (d, J=9.4 Hz, 2H), 7.40 (d, J=9.3 Hz, 2H), 5.05 (br s, 1H), 4.48-4.41 (m, 2H), 4.16 (d, J=8.0 Hz, 2H), 3.81-3.75 (m, 2H), 3.61 (t, J=5.0 Hz, 2H), 3.42 (q, J=5.4 Hz, 2H), 2.35-2.16 (m, 6H), 1.66-1.50 (m, 2H), 1.35 (quintet, J=8.6 Hz, 1H), 1.02-0.88 (m, 2H). LCMS (ESI+) calculated for C₂₂H₂₆N₂NaO₈⁺ (M+Na⁺) 469.16 found 469.36.

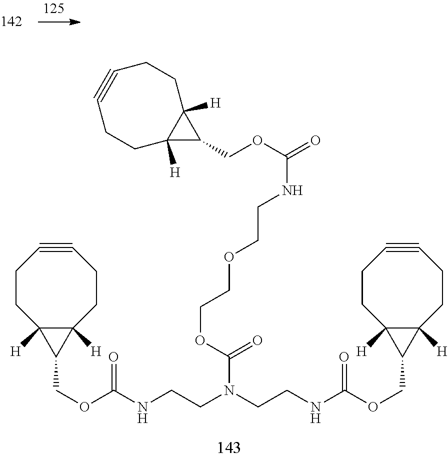 [SMILES: O=C(NCCOCCOC(=O)N(CCNC(=O)OC[C@@H]1[C@@H]2CCC#CCC[C@@H]21)CCNC(=O)OC[C@@H]1[C@@H]2CCC#CCC[C@@H]21)OC[C@@H]1[C@@H]2CCC#CCC[C@@H]21]

### Example 24. Synthesis of Compound 143

A solution of 142 (2.7 mg, 6.0 μmol) in DMF (48 μL) and Et₃N (2.1 μL, 1.5 mg, 15 μmol) were added to a solution of 125 (2.3 mg, 5.0 μmol) in DMF (0.32 mL). The mixture was left standing for 4 d, diluted with DMF (100 μL) and purified by RP HPLC (C18, 30%→100% MeCN (1% AcOH) in water (1% AcOH). The product 143 was obtained as a colorless film (2.8 mg, 3.7 μmol, 74%). LCMS (ESI+) calculated for C₄₂H₅₉N₄O₉⁺ (M+H⁺) 763.43 found 763.53.

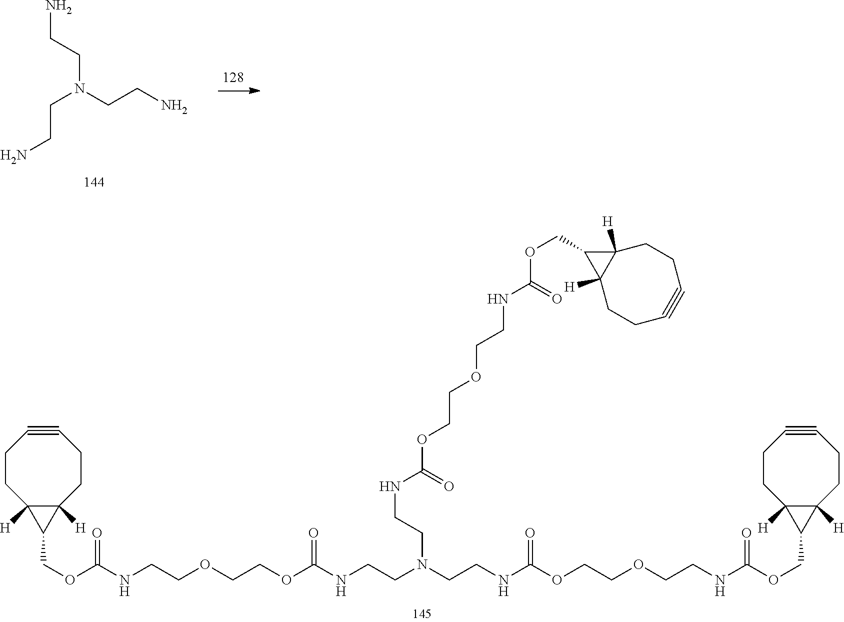 [SMILES: NCCN(CCN)CCN ; O=C(NCCN(CCNC(=O)OCCOCCNC(=O)OC[C@@H]1[C@@H]2CCC#CCC[C@@H]21)CCNC(=O)OCCOCCNC(=O)OC[C@@H]1[C@@H]2CCC#CCC[C@@H]21)OCCOCCNC(=O)OC[C@@H]1[C@@H]2CCC#CCC[C@@H]21]

### Example 25. Synthesis of Compound 145

To a solution of 128 (200 mg, 0.45 mmol) in DCM (1 mL) were added triethylamine (41.6 uL, 0.30 mmol) and tris(2-aminoethyl)amine 144 (14.9 uL, 0.10 mmol). After stirring the mixture for 150 minutes, it was concentrated in vacuo. The residue was purified by silica gel column chromatography (25%→100% EtOAc in DCM then 0%→10% MeOH in DCM) and gave 145 in 43% yield (45.4 mg, 42.5 umol) as a yellow oil. ¹H NMR (400 MHz, CDCl₃): δ (ppm) 5.68-5.18 (m, 6H), 4.32-4.18 (m, 6H), 4.18-4.11 (d, J=7.9 Hz, 6H), 3.74-3.61 (m, 6H), 3.61-3.51 (m, 6H), 3.43-3.29 (m, 6H), 3.29-3.15 (m, 6H), 2.65-2.47 (m, 6H), 2.37-2.16 (m, 18H), 1.69-1.49 (m, 6H), 1.35 (quintet, J=8.9 Hz, 3H), 1.03-0.87 (m, 6H).

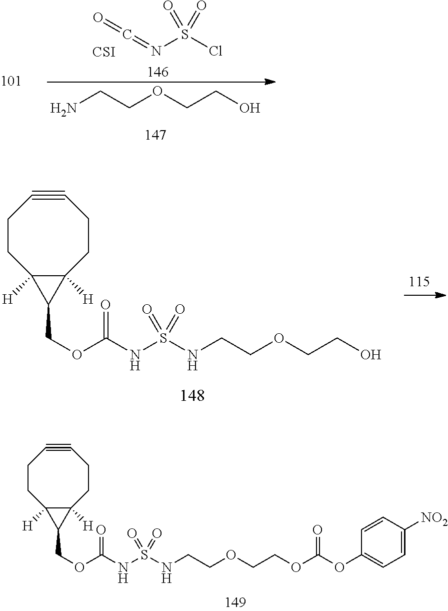

### Example 26. Synthesis of Compound 148

To a solution of BCN-OH (101) (3.0 g, 20 mmol) in DCM (300 mL) was added CSI (146) (1.74 mL, 2.83 g, 20 mmol). After the mixture was stirred for 15 min, Et₃N (5.6 mL, 4.0 g, 40 mmol) was added. The mixture was stirred for 5 min and 2-(2-aminoethoxy)ethanol (147) (2.2 mL, 2.3 g, 22 mmol) was added. The resulting mixture was stirred for 15 min and saturated aqueous NH₄Cl (300 mL) was added. The layers were separated, and the aqueous phase was extracted with DCM (200 mL). The combined organic layers were dried (Na₂SO₄) and concentrated. The residue was purified by silica gel chromatography (0% to 10% MeOH in DCM). The fractions, containing the desired product, were concentrated. The residue was taken up in EtOAc (100 mL) and concentrated. The desired product 148 was obtained as a slightly yellow oil (4.24 g, 11.8 mmol, 59%). ¹H NMR (400 MHz, CDCl₃) δ (ppm) 5.99-5.79 (bs, 1H), 4.29 (d, J=8.3 Hz, 2H), 3.78-3.74 (m, 2H), 3.66-3.56 (m, 4H), 3.37-3.30 (m, 2H), 2.36-2.16 (m, 6H), 1.63-1.49 (m, 2H), 1.40 (quintet, J=8.7 Hz, 1H), 1.05-0.94 (m, 2H).

### Example 27. Synthesis of Compound 149

To a solution of 148 (3.62 g, 10.0 mmol) in DCM (200 mL) were added 4-nitrophenyl chloroformate (15) (2.02 g, 10.0 mmol) and Et₃N (4.2 mL, 3.04 g, 30.0 mmol). The mixture was stirred for 1.5 h and concentrated. The residue was purified by silica gel chromatography (20%→70% EtOAc (1% AcOH) in heptane (1% AcOH). The product 149 was obtained as a white foam (4.07 g, 7.74 mmol, 74%). ¹H NMR (400 MHz, CDCl₃) δ (ppm) 8.32-8.26 (m, 2H), 7.45-7.40 (m, 2H), 5.62-5.52 (m, 1H), 4.48-4.42 (m, 2H), 4.28 (d, J=8.2 Hz, 2H), 3.81-3.76 (m, 2H), 3.70-3.65 (m, 2H), 3.38-3.30 (m, 2H), 2.35-2.16 (m, 6H), 1.62-1.46 (m, 2H), 1.38 (quintet, J=8.7 Hz, 1H), 1.04-0.93 (m, 2H).

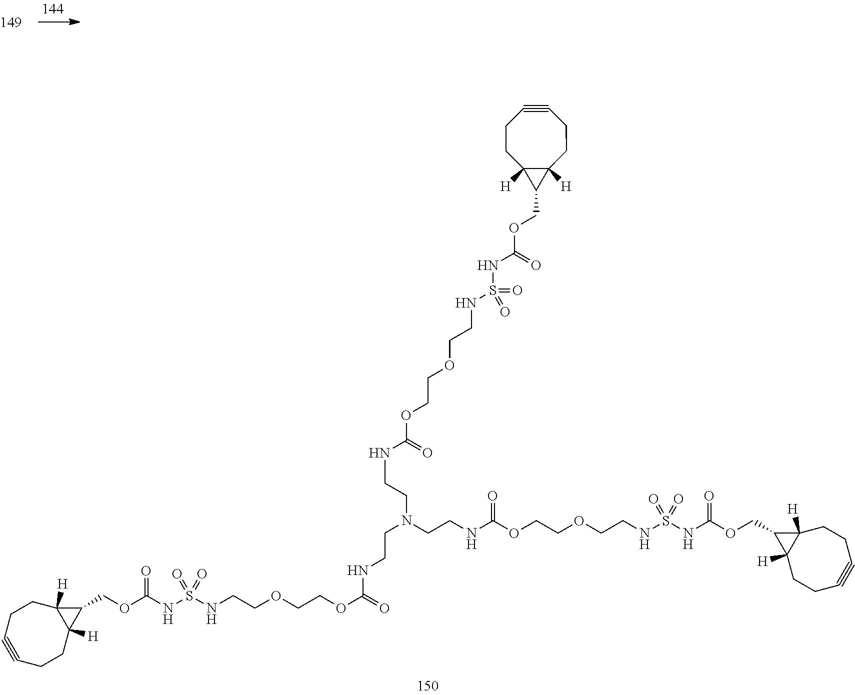 [SMILES: O=C(NCCN(CCNC(=O)OCCOCCNS(=O)(=O)NC(=O)OC[C@@H]1[C@@H]2CCC#CCC[C@@H]21)CCNC(=O)OCCOCCNS(=O)(=O)NC(=O)OC[C@@H]1[C@@H]2CCC#CCC[C@@H]21)OCCOCCNS(=O)(=O)NC(=O)OC[C@@H]1[C@@H]2CCC#CCC[C@@H]21]

### Example 28. Synthesis of Compound 150

To a solution of 149 (200 mg, 0.38 mmol) in DCM (1 mL) were added triethylamine (35.4 uL, 0.24 mmol) and tris(2-aminoethyl)amine (144) (12.6 uL, 84.6 umol). The mixture was stirred for 120 min and concentrated in vacuo. The residue was purified by silica gel column chromatography (25%→100% EtOAc in DCM then 0%→10% MeOH in DCM) and gave 150 in 36% yield (40.0 mg, 30.6 umol) as a white foam. ¹H NMR (400 MHz, CDCl₃): δ (ppm) 6.34-5.72 (m, 6H), 4.34-4.18 (m, 12H), 3.76-3.58 (m, 12H), 3.43-3.30 (m, 6H), 3.30-3.18 (m, 6H), 2.64-2.49 (m, 6H), 2.38-2.14 (m, 18H), 1.65-1.47 (m, 6H), 1.39 (quintet, J=9.1 Hz, 3H), 1.06-0.90 (m, 6H).

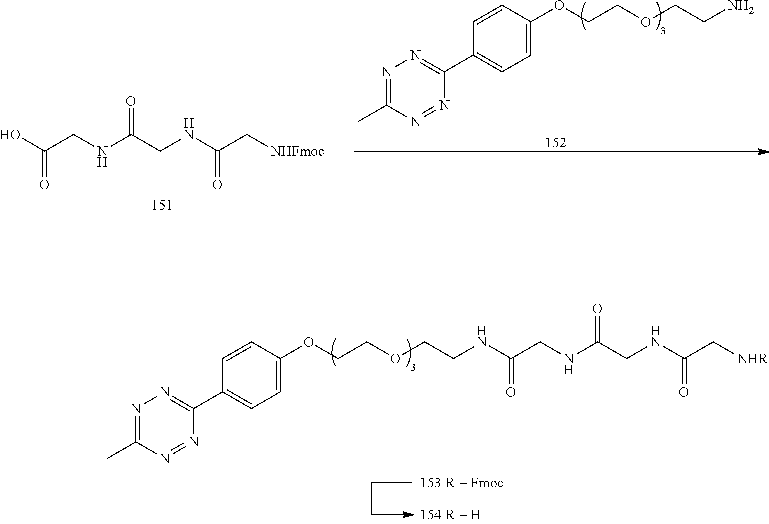

### Example 29. Synthesis of Compound 153

To a mixture of Fmoc-Gly-Gly-Gly-OH (151) (31.2 mg, 75.8 μmol) in anhydrous DMF (1 mL) were added N,N-diisopropylethylamine (40 μL, 29 mg, 0.23 mmol) and HATU (30.3 mg, 79.6 μmol). After 10 min tetrazine-PEG₃-ethylamine (152) (30.3 mg, 75.8 μmol) was added and the mixture was vortexed. After 2 h, the mixture was purified by RP HPLC (C18, 30%→90% MeCN (1% AcOH) in water (1% AcOH). The desired product was obtained as a pink film (24.1 mg, 31.8 μmol, 42%). LCMS (ESI+) calculated for C₃₈H₄₅N₈O₉⁺ (M+H⁺) 757.33 found 757.46.

### Example 30. Synthesis of Compound 154

To a solution of 153 (24.1 mg, 31.8 μmol) in DMF (500 μL) was added diethylamine (20 μL, 14 mg, 191 μmol). The mixture was left standing for 2 h and purified by RP HPLC (C18, 5%→90% MeCN (1% AcOH) in water (1% AcOH). The desired product 154 was obtained as a pink film (17.5 mg, 32.7 μmol, quant). LCMS (ESI+) calculated for C₂₃H₃₅N₈O₇⁺ (M+H⁺) 535.26 found 535.37.

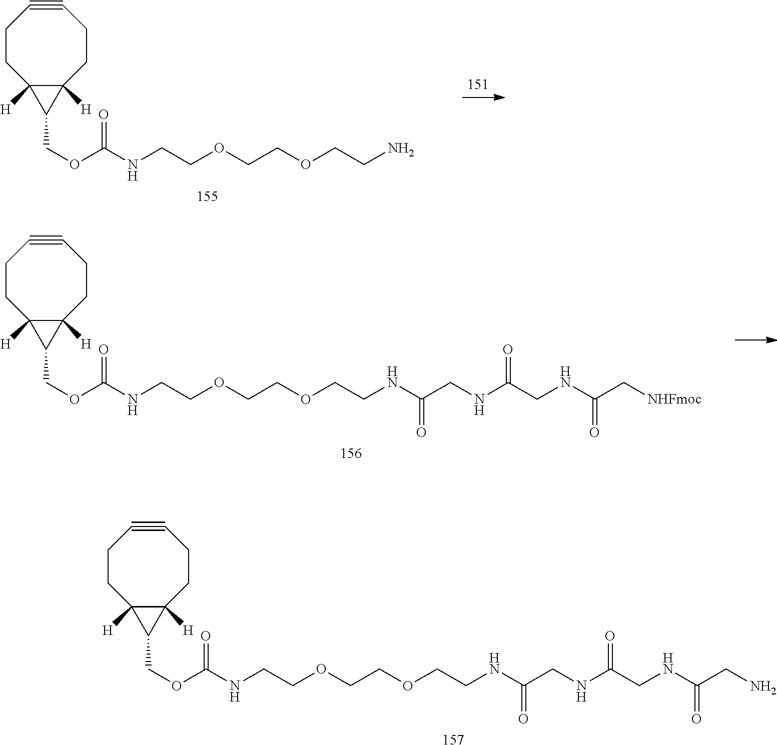

### Example 31. Synthesis of Compound 156

A solution of N-[(1R,8S,9s)-bicyclo[6.1.0]non-4-yn-9-ylmethyloxycarbonyl]-1,8-diamino-3,6-dioxaoctane (155) (68 mg, 0.21 mmol) in dry DMF (2 mL) was transferred to a solution of Fmoc-Gly-Gly-Gly-OH (151) (86 mg, 0.21 mmol) in dry DMF (2 mL). DIPEA (100 μL, 0.630 mmol) and HATU (79 mg, 0.21 mmol) were added. After 1.5 h, the mixture was concentrated and the residue was purified by silica gel column chromatography (0→11% MeOH in DCM) which gave the desired compound 156 in 34% yield (52 mg, 0.072 mmol). LCMS (ESI+) calculated for C₃₅H₄₇N₅O₉⁺ (M+H⁺) 717.34 found 718.39.

### Example 32. Synthesis of Compound 157

Compound 156 (21 mg, 0.029 mmol) was dissolved in DMF (2.4 mL) and piperidine (600 μL) was added. After 20 minutes, the mixture was concentrated and the residue was purified by preparative HPLC, which gave the desired compound 157 as a white solid (9.3 mg, 0.018 mmol, 64%). LCMS (ESI+) calculated for C₂₃H₃₇N₅O₇⁺ (M+H⁺) 495.27 found 496.56.

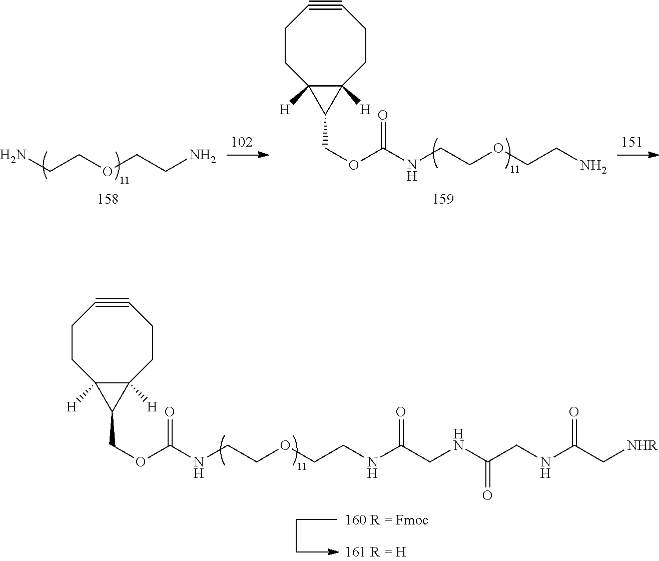

### Example 33. Synthesis of Compound 159

To a solution of amino-PEG₁₁-amine (158) (143 mg, 0.260 mmol) in DCM (5 mL) was slowly added (1R,8S,9s)-bicyclo[6.1.0]non-4-yn-9-ylmethyl (4-nitrophenyl) carbonate (102) (41 mg, 0.13 mmol) dissolved in DCM (5 mL). After 1.5 h, the mixture was reduced and the residue was purified by silica gel column chromatography (0→20% 0.7 N NH₃ MeOH in DCM) which gave the desired compound 159 as a clear oil (62 mg, 0.086 mmol, 66%). LCMS (ESI+) calculated for C₃₅H₄₆N₂O₁₃⁺ (M+H⁺) 720.44 found 721.56.

### Example 34. Synthesis of Compound 160

A solution of 159 (62 mg, 0.086 mmol) in dry DMF (2 mL) was transferred to a solution of Fmoc-Gly-Gly-Gly-OH (151) (36 mg, 0.086 mmol) in dry DMF (2 mL). DIPEA (43 μL, 0.25 mmol) and HATU (33 mg, 0.086 mmol) were added. After 18 h, the mixture was concentrated and the residue was purified by silica gel column chromatography (0→20% MeOH in DCM) which gave the desired compound 160 in 62% yield (60 mg, 0.054 mmol). LCMS (ESI+) calculated for C₅₆H₈₃N₅O₁₈⁺ (M+H⁺) 1113.57 found 1114.93.

### Example 35. Synthesis of Compound 161

Compound 160 (36 mg, 0.032 mmol) was dissolved in DMF (2 mL) and piperidine (200 μL) was added. After 2 h, the mixture was concentrated and the residue was purified by silica gel column chromatography (0→40% 0.7 N NH₃ MeOH in DCM) which gave the desired compound 161 as a yellow oil (16.7 mg, 0.0187 mmol, 58%). LCMS (ESI+) calculated for C₄₁H₇₃N₅O₁₆⁺ (M+H⁺) 891.51 found 892.82.

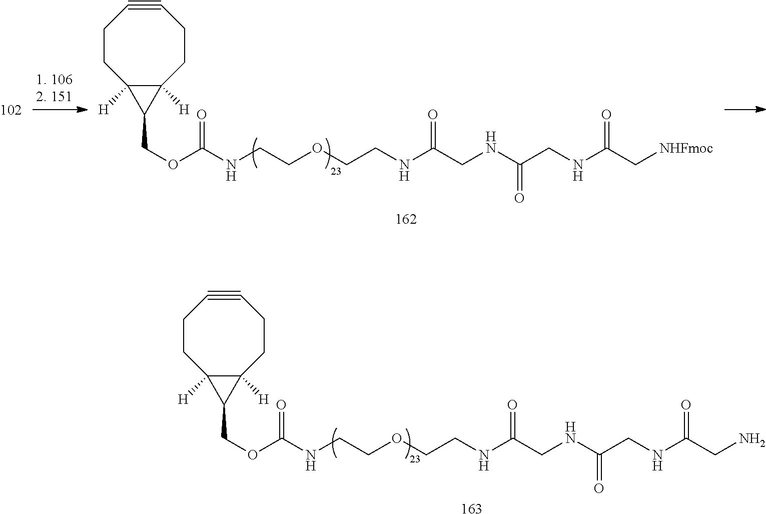

### Example 36. Synthesis of Compound 162

To a solution of amino-PEG23-amine (106) (60 mg, 0.056 mmol) in DCM (3 mL) was slowly added (1R,8S,9s)-bicyclo[6.1.0]non-4-yn-9-ylmethyl (4-nitrophenyl) carbonate (102) (12 mg, 0.037 mmol) dissolved in DCM (5 mL). After 4 h, the mixture was concentrated and redissolved in DMF (2 mL), after which Fmoc-Gly-Gly-Gly-OH (51) (23 mg, 0.056 mmol), HATU (21 mg, 0.056 mmol), and DIPEA (27 μL, 0.16 mmol) were added. After 20 h, the mixture was concentrated and the residue was purified by silica gel column chromatography (0→27% MeOH in DCM) which gave the desired compound 162 in 93% (57 mg, 0.043 mmol). LCMS (ESI+) calculated for C₈₀H₁₃₁N₅O₃₀⁺ (M+NH₄⁺) 1641.89 found 1659.92.

### Example 37. Synthesis of Compound 163

Compound 162 (57 mg, 0.034 mmol) was dissolved in DMF (1 mL) and piperidine (120 μL) was added. After 2 h, the mixture was concentrated, redissolved in water and the Fmoc-piperidine by-product was removed with extraction with diethyl ether (3×10 mL). After freeze dry, 163 was obtained as an yellow oil (46.1 mg, 0.032 mmol, 95%). LCMS (ESI+) calculated for C₆₅H₁₂₁N₅O₂₈⁺ (M+H⁺) 1419.82 found 1420.91.

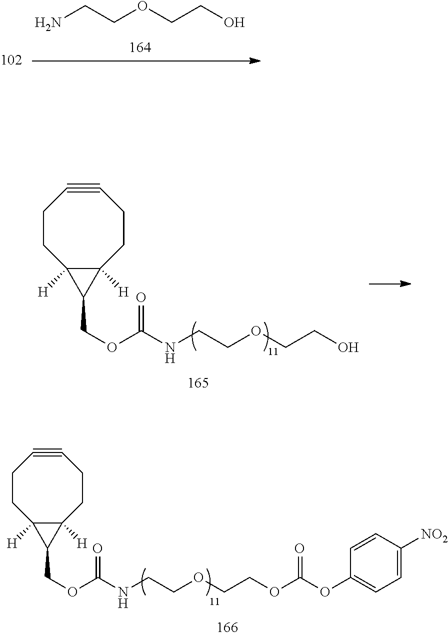

### Example 38. Synthesis of Compound 165

To a solution of (1R,8S,9s)-bicyclo[6.1.0]non-4-yn-9-ylmethyl (4-nitrophenyl) carbonate (102) (204 mg, 0.650 mmol) were added amino-PEG12-alcohol (164) (496 mg, 0.908 mmol) and triethyl amine (350 μL, 2.27 mmol). After 19 h, the mixture was concentrated and the residue was purified by silica gel column chromatography (2→20% MeOH in DCM) which gave 165 as a clear yellow oil (410 mg, 0.560 mmol, 87%). LCMS (ESI+) calculated for C₃₅H₆₃NO₁₄⁺ (M+Na⁺) 721.42 found 744.43.

### Example 39. Synthesis of Compound 166

To a solution of 165 (410 mg, 0.560 mmol) in DCM (6 mL) were added 4-nitrophenyl chloroformate (171, 0.848 mmol) and triethyl amine (260 μL, 1.89 mmol). After 18 h, the mixture was concentrated and the residue was purified by silica gel column chromatography (0→7% MeOH in DCM) which gave the desired compound 166 as a clear oil (350 mg, 0.394 mmol, 70%). LCMS (ESI+) calculated for C₄₂H₆₆N₂O₁₈⁺ (M+Na⁺) 886.43 found 909.61.

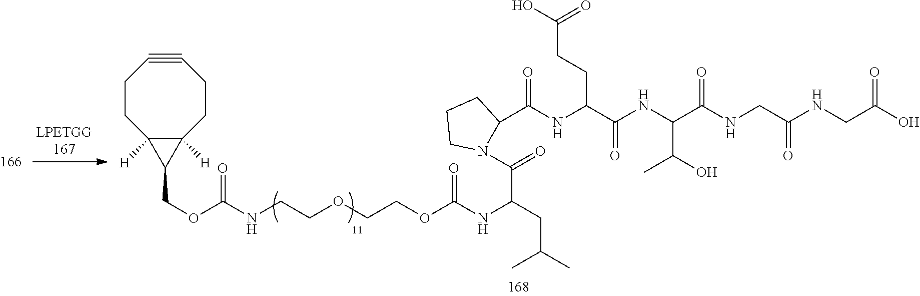

### Example 40. Synthesis of Compound 168

To a solution of 166 (15 mg, 0.017 mmol) in DMF (2 mL) were added peptide LPETGG (167) (9.7 mg, 0.017 mmol) and triethylamine (7 μL, 0.05 mmol). After 46 h, the mixture was concentrated and the residue was purified by preparative HPLC, which gave the desired compound 168 in 63% (14 mg, 0.010 mmol). LCMS (ESI+) calculated for C₆₀H₁₀₁N₇O₂₅⁺ (M+H⁺) 1319.68 found 1320.92.

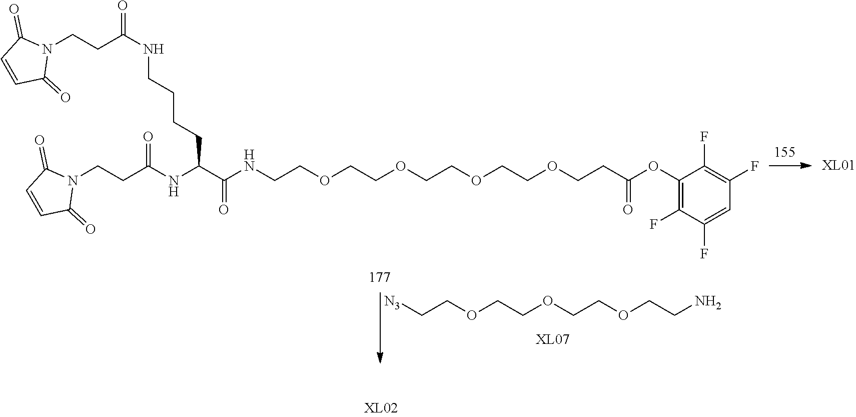 [SMILES: O=C(CCN1C(=O)C=CC1=O)NCCCC[C@H](NC(=O)CCN1C(=O)C=CC1=O)C(=O)NCCOCCOCCOCCOCCC(=O)Oc1c(F)c(F)cc(F)c1F ; [N-]=[N+]=NCCOCCOCCOCCN]

### Example 41. Synthesis of XL01

To a solution of 155 (9.7 mg, 0.03 mmol) in anhydrous DMF (170 μL) were added 177 (bis-maleimide-lysine-PEG₄-TFP, Broadpharm) (20 mg, 0.024 mmol) and Et₃N (9.9 μL, 0.071 mmol). After stirring at room temperature for 42 h, the mixture was diluted with DCM (0.4 mL) and purified by flash column chromatography over silicagel (0%→18% MeOH in DCM) to give XL01 as a clear oil (10.2 mg, 0.010 mmol, 43%). LCMS (ESI+) calculated for C₄₉H₇₂N₇O₁₆⁺ (M+H⁺) 1003.12 found 1003.62.

### Example 42. Synthesis of bis-maleimide azide XL02

To a vial containing 177 (32.9 mg, 39.0 μmol, 1.0 equiv.) in dry DMF (400 μL) was added XL07 (9.2 mg, 42.1 μmol, 1.08 equiv.) and the solution was mixed and left at rt for circa 50 min. Next, DiPEA was added and the resulting solution was mixed and left at rt for circa 2 hours. The reaction mixture was then purified directly by silica gel chromatography (DCM→14% MeOH in DCM). The desired product XL02 was obtained as a colorless oil (28.9 mg, 32.2 μmol, 83% yield). LCMS (ESI+) calculated for C₃₉H₆₂N₉O₁₅⁺ (M+H⁺) 896.97. found 896.52.

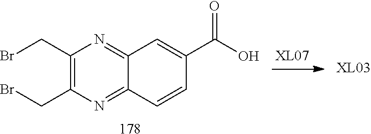

### Example 43. Synthesis of XL03

To a vial containing 2,3-bis(bromomethyl)-6-quinoxalinecarboxylic acid 178 (51.4 mg, 142.8 μmol, 1.00 equiv.) in dry DCM (7.5 mL) was added DIC (9.0 mg, 71.4 μmol, 0.5 equiv.). The resulting mixture was left at rt for 30 minutes, followed by the addition of a solution of XL07 (17.7 mg, 78.5 μmol, 0.55 equiv.) in dry DCM (0.5 mL). The reaction mixture was stirred at rt for circa 35 minutes and then purified directly by silica gel chromatography (DCM→10% MeOH in DCM) to give impure product (72 mg) as a white solid. The impure product was taken up in 1.0 mL DMF and 50% of this solution was co-evaporated with toluene (2×). The residue was purified by silica gel chromatography (12→30% acetone in toluene). The desired product XL03 was obtained as a colorless oil (20.1 mg, 35.9 μmol). LCMS (ESI+) calculated for C₁₉H₂₅Br₂N₆O₄⁺ (M+H⁺) 561.03. found 561.12

### Example 44. Synthesis of XL05

To a solution of 178 (30 mg, 0.09 mmol), in DCM (0.3 mL) were added 3-maleimidopropionic NHS ester (27 mg, 0.10 mmol) and Et₃N (38 μL, 0.27 mmol). After stirring at room temperature for 28 h, the crude mixture was concentrated in vacuo and purified by flash column chromatography over silicagel (0%→15% MeOH in DCM) to give XL05 as a clear oil (27 mg, 0.056 mmol, 62%). LCMS (ESI+) calculated for C₂₄H₃₄N₃O₇⁺ (M+H⁺) 476.54 found 476.46.

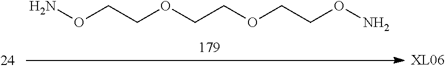 [SMILES: NOCCOCCOCCON]

### Example 45. Synthesis of XL06

To a vial containing 24 (17.2 mg, 88 wt % by ¹-H-qNMR, 18.4 μmol, 1.00 equiv.) was added a solution of 179 in dry DMF (60 μL). To the resulting colorless solution was added triethylamine (40.6 μL, 15.8 equiv., 291 μmol), generating a yellow solution immediately. The reaction mixture was left at room temperature for circa 28 hours and was then conc. in vacuo until most of the Et₃N had evaporated. The residue was then diluted with DCM (1 mL) and purified directly by silica gel chromatography (1^{st} column: DCM→20% MeOH in DCM, 2^{nd} column: DCM→20% MeOH in DCM). The desired product (XL06) was obtained as a colorless oil (4.3 mg, 18.4 μmol, 26% yield). LCMS (ESI+) calculated for C₃₄H₆₂N₇O₁₉S⁺ (M+H⁺) 904.38. found 904.52.

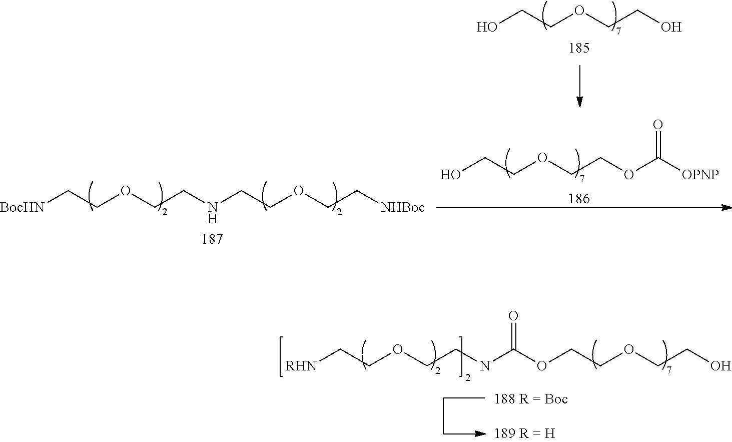

### Example 46. Synthesis of 186

To a solution of octa-ethylene glycol 185 in DCM (10 mL) was added triethylamine (1.0 mL, 7.24 mmol, 2.5 equiv.) followed by dropwise addition of a 4-nitrophenyl chloroformate (0.58 g, 2.90 mmol, 1 equiv.) solution in DCM (5 mL) in 28 minutes. After stirring the mixture for 90 minutes, it was concentrated in vacuo. The residue was purified by silicagel column chromatography (75%→0% EtOAc in DCM followed by 0%→7% MeOH in DCM). The product 186 was obtained in 38% yield as a colorless oil (584.6 mg, 1.09 mmol). LCMS (ESI+) calculated for C₂₃H₃₈NO₁₃⁺ (M+H⁺) 536.23, found 536.93. ¹H-NMR (400 MHz, CDCl₃): δ (ppm) 8.28 (d, J=12.0 Hz, 2H), 7.40 (d, J=12.0 Hz, 2H), 4.47-4.42 (m, 2H), 3.84-3.79 (m, 2H), 3.75-3.63 (m, 26H), 3.63-3.59 (m, 2H), 2.70-2.55 (bs, 1H).

### Example 47. Synthesis of 188

To a solution of 187 (BocNH-PEG₂)₂NH, 202 mg, 0.42 mmol) in DCM (1 mL) was added part (0.5 mL, 0.54 mmol 1.3 equiv.) of a prepared stock solution of 186 (584 mg in DCM (1 mL)) followed by triethylamine (176 μL, 1.26 mmol, 3 equiv.) and HOBt (57 mg, 0.42 mmol, 1 equiv.). After stirring the mixture for 8 days, it was concentrated in vacuo. The residue was taken up in a mixture of acetonitrile (4.2 mL) and 0.1 N NaOH_{(aq)} (4.2 mL, 1 equiv.) and additional amount of solid NaOH (91.5 mg). After stirring the mixture for another 21.5 hours the mixture was extracted with DCM (3×40 mL). The combined organic layers were concentrated in vacuo and the residue was purified by silicagel column chromatography (0%→15% MeOH in DCM). Product 188 was obtained in 87% yield as a pale yellow oil (320.4 mg, 0.37 mmol). LCMS (ESI+) calculated for C₃₉H₇₈N₃O₁₈⁺ (M+H⁺) 876.53, found 876.54.

¹H-NMR (400 MHz, CDCl₃): δ (ppm) 5.15-5.02 (bs, 2H), 4.25-4.19 (m, 2H), 3.76-3.46 (m, 50H), 3.35-3.26 (m, 4H), 2.79-2.69 (br. s, 1H), 1.44 (s, 18H).

### Example 48. Synthesis of 189

188 (320 mg, 0.37 mmol) was dissolved in DCM (1 mL). Then 4M HCl in dioxane (456 μL, 1.83 mmol, 5 equiv.) was added. After stirring the mixture for 3.5 hours, additional 4M HCl in dioxane (450 μL, 1.80 mmol, 4.9 equiv.) was added. After stirring the mixture for another 3.5 hours, additional 4M HCl in dioxane (450 μL, 1.80 mmol, 4.9 equiv.) was added. After stirring the mixture for 16.5 hours the mixture was concentrated in vacuo. Product 189 was obtained in quantitative yield as a white sticky solid. This was used directly in the next step. ¹H-NMR (400 MHz, DMSO-d6): δ (ppm) 8.07-7.81 (bs, 6H), 4.15-4.06 (m, 2H), 3.75-3.66 (m, 2H), 3.65-3.48 (m, 48H), 3.03-2.92 (m, 4H).

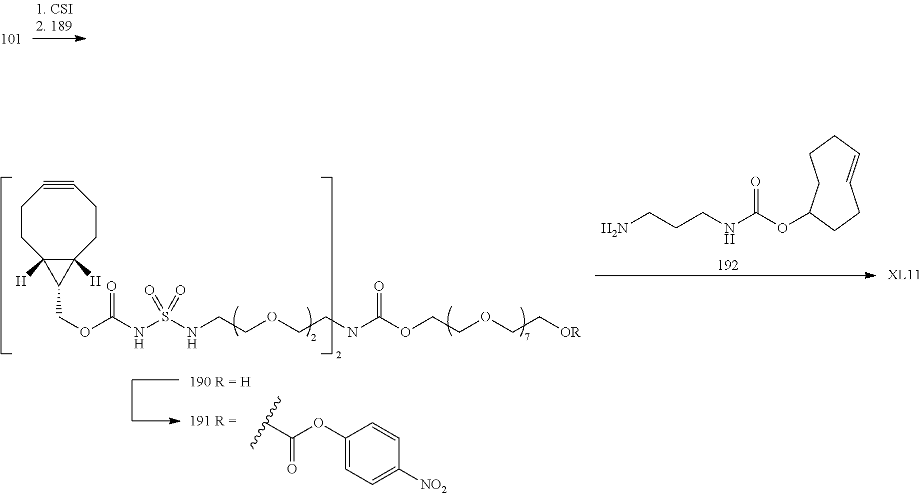

### Example 49. Synthesis of 190

To a solution of BCN-OH (164 mg, 1.10 mmol, 3 equiv.) in DCM (3 mL) was added CSI (76 μL, 0.88 mmol, 2.4 equiv.). After stirring for 15 minutes triethylamine (255 μL, 5.50 mmol, 5 equiv.) was added. A solution of 189 was prepared by adding DCM (3 mL) and triethylamine (508 μL, 11.0 mmol, 10 equiv.). This stock solution was added to the original reaction mixture after 6 minutes. After stirring the mixture for 21.5 hours, it was concentrated in vacuo. The residue was purified by silicagel column chromatography (0%→10% MeOH in DCM). Product 190 was obtained in 39% yield as pale yellow oil (165.0 mg, 139 μmol). LCMS (ESI+) calculated for C₄₃H₇₂N₅O₁₈S₂⁺ (M+H⁺) 1186.54, found 1186.65.

¹H-NMR (400 MHz, CDCl₃): δ (ppm) 6.09-5.87 (m, 2H), 4.31-4.19 (m, 6H), 3.76-3.50 (m, 50H), 3.40-3.29 (m, 4H), 2.38-2.16 (m, 12H), 1.66-1.47 (m, 4H), 1.40 (quintet, J=8.0 Hz, 2H), 1.04-0.94 (m, 4H).

### Example 50. Synthesis of 191

To a solution of 190 (101 mg, 0.085 mmol) in DCM (2.0 mL) were added bis(4-nitrophenyl) carbonate (39 mg, 0.127 mmol) and Et₃N (36 uL, 0.25 mmol). After stirring at room temperature for 42 h, the crude mixture was concentrated in vacuo and purified by flash column chromatography over silicagel (A. 0%→25% EtOAc in DCM (till p-nitrophenol was eluded), followed by gradient B. 0%→12% MeOH in DCM) to give 191 as a clear oil (49 mg, 0.036 mmol, 42%). LCMS (ESI+) calculated for C₅₈H₉₁N₆O₂₆S₂⁺ (M+H⁺) 1352.50 found 1352.78.

### Example 51. Synthesis of XL11

To a solution of 191 (7 mg, 0.0059 mmol) in anhydrous DMF (130 μL) were added Et₃N (2.2 uL, 0.015 mmol) and TCO-amine hydrochloride (Broadpharm) (1.8 mg, 0.0068 mmol). After stirring at room temperature for 19 h, the crude mixture was purified by flash column chromatography over silicagel (0%→15% MeOH in DCM) to give XL11 as a clear oil (1.5 mg, 0.001 mmol, 17%). LCMS (ESI+) calculated for C₆₄H₁₁₁N₈O₂₅S₂⁺ (M+NH₄⁺) 1456.73 found 1456.81.

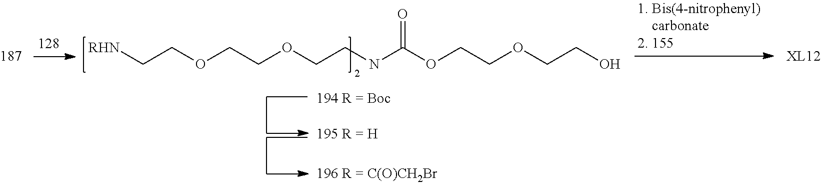

### Example 52. Synthesis of 194

To a solution of available 187 (638 mg, 1.33 mmol) in DCM (8.0 mL) were added 128 (470 mg, 1.73 mmol), Et₃N (556.0 μL, 4.0 mmol), and 1-hydroxybenzotriazole (179.0 mg, 1.33 mmol). After stirring for 41 h at ambient temperature, the mixture was concentrated in vacuo and redissolved in MeCN (10 mL) followed by the addition of aqueous 0.1 M NaOH solution (10 mL) and solid NaOH pellets (100.0 mg). After 1.5 h, DCM (20 mL) was added and the desired compound was extracted four times. The organic layers were concentrated in vacuo and the residue was purified by flash column chromatography over silicagel (0%→12% MeOH in DCM) to give 194 as a clear yellow oil (733 mg, 1.19 mmol, 90%). ¹H NMR (400 MHz, CDCl₃) δ (ppm) 4.29-4.23 (m, 2H), 3.77-3.68 (m, 4H), 3.65-3.56 (m, 14H), 3.56-3.49 (m, 8H), 3.37-3.24 (m, 4H), 1.45 (s, 18H). LCMS (ESI+) calculated for C₂₇H₅₄N₃O₁₂⁺ (M+H⁺) 612.73 found 612.55.

### Example 53. Synthesis of 195

To a solution of 194 (31.8 mg, 0.052 mmol) in DCM (1.0 mL) was added 4.0 M HCl in dioxane (0.4 mL). After stirring for 2.5 h at ambient temperature, the reaction mixture was concentrated in vacuo and in between redissolved in DCM (2 mL) and concentrated. Compound 195 was obtained as a clear oil in quantitative yield. LCMS (ESI+) calculated for C₁₇H₃₈N₃O₈⁺ (M+H⁺) 412.50 found 412.45

### Example 54. Synthesis of 196

To a cold solution (0° C.) of 195 (21.4 mg, 0.052 mmol) in DCM (1.0 mL) were added Et₃N (36 μL, 0.26 mmol) and 2-bromoacetyl bromide (10.5 μL, 0.12 mmol). After stirring for 10 min on ice, the ice bath was removed and aqueous 0.1 M NaOH solution (0.8 mL) was added. After stirring at room temperature for 20 min, the water layer was extracted with DCM (2×5 mL). The organic layers were combined and concentrated in vacuo. The crude brown oil was purified by flash column chromatography over silicagel (0%→18% MeOH in DCM) to give 196 as a clear oil (6.9 mg, 0.011 mmol, 20%). LCMS (ESI+) calculated for C₂₁H₄₀Br₂N₃O₁₀⁺ (M+H⁺) 654.36 found 654.29.

### Example 55. Synthesis of XL12

To a solution of 196 (6.9 mg, 0.011 mmol) in DCM (0.8 mL) were added bis(4-nitrophenyl) carbonate (3.8 mg, 0.012 mmol) and Et₃N (5 μL, 0.03 mmol). After stirring at room temperature for 18 h, 155 (BCN-PEG₂-NH₂, 3.3 mg, 0.01 mmol) dissolved in DCM (0.5 mL) was added. After stirring for an additional of 2 h, the mixture was concentrated in vacuo and purified by flash column chromatography over silica gel (gradient: A. 0%→30% EtOAc in DCM (till p-nitrophenol was eluded), followed by gradient B. 0%→20% MeOH in DCM) to give XL12 as a clear oil (1.0 mg, 0.001 mmol, 9%). LCMS (ESI+) calculated for C₃₉H₆₆Br₂N₅O₁₅⁺ (M+H⁺) 1004.77 found 1004.51.

### Example 56. Synthesis of XL14

To a solution of 152 (methyltetrazine-PEG₄-NH₂·HCl, 7.8 mg, 0.02 mmol) in dry DMF (80 μL) was added triethylamine (9 μL, 0.06 mmol) followed by 338 (25 mg, 0.02 mmol). After stirring at room temperature for 6.5 h, the reaction mixture was further diluted with DMF (400 μL) and purified by RP HPLC (Column Xbridge prep C18 5 um OBD, 30×100 mm, 5%→90% MeCN in H₂O (both containing 1% acetic acid)). XL14 was obtained as a pink oil (12.8 mg, 0.008 mmol, 45%). LCMS (ESI+) calculated for C₆₉H₈₆N₁₁O₂₁S₂⁺ (M+NH₄⁺) 1469.61, found 1469.69.

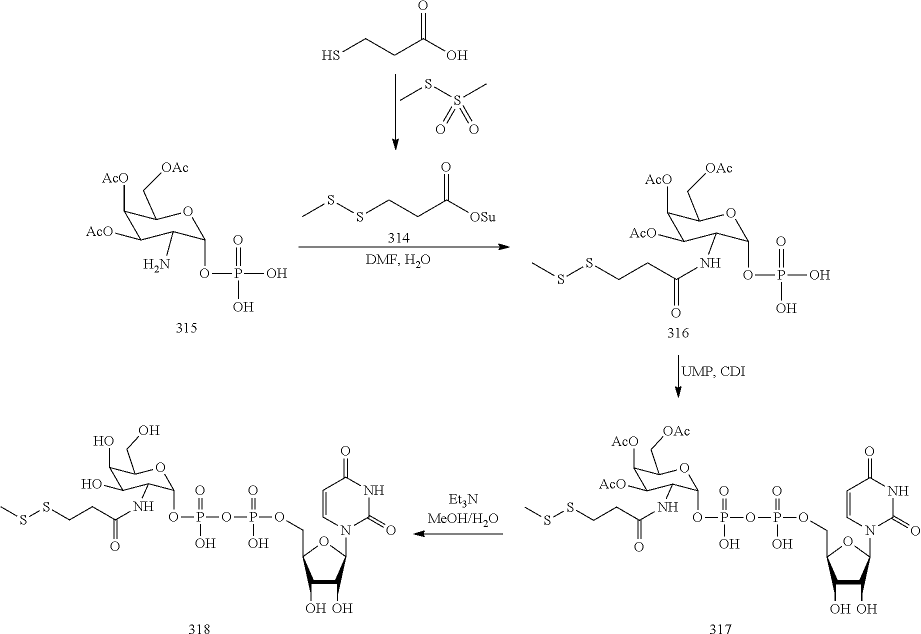

### Example 57. Synthesis of 314

A solution of 3-mercaptopropanoic acid (200 mg, 1.9 mmol) in water (6 mL) was cooled to 0° C., followed by the addition of methyl methanethiosulfonate (263 mg, 2.1 mmol) in ethanol (3 mL). The reaction was stirred overnight and warmed to room temperature. Subsequently, the reaction was quenched by saturated aqueous NaCl (10 mL) and Et₂O (20 mL). The water layer was extracted with Et₂O (3×20 mL), and the combined organic layers were dried over Na₂SO₄, filtrated and concentrated to yield the crude disulfide product (266 mg, 1.7 mmol, 93%). ¹H-NMR (400 MHz, CDCl₃): δ 7.00 (bs, 1H), 2.96-2.92 (m, 2H), 2.94-2.80 (m, 2H), 2.43 (s, 3H).

The crude disulfide derived from 3-mercaptopropanoic acid (266 mg, 1.7 mmol) was dissolved in CH₂Cl₂ (20 mL) followed by the addition of EDC·HCl (480 mg, 2.2 mmol) and N-hydroxy succinimide (270 mg, 2.1 mmol). The reaction was stirred for 90 minutes and quenched with water (20 mL). The organic layer was washed with saturated aqueous NaHCO₃ (2×20 mL). The organic layer was dried over Na₂SO₄, filtrated and concentrated to give crude 314 (346 mg, 1.4 mmol, 81%). ¹H-NMR (400 MHz, CDCl₃): δ 3.12-3.07 (m, 2H), 3.02-2.99 (m, 2H), 2.87 (bs, 4H), 2.44 (s, 3H).

### Example 58. Synthesis of 316

To a solution of 315 (prepared according WO2015057063 example 40, incorporated by reference) (420 mg, 1.14 mmol) in CH₂Cl₂/DMF (5 mL each) were added crude 314 (425 mg, 1.71 mmol) and Et₃N (236 μL, 1.71 mmol). The reaction mixture was stirred overnight followed by concentration under reduced pressure. Flash chromatography (1:0-6:4 MeCN:MeOH) afforded 316 (358 mg, 0.7 mmol, 60%). ¹H-NMR (400 MHz, CD₃OD): δ 5.46-5.45 (m, 1H), 5.33-5.27 (m, 1H), 5.15-5.11 (m, 1H), 4.43-4.41 (m, 1H), 4.17-4.06 (m, 2H), 3.97-3.88 (m, 1H), 2.89-2.83 (m, 2H), 2.69-2.53 (m, 2H), 2.32 (s, 3H), 2.04 (s, 3H), 1.91 (s, 3H), 1.86 (s, 3H).

### Example 59. Synthesis of UDP GalNProSSMe (318)

To a solution of UMP·NBu₃ (632 mg, 1.12 mmol) in DMF (5 mL) CDI (234 mg, 1.4 mmol) was added and stirred for 30 minutes. Methanol (25 μL, 0.6 mmol) is added and after 15 minutes the reaction is placed under high vacuum for 15 minutes. Subsequently, 316 (358 mg, 0.7 mmol) and NMI·HCl (333 mg, 2.8 mmol) are dissolved in DMF (2 mL) and added to the reaction mixture. After stirring overnight, the reaction mixture is concentrated under reduced pressure to give crude 317.

The crude product 317 is dissolved in MeOH:H₂O:Et₃N (7:3:3, 10 mL) and stirred overnight followed by the addition of additional MeOH:H₂O:Et₃N (7:3:3, 5 mL). After 48 h, total reaction time the reaction mixture was concentrated under reduced pressure. The crude product was purified via anion exchange column (Q HITRAP, 3×5 mL, 1×20 mL column) in two portions. First binding on the column was achieved via loading with buffer A (10 mM NaHCO₃) and the column was rinsed with 50 mL buffer A. Next a gradient to 70% B (250 mM NaHCO₃) was performed to elute UDP GalNProSSMe 318 (355 mg, 0.5 mmol, 72%). 1H-NMR (400 MHz, D₂O): δ 7.86-7.84 (m, 1H), 5.86-5.85 (m, 1H), 5.44 (bs, 1H), 4.26-4.22 (m, 2H), 4.17-4.08 (m, 6H), 3.92 (m, 1H), 3.84-3.83 (m, 1H), 3.66-3.64 (m, 2H), 2.88 (t, J=7.2 Hz, 2H), 2.68 (t, J=7.2 Hz, 2H), 2.31 (s, 3H).

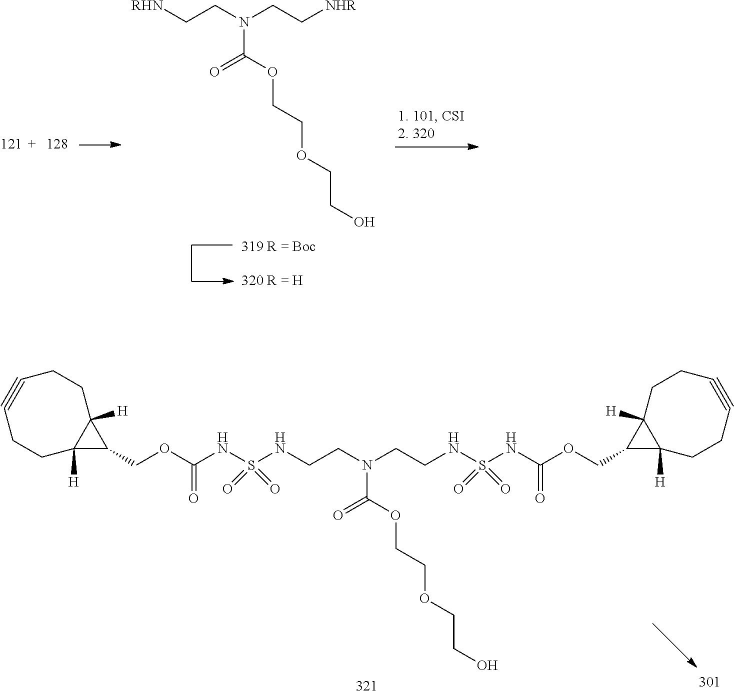

### Example 60. Synthesis of 319

To a solution of compound 121 (442 mg, 1.46 mmol) in DCM (1 mL) and DMF (200 μL) was added a solution of compound 128 in DCM (1 mL) and triethylamine (609 μL, 4.37 mmol). After stirring the mixture for 16 hours, it was concentrated in vacuo. The residue was purified by silica gel column chromatography (50%→100% EtOAc in heptane) and gave 319 (316 mg) This was further purified by RP HPLC (Column Xbridge prep C18 5 μm OBD, 30×100 mm, 5%→90% MeCN (1% AcOH) in water (1% AcOH). Product 319 was obtained in 17% yield as a colorless oil (110 mg, 0.25 mmol). LCMS (ESI+) calculated for C₁₉H₃₇N₃NaO₈⁺ ((M+Na⁺) 458.25, found 458.33. ¹H-NMR (400 MHz, CDCl₃): δ (ppm) 5.41-4.89 (m, 2H), 4.31-4.24 (m, 2H), 3.78-3.68 (m, 4H), 3.65-3.59 (m, 2H), 3.44-3.34 (m, 4H), 3.34-3.19 (m, 4H), 1.43 (s, 18H).

### Example 61. Synthesis of 320

Compound 319 (107 mg, 0.25 mmol) was dissolved in DCM (1 mL). Then 4 M HCl in dioxane (300 μL, 1.2 mmol, 4.8 equiv.) was added. After stirring the mixture for 15 hours, it was decanted from the precipitate and the precipitate was washed once with DCM (2 mL). Product 320 was obtained in quantitive yield as a white sticky solid (89.9 mg, 0.29 mmol). This was used directly in the next step.

### Example 62. Synthesis of 321

To a solution of 101 (75 mg, 0.50 mmol, 2 equiv.) in DCM (1 mL) was added CSI (41 μL, 0.48 mmol, 1.9 equiv.). After stirring for 6 minutes, triethylamine (139 μL, 1.0 mmol, 4 equiv.) was added. A stock solution of 320 was prepared by adding DMF (200 μL) and DCM (2 mL) followed by triethylamine (139 μL, 0.75 mmol, 3 equiv.). Part of this stock solution of 320 (32 μL, 0.25 mmol) was added to the original reaction mixture containing the CSI. After stirring the mixture for 16 hours, it was concentrated in vacuo. The residue was purified by silica gel column chromatography (0%→10% MeOH in DCM). Product 321 was obtained in 3% yield as a colorless oil (11 mg, 14.2 μmol). LCMS (ESI+) calculated for C₃₁H₄₈N₅O₁₂S₂⁺ ((M+H⁺) 746.27, found 746.96. ¹H-NMR (400 MHz, CDCl₃): δ (ppm) 6.36-5.94 (m, 2H), 4.38-4.17 (m, 6H), 3.84-3.79 (m, 2H), 3.77-3.72 (m, 2H), 3.68-3.63 (m, 2H), 3.54-3.45 (m, 4H), 3.39-3.27 (m, 4H), 2.38-2.16 (m, 12H), 1.67-1.47 (m, 5H), 1.40 (quintet, J=8.0 Hz, 2H), 1.05-0.93 (m, 4H).

### Example 63. Synthesis of 301 (LD01)

To a solution of 321 (10.6 mg, 14.2 μmol) in DCM (100 μL) were added bis(4-nitrophenyl) carbonate (4.3 mg, 14.2 μmol, 1.0 equiv.) and triethylamine (5.9 μL, 42.6 μmol, 3.0 equiv.). After stirring for 66 hours part of this mixture was treated with a stock solution of vc-PABC-MMAE. TFA in DMF (200 μL, 50 mg/mL) and an additional amount of triethylamine (5.9 μL, 42.6 μmol, 3.0 equiv.). After 24 hours it was concentrated partly in vacuo. The residue was purified by RP HPLC (Column Xbridge prep C18 5 μm OBD, 30×100 mm, 5%→90% MeCN (1% AcOH) in water (1% AcOH). Compound 301 was obtained in 28% yield as a film (3.4 mg, 1.9 μmol). LCMS (ESI+) calculated for C₉₀H₁₄₀N₁₅O₂₅S₂⁺ ((M+H⁺) 1894.96, found 1895.00.

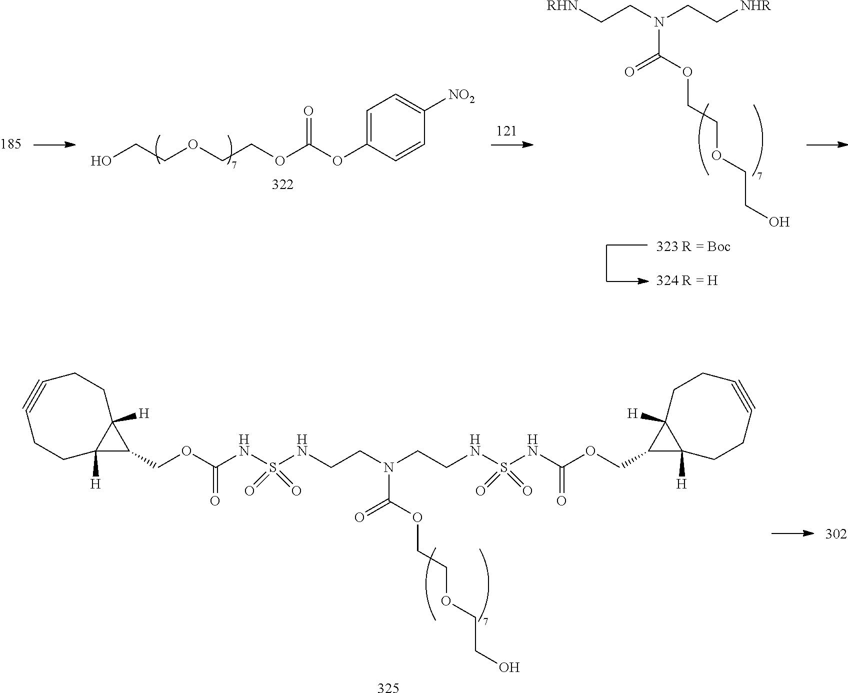

### Example 64. Synthesis of 322

To a solution of 185 (octaethylene glycol) in DCM (10 mL) was added triethylamine (1.0 mL, 7.24 mmol; 2.5 equiv.) followed by dropwise addition of a 4-nitrophenyl chloroformate (0.58 g; 2.90 mmol; 1 equiv.) solution in DCM (5 mL) in 28 minutes. After stirring the mixture for 90 minutes, it was concentrated in vacuo. The residue was purified by silica gel column chromatography (75%→0% EtOAc in DCM followed by 0%→7% MeOH in DCM). Product 322 was obtained in 38% yield as a colorless oil (584.6 mg; 1.09 mmol). LCMS (ESI+) calculated for C₂₃H₃₈NO₁₃⁺ (M+H⁺) 536.23, found 536.93. ¹H-NMR (400 MHz, CDCl₃): δ (ppm) 8.28 (d, J=12.0 Hz, 2H), 7.40 (d, J=12.0 Hz, 2H), 4.47-4.42 (m, 2H), 3.84-3.79 (m, 2H), 3.75-3.63 (m, 26H), 3.63-3.59 (m, 2H), 2.70-2.55 (br. s, 1H).

### Example 65. Synthesis of 323

To a solution of compound 121 (127 mg, 0.42 mmol) in DCM (1 mL) was added part (0.5 mL; 0.54 mmol; 1.3 equiv.) of a prepared stock solution of 322 (584 mg in DCM (1 mL)) followed by triethylamine (176 μL, 1.26 mmol; 3 equiv.) and HOBt (57 mg; 0.42 mmol; 1 equiv.). After stirring the mixture for 4.5 days, it was concentrated in vacuo. The residue was taken up in a mixture of acetonitrile (4.2 mL) and 0.1 N NaOH (4.2 mL, 1 equiv.). After stirring the mixture for 24 hours, additional solid NaOH (104.5 mg) was added. After stirring the mixture for another 5 hours, the mixture was extracted with DCM (2×10 mL). The combined organic layers were concentrated in vacuo and the residue was purified by silica gel column chromatography (0%→15% MeOH in DCM). Product 323 was obtained in 54% yield as a pale yellow oil (164.5 mg, 0.23 mmol). LCMS (ESI+) calculated for C₂₆H₅₄N₃O₁₂⁺ (M-BOC⁺) 600.36, found 600.49. ¹H-NMR (400 MHz, CDCl₃): δ (ppm) 5.27-5.05 (m, 2H), 4.26-4.21 (m, 2H), 3.76-3.59 (m, 30H), 3.43-3.33 (m, 4H), 3.33-3.22 (m, 4H), 1.43 (s, 18H).

### Example 66. Synthesis of 324

Compound 323 (164 mg, 0.23 mmol) was dissolved in DCM (1 mL). Then 4 M HCl in dioxane (293 μL, 1.17 mmol, 5 equiv.) was added. After stirring the mixture for 18 hours, additional 4 M HCl in dioxane (293 μL, 1.17 mmol, 5 equiv.) was added. After stirring the mixture for another 5 hours, the mixture was concentrated in vacuo. Product 324 was obtained in quantitative yield as a white sticky solid (132 mg, 0.23 mmol). This was used directly in the next step.

### Example 67. Synthesis of 325

To a solution of 101 (81 mg, 0.54 mmol, 2.3 equiv.) in DCM (2 mL) was added CSI (43 μL, 0.49 mmol, 2.1 equiv.). After stirring for 15 minutes triethylamine (164 μL, 1.17 mmol, 5 equiv.) was added. A solution of 324 was prepared by adding DCM (2 mL) and triethylamine (164 μL, 1.17 mmol, 5 equiv.). This stock solution was added to the original reaction mixture after 6 minutes. After stirring the mixture for 23 hours, it was concentrated in vacuo. The residue was purified by silica gel column chromatography (0%→12% MeOH in DCM). Product 325 was obtained in 31% yield as pale yellow oil (73.0 mg, 72.2 μmol). LCMS (ESI+) calculated for C₄₃H₇₂N₅O₁₈S₂⁺ (M+H⁺) 1010.43, found 1010.50.

¹H-NMR (400 MHz, CDCl₃): δ (ppm) 6.21-5.85 (m, 2H), 4.38-4.17 (m, 6H), 3.80-3.57 (m, 30H), 3.57-3.44 (m, 4H), 3.44-3.30 (m, 4H), 2.38-2.16 (m, 12H), 1.64-1.48 (m, 4H), 1.40 (quintet, J=8.0 Hz, 2H), 1.05-0.91 (m, 4H).

### Example 68. Synthesis of 302 (LD02)

To a solution of 325 (19.5 mg, 19.7 μmol) in DCM (100 μL) were added bis(4-nitrophenyl) carbonate (6.0 mg, 19.7 μmol, 1.0 equiv.) and triethylamine (8.2 μL, 59.1 μmol, 3.0 equiv.). After stirring for 66 hours part of this mixture was treated with a stock solution of vc-PABC-MMAE. TFA in DMF (200 μL, 50 mg/mL) and an additional amount of triethylamine (8.2 μL, 59.1 μmol, 3.0 equiv.). After 95 hours it was concentrated partly in vacuo. The residue was purified by RP HPLC (Column Xbridge prep C18 5 μm OBD, 30×100 mm, 5%→90% MeCN (1% AcOH) in water (1% AcOH). Compound 302 was obtained in 9% yield as a film (3.7 mg, 1.71 μmol). LCMS (ESI+) calculated for C₁₀₂H₁₆₅N₁₅O₃₁S₂²⁺ (M+2H⁺) 1080.56, found 1080.74.

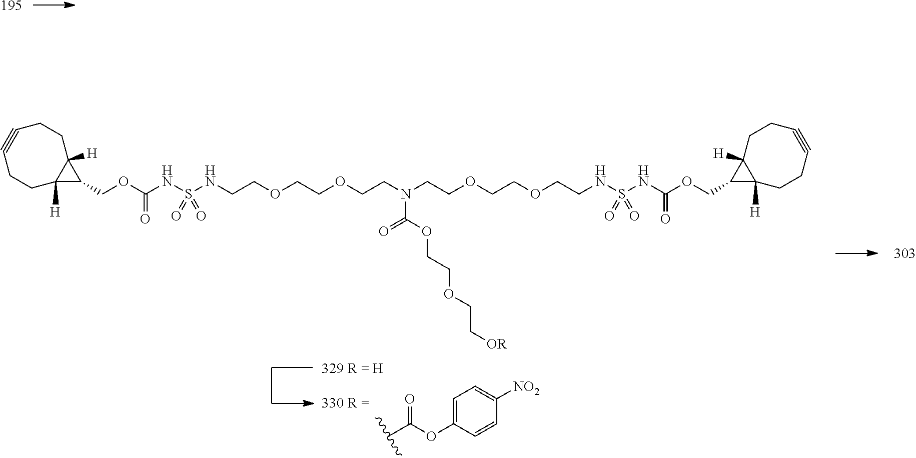

### Example 69. Synthesis of 329

To a solution of 101 (18 mg, 0.12 mmol) in DCM (1 mL) was added chlorosulfonyl isocyanate (CSI). After 30 min, Et₃N (37 μL, 27 mg, 0.27 mmol) was added. To a solution of 195 (26 mg, 0.054 mmol) in DCM (1 mL) was added Et₃N (37 μL, 27 mg, 0.27 mmol). This mixture was added to the reaction mixture. After 45 min, the reaction mixture was concentrated and the residue was purified by silica gel chromatography (DCM to 7% MeOH in DCM). Product 329 was obtained as a colorless film (27 mg, 0.029 mmol, 54%). LCMS (ESI+) calculated for C₃₉H₆₄N₅O₁₆S₂⁺ (M+H⁺) 922.38, found 922.50.

### Example 70. Synthesis of 330

To a solution of 329 in DCM (1 mL) was added bis(4-nitrophenyl) carbonate (8.9 mg, 29.3 μmol) and Et₃N (12.2 μL, 8.9 mg, 87.9 μmol). After 1 d, 0.28 mL was used for the preparation of compound 303. After 2 d, extra bis(4-nitrophenyl) carbonate (7.0 mg, 23 μmol) was added to the main reaction mixture. After 1 day, the reaction mixture was concentrated and the residue was purified by silica gel column chromatography. Product 330 was was obtained as a colorless film (17.5 mg, 0.016 mmol, 55% (76% corrected)). LCMS (ESI+) calculated for C₄₆H₆₇N₆O₂₀S₂⁺ (M+H⁺) 1087.38, found 1087.47.

### Example 71. Synthesis of 303 (LD03)

To the reaction mixture of 330 (0.28 mL, theoretically containing 8.8 mg, 8.1 μmol) was added Et₃N (3.4 μL, 2.5 mg, 24.3 μmol) and a solution of vc-PABC-MMAE. TFA (10 mg, 8.1 μmol) in DMF (200 μL). After 21 h, 2,2′-(ethylenedioxy)bis(ethylamine) (4.7 μL, 4.8 mg, 32 μmol) was added. After 45 min, the reaction mixture was concentrated under a stream of nitrogen gas. The residue was purified by RP-HPLC (Column Xbridge prep C18 5 μm OBD, 30×100 mm, 30% to 90% MeCN (1% AcOH) in water (1% AcOH). Product 303 was obtained as a colorless film (5.6 mg, 2.7 μmol). LCMS (ESI+) calculated for C₉₈H₁₅₇N₁₅O₂₉S₂²⁺ ((M+2H⁺)/2) 1036.53, found 1036.70.

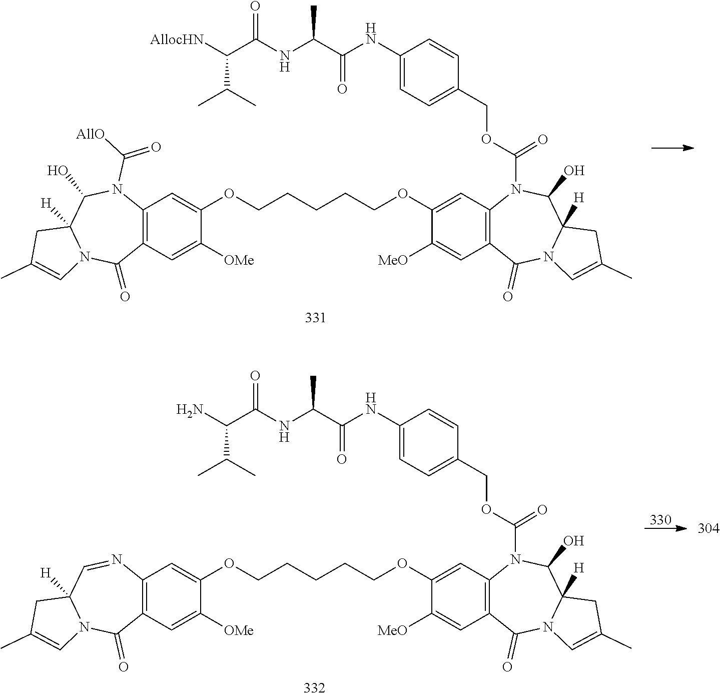

### Example 72. Synthesis of 332

To a solution of Alloc₂-va-PABC-PBD 331 (10.0 mg, 0.009 mmol) in degassed DCM (400 μL, obtained by purging N₂ through DCM for 5 minutes) were added pyrrolidine (1.9 μL, 0.027 mmol) and Pd(PPh₃)₄ (1.6 mg, 0.0014 mmol). After stirring for 15 min at ambient temperature, the reaction mixture was diluted with DCM (10 mL) and aqueous saturated NH₄Cl (10 mL) was added. The crude mixture was extracted with DCM (3×10 mL). The organic layers were combined, dried over Na₂SO₄, filtered, and concentrated in vacuo. The yellow residue was redissolved in DMF (450 μL) and MeCN (450 μL) and purified by RP HPLC (Column Xbridge prep C18 5 μm OBD, 30×100 mm, 5%→90% MeCN in H₂O (both containing 0.1% formic acid)). The pure fractions were neutralized over a SPE column (PL-HCO₃ MP, 500 mg/6 mL), concentrated and co-evaporated with MeCN (2×5 mL) to give 332 as a white solid (4.8 mg, 0.005 mmol, 58%). LCMS (ESI+) calculated for C₄₉H₆₀N₇O₁₁⁺ (M+H⁺) 923.04 found 923.61.

### Example 73. Synthesis of 304 (LD04)

To a solution of 332 (4.8 mg, 0.005 mmol) in anhydrous degassed DMF (60 μL, obtained by purging N₂ through DMF for 5 minutes) were added 330 (10 mg, 0.009 mmol, dissolved in 48 μL anhydrous degassed DMF), Et₃N (3.6 μL, 0.026 mmol), and HOBt (stock in anhydrous degassed DMF, 5.1 μL, 0.35 mg, 0.0026 mmol, 0.5 eq). After 41 h stirring at ambient temperature in the dark, the crude reaction mixture was diluted with DCM (300 μL) and purified by flash column chromatography over silicagel (0%→12% MeOH in DCM) to give 304 as a clear yellow oil (4.0 mg, 0.0021 mmol, 41%). LCMS (ESI+) calculated for C₈₉H₁₂₁N₁₂O₂₈S₂⁺ (M+H⁺) 1871.11 found 1871.09.

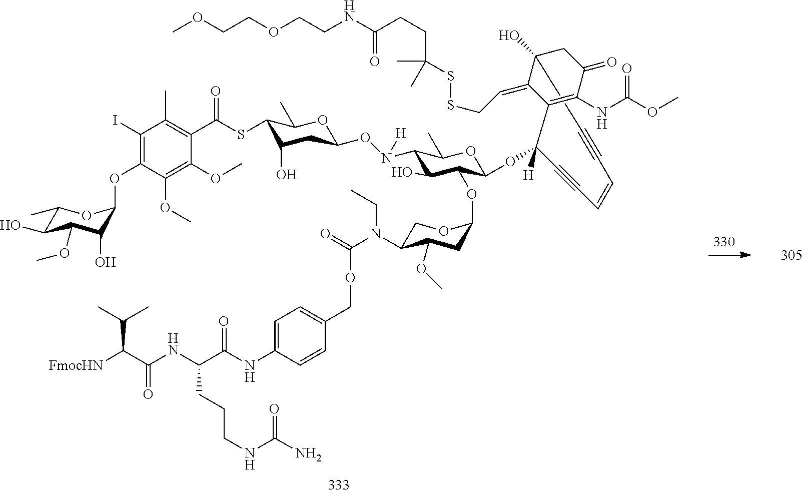

### Example 74. Synthesis of 305 (LD05)

To a solution of 333 (2.9 mg, 0.0013 mmol), prepared according to WO2019110725A1, Example 5-5, incorporated by reference, in anhydrous DMF (60 μL) were added 330 (1.45 mg, 0.0013 mmol) and Et₃N (1.2 μL, 0.023 mmol). After stirring at room temperature for 48 h, the reaction mixture was diluted with DMF (500 μL) and purified by RP HPLC (Column Xbridge prep C18 5 μm OBD, 30×100 mm, 30%→100% MeCN in H₂O (both containing 1% acetic acid)). The product 305 was obtained as a colorless film (0.6 mg, 0.207 μmol, 16%). LCMS (ESI+) calculated for C₁₂₄H₁₈₂IN₁₄O₄₆S₅⁺ (M/2+H⁺) 1447.03 found 1447.19.

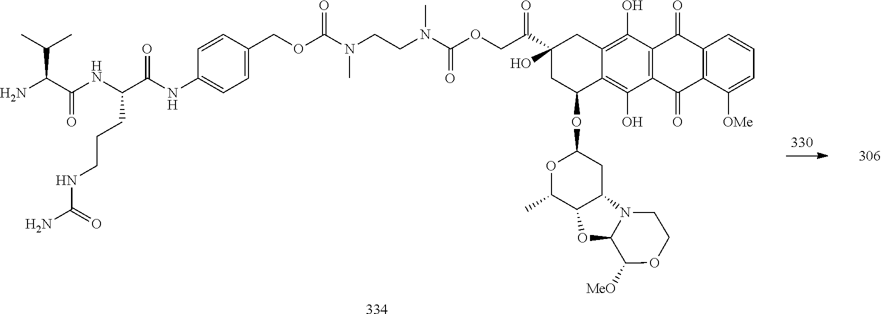 [SMILES: COc1cccc2c1C(=O)c1c(O)c3c(c(O)c1C2=O)C[C@@](O)(C(=O)COC(=O)N(C)CCN(C)C(=O)OCc1ccc(NC(=O)[C@H](CCCNC(N)=O)NC(=O)[C@@H](N)C(C)C)cc1)C[C@@H]3O[C@H]1C[C@H]2[C@H](O[C@@H]3[C@@H](OC)OCCN32)[C@H](C)O1 ; "334"]

### Example 75. Synthesis of 306 (LD06)

To a solution of 330 (7 mg, 0.006 mmol) in anhydrous DMF (150 μL) were added a stock of vc-PABC-DMEA-PNU (334) in anhydrous DMF (125 μL, 5.7 mg, 0.005 mmol) and Et₃N (2 μL, 0.015 mmol). After stirring at room temperature for 25 h, the reaction mixture was diluted with DCM (0.3 mL) and purified by flash column chromatography over silica gel (0%→20% MeOH in DCM) to give 306 as a red film (5 mg, 0.0024 mmol, 47%). LCMS (ESI+) calculated for C₉₆H₁₃₃N₁₃O₃₆S₃⁺ (M/2+H⁺) 1055.64 found 1055.50.

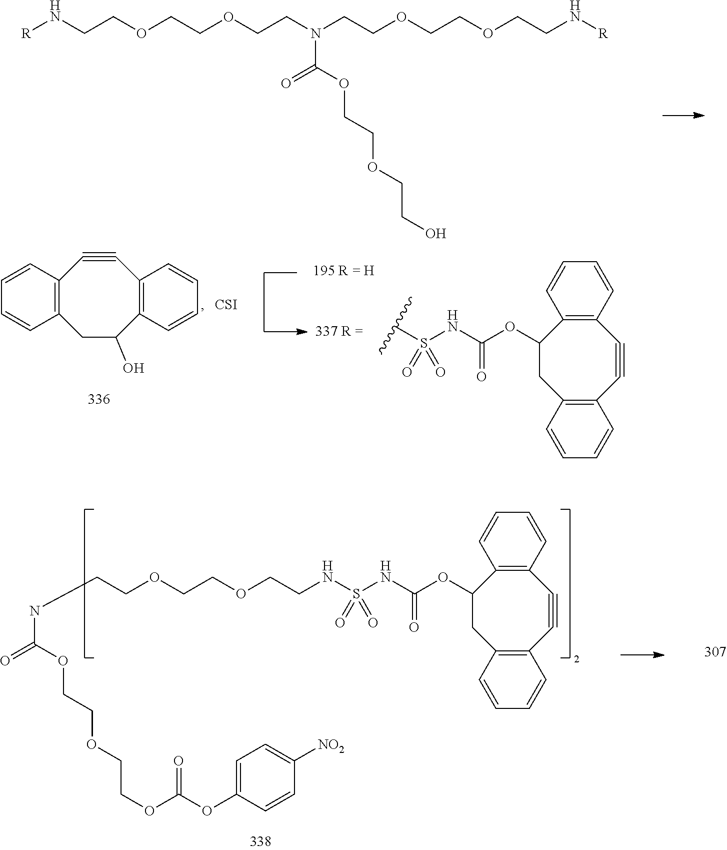

### Example 76. Synthesis of 337

Compound 336 (DIBO, 95 mg, 0.43 mmol) was dissolved in DCM (1.0 mL) and chlorosulfonyl isocyanate (33.0 μL, 0.37 mmol) was added at room temperature, and after 2 min insoluble material was formed. After stirring for an additional 15 min at room temperature, Et₃N (120.0 μL, 0.85 mmol) was added, all insoluble material disappeared, and addition of a mixture of 195 (71 mg, 0.0171) dissolved in DCM (1.0 mL) and Et₃N (120.0 μL, 0.85 mmol) was performed. After stirring at room temperature for 16 h, the crude mixture was concentrated in vacuo and purified by flash column chromatography over silicagel (0%→15% MeOH in DCM) after which it was co-evaporated with EtOAc (2×) to completely remove the MeOH. Product 337 was obtained as a waxy white solid (136.0 mg, 0.12 mmol, 75%). LCMS (ESI+) calculated for C₅₁H₆₃N₆O₁₆S₂⁺ (M+NH₄⁺) 1080.21 found 1080.59.

### Example 77. Synthesis of 338

To a solution of 337 (136.0 mg, 0.12 mmol) in DCM (2.0 mL) were added bis-(4-nitrophenyl) carbonate (47.0 mg, 0.15 mmol) and Et₃N (54.0 μL, 0.38 mmol). After stirring at room temperature for 18 h, the crude mixture was concentrated in vacuo and purified by flash column chromatography over silicagel (gradient: A. 0%→35% EtOAc in DCM (until p-nitrophenol was eluded), followed by gradient, B. 0%→13% MeOH in DCM) to give 338 as a light-yellow oil (89.0 mg, 0.07 mmol, 60%). LCMS (ESI+) calculated for C₄₈H₆₆N₇O₂₀S₂⁺ (M+NH₄⁺) 1245.31 found 1245.64.

### Example 78. Synthesis of 307 (LD07)

To a solution of 338 (6.95 mg, 0.005 mmol) in anhydrous DMF (93.0 μL) were added Et₃N (2.4 μL, 0.017 mmol) and stock solution of vc-PABC-MMAE. TFA (Levena Bioscience) in anhydrous DMF (70 μL, 7.0 mg, 0.005 mmol). After stirring at room temperature for 18 h, DMF (450 μL) was added and the crude mixture was purified by RP HPLC (Column Xbridge prep C18 5 μm OBD, 30×100 mm, 30%→100% MeCN in H₂O (both containing 1% acetic acid)). The product 307 was obtained as a colorless film (4.5 mg, 0.002 mmol, 36%). LCMS (ESI+) calculated for C₁₁₀H₁₅₂N₁₅O₂₉S₂⁺ (M/2+H⁺) 1106.30 found 1106.79.

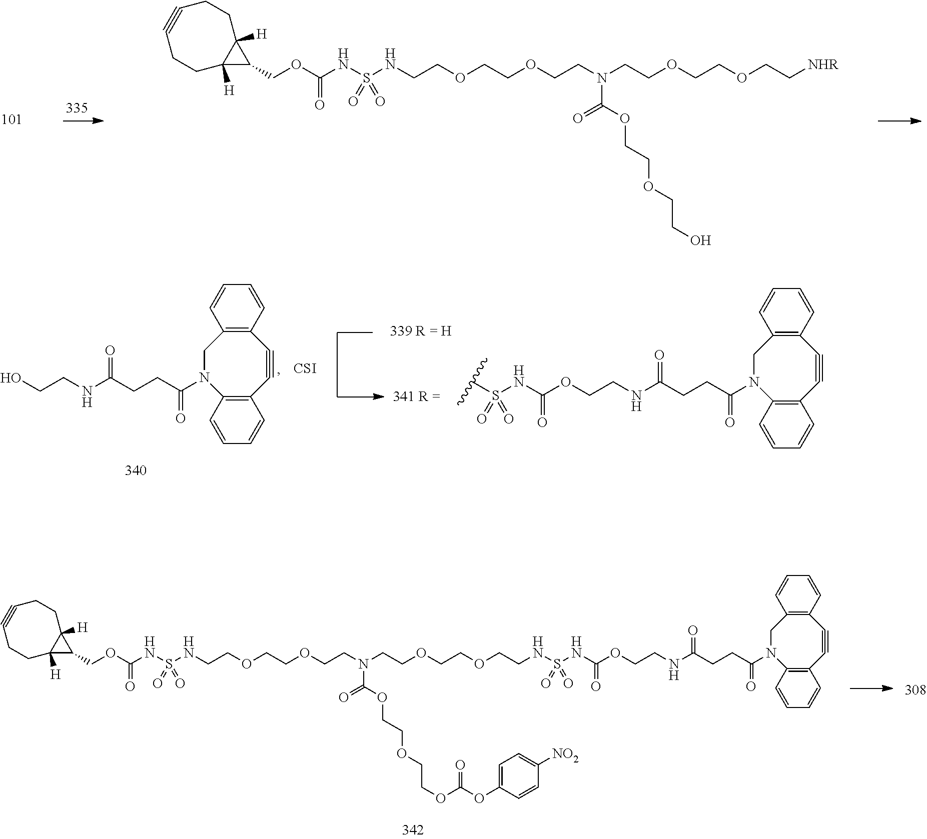

### Example 79. Synthesis of 341

Compound 101 (16.3 mg, 0.10 mmol) was dissolved in DCM (0.8 mL) and chlorosulfonyl isocyanate (8.6 μL, 0.099 mmol) was added at room temperature. After stirring for 15 min at room temperature, Et₃N (69.0 μL, 0.49 mmol) was added, followed by the addition of a mixture of 335 (40 mg, 0.099 mmol) dissolved in DCM (1.0 mL) and Et₃N (69.0 μL, 0.49 mmol). This mixture was stirred at room temperature for 1.5 h (mixture 1) to give crude 339. In another vial, 340 (DBCO-C₂—OH, Broadpharm) (34.0 mg, 0.099 mmol) was dissolved in DCM (0.8 mL) at room temperature and chlorosulfonyl isocyanate (7.75 μL, 0.089 mmol) was added. After stirring at room temperature for 15 min, Et₃N (69.0 μL, 0.49 mmol) was added followed by crude 339. After stirring at room temperature for another 2 h, the reaction mixture was concentrated in vacuo and purified by flash column chromatography over silicagel (0%→15% MeOH in DCM) after which it was co-evaporated with EtOAC (2×) to completely remove the MeOH. Product 341 was obtained as a clear yellow oil (20.0 mg, 0.017 mmol, 17%). LCMS (ESI+) calculated for C₅₀H₇₀N₇O₁₈S₂⁺ (M+H⁺) 1121.26 found 1121.59.

### Example 80. Synthesis of 342

To a solution of 341 (20.0 mg, 0.17 mmol) in DCM (1.0 mL) were added bis(4-nitrophenyl) carbonate (5.6 mg, 0.019 mmol) and Et₃N (7.5 μL, 0.053 mmol). After stirring at room temperature for 40 h, the crude mixture was concentrated in vacuo and purified by flash column chromatography over silicagel (gradient: A. 0%→30% EtOAc in DCM (until p-nitrophenol was eluded), followed by gradient B. 0%→20% MeOH in DCM) to give 342 as a clear light yellow oil (6.9 mg, 0.005 mmol, 30%). LCMS (ESI+) calculated for C₅₇H₇₃N₈O₂₂S₂⁺ (M+H⁺) 1286.36 found 1286.57.

### Example 81. Synthesis of 308 (LD08)

To a solution of 342 (3.6 mg, 0.0028 mmol) in anhydrous DMF (35.0 μL) were added Et₃N (1.2 μL, 0.008 mmol) and stock solution of vc-PABC-MMAE. TFA (Levena Bioscience) in anhydrous DMF (34 μL, 3.4 mg, 0.0028 mmol). After stirring at room temperature for 27 h, DCM (400 μL) was added and the crude mixture was purified by flash column chromatography over silicagel (0%→30% MeOH in DCM) to give 308 as a colorless film (3.7 mg, 0.0016 mmol, 58%). LCMS (ESI+) calculated for C₁₀₉H₁₆₁N₁₇O₃₁S₂⁺ (M/2+H⁺) 1135.84 found 1135.73.

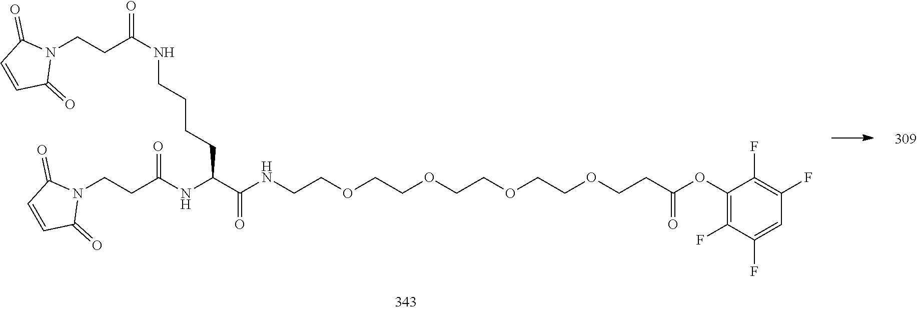 [SMILES: O=C(CCN1C(=O)C=CC1=O)NCCCC[C@H](NC(=O)CCN1C(=O)C=CC1=O)C(=O)NCCOCCOCCOCCOCCC(=O)Oc1c(F)c(F)cc(F)c1F]

### Example 82. Synthesis of 309 (LD09)

To a stock solution of vc-PABC-MMAE. TFA (Levena Bioscience) in anhydrous DMF (91 μL, 9.1 mg, 0.0073 mmol) were added Et₃N (5.1 μL, 0.037 mmol) and 343 (bis-maleimide-lysine-PEG₄-TFP, Broadpharm) (6.2 mg, 0.0073 mmol). After stirring at room temperature for 3 h, the mixture was diluted with DCM (0.4 mL) and purified by flash column chromatography over silicagel (0%→30% MeOH in DCM) to give 309 as a clear oil (9.1 mg, 0.0051 mmol, 69%). LCMS (ESI+) calculated for C₈₉H₁₃₈N₁₅O₂₄⁺ (M+H⁺) 1802.13 found 1802.11.

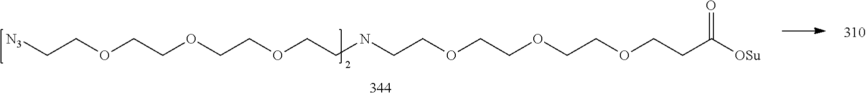

### Example 83. Synthesis of 310 (LD10)

To an Eppendorf vial containing 344 (4.3 mg, 6.0 μmol, 1.7 equiv.) was added was added a vc-PABC-MMAF. TFA salt in DMF (4.00 mg, 100 μL, 34.31 mmolar, 3.43 μmol, 1.0 equiv.), followed by triethylamine (1.43 μL, 10.3 μmol, 3.0 Eq). The mixture was mixed and the resulting colorless solution was left at rt for circa 3 hours. The reaction mixture was then purified directly via RP HPLC (Column Xbridge prep C18 5 μm OBD, 30×100 mm, 30%→90% MeCN (1% AcOH) in water (1% AcOH). The desired product 310 was obtained as a colorless residue (4.5 mg, 2.7 μmol, 79% yield).

LCMS (ESI+) calculated for C₈₀H₁₃₄N₁₅O₂₂⁺ (M+H⁺) 1656.98. found 1657.03.

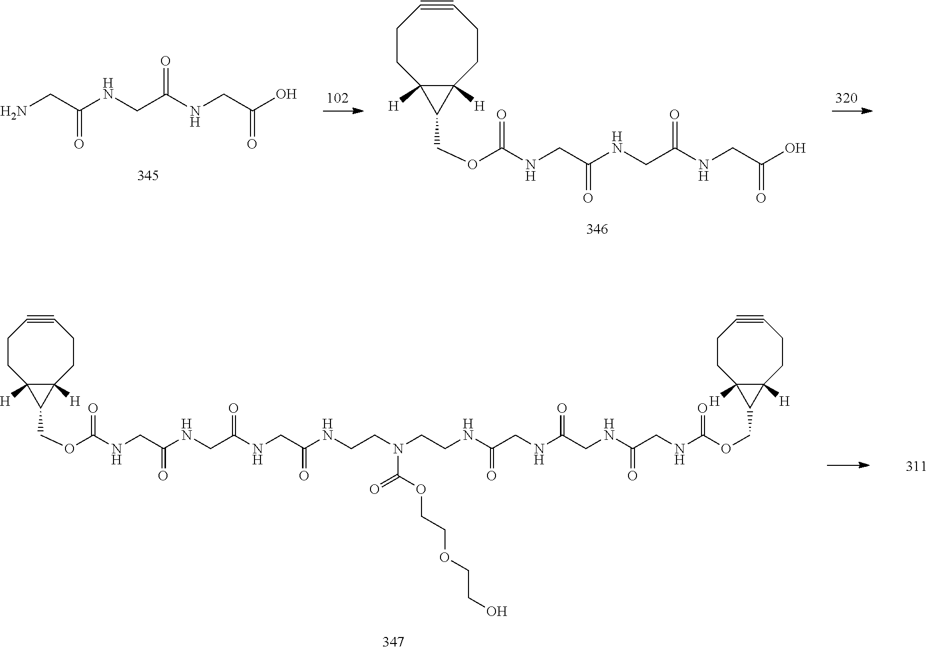

### Example 84. Synthesis of 346

To an Eppendorf vial containing 102 (54.7 mg, 1.00 Eq, 173 μmol) and 345 (triglycine, 28.8 mg, 0.878 equiv., 152 μmol) was added dry DMF (250 μL) and triethylamine (52.7 mg, 72.5 μL, 3 Eq, 520 μmol). The resulting yellow suspension was stirred at rt for 21 hours, followed by the addition of 50 μL H₂O to the RM. The reaction mixture was stirred at rt for another day upon which additional H₂O (200 μL) was added and the reaction mixture was stirred at rt for another 3 days. Next, MeCN (circa 0.5 mL) and additional Et₃N (circa 10 drops) were added and the resulting suspension was stirred for 1 hour at rt before conc. in vacuo. The yellow residue was taken up in DMF (600 μL) and the resulting yellow suspension was filtered over a membrane filter. The membrane-filter was washed with 200 μL additional DMF and the combined filtrates was purified directly via RP HPLC (Column Xbridge prep C18 5 μm OBD, 30×100 mm, 30%→90% MeCN (1% AcOH) in water (1% AcOH). The desired product 346 was obtained as a brown oil (41.5 mg, 114 μmol, 66% yield). LCMS (ESI+) calculated for C₁₇H₂₄N₃O₆⁺ (M+H⁺) 366.17. found 366.27.

### Example 85. Synthesis of 347

To a solution of 346 (21.6 mg, 0.056 mmol) in anhydrous DMF (0.3 mL) were added DIPEA (30 μL, 0.171 mmol) and HATU (21.6 mg, 0.056 mmol). After stirring at room temperature for 10 min, 320 (7.37 mg, 0.031 mmol) dissolved in DCM (310 μL) was added. After stirring at room temperature for 24 h, the mixture was purified by RP HPLC (Column Xbridge prep C18 5 μm OBD, 30×100 mm, 30%→100% MeCN in H₂O (both containing 1% AcOH)). The product 347 was obtained as an off-white oil (5.2 mg, 0.005 mmol, 20%). LCMS (ESI+) calculated for C₄₃H₆₄N₉O₁₄⁺ (M+H⁺) 931.02 found 931.68.

### Example 86. Synthesis of 311 (LD13)

To a solution of 347 (5.2 mg, 0.0056 mmol) in anhydrous DMF (200 μL) were added bis(4-nitrophenyl) carbonate (1.9 mg, 0.006 mmol) and Et₃N (2.4 μL, 0.016 mmol). After stirring at room temperature for 27 h, a stock solution of vc-PABC-MMAE. TFA (Levena Bioscience) (66 μL, 6.6 mg, 0.0053 mmol) and Et₃N (2 μL, 0.014 mmol) were added. After stirring for another 17 h at room temperature, the crude mixture was diluted with DMF (250 μL) and purified by RP HPLC (Column Xbridge prep C18 5 μm OBD, 30×100 mm, 5%→90% MeCN in H₂O (both containing 1% AcOH)). The product 311 was obtained as a clear oil (0.6 mg, 0.28 μmol, 5%). LCMS (ESI+) calculated for C₁₀₂H₁₅₆N₁₉O₂₇⁺ (M/2+H⁺) 1040.71 found 1040.85.

### Example 87. Synthesis of Compound 312

Compound 312 (LD11) was prepared according to the procedure described by Verkade et al., Antibodies 2018, 7, doi:10.3390/antib7010012, incorporated by reference.

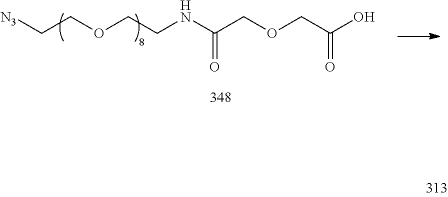

### Example 88. Synthesis of 313 (LD311)

To a vial containing 348 (2.7 mg, 1.1 Eq, 4.9 μmol) was added DMF (60 μL) and neat triethylamine (1.9 μL, 3 Eq, 13 μmol). Next, a solution of HBTU in dry DMF (2.0 mg, 11 μL, 472 mmolar, 1.2 Eq, 5.3 μmol) was added and the mixture was mixed. The reaction mixture was left at rt for 30 minutes, followed by the addition of va-PABC-MMAF. TFA salt (5.2 mg, 0.13 mL, 34.31 mmolar, 1 Eq, 4.4 μmol). The resulting mixture was mixed and left at rt for 110 minutes and was then purified directly via RP HPLC (Column Xbridge prep C18 5 μm OBD, 30×100 mm, 30%-90% MeCN (1% AcOH) in water (1% AcOH). The desired product 313 was obtained as a colorless oil (1.8 mg, 1.1 μmol, 26% yield). LCMS (ESI+) calculated for C₇₇H₁₂₇N₁₂O₂₃⁺ (M+H⁺) 1587.91. found 1588.05.

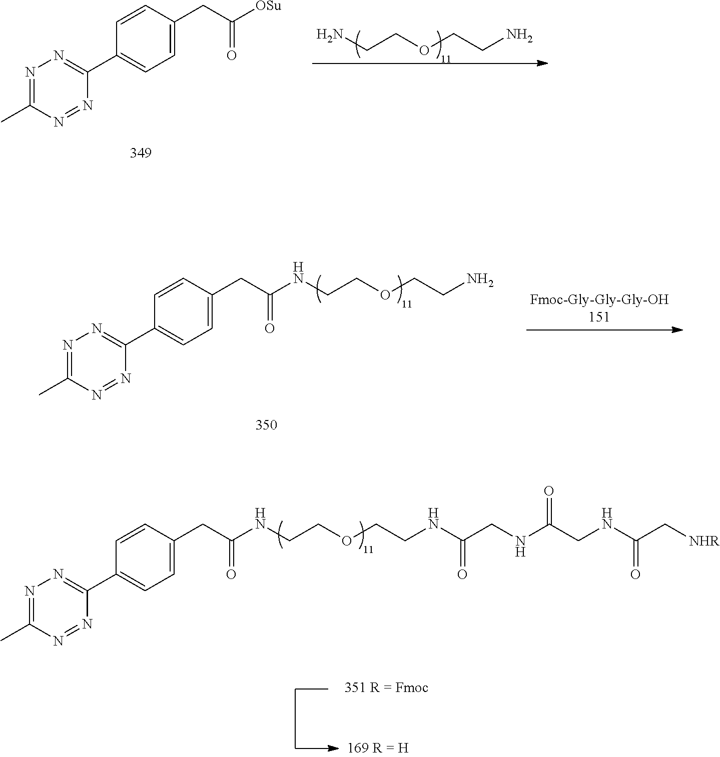

### Example 89. Synthesis of 350

To a solution of methyltetrazine-NHS ester 349 (19 mg, 0.057 mmol) in DCM (400 μL) was added amino-PEG₁₁-amine (47 mg, 0.086 mmol) dissolved in DCM (800 μL). After stirring at room temperature for 20 min, the mixture was concentrated in vacuo and purified by flash column chromatography over silicagel (0→50% MeOH (0.7 M NH₃) in DCM) which gave the desired compound 350 as a pink oil (17 mg, 0.022 mmol, 39%). LCMS (ESI+) calculated for C₃₅H₆₁N₆O₁₂⁺ (M+H⁺) 757.89 found 757.46.

### Example 90. Synthesis of 351

To a stirred solution of 151 (Fmoc-Gly-Gly-Gly-OH, 10 mg, 0.022 mmol) in anhydrous DMF (500 μL) were added DIPEA (11 μL, 0.067 mmol) and HATU (8.5 mg, 0.022 mmol). After 10 min, 350 (17 mg, 0.022 mmol) dissolved in anhydrous DMF (500 μL) was added. After stirring at room temperature for 18.5 h, the mixture was concentrated in vacuo and purified by flash column chromatography over silicagel (0→17% MeOH in DCM) which gave the desired compound 351 as a pink oil (26 mg, 0.022 mmol, quant.). LCMS (ESI+) calculated for C₅₆H₈₃N₁₀O₁₇⁺ (M+NH₄⁺) 1168.32 found 1168.67

### Example 91. Synthesis of 169

To a solution of 351 (26 mg, 0.022 mmol) in anhydrous DMF (500 μL) was added diethylamine (12 μL, 0.11 mmol). After stirring at room temperature for 1.5 h, the crude mixture was purified by RP HPLC (Column Xbridge prep C18 5 μm OBD, 30×100 mm, 5%→90% MeCN in H₂O (both containing 1% acetic acid)). The product 169 was obtained as a clear pink oil (10.9 mg, 0.011 mmol, 53%). LCMS (ESI+) calculated for C₄₁H₇₀N₉O₁₅⁺ (M+H⁺) 929.05 found 929.61.

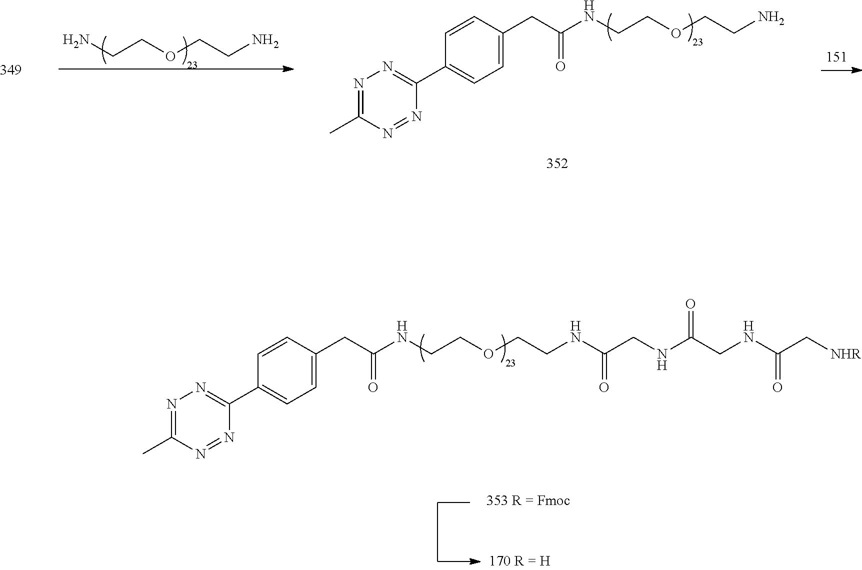

### Example 92. Synthesis of 352

To a solution of 349 (methyltetrazine-NHS ester, 10.3 mg, 0.031 mmol) in DCM (200 μL) was added amino-PEG₂₃-amine (50 mg, 0.046 mmol) dissolved in DCM (200 μL). After stirring at room temperature for 50 min, the mixture was concentrated in vacuo and purified by flash column chromatography over silicagel (0→60% MeOH (0.7 M NH₃) in DCM) which gave the desired compound 352 as a pink oil (17.7 mg, 0.013 mmol, 44%). LCMS (ESI+) calculated for C₅₉H₁₀₉N₆O₂₄⁺ (M+H⁺) 1286.52 found 1286.72.

### Example 93. Synthesis of 353

To a stirred solution of 151 (5.7 mg, 0.013 mmol) in anhydrous DMF (500 μL) were added DIPEA (7 μL, 0.04 mmol) and HATU (5.3 mg, 0.013 mmol). After 10 min, 352 (17.7 mg, 0.013 mmol) dissolved in anhydrous DMF (500 μL) was added. After stirring at room temperature for 6 h, the mixture was concentrated in vacuo and purified by flash column chromatography over silicagel (0→18% MeOH in DCM) which gave the desired compound 353 as a pink oil (21 mg, 0.012 mmol, 91%). LCMS (ESI+) calculated for C₈₀H₁₃₁N₁₀O₂₉⁺ (M/2+NH₄⁺) 857.45 found 857.08

### Example 94. Synthesis of 170

To a solution of 353 (21 mg, 0.012 mmol) in anhydrous DMF (500 μL) was added diethylamine (6.7 μL, 0.06 mmol). After stirring at room temperature for 4 h, the crude mixture was purified by RP HPLC (Column Xbridge prep C18 5 μm OBD, 30×100 mm, 5%→90% MeCN in H₂O (both containing 1% acetic acid)). The product 170 was obtained as a pink oil (11.6 mg, 0.008 mmol, 66%). LCMS (ESI+) calculated for C₆₅H₁₁₈N₉O₂₇⁺ (M+H⁺) 1457.68 found 1457.92.

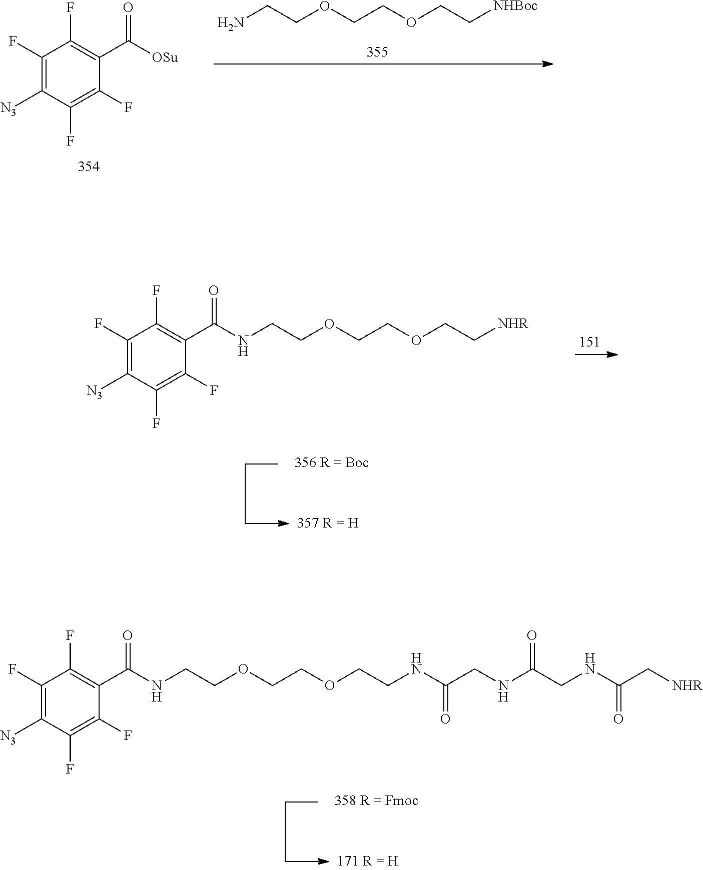

### Example 95. Synthesis of 356

To a solution of 354 (tetrafluorophenylazide-NHS ester, 40 mg, 0.12 mmol) in DCM (1 mL) were added 355 (Boc-NH-PEG₂-NH₂, 33 mg, 0.13 mmol) and Et₃N (50 μL, 0.36 mmol). After stirring in the dark at room temperature for 30 min, the mixture was concentrated in vacuo and purified by flash column chromatography over silicagel (0→7% MeOH in DCM) which gave the desired compound 356 as a clear oil (47 mg, 0.10 mmol, 84%). LCMS (ESI+) calculated for C₁₈H₂₄F₄N₅O₅⁺ (M+H⁺) 466.41 found 466.23.

### Example 96. Synthesis of 357

To a solution of 356 (47 mg, 0.10 mmol) in DCM (2 mL) was added 4.0 M HCl in dioxane (300 μL). After stirring in the dark at room temperature for 17.5 h, the mixture was concentrated and 357 was obtained as a white solid in quantitative yield (36 mg, 0.10 mmol). LCMS (ESI+) calculated for C₁₃H₁₆F₄N₅O₃⁺ (M+H⁺) 366.29 found 366.20.

### Example 97. Synthesis of 358

To a stirred solution of 151 (Fmoc-Gly-Gly-Gly-OH, 42 mg, 0.10 mmol) in anhydrous DMF (600 μL) were added DIPEA (50 μL, 0.30 mmol) and HATU (39 mg, 0.10 mmol). After 15 min in the dark, 357 (36 mg, 0.10 mmol) dissolved in anhydrous DMF (500 μL) was added. After stirring in the dark at room temperature for 41 h, the mixture was concentrated in vacuo and purified by flash column chromatography over silicagel (0→20% MeOH in DCM) which gave the desired compound 358 as a clear oil (36 mg, 0.047 mmol, 47%). LCMS (ESI+) calculated for C₃₄H₃₅F₄N₈O₈⁺ (M+H⁺) 759.68 found 759.38.

### Example 98. Synthesis of 171

To a solution of 358 (36 mg, 0.047 mmol) in anhydrous DMF (750 μL) was added diethylamine (24 μL, 0.24 mmol). After stirring in the dark at room temperature for 55 min, the crude mixture was purified by RP HPLC (Column Xbridge prep C18 5 μm OBD, 30×100 mm, 5%→90% MeCN in H₂O (both containing 1% acetic acid)). The product 171 was obtained as a clear oil (18.7 mg, 0.034 mmol, 74%). LCMS (ESI+) calculated for C₁₉H₂₅F₄N₈O₆⁺ (M+H⁺) 537.45 found 537.29.

### Example 99. Synthesis of BCN-LPETGG (172)

To a solution of 102 (10 mg, 0.031 mmol) in anhydrous DMF (500 μL) were added peptide 167 (H-LPETGG-OH, 18 mg, 0.031 mmol) and Et₃N (13 μL, 0.095 mmol). After stirring at room temperature for 93 h, the crude mixture was purified by RP HPLC (Column Xbridge prep C18 5 μm OBD, 30×100 mm, 5%→90% MeCN in H₂O (both containing 1% acetic acid)). The product 172 was obtained as a clear oil (16.8 mg, 0.022 mmol, 72%). LCMS (ESI+) calculated for C₃₅H₅₃N₆O₁₂⁺ (M+H⁺) 749.83 found 749.39.

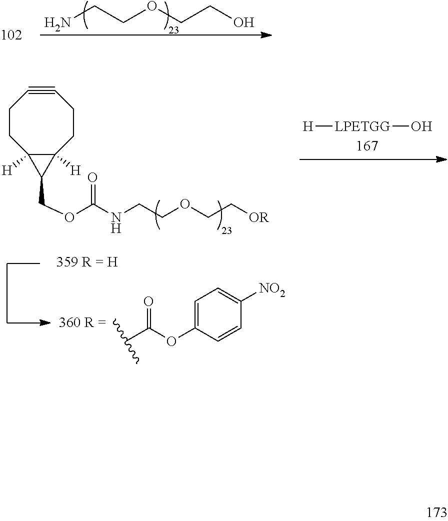

### Example 100. Synthesis of 359

To a solution of 102 (56 mg, 0.17 mmol) in DCM (8 mL) were added amino-PEG₂₄-alcohol (214 mg, 0.199 mmol) and Et₃N (80 μL, 0.53 mmol). After stirring at room temperature for 20 h, solvent was reduced in vacuo and the residue was purified by flash silica gel column chromatography (2→30% MeOH in DCM) which gave the desired compound 359 as a yellow oil in 95% yield (210 mg, 0.168 mmol). LCMS (ESI+) calculated for C₅₉H₁₁₁NO₂₆Na⁺ (M+Na⁺) 1273.50 found 1273.07.

### Example 101. Synthesis of 360

To a solution of 359 (170 mg, 0.136 mmol) and 4-nitrophenyl chloroformate (44 mg, 0.22 mmol) in DCM (7 mL) was added Et₃N (63 μL, 0.40 mmol). After stirring at room temperature for 41 h, solvent was reduced and the residue was purified by flash silica gel column chromatography (0→10% MeOH in DCM) which gave the desired compound 360 as a clear oil in 67% yield (129 mg, 0.091 mmol). LCMS (ESI+) calculated for C₆₆H₁₁₄N₂O₃₀Na⁺ (M+Na⁺) 1438.59 found 1438.13.

### Example 102. Synthesis of 173

To a solution of 360 (16 mg, 0.011 mmol) in anhydrous DMF (800 μL) were added 167 (peptide H-LPETGG-OH, 6.5 mg, 0.011 mmol) and Et₃N (5 μL, 0.04 mmol). After stirring at room temperature for 95 h, the crude mixture was purified by RP HPLC (Column Xbridge prep C18 5 μm OBD, 30×100 mm, 5%→90% MeCN in H₂O (both containing 1% acetic acid)). The product 173 was obtained as a clear oil (12.6 mg, 0.0068 mmol, 62%). LCMS (ESI+) calculated for C₈₄H₁₅₃N₈O₃₇⁺ (M/2+NH₄⁺) 942.55 found 924.26.

### Example 103. Synthesis of 174

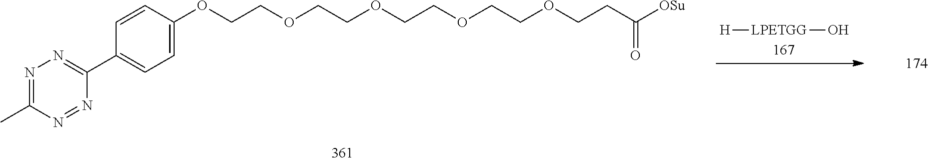

To a solution of 361 (methyltetrazine-PEG₅-NHS ester, 6.1 mg, 0.011 mmol) in anhydrous DMF (230 μL) were added peptide H-LPETGG-OH (6.5 mg, 0.011 mmol) and Et₃N (4 μL, 0.028 mmol). After stirring at room temperature for 22 h, the crude mixture was purified by RP HPLC (Column Xbridge prep C18 5 μm OBD, 30×100 mm, 5%→90% MeCN in H₂O (both containing 1% acetic acid)). The product 174 was obtained as a clear pink oil (9.9 mg, 0.01 mmol, 91%). LCMS (ESI+) calculated for C₄₄H₇₀N₁₁O₁₆⁺ (M+NH₄⁺) 1009.09 found 1009.61.

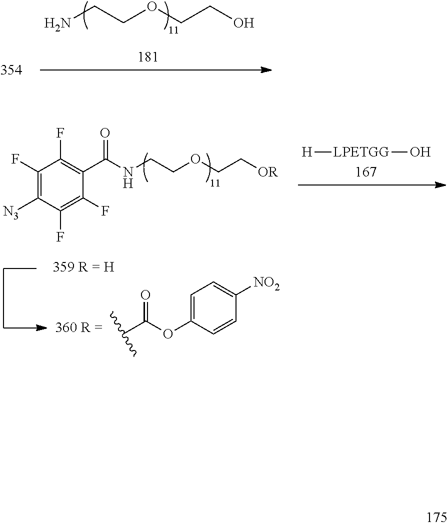

### Example 104. Synthesis of 362

To a solution of 354 (31 mg, 0.093 mmol) in DCM (1 mL) were added 181 (56 mg, 0.10 mmol) and Et₃N (40 μL, 0.28 mmol). After stirring in the dark at room temperature for 25 min, the mixture was concentrated in vacuo and purified by flash column chromatography over silicagel (0→15% MeOH in DCM) which gave the desired compound 362 as a clear oil (55 mg, 0.072 mmol, 77%). LCMS (ESI+) calculated for C₃₁H₅₁F₄N₄O₁₃⁺ (M+H⁺) 763.75 found 763.08.

### Example 105. Synthesis of 363

To a solution of 362 (55 mg, 0.072 mmol) in DCM (2 mL) were added 4-nitrophenyl chloroformate (13 mg, 0.064 mmol) and Et₃N (30 μL, 0.21 mmol). After stirring in the dark at room temperature for 21 h, the mixture was concentrated in vacuo and purified by RP HPLC (Column Xbridge prep C18 5 μm OBD, 30×100 mm, 5%→90% MeCN (1% AcOH) in water (1% AcOH). The product 363 was obtained as a yellow oil (13.3 mg, 0.014 mmol, 20%). LCMS (ESI+) calculated for C₃₈H₅₄F₄N₅O₁₇⁺ (M+H⁺) 928.85 found 928.57.

### Example 106. Synthesis of 175

To a solution of 363 (13.3 mg, 0.014 mmol) in anhydrous DMF (300 μL) were added 167 (peptide H-LPETGG-OH, 8.2 mg, 0.014 mmol) and Et₃N (6 μL, 0.043 mmol). After 26 h in the dark, the crude mixture was purified by RP HPLC (Column Xbridge prep C18 5 μm OBD, 30×100 mm, 5%→90% MeCN in H₂O (both containing 1% acetic acid)). The product 175 was obtained as a clear oil (11.4 mg, 0.0084 mmol, 59%). LCMS (ESI+) calculated for C₅₆H₈₉F₄N₁₀O₂₄⁺ (M+H⁺) 1362.35 found 1362.81.

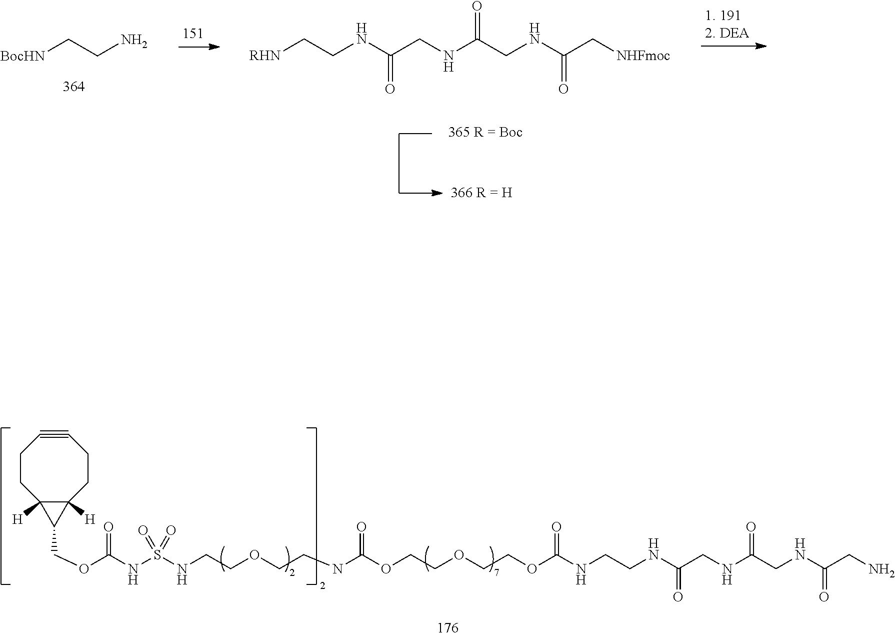

### Example 107. Synthesis of 365

To a stirred solution of 151 (Fmoc-Gly-Gly-Gly-OH, 20 mg, 0.049 mmol) in anhydrous DMF (350 μL) were added DIPEA (25 μL, 0.15 mmol) and HATU (18 mg, 0.049 mmol). After 10 min, compound 364 (N-Boc-ethylenediamine, 7.8 mg, 0.049 mmol) dissolved in anhydrous was added. After stirring at room temperature for 45 min, the mixture was concentrated in vacuo and purified by flash column chromatography over silicagel (0→30% MeOH in DCM) which gave the desired compound 365 as a clear oil (12.4 mg, 0.022 mmol, 46%). LCMS (ESI+) calculated for C₂₈H₃₆N₅O₇⁺ (M+H⁺) 554.61 found 554.46.

### Example 108. Synthesis of 366

To a stirred solution of 365 (12.4 mg, 0.022 mmol) in DCM (0.7 mL) was added 4.0 M HCl in dioxane (400 μL). After stirring at room temperature for 1 h, the mixture was concentrated and 366 was obtained as a white solid (11 mg, 0.022 mmol, quant.). LCMS (ESI+) calculated for C₂₃H₂₈N₅O₇⁺ (M+H⁺) 545.50 found 454.33.

### Example 109. Synthesis of 176

To a solution of 191 (8 mg, 0.0059 mmol) in anhydrous DMF (300 μL) were added Et₃N (2.5 μL, 0.017 mmol) and stock of 366 in anhydrous DMF (110 μL, 3.0 mg, 0.0059 mmol). After stirring at room temperature for 18 h, diethylamine (2 uL) was added. After an additional of 2 h, the mixture was purified by RP HPLC (Column Xbridge prep C18 5 μm OBD, 30×100 mm, 5%→90% MeCN in H₂O (both containing 1% acetic acid)). The product 176 was obtained as a clear oil (1.3 mg, 0.0009 mmol, 15%). LCMS (ESI+) calculated for C₆₀H₁₀₃N₁₀O₂₆S₂⁺ (M+H⁺) 1444.64 found 1444.75.

Expression of Cytokines and scFv's

### Example 110. Humanized OKT3 200

Humanized OKT3 (hOKT3) with C-terminal sortase A recognition sequence (C-terminal tag being identified by SEQ ID NO: 1) was obtained from Absolute Antibody Ltd (Oxford, United Kingdom). Mass spectral analysis showed one major product (observed mass 28836 Da).

### Example 111. Anti-4-1BB PF31

Anti-4-1 BB scFv was designed with a C-terminal sortase A recognition sequence followed by a His tag (amino acid sequence being identified by SEQ ID NO: 4). Anti-4-1BB scFv was transiently expressed in HEK293 cells followed by IMAC purification by Absolute Antibody Ltd (Oxford, United Kingdom). Mass spectral analysis showed one major product (observed mass 28013 Da, expected mass 28018 Da).

### Example 112. Cloning of SYR-(G₄S)₃-IL15 PF18 into pET32a Expression Vector

The SYR-(G₄S)₃-IL15 (PF18) (amino acid sequence being identified by SEQ ID NO: 5) was designed with an N-terminal (M)SYR sequence, where the methionine will be cleaved after expression leaving an N-terminal serine, and a flexible (G₄S)₃ spacer between the SYR sequence and IL15. The codon-optimized DNA sequence was inserted into a pET32A expression vector between NdeI and XhoI, thereby removing the sequence encoding the thioredoxin fusion protein, and was obtained from Genscript, Piscataway, USA.

### Example 113. E. coli Expression of SYR-(G4S)3-IL15 PF18 and Inclusion Body Isolation

Expression of SYR-(G₄S)₃—IL15 (PF18) starts with the transformation of the plasmid (pET32a-SYR-(G4S)3-IL15) into BL21 cells (Novagen). Transformed cells were plated on LB-agar with ampicillin and incubated overnight at 37° C. A single colony was picked and used to inoculate 50 mL of TB medium+ampicillin followed by incubated overnight at 37° C. Next, the overnight culture was used to inoculation 1000 mL TB medium+ampicillin. The culture was incubated at 37° C. at 160 RPM and, when OD600 reached 1.5, induced with 1 mM IPTG (1 mL of 1M stock solution). After >16 hour induction at 37° C. at 160 RPM, the culture was pelleted by centrifugation (5000×g-5 min).

The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000×g). Half of the insoluble fraction was dissolved in 30 mL BugBuster™ with lysozyme (final concentration: 200 μg/mL) and incubated on the roller bank for 10 min. Next the solution was diluted with 6 volumes of 1:10 diluted BugBuster™ and centrifuged 15 min, 15000×g. The pellet was resuspended in 200 mL of 1:10 diluted BugBuster™ by using the homogenizer and centrifuged at 10 min, 12000×g. The last step was repeated 3 times.

### Example 114. Refolding of SYR-(G₄S)₃—IL15 PF18 from Isolated Inclusion Bodies

The purified inclusion bodies containing SYR-(G4S)3-IL15 PF18, were dissolved and denatured in 30 mL 5 M guanidine with 40 mM Cysteamine and 20 mM Tris pH 8.0. The suspension was centrifuged at 16.000×g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCl, 2.2 mM MgCl₂, 2.2 mM CaCl₂), 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1× overnight and 2×4 hours, using a Spectrum™ Spectra/Por™ 3 RC Dialysis Membrane Tubing 3500 Dalton MWCO. Refolded SYR-(G4S)3-IL15 (PF18) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 14122 Da (expected mass: 14122 Da) corresponding to PF18. The purified SYR-(G4S)3-IL15 (PF18) was buffer exchanged to PBS using HiPrep™ 26/10 Desalting column (Cytiva) on a AKTA Purifier-10 (GE Healthcare).

### Example 115. Cloning of H₆-SSGENLYFQ-GGG-IL15Rα-IL15 207 into pET32a Expression Vector

The IL15Rα-IL15 fusion protein 207 (amino acid sequence being identified by SEQ ID NO: 3) was designed with an N-terminal His-tag (HHHHHH), TEV protease recognition sequence (SSGENLYFQ) followed by a sortase A recognition sequence (GGG). The codon-optimized DNA sequence was inserted into a pET32A expression vector between NdeI and XhoI, thereby removing the sequence encoding the thioredoxin fusion protein, and was obtained from Genscript, Piscataway, USA.

### Example 116. E. coli Expression of His₆-SSGENLYFQ-GGG-IL15Rα-IL15 207 and Inclusion Body Isolation

Expression of His₆-SSGENLYFQ-GGG-IL15Rα-IL15 207 starts with the transformation of the plasmid (pET32a-IL15Rα-IL15) into BL21 cells (Novagen). Next step was the inoculation of 500 mL culture (LB medium+ampicillin) with BL21 cells. When OD600 reached 0.7, cultures were induced with 1 mM IPTG (500 μL of 1M stock solution). After 4 hour induction at 37° C., the culture was pelleted by centrifugation. The cell pellet gained from 500 mL culture was lysed in 25 mL BugBuster™ with 625 units of benzonase and incubated on roller bank for 20 min at room temperature. After lysis the insoluble fraction was separated from the soluble fraction by centrifugation (20 minutes, 12000×g, 4° C.). The insoluble fraction was dissolved in 25 mL BugBuster™ with lysozyme (final concentration: 200 μg/mL) and incubated on the roller bank for 5 min. Next the solution was diluted with 6 volumes of 1:10 diluted BugBuster™ and centrifuged 15 min, 9000×g at 4° C. The pellet was resuspended in 250 mL of 1:10 diluted BugBuster™ by using the homogenizer and centrifuged at 15 min, 9000×g at 4° C. The last step was repeated 3 times.

### Example 117. Refolding of His₆-SSGENLYFQ-GGG-IL15R a-IL15 207 from Isolated Inclusion Bodies

The purified inclusion bodies containing His₆-SSGENLYFQ-GGG-IL15Rα-IL15 207, were sulfonated o/n at 4° C. in 25 mL denaturing buffer (5 M guanidine, 0.3 M sodium sulphite) and 2.5 mL 50 mM disodium 2-nitro-5-sulfobenzonate. The solution was diluted with 10 volumes of cold Milli-Q and centrifuged (10 min at 8000×g). The pellet was solved in 125 mL cold Milli-Q using a homogenizer and centrifuged (10 min at 8000×g). The last step was repeated 3 times. The purified His₆-SSGENLYFQ-GGG-IL15Rα-IL15 207 was denatured in 5 M guanidine and diluted to a concentration of 1 mg/mL of protein. Using a syringe with a diameter of 0.8 mm, the denatured protein was added dropwise to 10 volumes refolding buffer (50 mM Tris, 10.53 mM NaCl, 0.44 mM KCl, 2.2 mM MgCl₂, 2.2 mM CaCl₂, 0.055% PEG-4000, 0.55 M L-arginine, 8 mM cysteamine, 4 mM cystamine, at pH 8.0) on ice and was incubate 48 hours at 4° C. (stirring not required). The refolded His₆-SSGENLYFQ-GGG-IL15Rα-IL15 207 was loaded on a 20 mL HisTrap excel column (GE health care) on an AKTA Purifier-10 (GE Healthcare). The column was first washed with buffer A (5 mM Tris buffer, 20 mM imidazole, 500 mM NaCl, pH 7.5). Retained protein was eluted with buffer B (20 mM Tris buffer, 500 mM imidazole, 500 mM NaCl, pH 7.5) on a gradient of 25 mL from buffer A to buffer B. Fractions were analysed by SDS-PAGE on polyacrylamide gels (16%). The fractions that contained purified target protein were combined and the buffer was exchanged against TBS (20 mM Tris pH 7.5 and 150 mM NaCl₂) by dialysis performed overnight at 4° C. The purified protein was concentrated to at least 2 mg/mL using Amicon Ultra-0.5, MWCO 3 kDa (Merck-Millipore). Mass spectral analysis showed a weight of 25044 Da (expected: 25044 Da). The product was stored at −80° C. prior to further use.

### Example 118. TEV Cleavage of His₆-SSGENLYFQ-GGG-IL15Rα-IL15 207 to Obtain GGG-IL15Rα-IL15 208

To a solution of His₆-SSGENLYFQ-GGG-IL15Rα-IL15 (207, 330 μL, 2.3 mg/mL in TBS pH 7.5), was added TEV protease (50.5 μL, 10 Units/μL in 50 mM Tris-HCl, 250 mM NaCl, 1 mM TCEP, 1 mM EDTA, 50% glycerol, pH 7.5, New England Biolabs). The reaction was incubated for 1 hour at 30° C. After TEV cleavage, the solution was purified using size exclusion chromatography. The reaction mixture was loaded on to a Superdex 75 10/300 GL column (GE Healthcare) on an AKTA Purifier-10 (GE Healthcare) using TBS pH 7.5 as mobile phase and a flow of 0.5 mL/min. GGG-IL15Rα-IL15 208 was eluted at a retention time of 12 mL. The purified protein was concentrated to at least 2 mg/mL using an Amicon Ultra-0.5, MWCO 3 kDa (Merck Millipore). The product was analysed with mass spectrometry (observed mass: 22965 Da, expected mass: 22964 Da), corresponding to GGG-IL15Rα-IL15 208. The product was stored at −80° C. prior to further use.

### Example 119. Cloning of SYR-(G4S)3-IL15Ra-Linker-IL15 PF26 into pET32a Expression Vector

The SYR-(G4S)3-IL15Rα-linker-IL15 (PF26) (amino acid sequence being identified by SEQ ID NO: 6) was designed with an N-terminal (M)SYR sequence, where the methionine will be cleaved after expression leaving an N-terminal serine, and a flexible (G₄S)₃ spacer between the SYR sequence and IL15Rα-linker-IL15. The codon-optimized DNA sequence was inserted into a pET32A expression vector between NdeI and XhoI, thereby removing the sequence encoding the thioredoxin fusion protein, and was obtained from Genscript, Piscataway, USA.

### Example 120. E. coli Expression of SYR-(G4S)3-IL15Rα-linker-IL15 PF26 and Inclusion Body Isolation

Expression of SYR-(G4S)3-IL15Rα-linker-IL15 PF26 starts with the transformation of the plasmid (pET32a-SYR-(G4S)3-IL15Rα-linker-IL15) into BL21 cells (Novagen). Next step was the inoculation of 1000 mL culture (TB medium+ampicillin) with BL21 cells. When OD600 reached 1.5, cultures were induced with 1 mM IPTG (1 mL of 1 M stock solution). After >16 hour induction at 37° C. at 160 RPM, the culture was pelleted by centrifugation (5000×g-5 min). The cell pellet gained from 1000 mL culture was lysed in 60 mL BugBuster™ with 1500 units of Benzonase and incubated on roller bank for 30 min at room temperature. After lysis the insoluble fraction was separated from the soluble fraction by centrifugation (15 minutes, 15000×g). Half of the insoluble fraction was dissolved in 30 mL BugBuster™ with lysozyme (final concentration: 200 μg/mL) and incubated on the roller bank for 10 min. Next the solution was diluted with 6 volumes of 1:10 diluted BugBuster™ and centrifuged 15 min, 15000×g. The pellet was resuspended in 200 mL of 1:10 diluted BugBuster™ by using the homogenizer and centrifuged at 10 min, 12000×g. The last step was repeated 3 times.

### Example 121. Refolding of SYR-(G4S)3-IL15Rα-Linker-IL15 PF26 from Isolated Inclusion Bodies

The purified inclusion bodies containing SYR-(G4S)3-IL15Rα-linker-IL15 (PF26), were dissolved and denatured in 30 mL 5 M guanidine with 40 mM Cysteamine and 20 mM Tris pH 8.0. The suspension was centrifuged at 16.000×g for 5 min to pellet the remaining cell debris. The supernatant was diluted to 1 mg/mL with 5 M guanidine with 40 mM Cysteamine and 20 mM Tris pH 8.0, and incubated for 2 hours at RT on a rollerbank. The 1 mg/mL solution is added dropwise to 10 volumes of refolding buffer (50 mM Tris, 10.53 mM NaCl, 0.44 mM KCl, 2.2 mM MgCl₂, 2.2 mM CaCl₂, 0.055% PEG-4000, 0.55 M L-arginine, 4 mM cysteamine, 4 mM cystamine, at pH 8.0) in a cold room at 4° C., stirring required. Leave solution at least 24 hours at 4° C. Dialyze the solution to 10 mM NaCl and 20 mM Tris pH 8.0, 1× overnight and 2×4 hours using a Spectrum™ Spectra/Por™ 3 RC Dialysis Membrane Tubing 3500 Dalton MWCO. Refolded SYR-(G4S)3-IL15Rα-linker-IL15 (PF26) was loaded onto a equilibrated Q-trap anion exchange column (GE health care) on an AKTA Purifier-10 (GE Healthcare). The column was first washed with buffer A (20 mM Tris, 10 mM NaCl, pH 8.0). Retained protein was eluted with buffer B (20 mM Tris buffer, 1 M NaCl, pH 8.0) on a gradient of 30 mL from buffer A to buffer B. Mass spectrometry analysis showed a weight of 24146 Da (expected mass: 24146 Da) corresponding to PF26. The purified SYR-(G4S)3-IL15Rα-linker-IL15 (PF26) was buffer exchanged to PBS using HiPrep™ 26/10 Desalting column from cytiva on a AKTA Purifier-10 (GE Healthcare).

Modification of Cytokines and scFv's

### Example 122. C-Terminal Sortagging of Compound GGG-PEG₂-BCN (157) to hOKT3 200 Using Sortase A to Obtain hOKT3-PEG₂-BCN 201

A bioconjugate according to the invention was prepared by C-terminal sortagging using sortase A (identified by SEQ ID NO: 2). To a solution of hOKT3 200 (500 μL, 500 μg, 35 μM in PBS pH 7.4) was added sortase A (58 μL, 384 μg, 302 μM in TBS pH 7.5+10% glycerol), GGG-PEG₂-BCN (157, 28 μL, 50 mM in DMSO), CaCl₂) (69 μL, 100 mM in MQ) and TBS pH 7.5 (39 μL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27829 Da), corresponding to 201. The sample was dialyzed against PBS pH 7.4 and concentrated by spinfiltration (Amicon Ultra-0.5, Ultracel-10 Membrane, Millipore) to obtain hOKT3-PEG₂-BCN 201 (60 μL, 169 μg, 101 μM in PBS pH 7.4).

### Example 123. C-Terminal Sortagging of Compound GGG-PEG₂-BCN (157) to hOKT3 200 Using Sortase A Pentamutant to Obtain hOKT3-PEG₂-BCN 201

A bioconjugate according to the invention was prepared by C-terminal sortagging using sortase A pentamutant (BPS Bioscience, catalog number 71046). To a solution of hOKT3 200 (14.3 μL, 14 μg, 35 μM in PBS pH 7.4) was added sortase A pentamutant (0.5 μL, 1 μg, 92 μM in 40 mM Tris pH8.0, 110 mM NaCl, 2.2 mM KCl, 400 mM imidazole and 20% glycerol), GGG-PEG₂-BCN (157, 2 μL, 20 mM in DMSO:MQ=2:3), CaCl₂) (2 μL, 100 mM in MQ) and TBS pH 7.5 (1.2 μL). The reaction was incubated at 37° C. overnight. Mass spectral analysis showed one major product (observed mass 27829 Da), corresponding to hOKT3-PEG₂-BCN 201.

### Example 124. C-Terminal Sortagging of GGG-PEG₁₁-Tetrazine (169) to hOKT3 200 with sortase A to obtain hOKT3-PEG₁₁-Tetrazine PF01

A bioconjugate according to the invention was prepared by C-terminal sortagging with sortase A (identified by SEQ ID NO: 2). To a solution of hOKT3 200 (1908 μL, 5 mg, 91 μM in PBS pH 7.4) was added sortase A (81 μL, 948 μg, 533 μM in TBS pH 7.5+10% glycerol), GGG-PEG₁₁-tetrazine (169, 347 μL, 20 mM in MQ), CaCl₂) (347 μL, 100 mM in MQ) and TBS pH 7.5 (789 μL). The reaction was incubated at 37° C. overnight. Mass spectral analysis showed one major product (observed mass 28258 Da), corresponding to hOKT3-PEG₁₁-tetrazine PF01. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and buffer exchanged to PBS pH 6.5 using a HiPrep 26/10 desalting column (GE Healthcare). Addition dialysis was performed to PBS pH 6.5 for 3 days at 4° C. to remove residual 169.

### Example 125. C-Terminal Sortagging of GGG-PEG₂₃-Tetrazine (170) to hOKT3 200 with sortase A to obtain hOKT3-PEG₂₃-Tetrazine PF02

A bioconjugate according to the invention was prepared by C-terminal sortagging with sortase A (identified by SEQ ID NO: 2). To a solution of hOKT3 200 (1908 μL, 5 mg, 91 μM in PBS pH 7.4) was added sortase A (81 μL, 948 μg, 533 μM in TBS pH 7.5+10% glycerol), GGG-PEG₂₃-tetrazine (170, 347 μL, 20 mM in MQ), CaCl₂) (347 μL, 100 mM in MQ) and TBS pH 7.5 (789 μL). The reaction was incubated at 37° C. overnight. Mass spectral analysis showed one major product (observed mass 28787 Da), corresponding to hOKT3-PEG₂₃-tetrazine PF02. The reaction was purified on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was dialyzed to PBS pH 6.5 followed by purification on a Superdex75 10/300 GL column (GE Healthcare) on an AKTA Purifier-10 (GE Healthcare) using PBS pH 6.5 as mobile phase.

### Example 126. C-Terminal Sortagging of GGG-PEG₂-Arylazide (171) to hOKT3 200 with Sortase A to Obtain hOKT3-PEG₂-Arylazide PF03

A bioconjugate according to the invention was prepared by C-terminal sortagging with sortase A (identified by SEQ ID NO: 2). To a solution of hOKT3 200 (2092 μL, 5 mg, 83 μM in PBS pH 7.4) was added sortase A (95 μL, 950 μg, 456 μM in TBS pH 7.5+10% glycerol), GGG-PEG₂-arylazide (171, 347 μL, 20 mM in MQ), CaCl₂) (347 μL, 100 mM in MQ) and TBS pH 7.5 (591 μL). The reaction was incubated at 37° C. overnight. Mass spectral analysis showed one major product (observed mass 27865 Da), corresponding to hOKT3-PEG₂-arylazide PF03. The reaction was purified on a His-trap excel 1 mL column (GE Healthcare) on an AKTA Purifier-10 (GE Healthcare). The column was equilibrated with buffer A (20 mM Tris, 200 mM NaCl, 20 mM Imidazole, pH 7.5) and the sample was loaded with 1 mL/min. The flowthrough purified on a Superdex75 10/300 GL column (GE Healthcare) on an AKTA Purifier-10 (GE Healthcare) using PBS pH 7.4 as mobile phase.

### Example 127. C-Terminal Sortagging of Compound GGG-PEG₂₃-BCN (163) Anti-4-1BB PF31 with Sortase a to Obtain Anti-4-1BB PF07

A bioconjugate according to the invention was prepared by C-terminal sortagging with sortase A (identified by SEQ ID NO: 2). To a solution of anti-4-1BB-PF31 (665 μL, 2 mg, 107 μM in PBS pH 7.4) was added sortase A (100 μL, 1 mg, 357 μM in TBS pH 7.5+10% glycerol), GGG-PEG₂₃-BCN (163, 140 μL, 20 mM in MQ), CaCl₂) (140 μL, 100 mM in MQ) and TBS pH 7.5 (355 μL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and after concentration purified on a Superdex75 10/300 column (Cytiva). Mass spectral analysis showed one major product (observed mass 28478 Da) corresponding to anti-4-1BB-BCN PF07.

### Example 128. C-Terminal Sortagging of Compound GGG-PEG₂-Arylazide (171) Anti-4-1BB-PF31 with Sortase a to Obtain Anti-4-1BB PF09

A bioconjugate according to the invention was prepared by C-terminal sortagging with sortase A (identified by SEQ ID NO: 2). To a solution of anti-4-1BB-PF31 (665 μL, 2 mg, 107 μM in PBS pH 7.4) was added sortase A (100 μL, 1 mg, 357 μM in TBS pH 7.5+10% glycerol), GGG-PEG₂-arylazide (171, 140 μL, 20 mM in MQ), CaCl₂) (140 μL, 100 mM in MQ) and TBS pH 7.5 (355 μL). The reaction was incubated at 37° C. overnight followed by purification on a His-trap excel 1 mL column (GE Healthcare) on an AKTA Explorer-100 (GE Healthcare). The column was equilibrated with buffer A (20 mM Tris, 200 mM NaCl, 20 mM Imidazole, pH 7.5) and the sample was loaded with 1 mL/min. The flowthrough was collected and mass spectral analysis showed one major product (observed mass 27592 Da) corresponding to anti-4-1 BB-azide PF09.

### Example 129. N-Terminal BCN Functionalization of IL15Rα-IL15 PF26 by SPANC to Obtain BCN-IL15Rα-IL15 PF15

To IL15Rα-IL15 PF26 (2.9 mg, 50 μM in PBS) was added 2 eq NalO₄ (4.8 μL of 50 mM stock in PBS) and 10 eq L-Methionine (12.5 μL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the elute (2.6 mg, 50 μM in PBS) was added 160 eq N-methylhydroxylamine·HCl (340 μL of 50 mM stock in PBS) and 160 eq p-Anisidine (340 μL of 50 mM stock in PBS). The reaction mixture was incubated for 3 hours at 25° C. Mass spectral analysis showed one single peak (observed mass 24143 Da) corresponding to N-methyl-imine-oxide-IL15. The reaction mixture was purified using PD-10 desalting columns packed with Sephadex G-25 resin (Cytiva) and eluted using PBS. To the elute (2.47 mg, 50 μM in PBS) was added 25 eq Bis-BCN-PEG₁₁ (105) (51 μL, 50 mM in DMSO) and 150 μL DMF. The reaction was incubated overnight at room temperature. The reaction was purified using a Superdex75 10/300 column (Cytiva). Mass spectral analysis showed one major peak corresponding to BCN-IL15Rα-IL15 PF15 (observed mass 25041 Da).

### Example 130. N-Terminal Diazotransfer Reaction of IL15 PF18 to Obtain Azido-IL15 PF19

To IL15 PF18 (5 mg, 50 μM in 0.1 M TEA buffer pH 8.0) imidazole-1-sulfonylazide hydrochloride (708 μL, 50 mM in 50 mM NaOH) was added and incubated overnight at 37° C. The reaction was purified using a HiPrep™ 26/10 Desalting column (Cytiva). Mass spectral analysis showed one main peak (observed mass 14147 Da) corresponding to azido-IL15 PF19.

### Example 131. Conjugation of Tri-BCN (150) to hOKT3-PEG₂-Arylazide PF03 to Obtain Bis-BCN-hOKT3 PF22

To a solution of hOKT3-PEG₂-arylazide PF03 (87 μL, 1 mg, 411 μM in PBS pH 7.4) was added PBS pH 7.4 (559 μL), DMF (49 μL) and compound 150 (22 μL, 40 mM solution in DMF, 25 equiv.). The reaction was incubated overnight at RT. Mass spectral analysis showed one major product (observed mass 29171 Da), corresponding to bis-BCN-hOKT3 PF22. The reaction was purified on a Superdex75 10/300 GL column (GE Healthcare) on an AKTA Purifier-10 (GE Healthcare) using PBS pH 7.4 as mobile phase.

### Example 132. N-Terminal BCN Functionalization of IL15 PF18 by SPANC to Obtain BCN-PEG₁₁-IL15 PF33

To I15 PF18 (8070 μL, 50 μM in PBS) was added 2 eq NalO₄ (16.4 μL of 50 mM stock in PBS).

The reaction was incubated for 5 minutes at 4° C. Mass spectral analysis showed oxidation of the serine into the corresponding hydrate (observed masses 14109 Da). The reaction mixture was purified using PD-10 desalting columns packed with Sephadex G-25 resin (Cytiva) and eluted using PBS. To the elute (11500 μL, 32 μM in PBS) was added 160 eq N-methylhydroxylamine HCl (558 μL of 100 mM stock in PBS) and 160 eq p-Anisidine (558 μL of 100 mM stock in PBS). The reaction mixture was incubated for 3 hours at 25° C. Mass spectral analysis showed one single peak (observed mass 14119 Da) corresponding to N-methyl-imine-oxide-IL15. The reaction mixture was purified using PD-10 desalting columns packed with Sephadex G-25 resin (Cytiva) and eluted using PBS. To the concentrated elute (5370, 66 μM μM in PBS) was added 25 eq BCN-PEG₁₁-BCN (105) (142 μL, 50 mM in DMSO) and 1576 μL PBS. The reaction was incubated overnight at room temperature. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed one major peak corresponding to BCN-PEG₁₁-IL15 PF33 (observed mass 15016 Da).

### Example 133. N-Terminal Sortagging of Arylazide-PEG₁-LPETGG (175) to GGG-IL15Rα-IL15 208 with Sortase a to Obtain Arylazide-IL15Rα-IL15 PF13

To a solution containing protein 208 (2000 μL, 140 μM in TBS pH 7.5) was added TBS pH 7.5 (2686 μL), CaCl₂ (559 μL, 100 mM) and 175 (83 μL, 50 mM in DMSO) and Sortase A (260 μL, 537 μM in TBS pH 7.5) and incubated 3 hours at 37° C. (shielded from light). After incubation, Sortase A was removed from the solution using Ni-NTA beads (500 μL Beads=1 mL slurry). The solution was incubated ON at 4° C. with Ni-NTA beads on a roller bank, whereafter the solution was centrifuged (5 min, 7.000×g). The supernatant, which contained the product PF13, was collected by separation of the supernatant from the pellet. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectrometry analysis showed a weight of 24193 Da (expected mass: 24193 Da) corresponding to PF13.

### Example 134. N-Terminal Incorporation of Tri-BCN (150) in SYR-(G₄S)₃—IL15Rα-IL15 PF26 Using Strain-Promoted Alkyne-Nitrone Cycloaddition to Obtain Bis-BCN-IL15Rα-IL15 PF27

To IL15Rα-IL15 PF26 (3840 μL, 50 μM in PBS) was added 2 eq NalO₄ (7.7 μL of 50 mM stock in PBS) and 10 eq L-Methionine (19.2 μL of 100 mM stock in PBS). The reaction was incubated for 5 minutes at 4° C. Mass spectral analysis showed oxidation of the serine into the corresponding aldehyde and hydrate (observed masses 24114 Da and 24132 Da). The reaction mixture was purified using PD-10 desalting columns packed with Sephadex G-25 resin (Cytiva) and eluted using PBS. To the concentrated elute (1800 μL, 50 μM in PBS) was added 160 eq N-methylhydroxylamine·HCl (320 μL of 90 mM stock in PBS) and 160 eq p-Anisidine (288 μL of 100 mM stock in PBS). The reaction mixture was incubated for 3 hours at 25° C. Mass spectral analysis showed one single peak (observed mass 24143 Da) corresponding to N-methyl-imine-oxide-IL15Rα-IL15. The reaction mixture was purified using PD-10 desalting columns packed with Sephadex G-25 resin (Cytiva) and eluted using PBS. To the concentrated elute (3100 μL, 60 μM in PBS) was added 25 eq tri-BCN (150) (116 μL, 40 mM in DMSO), 256 μL DMF and PBS pH 7.4 (248 μL). The reaction was incubated o/n at RT. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed the desired Bis-BCN-IL15Rα-IL15 PF27 (observed mass 25448 Da, expected mass 25447). RP-HPLC showed a labeling efficiency of 60%.

### Example 135. Conjugation of Bis-BCN-TCO XL11 to hOKT3-PEG₂-Arylazide PF03 to Obtain hOKT3-(TCO)₁—(BCN)₁ PF32

To a solution containing hOKT3-PEG₂-arylazide PF03 (35 μL, 411 μM in PBS pH 7.4) was added PBS pH 7.4 (224.2 μL) and bis-BCN-TCO XL11 (28.8 μL, 10 mM in DMF, 20 equiv.). The reaction was incubated 16 hours at room temperature. Excess bis-BCN-TCO XL11 was removed using centrifugal filters (Amicon Ultra-0.5 mL MWCO 10 kDa, Merck Millipore) to obtain the final sample at a concentration of 3.39 mg/mL in PBS pH 7.4. Mass spectral analysis showed one major product (observed mass 29305 Da) corresponding to the expected product hOKT3-(TCO)₁—(BCN)₁ PF32.

### Example 136. Conjugation of Azido-IL15 PF19 to hOKT3-(TCO)₁—(BCN)₁ PF32 to Obtain hOKT3-(IL15)₁-(TCO)₁ PF34

To a solution containing hOKT3-(TCO)₁—(BCN)₁ PF32 (50 μL, 116 μM in PBS pH 7.4) was added azido-IL15 PF19 (52 μL, 225 μM in PBS pH 7.4, 2 equiv.). The reaction was incubated 16 hours at 37° C. Mass spectral analysis showed one major product (observed mass 43453 Da) corresponding to the expected product hOKT3-(IL15)₁-(TCO)₁ PF34.

### Example 137. Conjugation of Anti-4-1BB-PEG₂-Arylazide PF09 to bis-BCN-IL15Rα-IL15 PF27 to Obtain IL15Rα-IL15-(Anti-4-1-BB)₁—(BCN)₁ PF35

To a solution containing anti-4-1BB-PEG₂-arylazide PF09 (52 μL, 200 μM in PBS pH 7.4) was added Bis-BCN-IL15Rα-IL15 PF27 (80 μL, 169 μM in PBS pH 7.4). Reaction was incubated 16 hours at room temperature. The reaction mixture was loaded on to a Superdex 75 10/300 GL column (GE Healthcare) on an AKTA Purifier-10 (GE Healthcare) using PBS pH 7.4 as mobile phase and a flow of 0.5 mL/min. Mass spectral analysis showed a weight of 53043 Da (expected mass: 53050 Da) corresponding to IL15Rα-IL15-(anti-4-1-BB)₁—(BCN)₁ PF35.

### Antibody Modifications Example 138. Enzymatic Remodeling of Adcetris to Adcetris(6-N₃GalNAc)₂ Adcetris-v1a

Adcetris (sourced from the pharmacy) was buffer exchanged and concentrated to 35.6 mg/mL in 20 mM Histidine, 150 mM NaCl pH 7.5 using centrifugal filters (Amicon Ultra-0.5 mL MWCO 10 kDa, Merck Millipore). Adcetris (84 μL, 3 mg, 35.6 mg/mL in 20 mM Histidine, 150 mM NaCl pH 7.5) was incubated with EndoSH (6 μL, 31 μg, 5.2 mg/mL), described in WO2017137459A1, TnGalNAcT (32 μL, 106 μg, 3.3 mg/mL), 6-N3GalNAc-UDP (5 μL, 100 mM in MQ), both described in WO2016170186, MnCl₂ (1 μL, 1 M in MQ) and 20 mM Histidine, 150 mM NaCl pH 7.5 (22 μL). The reaction was incubated overnight at 30° C. and buffer exchanged and concentrated to 35.6 mg/mL in PBS pH 7.4 using centrifugal filters (Amicon Ultra-0.5 mL MWCO 10 kDa, Merck Millipore). Mass spectral analysis of a sample after IdeS treatment showed one major Fc/2 product (observed mass 24333 Da) corresponding to the expected product Adcetris-v1a. Furthermore, RP-HPLC analysis confirmed MMAE remained conjugated to the antibody (average DAR of 3.8 was observed both for Adcetris and Adcetris-v1a).

### Example 139. Enzymatic Remodeling of Kadcyla to Kadcyla(6-N₃GalNAc)₂ Kadcyla-v1a

Kadcyla (sourced from the pharmacy) was buffer exchanged and concentrated to 17.8 mg/mL in 20 mM Histidine, 150 mM NaCl pH 7.5 using centrifugal filters (Amicon Ultra-0.5 mL MWCO 10 kDa, Merck Millipore). Kadcyla (224 μL, 4 mg, 17.8 mg/mL in 20 mM Histidine, 150 mM NaCl pH 7.5) was incubated with EndoSH (8 μL, 42 μg, 5.2 mg/mL), described in WO2017137459A1, TnGalNAcT (42 μL, 139 μg, 3.3 mg/mL), 6-N3GalNAc-UDP (7 μL, 100 mM in MQ), both described in WO2016170186, and MnCl₂ (2 μL, 1 M in MQ). The reaction was incubated overnight at 30° C. and buffer exchanged and concentrated to 10 mg/mL in PBS pH 7.4 using centrifugal filters (Amicon Ultra-0.5 mL MWCO 10 kDa, Merck Millipore). Mass spectral analysis of a sample after IdeS treatment showed one major Fc/2 product (observed mass 24363 Da, approximately 80% of total Fc/2-fragments), corresponding to the azido-modified Fc/2-fragment, and one minor Fc/2 product (observed mass 25321 Da, approximately 20% of total Fc/2-fragments), corresponding to the azido-modified Fc/2-fragment with DM1, thereby confirming formation of Kadcyla-v1a.

### Example 140. Enzymatic Remodeling of Trastuzumab-S239C to Trastuzumab-S239C(6-N₃GalNAc)₂ Trast-v9a

Trastuzumab-S239C mutant (transient expressed in CHO by Evitria, heavy chain mutation S239C) was buffer exchanged and concentrated to 27.0 mg/mL in 20 mM Histidine, 150 mM NaCl pH 7.5 using centrifugal filters (Amicon Ultra-0.5 mL MWCO 10 kDa, Merck Millipore). trastuzumab-S239C (148 μL, 4 mg, 27.0 mg/mL in 20 mM Histidine, 150 mM NaCl pH 7.5) was incubated with EndoSH (8 μL, 42 μg, 5.2 mg/mL), described in WO2017137459A1, TnGalNAcT (42 μL, 139 μg, 3.3 mg/mL), 6-N3GalNAc-UDP (7 μL, 100 mM in MQ), both described in WO2016170186, and MnCl₂ (1 μL, 1 M in MQ). The reaction was incubated overnight at 30° C. and buffer exchanged and concentrated to 10 mg/mL in PBS pH 7.4 using centrifugal filters (Amicon Ultra-0.5 mL MWCO 10 kDa, Merck Millipore). Mass spectral analysis of a sample after IdeS treatment showed one major Fc/2 product (observed mass 24499 Da, approximately 80% of total Fc/2-fragments), corresponding to the azido-modified Fc/2-fragment with Cys239 forming a disulfide with Cysteine, and two minor Fc/2 product (observed masses 24625 and 24685 Da, approximately 15% and 5% of total Fc/2-fragments), corresponding to the azido-modified Fc/2-fragment with C-terminal Lysine and the azido-modified Fc/2-fragment with Cys239 forming a disulfide with glutathione, thereby confirming formation of trast-v9a.

### Example 141. Intramolecular Cross-Linking of Trastuzumab(6-N₃GalNAc)₂ Trast-v1a with Bis-DIBO-Tetrazine XL14 to Give Trastuzumab-Tetrazine Trast-v1a-XL14

To a solution of trastuzumab(6-N₃GalNAc)₂ (trast-v1a) (149 μL, 5 mg, 33.6 mg/mL in PBS pH 7.4), prepared according to WO2016170186, was added PBS pH 7.4 (351 μL) propylene glycol (497 μL) and bis-DIBO-tetrazine XL14 (3.3 μL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Mass spectral analysis of the IdeS-digested sample showed one major products (observed mass 50177 Da), corresponding to intramolecularly cross-linked Fc/2-fragment, thereby confirming formation of trast-v1a-XL14. Intermolecular cross-linking could be excluded, because mass spectral analysis was performed on purified monomeric fractions. The final sample was concentrated to 11.98 mg/mL using centrifugal filters (Amicon Ultra-0.5 mL MWCO 10 kDa, Merck Millipore).

### Example 142. Intramolecular Cross-Linking of Trastuzumab(6-N₃GalNAc)₂ Trast-v1a with Tri-BCN 145 to Give Trastuzumab-BCN Trast-v1a-145

To a solution of trastuzumab(6-N₃GalNAc)₂ trast-v1a (320 μL, 2 mg, 5.56 mg/mL in PBS pH 7.4), prepared according to WO2016170186, was added compound 145 (80 μL, 1.66 mM solution in DMF, 10 equiv. compared to IgG). The reaction was incubated for 1 day at RT followed by buffer exchange to PBS pH 7.4 using centrifugal filters (Amicon Ultra-0.5 mL MWCO 10 kDa, Merck Millipore). Mass spectral analysis of the IdeS digested sample showed one major product (calculated mass 49796 Da, observed mass 49807 Da), corresponding to intramolecularly cross-linked trastuzumab derivative trast-v1a-145. HPLC-SEC showed <4% aggregation, hence excluding intermolecular cross-linking.

### Example 143. Intramolecular Cross-Linking of Rituximab(6-N₃GalNAc)₂ Rit-v1a with Tri-BCN 145 to Give Rituximab-BCN Rit-v1a-145

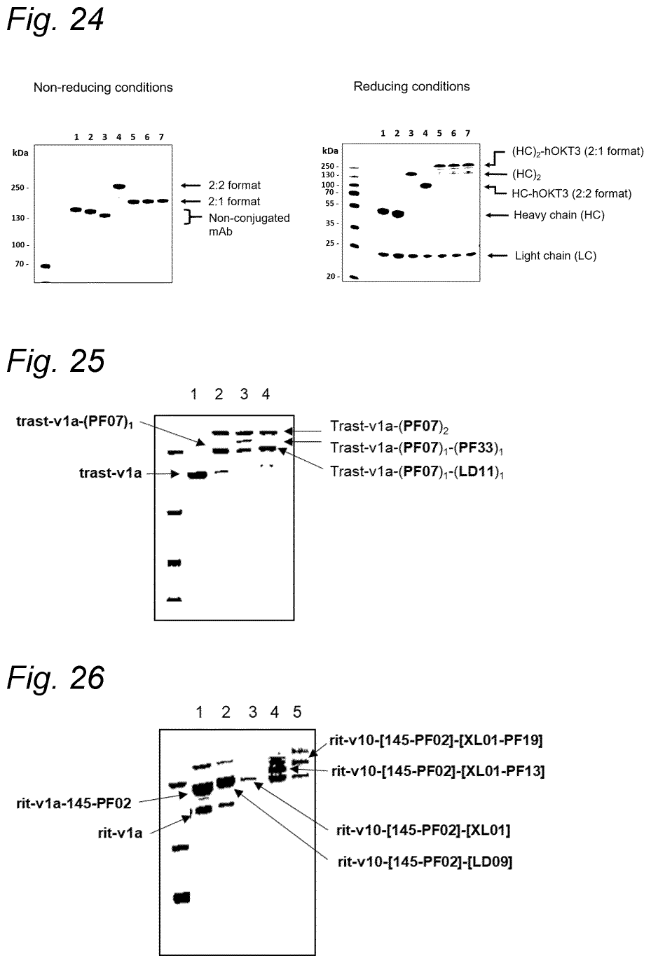

To a solution of rit-v1a (494 μL, 30 mg, 60.7 mg/mL in PBS pH 7.4), prepared according to WO2016170186, was added PBS pH 7.4 (2506 μL), propylene glycol (2980 μL) and trivalent linker 145 (20 μL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Reducing SDS-PAGE showed one major HC product, corresponding to the crosslinked heavy chain (see FIG. 24, right panel, lane 3), indicating formation of rit-v1a-145. Furthermore, non-reducing SDS-PAGE showed one major band around the same height as rit-v1a (see FIG. 24, left panel, lane 3), demonstrating that only intramolecular cross-linking occurred.

### Example 144. Intramolecular Cross-Linking of Kadcyla(6-N₃GalNAc)₂ Kadcyla-v1a with Tri-BCN 145 to Give Kadcyla-BCN Kadcyla-v1a-145

To a solution of Kadcyla-v1a (400 μL, 4 mg, 10 mg/mL in PBS pH 7.4) was added propylene glycol (397 μL) and trivalent linker 145 (2.7 μL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Mass spectral analysis of the IdeS-digested sample showed three major products (observed masses: 49795, 50752 and 51711 Da), corresponding to intramolecularly cross-linked Fc/2-fragment with 0, 1 and 2 DM1 moieties, thereby confirming formation of Kadcyla-v1a-145. Intermolecular cross-linking could be excluded, because mass spectral analysis was performed on purified monomeric fractions. The final sample was concentrated to 6.88 mg/mL using centrifugal filters (Amicon Ultra-0.5 mL MWCO 10 kDa, Merck Millipore).

### Example 145. Intramolecular Cross-Linking of Trastuzumab-S239C(6-N₃GalNAc)₂ Trast-v9a with Tri-BCN 145 to Give Trastuzumab-S239C-BCN Trast-v9a-145

To a solution of trast-v9a (400 μL, 4 mg, 10 mg/mL in PBS pH 7.4) was added propylene glycol (397 μL) and trivalent linker 145 (2.7 μL, 40 mM solution in DMF, 4.0 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Mass spectral analysis of the IdeS-digested sample showed one major products (observed mass 50251 Da), corresponding to intramolecularly cross-linked Fc/2-fragment with one Cys239 forming a disulfide with Cysteine and one Cys239 forming a disulfide with glutathione, thereby confirming formation of trast-v9a-145. Intermolecular cross-linking could be excluded, because mass spectral analysis was performed on purified monomeric fractions. The final sample was concentrated to 7.88 mg/mL using centrifugal filters (Amicon Ultra-0.5 mL MWCO 10 kDa, Merck Millipore).

### Generation of Bifunctional and Multifunctional Antibodies

### Example 146. Conjugation of Anti-4-1-BB-PEG₂₃-BCN PF07 to Trast-v1a to Obtain Trast-v1a-(PF07)₁ with 2:1 Molecular Format (CDR:Anti-4-1-BB)

Trastuzumab(6-N₃GalNAc)₂ trast-v1a (1 mg, 33.6 mg/mL in PBS), prepared according to WO2016170186, was incubated with anti-4-1-BB-PEG₂₃-BCN PF07 (55.8 μL, 6.8 mg/mL in PBS pH 7.5) overnight at 37° C. followed by removal of excess PF07 and buffer exchange to PBS pH 7.4 using centrifugal filters (Amicon Ultra-0.5 mL MWCO 100 kDa, Merck Millipore) to a concentration of 17.5 mg/mL. Mass spectral analysis of a sample after IdeS treatment showed two major Fc/2 products (observed mass 24362 Da and 52840 Da), corresponding to unreacted Fc/2 and Fc/2-PF07 respectively. This confirmed the formation of the expected product trast-v1a-(PF07)₁. SDS-PAGE analysis supported this conclusion (see FIG. 25).

### Example 147. Conjugation of BCN-MMAE (LD11) to Trast-v1a-(PF07)₁ to Obtain trast-v1a-(PF07)₁-(LD11)₁ with 2:1:1 Molecular Format (CDR:Anti-4-1-BB:MMAE)

Trast-v1a-(PF07)₁ (5.56 μL, 17.5 mg/mL in PBS) was incubated with BCN-MMAE LD11 (0.57 μL, 10 mM in DMF) overnight at room temperature. Mass spectral analysis of a sample after IdeS treatment showed two major Fc/2 products (observed mass 25872 Da and 52840 Da) corresponding to Fc/2-LD11 and Fc/2-PF07 respectively. This confirmed the formation of the expected product trast-v1a-(PF07)₁-(LD11)₁. SDS-PAGE analysis supported this conclusion (see FIG. 25).

### Example 148. Conjugation of BCN-IL15 PF33 to Trast-v1a-(PF07)₁ to Obtain trast-v1a-(PF07)₁—(PF33)₁ with 2:1:1 Molecular Format (CDR:Anti-4-1-BB:IL15)

Trast-v1a-(PF07)₁ (5.56 μL, 17.5 mg/mL in PBS) was incubated with BCN-IL15 PF33 (4.13 μL, 8.2 mg/mL in PBS pH 7.5) overnight at 37° C. Mass spectral analysis of a sample after IdeS treatment showed two major Fc/2 products (observed mass 39376 Da and 52842 Da) corresponding to Fc/2—PF33 and Fc/2-PF07 respectively. This confirmed the formation of the expected product trast-v1a-(PF07)₁—(PF33)₁. SDS-PAGE analysis supported this conclusion (see FIG. 25).

### Example 149. Conjugation of hOKT3-(IL15)₁-(TCO)₁ PF34 to Trast-v1a-XL14 to Give Immune Cell Engager Trast-v1a-XL14-PF34 with 2:1:1 Molecular Format (CDR:hOKT3:IL15)

To a solution of trast-v1a-XL14 (4.2 μL, 50 μg, 11.98 mg/mL in PBS pH 7.4) was added hOKT3-(IL15)₁-(TCO)₁ PF34 (17.1 μL, 29 μg, 39 μM in PBS pH 6.5, 2 equiv. compared to IgG). The reaction was incubated overnight at rt. Mass spectral analysis of the IdeS digested sample showed one major Fc/2 product (observed mass 93608 Da), corresponding to the crosslinked Fc/2-fragment conjugated to PF34, thereby confirming formation of trast-v1a-XL14-PF34.

### Example 150. Conjugation of IL15Rα-IL15-(Anti-4-1-BB)₁—(BCN)₁ PF35 to trast-v1a to obtain trast-v1a-(PF35)₂ with 2:2:2 Molecular Format (CDR:IL15Rα-IL15:Anti-4-1-BB)

To a solution of trastuzumab(6-N₃GalNAc)₂trast-v1a (1 μL, 56 μg, 56.1 mg/mL in PBS pH 7.4), prepared according to WO2016170186, was added IL15Rα-IL15-(anti-4-1-BB)₁—(BCN)₁ PF35 (22 μL, 4 mg/mL in PBS pH 7.4, 4 eq compared to IgG). The reaction was incubated for 16 hours at room temperature. Mass spectral analysis of the IdeS-digested sample showed a peak of 77413 Da (expected mass 77413 Da), corresponding to the conjugate trast-v1a-(PF35)₂.

### Example 151. Conjugation of IL15Rα-IL15-(Anti-4-1-BB)₁—(BCN)₁ PF35 to Trastuzumab-Tetrazine Trast-v1a-XL14 to Obtain Trast-v1a-XL14-PF35 with 2:1:1 Molecular Format (CDR:IL15Rα-IL15:Anti-4-1-BB)

To a solution of trast-v1a-XL14 (3.2 μL, 38 μg, 11.98 mg/mL in PBS pH 7.4) was added IL15Rα-IL15-(anti-4-1-BB)₁—(BCN)₁ (PF35 7 μL, 4 mg/mL, 2 eq compared to IgG). The reaction was incubated for 4 hours at room temperature. Mass spectral analysis of the IdeS digested sample showed a peak of 103199 (expected mass 103197 Da), corresponding to the conjugate trast-v1a-XL14-PF35.

### Example 152. Conjugation of Bis-BCN-hOKT3 PF22 to Adcetris-v1a to Give Immune Cell Engager Adcetris-v1a-PF22 with 2:4:1 Molecular Format (CDR:MMAE:hOKT3)

To a solution of Adcetris-v1a (3.6 μL, 75 μg, 20.75 mg/mL in PBS pH 7.4) was added bis-BCN-hOKT3 PF22 (5.1 μL, 29 μg, 194 μM in PBS pH 7.4, 2 equiv. compared to IgG). The reaction was incubated overnight at 37° C. Mass spectral analysis of an IdeS-digested sample showed one major product (observed mass 77837 Da) corresponding to the crosslinked Fc/2 fragment of Adcetris-v1a-PF22.

### Example 153. Conjugation of Bis-BCN-IL15Rα-IL15 PF27 to Adcetris-v1a to Give Immune Cell Engager Adcetris-v1a-PF27 with 2:4:1 Molecular Format (CDR:MMAE:IL15Rα-IL15)

To a solution of Adcetris-v1a (3.6 μL, 75 μg, 20.75 mg/mL in PBS pH 7.4) was added bis-BCN-IL15Rα-IL15 PF27 (3.5 μL, 25 μg, 285 μM in PBS pH 7.4, 2 equiv. compared to IgG). The reaction was incubated overnight at 37° C. Mass spectral analysis of an IdeS-digested sample showed one major product (observed mass 74126 Da) corresponding to the crosslinked Fc/2 fragment of Adcetris-v1a-PF27.

### Example 154. Conjugation of hOKT3-PEG11-Tetrazine PF01 to Kadcyla-v1a-145 to Give Immune Cell Engager Kadcyla-v1a-145-PF01 with 2:4:1 Molecular Format (CDR:DM1:hOKT3)

To a solution of Kadcyla-v1a-145 (22 μL, 150 μg, 6.88 mg/mL in PBS pH 7.4) was added hOKT3-PEG11-tetrazine PF01 (8.8 μL, 57 μg, 230 μM in PBS pH 7.4, 2 equiv. compared to IgG). The reaction was incubated overnight at rt. Mass spectral analysis of the IdeS-digested sample showed two major products, corresponding to the intramolecularly cross-linked Fc/2-fragment with PF01 (observed mass 78024 Da, approximately 40% of total Fc/2 fragments), and the intramolecularly cross-linked Fc/2-fragment with PF01 and DM1 (observed mass 78985 Da, approximately 60% of total Fc/2 fragments), thereby confirming formation of Kadcyla-v1a-145-PF01.

### Example 155. Conjugation of hOKT3-PEG₂₃-Tetrazine PF02 to Rituximab-BCN Rit-v1a-145 to Give T Cell Engager Rit-v1a-145-PF02 with 2:1 Molecular Format (CDR:hOKT3)

To a solution of rit-v1a-145 (247 μL, 6.3 mg, 171 μM in PBS pH 7.4) was added hOKT3-PEG23-tetrazine PF02 (262 μL, 2.0 mg, 267 μM in PBS pH 6.5, 1.7 equiv. compared to IgG). The reaction was incubated overnight at rt followed by purification on a Superdex200 10/300 GL column (GE Healthcare) on an AKTA Purifier-10 (GE Healthcare) using PBS pH 7.4 as mobile phase. Non-reducing SDS-PAGE analysis showed one major product consisting of an antibody conjugated to a single hOKT3 (see FIG. 24, left panel, lane 7), thereby confirming formation of rit-v1a-145-PF02. Furthermore, reducing SDS-PAGE confirms one major HC product, corresponding to two heavy chains conjugated to a single hOKT3 (see FIG. 24, right panel, lane 7).

### Example 156. Maleimide Conjugation of Bis-Maleimide-MMAE (LD09) to Rit-v1a-145-PF02 to Obtain Rit-v10-[145-PF02]-[LD09] with 2:1:1 Molecular Format (CDR:hOKT3:MMAE)

Rit-v1a-145-PF02 (0.7 mg, 7.34 mg/mL in PBS+10 mM EDTA) was incubated with 0.8 μL 10 mM TCEP for 2 hours at 37° C. To a part of the reaction mixture (13.6 μL) bis-maleimide-MMAE (LD09) (1.7 μL, 1 mM in DMF) was added and incubated for 3 hours at room temperature. Mass spectral analysis of a sample after DTT reduction showed one major HC product (observed mass 130895 Da) corresponding to the expected product Rit-v10-[145-PF02]-[LD09].

### Example 157. Maleimide Conjugation of Bis-Maleimide-BCN (XL01) to Rit-v1a-145-PF02 to Obtain Intermediate Rit-v10-[145-PF02]—[XL01]

Rit-v1a-145-PF02 (0.7 mg, 7.34 mg/mL in PBS+10 mM EDTA) was incubated with 0.8 μL 10 mM TCEP for 2 hours at 37° C. To a part of the reaction mixture (84 μL) bis-maleimide-BCN (XL01) (10.3 μL, 1 mM in DMF) was added and incubated for 3 hours at room temperature followed by buffer exchange to PBS pH 7.4 using centrifugal filters (Amicon Ultra-0.5 mL MWCO 100 kDa, Merck Millipore) to a concentration of 9.8 mg/mL. SDS-PAGE confirmed the formation of the expected product Rit-v10-[145-PF02]—[XL01](see FIG. 26).

### Example 158. Conjugation of Azido-IL15 PF19 to Rit-v10-[145-PF02]—[XL01] to Obtain Rit-v10-[145-PF02]—[XL01-PF19] with 2:1:1 Molecular Format (CDR:hOKT3:IL15)

Rit-v10-[145-PF02]—[XL01](9.8 μL, 9.8 mg/mL in PBS) was incubated with azido-IL15 PF19 (4.49 μL, 7.2 mg/mL in PBS pH 7.5) overnight at room temperature. SDS-PAGE analysis confirmed the formation of the expected product Rit-v10-[145-PF02]—[XL01-PF19](see FIG. 26).

### Example 159. Conjugation of Arylazide-IL15Rα-IL15 PF13 to Rit-v10-[145-PF02]—[XL01] to Obtain Rit-v10-[145-PF02]—[XL01-PF13] with 2:1:1 Molecular Format (CDR:hOKT3: IL15Rα-IL15)

Rit-v10-[145-PF02]—[XL01](9.8 μL, 9.8 mg/mL in PBS) was incubated with arylazide-IL15Rα-IL15 PF13 (10.6 μL, 2.6 mg/mL in PBS pH 7.5) overnight at room temperature. SDS-PAGE analysis confirmed the formation of the expected product Rit-v10-[145-PF02]—[XL01-PF13](see FIG. 26).

### Example 160. Conjugation of Bis-Azido-MMAF LD10 to Trast-v1a-145 to Obtain Intermediate Trast-v1a-145-(LD10)₁ with 2:1 Molecular Format (CDR:MMAF) and Unreacted Azide

To a solution of trast-v1a-145 (150 μL, 1 mg, 6.7 mg/mL in PBS pH 7.4) was added bis-azido-MMAF (LD10, 50 μL, 1.33 mM solution in DMF, 10 eq compared to IgG). The reaction was incubated for 16 hours at room temperature followed by buffer exchange to PBS pH 7.4 using centrifugal filters (Amicon Ultra-0.5 mL MWCO 10 kDa, Merck Millipore) to a concentration of 20.7 mg/mL. Mass spectral analysis of the IdeS digested sample showed one major product (observed mass 51455 Da), corresponding to the conjugated ADC trast-v1a-145-(LD10)₁ obtained v1a intramolecular cross-linking.

### Example 161. Conjugation of BCN-IL15 PF33 to Trast-v1a-145-(LD10), to Obtain Trast-v1a-145-(LD10)₁—(PF33)₁ with 2:1:1 Molecular Format (CDR:MMAF:IL15)

Trast-v1a-145-(LD10)₁ (0.075 mg, 20.7 mg/mL in PBS) was incubated with BCN-IL15 (PF33) (3.69 μL, 8.2 mg/mL in PBS pH 7.5) overnight at room temperature. Mass spectral analysis of a sample after IdeS treatment showed one major Fc/2 product (observed mass 66472 Da) corresponding to the expected product trast-v1a-145-(LD10)₁—(PF33)₁.

### Example 162. Conjugation of BCN-IL15Rα-IL15 PF15 to Trast-v1a-145-(LD10), to Obtain Trast-v1a-145-(LD10)₁—(PF15)₁ with 2:1:1 Molecular Format (CDR:MMAF:IL15Rα-IL15)

Trast-v1a-145-(LD10)₁ (0.075 mg, 20.7 mg/mL in PBS) was incubated with BCN-IL15Rα-IL15 PF15 (7.47 μL, 6.7 mg/mL in PBS pH 7.5) overnight at room temperature. Mass spectral analysis of a sample after IdeS treatment showed one major Fc/2 product (observed mass 76494 Da) corresponding to the expected product trast-v1a-145-(LD10)-(PF15)₁.

### Example 163. Conjugation of hOKT3-BCN (201) to Trast-v1a-145-(LD10)₁ to Obtain Trast-v1a-145-(LD10)₁-(201), with 2:1:1 Molecular Format (CDR:MMAF:hOKT3)

Trast-v1a-145-(LD10)₁ (0.075 mg, 20.7 mg/mL in PBS) was incubated with hOKT3-BCN 201 (5.25 μL, 11 mg/mL in PBS pH 5.5) overnight at room temperature. Mass spectral analysis of a sample after IdeS treatment showed one major Fc/2 product (observed mass 79280 Da) corresponding to the expected product trast-v1a-145-(LD10)₁-(201)₁.

### Example 164. Conjugation of hOKT3-PEG11-Tetrazine PF01 to Trast-v9a-145 to Give Intermediate Trast-v9a-145-PF01 with 2:1 Molecular Format (CDR:hOKT3)

To a solution of trast-v9a-145 (127 μL, 1.2 mg, 7.88 mg/mL in PBS pH 7.4) was added hOKT3-PEG11-tetrazine PF01 (59 μL, 0.4 mg, 230 μM in PBS pH 6.5, 2 equiv. compared to IgG). The reaction was incubated overnight at rt. Mass spectral analysis of an IdeS-digested sample showed one major Fc/2 product (observed mass 78297 Da) corresponding to the crosslinked Fc/2 fragment of trast-v9a-145-PF01 with both Cys239 residues forming a disulfide with Cysteine. trast-v9a-145-PF01 was spinfiltered with PBS+10 mM EDTA using centrifugal filters (Amicon Ultra-0.5 mL MWCO 100 kDa, Merck Millipore) to remove residual hOKT3-PEG11-tetrazine PF01.

Intramolecular Cross-Linking of Trast-v9a-145-PF01 with Bis-Mal-MMAE LD09 to Obtain Trast-v9b-[145-PF01]-[LD09] with 2:1:1 Molecular Format (CDR:hOKT3;MMAE)

Trast-v9a-145-PF01 (0.85 mg, 17 mg/mL in PBS+10 mM EDTA) was incubated with TCEP (4.9 μL, 10 mM in MQ) for 2 hours at 37° C. The reduced antibody was spinfiltered with PBS+10 mM EDTA using centrifugal filters (Amicon Ultra-0.5 mL MWCO 10 kDa, Merck Millipore) and diluted to 50 μL. Subsequent DHA (4.9 μL, 10 mM in MQ:DMSO (9:1)) was added and the reaction was incubated for 3 hours at room temperature. To a part of the reaction mixture (0.17 mg, 10 μL) bis-mal-MMAE LD09 (1.5 μL, 2 mM in DMF, 3 eq) was added followed by incubation for 1.5 hours at room temperature. Mass spectral analysis of a sample after IdeS treatment showed one major Fc/2 product (observed mass 79857 Da) corresponding to the expected product trast-v9b-[145-PF01]-[LD09].

1. A multifunctional antibody construct containing at least one antibody Ab and two distinct payloads D¹ and D², wherein the construct has structure (1) or (2): 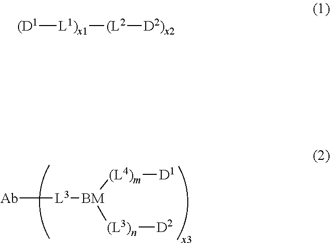
wherein:
Ab is an antibody;
L¹, L², L³, L⁴ and L⁵ are linkers;
x1 and x2 are each individually an integer in the range of 1-8, wherein x1+x2=2-10;
BM is a branching moiety;
m and n are each independently 0 or 1;
x3 is an integer in the range of 1-4;
D¹ and D² are two distinct payloads selected from the group consisting of polypeptides, small molecules, cytotoxins and oligonucleotides, wherein at least one of D¹ and D² is a polypeptide.

2. The multifunctional antibody construct according to claim 1, wherein the polypeptide is selected from the group consisting of Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides.

3. The multifunctional antibody construct according to claim 1, wherein the polypeptide is selected from an immune cell engager, a checkpoint inhibitor and a binder of a cell surface receptor, preferably wherein the polypeptide is an immune cell-engaging polypeptide or a checkpoint inhibiting polypeptide.

4. The multifunctional antibody construct according to claim 3, wherein the polypeptide is an immune cell-engaging polypeptide or a checkpoint inhibiting polypeptide, preferably wherein:
the immune cell-engaging polypeptide is specific for a cellular receptor on a T cell, preferably wherein the cellular receptor on a T cell is selected from the group consisting of CD3, CD28, CD137 (4-1BB), CD134, CD27, V119V112 and ICOS; or
the immune cell-engaging polypeptide is specific for a cellular receptor on an NK cell, preferably wherein the cellular receptor on a NK cell is selected from the group consisting of CD16, CD56, CD335, CD336, CD337, CD28, NKG2A, NKG2D, NKp46, KIR, DNAM-1 and CD161; or
the immune cell-engaging polypeptide is specific for a cellular receptor on a monocyte or a macrophage, preferably wherein the cellular receptor on the monocyte or macrophage is CD64; or
the immune cell-engaging polypeptide is specific for a cellular receptor on a granulocyte, preferably wherein the cellular receptor on the granulocyte is CD89;
the immune cell-engaging polypeptide is specific for IL2 or IL15; or
the checkpoint inhibiting polypeptide is specific for CTLA-4, PD-1, PD-L1, TIGIT, TIM-3, LAG-3 or VISTA.

5. The multifunctional antibody construct according to claim 1, wherein the polypeptide is selected from the group consisting of OKT3, L²K, UCHT1, BMA031, VHH 6H4, IL2, IL15, IL15/IL15R complex, IL15/IL15R fusion, an antibody specific for IL2 and an antibody specific for IL15, preferably wherein the polypeptide is OKT3, L²K, IL15/IL15R fusion, IL15, mAb602, Nara1 or TCB2.

6. The multifunctional antibody construct according to claim 1, wherein L¹ and/or L², or L³ has the structure (L-A): 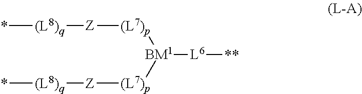
wherein:
the bonds labelled * are connected to two distinct amino acids of the antibody Ab, and the bond labelled ** is connected to D¹; D² or BM;
L⁶, L⁷ and L⁸ are linkers;
p and q are each individually 1 or 0;
BM¹ is a branching moiety;
Z are connecting groups.

7. The multifunctional antibody construct according to claim 1, wherein each linker is individually selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups and combinations thereof, wherein the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted and may be interrupted by one or more heteroatoms, preferably wherein the heteroatoms are selected from the group consisting of O, S(O)_{y} and NR¹², wherein y is 0, 1 or 2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups.

8. The multifunctional antibody construct according to claim 1, wherein L¹ and/or L² or L⁴ and/or L⁵ is cleavable in the tumour lysosome or the tumour microenvironment, wherein the cleavable linker preferably contains a peptide spacer of structure (26): 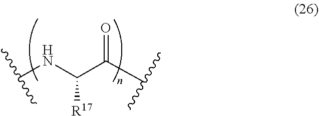
wherein R¹⁷ represents an amino acid side chain and n is an integer in the range of 1-10, preferably wherein the linker is cleavable by a proteolytic enzyme present selected from the group of FAP, cathepsins, granzymes, caspases, kallikereins, proprotein convertase subtilisins, furins, elastases, legumains, fibroblast activation proteins, tissue-type plasminogen activators, urokinases, matrix metallo proteases and matriptases, and/or wherein the peptide spacer is selected from Val-Cit, Val-Ala, Val-Lys, Val-Arg, Val-Gln, AcLys-Val-Cit, AcLys-Val-Ala, Phe-Cit, Phe-Ala, Phe-Lys, Phe-Arg, Phe-Gln, Ala-Lys, Leu-Cit, Leu-Gln, Ile-Cit, Trp-Cit, Ala-Ala-Asn, Ala-Asn, Pro-Leu-Gly, Asn-Pro-Val, Lys-Ser-Gly-Arg-Ser-Asp-Asn-His, Pro-Val-Gly-Leu-Ile-Gly, Val-Lys-Gly, Gly-Gly-Gly, Gly-Gly-Phe-Gly and Lys.

9. The multifunctional antibody construct according to claim 6, wherein each of L⁴, L⁵, L⁶ and L⁷ has the structure -(L²¹)ₙ-(L²²)ₒ-(L²³)ₚ-(L²⁴)_{q}-, wherein L²¹, L²², L²³ and L²⁴ are linkers that together form linker L⁴, L⁵, L⁶ and L⁷; n, o, p and q are individually 0 or 1, wherein:
(a) linker L²¹ is represented by —(W)ₖ₁-(A)_{d1}-(B)ₑ₁-(A)ₙ-(B)_{g1}—C(O)—, wherein:
d1=0 or 1;
e1=an integer in the range 1-10;
f1=0, or 1;
g1=an integer in the range 0-10;
k1=0 or 1 with the proviso that if k1=1 then d1=0;
A is a sulfamide group according to structure (23) 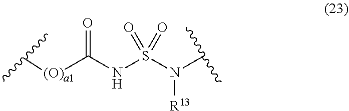
wherein a1=0 or 1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is D connected to N, possibly via a spacer moiety;
B is a —CH₂—CH₂—O— or a —O—CH₂—CH₂— moiety, or (B)ₑ₁ is a —(CH₂—CH₂—O)ₑ₃—CH₂—CH₂— moiety, wherein e3 is defined the same way as e1;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—, wherein m is an integer in the range 0-10;
(b) linker L²² is a peptide spacer, preferably a dipeptide wherein L²² is represented by general structure (27): 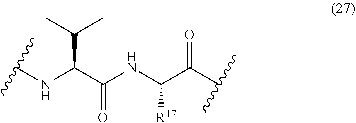
wherein R¹⁷=CH₃ or CH₂CH₂CH₂NHC(O)NH₂;
(c) linker L²³ is a self-immolative spacer, preferably a para-aminobenzyloxy (PAB) derivative according to structure (25), 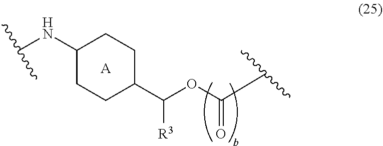
wherein:
A is an optionally substituted 5- or 6-membered aromatic or heteroaromatic ring;
b is 0 or 1;
R³ is H, R⁴ or C(O)R⁴, wherein R⁴ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR⁵ wherein R⁵ is independently selected from the group consisting of hydrogen and C₁-C₄ alkyl groups,
preferably wherein R³ is H or C(O)R⁴, wherein R⁴=4-methyl-piperazine or morpholine, most preferably wherein R³ is H; and
(d) linker L²⁴ is an aminoalkanoic acid spacer according to the structure —N—(Cₓ-alkylene)-C(O)—, wherein x is an integer in the range 1-10; or
linker L²⁴ is a an ethyleneglycol spacer according to the structure —N—(CH₂—CH₂—O)ₑ₆—(CH₂)ₑ₇—C(O)—, wherein e6 is an integer in the range 1-10 and e7 is an integer in the range 1-3.

10. The multifunctional antibody construct according to claim 1, wherein linkers L¹, L² and L³ contain a connecting group Z that is obtainable by a conjugation reaction, preferably wherein individually obtainable by a conjugation reaction, preferably a conjugation reaction selected from nucleophilic reaction and a cycloaddition, preferably wherein the cycloaddition is a [4+2]cycloaddition or a 1,3-dipolar cycloaddition or the nucleophilic reaction is a Michael addition or a nucleophilic substitution.

11. The multifunctional antibody construct according to claim 10, wherein each connecting group contains a triazole, a cyclohexene, a cyclohexadiene, a [2.2.2]-bicyclooctadiene, a [2.2.2]-bicyclooctene, an isoxazoline, an isoxazolidine, a pyrazoline, a piperazine, a thioether, a succinimidyl ring or a ring-opened succinic acid amide, an amide or an imide group.

12. The multifunctional antibody construct according to claim 1, wherein at least one of L¹ and L², preferably both, has the structure (L-B) or (L-C):
*-GlcNAc(Fuc)_{d}-(G)ₑ-Su-Z-(L²¹)ₙ-(L²²)ₒ-(L²³)ₚ-(L²⁴)_{q}-** (L-B)
*—Z-(L²¹)ₙ-(L²²)ₒ-(L²³)ₚ(L²⁴)_{q}-** (L-C)
wherein:
the bonds labelled * are connected to two distinct amino acids of the antibody Ab, and the bond labelled ** is connected to D¹ or D²;
d, n, o, p and q are each independently 0 or 1;
e is an integer in the range of 0-10;
Su is a monosaccharide;
G is a monosaccharide moiety;
GlcNAc is an N-acetylglucosamine moiety;
Fuc is a fucose moiety;
Z is a connecting group,
(a) linker L²¹ is represented by —(W)ₖ₁-(A)_{d1}-(B)ₑ₁-(A)_{f1}-(B)_{g1}—C(O)—, wherein:
d1=0 or 1;
e1=an integer in the range 1-10;
f1=0, or 1:
g1=an integer in the range 0-10;
k1=0 or 1 with the proviso that if k1=1 then d1=0;
A is a sulfamide group according to structure (23) 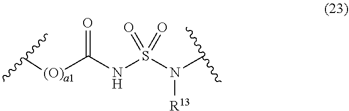
wherein a1=0 or 1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is D connected to N, possibly via a spacer moiety;
B is a —CH₂—CH₂—O— or a —O—CH₂—CH₂— moiety, or (B)ₑ₁ is a —(CH₂—CH₂—O)ₑ₃—CH₂—CH₂— moiety, wherein e3 is defined the same way as e1;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—, wherein m is an integer in the range 0-10;
(b) linker L²² is a peptide spacer, preferably a dipeptide wherein L²² is represented by general structure (27): 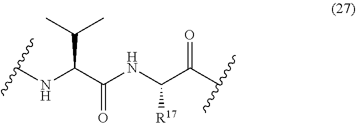
wherein R¹⁷=CH₃ or CH₂CH₂CH₂NHC(O)NH₂;
(c) linker L²³ is a self-immolative spacer, preferably a para-aminobenzyloxy (PAB) derivative according to structure (25), 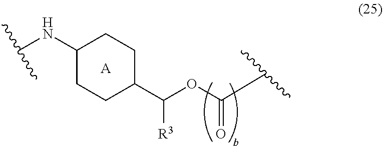
wherein:
A is an optionally substituted 5- or 6-membered aromatic or heteroaromatic ring;
b is 0 or 1:
R³ is H, R⁴ or C(O)R⁴, wherein R⁴ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR⁵ wherein R⁵ is independently selected from the group consisting of hydrogen and C₁-C₄ alkyl groups,
preferably wherein R³ is H or C(O)R⁴, wherein R⁴=4-methyl-piperazine or morpholine, most preferably wherein R³ is H; and
(d) linker L²⁴ is an aminoalkanoic acid spacer according to the structure —N—(Cₓ-alkylene)-C(O)—, wherein x is an integer in the range 1-10; or
linker L²⁴ is a an ethyleneglycol spacer according to the structure —N—(CH₂—CH₂—O)ₑ₆—(CH₂)ₑ₇—C(O)—, wherein e6 is an integer in the range 1-10 and e7 is an integer in the range 1-3.

13. The multifunctional antibody construct according to claim 1, wherein L⁴ and L⁵ have the structure (L-C)
**-(L²¹)ₙ-(L²²)ₒ-(L²³)ₚ-(L²⁴)_{q}-*** (L-D)
wherein
the bond labelled ** is connected to BM, and the bond labelled *** is connected to D¹ or D²;
n, o, p and q are each independently 0 or 1;
wherein the linker L⁴ and L⁵ may further contain a connecting group Z at the junction any of the linking units L²¹, L²², L²³ and L²⁴;
(a) linker L²¹ is represented by —(W)ₖ₁-(A)_{d1}-(B)ₑ₁-(A)_{f1}-(B)_{g1}—C(O)—, wherein:
d1=0 or 1;
e1=an integer in the range 1-10;
f1=0, or 1:
g1=an integer in the range 0-10;
k1=0 or 1 with the proviso that if k1=1 then d1=0;
A is a sulfamide group according to structure (23) 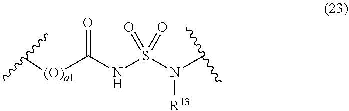
wherein a1=0 or 1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is D connected to N, possibly via a spacer moiety;
B is a —CH₂—CH₂—O— or a —O—CH₂—CH₂— moiety, or (B)ₑ₁ is a —(CH₂—CH₂—O)ₑ₃—CH₂—CH₂— moiety, wherein e3 is defined the same way as e1;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—,
wherein m is an integer in the range 0-10;
(b) linker L²² is a peptide spacer, preferably a dipeptide wherein L²² is represented by general structure (27): 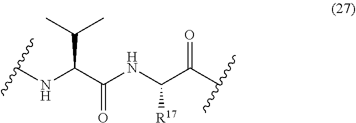
wherein R¹⁷=CH₃ or CH₂CH₂CH₂NHC(O)NH₂;
(c) linker L²³ is a self-immolative spacer, preferably a para-aminobenzyloxy (PAB) derivative according to structure (25), 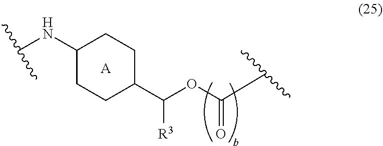
wherein:
A is an optionally substituted 5- or 6-membered aromatic or heteroaromatic ring;
b is 0 or 1:
R³ is H, R⁴ or C(O)R⁴, wherein R⁴ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR⁵ wherein R⁵ is independently selected from the group consisting of hydrogen and C₁-C₄ alkyl groups,
preferably wherein R³ is H or C(O)R⁴, wherein R⁴=4-methyl-piperazine or morpholine, most preferably wherein R³ is H; and
(d) linker L²⁴ is an aminoalkanoic acid spacer according to the structure —N—(Cₓ-alkylene)-C(O)—, wherein x is an integer in the range 1-10; or
linker L²⁴ is a an ethyleneglycol spacer according to the structure —N—(CH₂—CH₂—O)ₑ₆—(CH₂)ₑ₇—C(O)—, wherein e6 is an integer in the range 1-10 and e7 is an integer in the range 1-3.

14. The multifunctional antibody construct according to claim 1, which has structure (3): 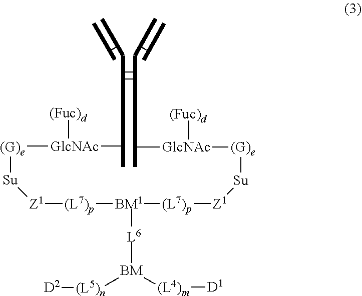
wherein:
d and p are each independently 0 or 1;
e is an integer in the range of 0-10;
L⁶ and L⁷ are linkers;
BM¹ is a branching moiety;
Su is a monosaccharide;
G is a monosaccharide moiety;
GlcNAc is an N-acetylglucosamine moiety;
Fuc is a fucose moiety;
Z¹ is a connecting groups.

15. The multifunctional antibody construct according to claim 1, which has structure (4): 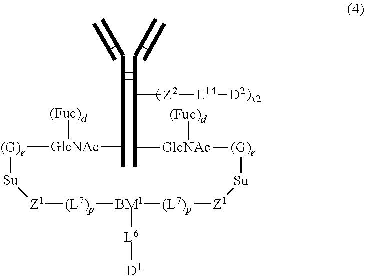
wherein:
d and p are each independently 0 or 1;
e is an integer in the range of 0-10;
L⁶, L⁷ and L¹⁴ are linkers;
BM¹ is a branching moiety;
Su is a monosaccharide;
G is a monosaccharide moiety;
GlcNAc is an N-acetylglucosamine moiety;
Fuc is a fucose moiety;
Z¹ and Z² are connecting groups.

16. The multifunctional antibody construct according to claim 1, which has structure (5): 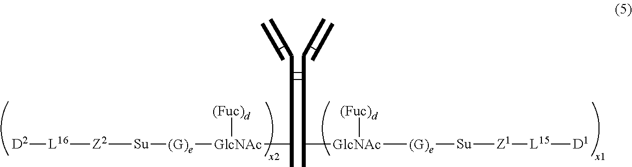
wherein:
each d is independently 0 or 1;
e is an integer in the range of 0-10;
L¹⁵ and L¹⁶ are linkers;
Su is a monosaccharide;
G is a monosaccharide moiety;
GlcNAc is an N-acetylglucosamine moiety;
Fuc is a fucose moiety;
Z¹ and Z² are connecting groups; and
x1 and x2 are each independently 1, 2 or 3.

17. The multifunctional antibody construct according to claim 1, which has structure (6): 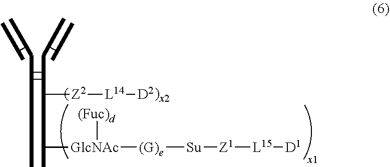
wherein:
each d is independently 0 or 1;
e is an integer in the range of 0-10;
L¹⁴ and L¹⁵ are linkers;
Su is a monosaccharide;
G is a monosaccharide moiety;
GlcNAc is an N-acetylglucosamine moiety;
Fuc is a fucose moiety;
Z¹ and Z² are connecting groups;
x1 is an integer in the range 1-4.

18. The multifunctional antibody construct according to claim 1, which has structure (7): 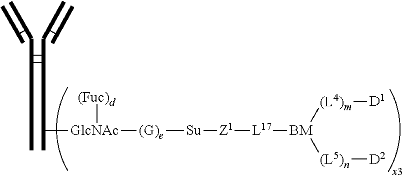
wherein:
each d is independently 0 or 1;
e is an integer in the range of 0-10;
L¹⁷ is a linker;
BM is a branching moiety;
Su is a monosaccharide;
G is a monosaccharide moiety;
GlcNAc is an N-acetylglucosamine moiety;
Fuc is a fucose moiety;
Z is a connecting group.

19. The multifunctional antibody construct according to claim 1, wherein:
(i) D¹ is a CD3-targeting polypeptide and D² is a CD28-targeting polypeptide;
(ii) D¹ is IL15 or an IL15-targeting polypeptide and D² is a CD16-targeting polypeptide;
(iii) D¹ is IL2 or an IL2-targeting polypeptide and D² is a CD16-targeting polypeptide;
(iv) D¹ is an NKp46-targeting polypeptide and D² is a CD16-targeting polypeptide;
(v) D¹ is a cytotoxin and D² is a checkpoint inhibitor, preferably selected from polypeptides targeting CTLA-4, TIGIT, LAG-3, TIM-3, VISTA, PD-1 or PD-L1;
(vi) D¹ is an OX40-targeting polypeptide and D² is a CD137-targeting polypeptide;
(vii) D¹ is a PD-L1-targeting polypeptide and D² is a CD137-targeting polypeptide;
(viii) D¹ is a cytotoxin and D² is IL15 or an IL15-targeting polypeptide;
(ix) D¹ is a cytotoxin and D² is IL2 or an IL2-targeting polypeptide;
(x) D¹ is a cytotoxin and D² is a CD16-targeting polypeptide; or
(xi) D¹ is a TLR7-agonist or a TRL8-agonist and D² is a CD16-targeting polypeptide.

20. The multifunctional antibody construct according to claim 1, wherein the antibody Ab is specific for an extracellular receptor on a tumour cell, preferably wherein the extracellular receptor on the tumour cell is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Ra, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, β-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin.

21. The multifunctional antibody construct according to claim 1 for use in medicine.

22. The multifunctional antibody construct according to claim 1 for use in the treatment of cancer, a viral infection, a bacterial infection, a neurological disease, an autoimmune disease, an eye disease, hypercholesterolemia and amyloidosis, preferably in the treatment of cancer.

23. A pharmaceutical composition comprising the multifunctional antibody construct according to claim 1 and a pharmaceutically acceptable carrier.
